(19) United States
(12) Patent Application Publication
(10) Pub. No.: US 2026/0306496 A1
(43) Pub. Date: Oct. 8, 2026

(54) CDK2 INHIBITOR PYRAZOLOPYRIMIDINE COMPOUNDS

(71) Applicant: ALEKSIA THERAPEUTICS, INC., Golden, CO (US)

(72) Inventors: Amy HART, Littleton, CO (US); Claude QUESNELLE, Denver, CO (US); Kiel LAZARSKI, Waltham, MA (US); Andrew PHILLIPS, Littleton, CO (US); Rachelle QUACH, Somerville, MA (US); Santosh S. Kulkarni, Bangalore (IN); Swapan Kumar Samanta, Hyderabad (IN)

(21) Appl. No.: 19/483,940

(22) PCT Filed: May 14, 2024
(86) PCT No.: PCT/US2024/029318

§ 371 Date: Nov. 12, 2025

Related U.S. Application Data
(60) Provisional application No. 63/502,379, filed on May 15, 2023.

(51) Int. Cl. A61K 31/519; C07D 487/04

(52) U.S. Cl. CPC A61K 31/519; C07D 487/04

(57) ABSTRACT

CDK2 inhibitor compounds having a core structure including a fused 5-6 heteroaryl ring system where the 6-membered ring of the system is a pyridine or pyrimidine, and the 5-membered ring contains at least one nitrogen atom, such as a pyrrole, imidazole or pyrazole ring are provided. The compounds can have a pyrazolopyrimidine core structure having a 6-amino group that is further substituted. The 6-amino group can be linked to a cyclic group having an appended hydrophilic group or substituent. Also provided herein are compositions including the CDK2 inhibitor compounds, and methods for using compounds in research and as therapeutics.

### 1. CROSS-REFERENCE TO RELATED APPLICATION

This application claims benefit of U.S. Provisional Application No. 63/502,379 filed on May 15, 2023, which is herein incorporated by reference in its entirety.

### 2. INTRODUCTION

Cyclin-dependent kinases (CDKs) are a family of serine/threonine kinases. Heterodimerized with regulatory subunits known as cyclins, CDKs become fully activated and regulate key cellular processes including cell cycle progression and cell division. Uncontrolled proliferation is a hallmark of cancer cells. The deregulation of the CDK activity is associated with abnormal regulation of cell-cycle, and is detected in virtually all forms of human cancers.

CDK2 is a cyclin-dependent kinase that plays a critical role in regulating the transition from the G1 to S phase of the cell cycle. It is involved in several other cellular processes, including DNA replication and repair, transcription, RNA processing, and cytoskeletal organization, making it an attractive target for cancer therapy. CDK2 is of particular interest because deregulation of CDK2 activity occurs frequently in a variety of human cancers. Although CDK2 is mostly dispensable in the cell cycle of normally functioning cells, it is critical to the abnormal growth processes of cancer cells. The CCNE1 gene produces cyclin E, one of the two major protein binding partners of CDK2. Overexpression of CCNE1 occurs in many tumor cells, causing the cells to become dependent on CDK2 and cyclin E. Abnormal cyclin E activity is also observed in breast, lung, colorectal, gastric, and bone cancers, as well as in leukemia and lymphoma. Likewise, abnormal expression of cyclin A2 is associated with chromosomal instability and tumor proliferation, while inhibition leads to decreased tumor growth.

Therefore, CDK2 and its cyclin binding partners represent possible therapeutic targets for new cancer therapeutics. CDK2 inhibitors are a class of small molecule drugs that block the activity of CDK2, leading to cell cycle arrest and potentially inducing cell death. However, identifying selective CDK2 inhibitors is difficult due to the extreme similarity between the active sites of CDK2 and other CDKs, for example CDK1. CDK1 is the only essential cyclin dependent kinase in the cell cycle, and its inhibition could lead to unintended side effects.

### 3. SUMMARY

Provided herein are CDK inhibitor compounds having a core structure including a fused 5-6 heteroaryl or partially unsaturated heterocycle ring system where the 6-membered ring of the system can be a pyridine or pyrimidine, and the 5-membered ring contains at least one heteroatom (e.g., nitrogen atom), such as a pyrrole, furan, thiophene, imidazole, pyrazole, or triazole ring. In some embodiments, the compounds have a pyrazolopyrimidine core structure having a 6-amino group that is further substituted. The 6-amino group can be linked to a cyclic group having an appended hydrophilic group or substituent. The CDK inhibitor compounds can be CDK2 inhibitors. Also provided herein are pharmaceutical compositions including the subject CDK (e.g., CDK2) inhibitor compounds. Also provided are methods for using compounds and compositions of the disclosure in research and as therapeutics.

### 4. DETAILED DESCRIPTION

The CDK inhibitor compounds, compositions and methods of this disclosure are described in greater detail below. A particular class of CDK2 inhibitor compounds is described. In some embodiments, compounds of this disclosure have inhibition activity at one or more other CDK kinase such as CDK4. Also described are pharmaceutical compositions that include the subject CDK (e.g., CDK2) inhibitor compounds. Methods in which the CDK2 inhibitor compounds and compositions find use are also described.

### 3.1 CDK2 Inhibitor Compounds

As summarized above, this disclosure provides CDK (e.g., CDK2 or CDK4) inhibitor compounds having a core structure including a fused 5-6 heteroaryl or partially unsaturated heterocycle ring system where the 6-membered ring of the system is a pyridine or pyrimidine, and the 5-membered ring contains at least one heteroatom (e.g., nitrogen atom), such as a pyrrole, furan, thiophene, imidazole, pyrazole, or triazole ring. The 6-membered pyridine or pyrimidine ring is substituted with an amino group, e.g., at the position beta to the fused ring, wherein the amino group is linked to a cyclic group via an optional linker, and the cyclic group can be further substituted.

The cyclic group (e.g., A, as described herein) can be a 6-membered cycloalkyl, heterocyclyl, phenyl or heteroaryl. The cyclic group can have one or more appended substituent(s) including an optional hydrophilic group attached via an optional linker. In some embodiments, the hydrophilic group is a non-electrophilic group. In some embodiments the CDK2 inhibitor is a classic inhibitor, where the inhibitor compound binds to a CDK2 receptor and prevents other molecules from binding to the receptor. Classic inhibitors may not permanently bind to the receptor, i.e., they associate to the receptor and dissociate from the receptor reversibly, for example via hydrogen bonding or Van der Waals interactions.

In some embodiments, the hydrophilic group is an electrophilic reactive moiety. In general, an electrophilic reactive moiety is a chemical group that is capable of covalently conjugating with a biological target. In some embodiments, the CDK2 inhibitor that includes an electrophilic reactive moiety is referred to as a covalent inhibitor, where the covalent inhibitor can bind to the CDK2 receptor target at a desired binding site, and form a covalent, chemical bond to the receptor. In some embodiments, the covalent, chemical bond formed is not reversible. In some embodiments, the CDK2 inhibitor is a covalent reversible inhibitor. Such inhibitors can form a covalent bond with the receptor, but this chemical conjugation reaction is reversible.

Accordingly, this disclosure provides CDK (e.g., CDK2 or CDK4) inhibitor compounds of Formula α:

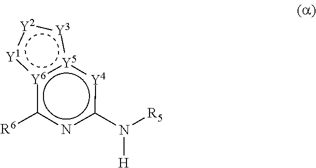

or a pharmaceutically acceptable salt, solvate or prodrug thereof, wherein:
Y¹ is N, NR¹, O, S, or CR¹;
Y² is N, O, S, NR², NR³, or CR²; or
Y¹ [symbol] Y² is an optionally substituted, fused 5-membered or 6-membered ring B:

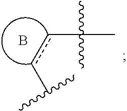

Y³ is NR⁹, CR³, or CR³R^{3′};
Y⁴ is N or CR⁴;
Y⁵ is N or C;
Y⁶ is N or C;
R¹ is selected from H, (C₁₋₅)alkyl, substituted (C₁₋₅)alkyl, (C₁₋₅)alkoxyl, substituted (C₁₋₅)alkoxyl, haloalkyl, halogen, OH, and CN;
R² is selected from H, (C₁₋₅)alkyl, substituted (C₁₋₅)alkyl, (C₁₋₅)alkoxyl, substituted (C₁₋₅)alkoxyl, haloalkyl, halogen, OH, and CN;
R³ is selected from H, alkyl, carbocycle, heterocycle, aryl, heteroaryl, alkylene-carbocycle, alkylene-heterocycle, alkylene-aryl, alkylene-heteroaryl, and substituted versions thereof; and
R^{3′} is H, or (C₁₋₅)alkyl;
R⁴ is selected from H, (C₁₋₅)alkyl, substituted (C₁₋₅)alkyl, (C₁₋₅)alkoxyl, substituted (C₁₋₅)alkoxyl, haloalkyl, halogen, OH, and CN;
R⁵ is

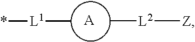

wherein:
   A is a cyclic group selected from carbocyclyl, heterocyclyl, aryl, heteroaryl, and substituted versions thereof;
   Z is an optional hydrophilic group;
   L¹ and L² are optional linkers; and
      * represents the point of attachment of R⁵ to the nitrogen atom; and
R⁶ is selected from H, (C₁₋₅)alkyl, substituted (C₁₋₅)alkyl, (C₁₋₅)alkoxyl, substituted (C₁₋₅)alkoxyl, haloalkyl, halogen, OH, and CN.

In some embodiments of Formula α, the CDK inhibitor compounds are of Formula β:

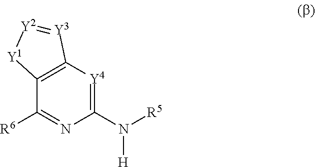

or a pharmaceutically acceptable salt, solvate or prodrug thereof, wherein:
Y¹ is N or C(R¹)₂;
Y² is N or CR²; or
Y¹-Y² is an optionally substituted, fused 5-membered or 6-membered ring B:

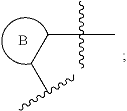

Y³ is CR³;
Y⁴ is N or CR⁴;
R¹, R², R³, R^{3′}, R⁴, R⁵, and R⁶ are as defined herein.

In some embodiments of Formula α, the CDK inhibitor compounds are of Formula γ:

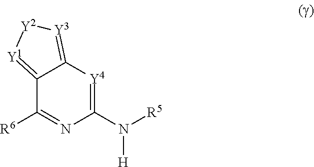

or a pharmaceutically acceptable salt, solvate or prodrug thereof, wherein:
Y¹ is N or CR¹;
Y² is NR², NR³, O, S, or CR³R^{3′}; or
═Y¹-Y²— is an optionally substituted, fused 5-membered or 6-membered ring B:

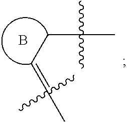

Y³ is N or CR³;
Y⁴ is N or CR⁴;
R¹, R², R³, R^{3′}, R⁴, R⁵, and R⁶ are as defined herein.

In some embodiments of Formula α, the CDK inhibitor compounds are of Formula δ:

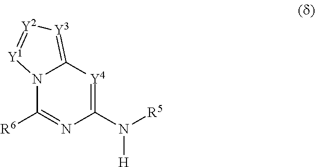

or a pharmaceutically acceptable salt, solvate or prodrug thereof, wherein:
Y¹ is N or CR¹;
Y² is N or CR²; or
—Y¹═Y²— is an optionally substituted, fused 5-membered or 6-membered ring B:

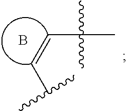

Y³ is N or CR³;
Y⁴ is N or CR⁴;
R¹, R², R³, R^{3′}, R⁴, R⁵, and R⁶ are as defined herein.

In some embodiments of Formula α, the CDK inhibitor compounds are of Formula F:

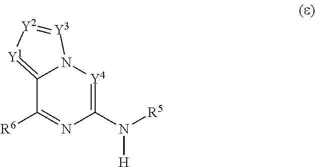

or a pharmaceutically acceptable salt, solvate or prodrug thereof, wherein:
Y¹ is N or CR¹;
Y² is N or CR²; or
═Y¹-Y²— is an optionally substituted, fused 5-membered or 6-membered ring B:

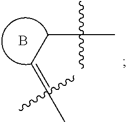

Y³ is N or CR³;
Y⁴ is N or CR⁴;
R¹, R², R³, R^{3′}, R⁴, R⁵, and R⁶ are as defined herein.

In some embodiments of Formula α, the CDK inhibitor compounds are of Formula I:

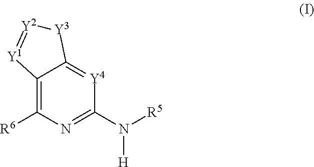

or a pharmaceutically acceptable salt, solvate or prodrug thereof, wherein:
Y¹ is N or CR¹, and
Y² is N or CR², or
—Y¹═Y²— is an optionally substituted, fused 5-membered or 6-membered ring B:

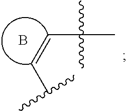

Y³ is NR³ or CR³R^{3′};
Y⁴ is N or CR⁴;
R¹ is selected from H, (C₁₋₅)alkyl, substituted (C₁₋₅)alkyl, (C₁₋₅)alkoxyl, substituted (C₁₋₅)alkoxyl, haloalkyl, halogen, OH, and CN;
R² is selected from H, (C₁₋₅)alkyl, substituted (C₁₋₅)alkyl, (C₁₋₅)alkoxyl, substituted (C₁₋₅)alkoxyl, haloalkyl, halogen, OH, and CN;
R³ is selected from H, alkyl, carbocycle, heterocycle, aryl, heteroaryl, alkylene-carbocycle, alkylene-heterocycle, alkylene-aryl, alkylene-heteroaryl, and substituted versions thereof; and
R^{3′} is H, or (C₁₋₅)alkyl;
R⁴ is selected from H, (C₁₋₅)alkyl, substituted (C₁₋₅)alkyl, (C₁₋₅)alkoxyl, substituted (C₁₋₅)alkoxyl, haloalkyl, halogen, OH, and CN;
R⁵ is

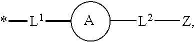

wherein:
   A is a cyclic group selected from carbocyclyl, heterocyclyl, aryl, heteroaryl, and substituted versions thereof;
   Z is an optional hydrophilic group;
   L¹ and L² are optional linkers; and
      * represents the point of attachment of R⁵ to the nitrogen atom; and
R⁶ is selected from H, (C₁₋₅)alkyl, substituted (C₁₋₅)alkyl, (C₁₋₅)alkoxyl, substituted (C₁₋₅)alkoxyl, haloalkyl, halogen, OH, and CN.

In some embodiments of Formula I, one or more of Y¹-Y³ is N or NR³. In some embodiments, Y³ is NR³. In some embodiments, Y² is N. In some embodiments Y¹ is N. In some embodiments of Formula I, two or three of Y¹-Y³ is N or NR³. In some embodiments Y¹ is N, and Y³ is NR³. In some embodiments Y² is N, and Y³ is NR³. In some embodiments Y¹ and Y² are each N.

In some embodiments of Formula I, two or more of Y¹-Y³ are CR¹, CR², and/or CR³R^{3′} respectively. In some embodiments of Formula I, Y¹-Y³ are CR¹, CR², and CR³R^{3′} respectively. In some embodiments, each of R¹-R^{3′} are H. In some embodiments, at least one of R¹-R^{3′} is not H. In some embodiments, Y³ is CHR³, where R³ is not H.

In some embodiments of Formula I, R¹ and R² are independently selected from H, (C₁₋₅)alkyl, substituted (C₁₋₅)alkyl, haloalkyl, halogen, and CN. In some embodiments of Formula I, R⁶ is selected from H, (C₁₋₅)alkyl, substituted (C₁₋₅)alkyl, haloalkyl, halogen, and CN. In some embodiments of Formula I, R⁴ is selected from H, (C₁₋₅)alkyl, substituted (C₁₋₅)alkyl, haloalkyl, halogen, and CN.

In some embodiments of Formula I, R³ is selected from aryl, heteroaryl, alkylene-aryl, alkylene-heteroaryl, and substituted versions thereof. In some embodiments of Formula (I), R³ is selected from phenyl, pyridyl, pyrimidinyl, alkylene-phenyl, alkylene-pyridyl, alkylene-pyrimidinyl, and substituted versions thereof. In some embodiments, R³ is optionally substituted cycloalkyl, optionally substituted aryl, optionally substituted phenyl, optionally substituted cycloalkenyl, optionally substituted alkylene-cycloalkyl, or optionally substituted alkylene-cycloalkenyl. In some embodiments, R³ is optionally substituted heterocyclyl or optionally substituted alkylene-heterocyclyl. In some embodiments R³ is optionally substituted cycloalkyl. In some embodiments R³ is optionally substituted cyclohexyl. In some embodiments R³ is optionally substituted cyclopentyl. In some embodiments R³ is optionally substituted cyclobutyl.

In some embodiments of Formula I, Y³ is N, Y¹ is CR¹, and Y² is CR². In some embodiments of Formula I, Y³ is N, Y¹ is N, and Y² is CR². In some embodiments of Formula I, Y³ is N, Y¹ is CR¹, and Y² is N.

In some embodiments of Formula I, Y³ is CR³R^{3′}. In some embodiments, Y³ is CR³R^{3′}, Y¹ is CR¹, and Y² is N. In some embodiments, Y³ is CR³R^{3′}, Y¹ is N, and Y² is CR². In some embodiments, Y³ is CR³R^{3′}, Y¹ is N, and Y² is N. In some embodiments, Y³ is CR³R^{3′}, Y¹ is CR¹, and Y² is CR².

In some embodiments of Formula I, —Y¹═Y²— is an optionally substituted, fused 5-membered or 6-membered ring B:

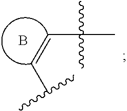

such that the compound is of formula B1:

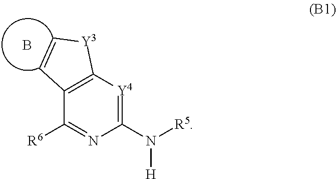

In some embodiments of Formula B1, B is a fused 5-membered or 6-membered ring that is a cycloalkenyl, aryl, heterocycle, heteroaryl, or a substituted version thereof. In some embodiments of Formula B1, B is a fused pyridine. In some embodiments of Formula B1, B is a fused benzo ring. In some embodiments of Formula B1, B is a fused pyrrole, imidazole or pyrazole ring.

In some embodiments of Formula I, Y⁴ is CR⁴. In some embodiments of Formula I, R⁴ is H. In some embodiments of Formula I, R⁴ is (C₁₋₅)alkyl, or substituted (C₁₋₅)alkyl. In some embodiments of Formula I, R⁴ is haloalkyl. In some embodiments of Formula I, R⁴ is halogen, or CN.

In some embodiments of Formula I, Y⁴ is N.

In some embodiments of Formulae α, β, γ, δ, ε, or I, the compound is of one of Formulae IA to IU:

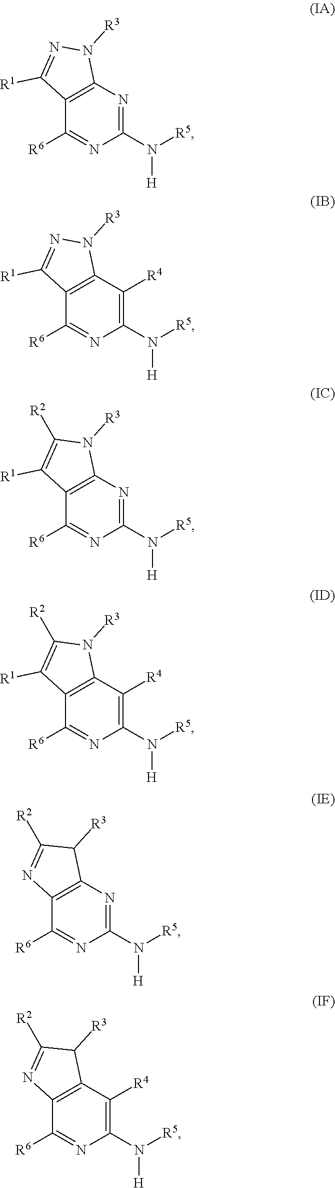

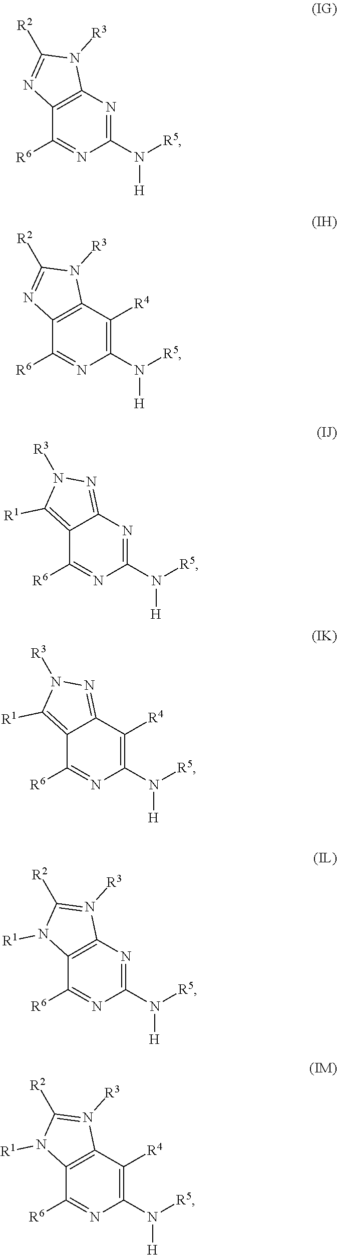

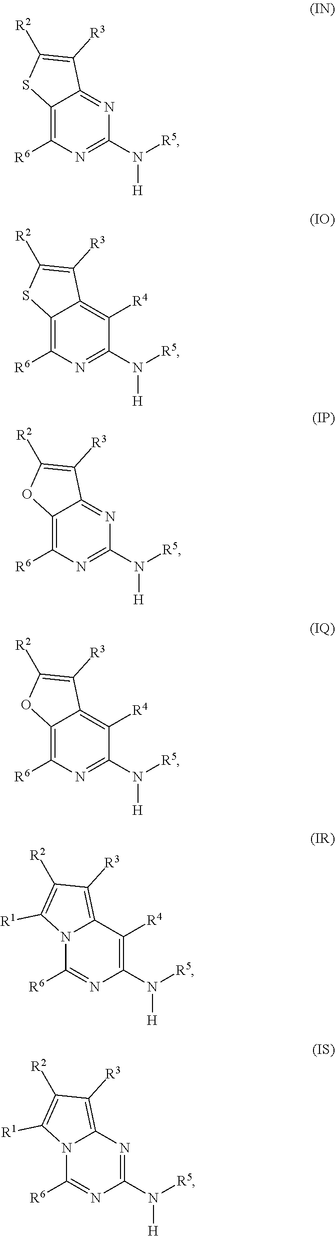

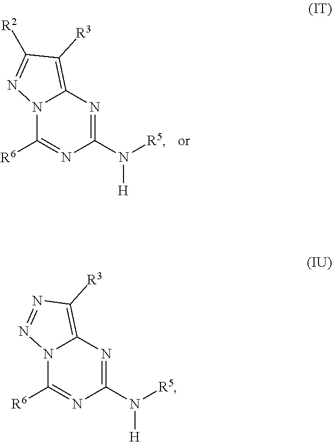

where R¹, R², R⁴, R⁵, and R⁶ are as defined herein.

In some embodiments of Formula I, the compound is of Formula IJ′, or Formula IK′:

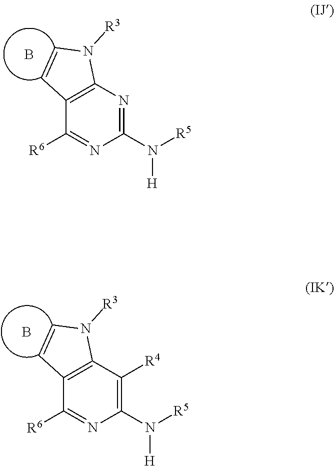

wherein the 5-membered or 6-membered fused ring B is selected from cycloalkenyl, aryl, heterocycle, heteroaryl, and substituted versions thereof. In some embodiments of Formula (IK′), R⁴ is H.

In some embodiments of Formula I, the compound is of Formula IA:

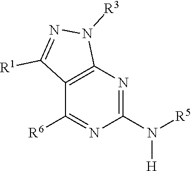

where R¹, R³, R⁵ and R⁶ are as defined herein.

In some embodiments of Formula I, the compound is of Formula IB:

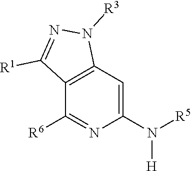

where R¹, R³, R⁵ and R⁶ are as defined herein, and R⁴ is H.

In some embodiments of Formula I, the compound is of Formula IC:

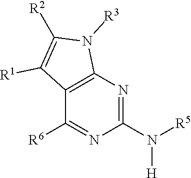

where R¹-R³, R⁵ and R⁶ are as defined herein.

In some embodiments of Formula I, the compound is of Formula ID:

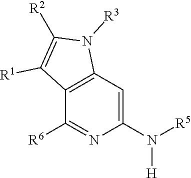

where R¹-R³, R⁵ and R⁶ are as defined herein, and R⁴ is H.

In some embodiments of Formula I, the compound is of Formula IE:

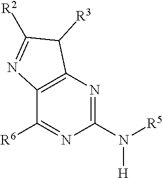

where R², R³, R⁵ and R⁶ are as defined herein.

In some embodiments of Formula I, the compound is of Formula IF:

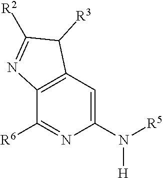

where R², R³, R⁵ and R⁶ are as defined herein, and R⁴ is H.

In some embodiments of Formula I, the compound is of Formula IG:

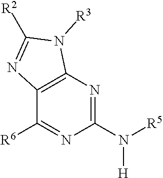

where R², R³, R⁵ and R⁶ are as defined herein.

In some embodiments of Formula I, the compound is of Formula IH:

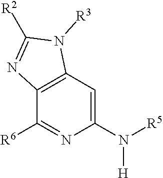

where R², R³, R⁵ and R⁶ are as defined herein, and R⁴ is H.

In some embodiments of Formula 7, the compound is of Formula IJ:

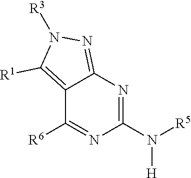

wherein R¹, R³, R⁵, and R⁶ are as defined herein.

In some embodiments of Formula 7, the compound is of Formula IK:

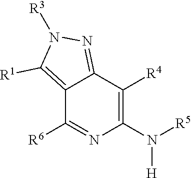

wherein R¹, R³, R⁴, R⁵, and R⁶ are as defined herein.

In some embodiments of Formula 3, the compound is a Formula IL:

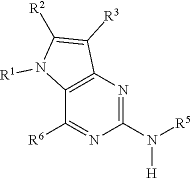

wherein R¹, R², R³, R⁵, and R⁶ are as defined herein.

In some embodiments of Formula 3, the compound is a Formula IM:

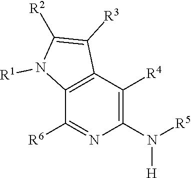

wherein R¹, R², R³, R⁴, R⁵, and R⁶ are as defined herein.

In some embodiments of Formula 3, the compound is a Formula IN:

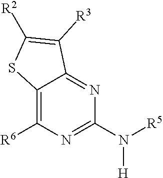

wherein R², R³, R⁵, and R⁶ are as defined herein.

In some embodiments of Formula 3, the compound is of Formula IO:

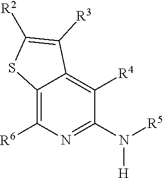

wherein R², R³, R⁴, R⁵, and R⁶ are as defined herein.

In some embodiments of Formula 3, the compound is of Formula IP

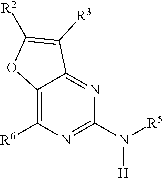

wherein R², R³, R⁴, R⁵, and R⁶ are as defined herein.

In some embodiments of Formula 3, the compound is of Formula IQ

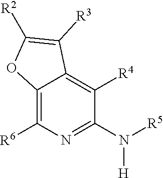

wherein R², R³, R⁴, R⁵, and R⁶ are as defined herein.

In some embodiments of Formula δ, the compound is of Formula IR

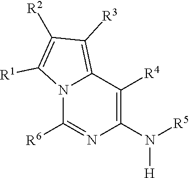

wherein R¹, R², R³, R⁴, R⁵, and R⁶ are as defined herein.

In some embodiments of Formula δ, the compound is of Formula IS

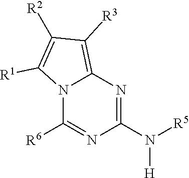

wherein R¹, R², R³, R⁵, and R⁶ are as defined herein.

In some embodiments of Formula δ, the compound is of Formula IT

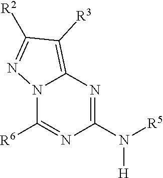

wherein R², R³, R⁵, and R⁶ are as defined herein.

In some embodiments of Formula δ, the compound is of Formula IU

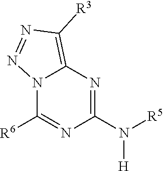

wherein R³, R⁵, and R⁶ are as defined herein.

In some embodiments of Formula α, β, γ, δ, ε, Formula I, or any one of Formulae IA-IU, R⁵ is

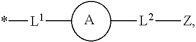

where:
A is a cyclic group selected from carbocyclyl, heterocyclyl, aryl, heteroaryl, and substituted versions thereof;
Z is a hydrophilic group;
R⁵ is L¹ and L² are optional linkers.

In some embodiments of Formula α, β, γ, δ, ε, Formula I, or any one of Formulae IA-IU, R⁵ is

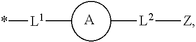

where the linker L¹ is an optionally substituted (C₁₋₃)alkylene, and L² is an optionally substituted (C₁₋₃)alkylene. In some embodiments, L¹ is CH₂. In some embodiments, L¹ is (CH₂)₂.

In some embodiments of Formula α, β, γ, δ, ε, Formula I, or any one of Formulae IA-IU, R⁵ is

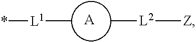

where the linker L¹ is an optionally substituted (C₁₋₃)alkylene, and L² is absent. In some embodiments, L¹ is CH₂. In some embodiments, L¹ is (CH₂)₂.

In some embodiments of Formula α, β, γ, δ, ε, Formula I, or any one of Formulae IA-IU, R⁵ is

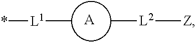

where the linker L¹ is absent, and L² is an optionally substituted (C₁₋₃)alkylene.

In some embodiments of Formula α, β, γ, δ, ε, Formula I, or any one of Formulae IA-IU, R⁵ is

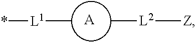

where the optional linkers L¹, and L² are absent.

In some embodiments of Formula α, β, γ, δ, ε, Formula I, or any one of Formulae IA-IU, A is selected from optionally substituted aryl, optionally substituted cycloalkyl, optionally substituted cycloalkenyl, optionally substituted heterocyclyl, and optionally substituted heteroaryl.

In some embodiments of Formula α, β, γ, δ, ε, Formula I, or any one of Formulae IA-IU, A is optionally substituted heterocycle or optionally substituted cycloalkyl. In some embodiments of Formula α, β, γ, δ, ε, Formula I, or any one of Formula IA-IU, A is an optionally substituted cycloalkyl. In some embodiments, the cycloalkyl is cyclohexyl. In some embodiments, the cycloalkyl is cyclopentyl. In some embodiments of Formula α, β, γ, δ, ε, Formula I, or any one of Formula IA-IU, A is an optionally substituted heterocycle. In some embodiments, the heterocycle includes one or more nitrogen atoms. In some embodiments, the heterocycle is a 6-membered heterocycle. In some embodiments the 6-membered heterocycle is selected from piperidine or piperazine.

In some embodiments of Formula α, β, γ, δ, ε, Formula I, or any one of Formulae IA-IU, the compound is of Formula IIA:

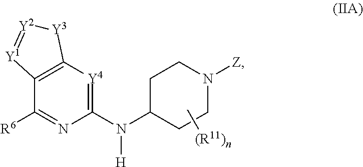

wherein,
Y¹-Y⁴, R⁶ and Z are as defined herein;
each R¹¹ is independently selected from optionally substituted alkyl, optionally substituted alkoxyl, OH, halogen, and CN; and
n is 0 to 9, such as 0, 1, 2, 3 or 4.

In some embodiments of Formula IIA, one or more of Y¹-Y³ is N, or NR³. In some embodiments, Y³ is NR³. In some embodiments, Y² is N. In some embodiments Y¹ is N. In some embodiments, two or three of Y¹-Y³ is N, or NR³. In some embodiments Y¹ is N, and Y³ is NR³. In some embodiments Y² is N, and Y³ is NR³. In some embodiments Y¹, and Y² are each N.

In some embodiments of Formula IIA, two or more of Y¹-Y³ are CR¹, CR², and/or CR³R^{3′}, respectively. In some embodiments of Formula IIA, Y¹-Y³ are CR¹, CR², and CR³R^{3′}, respectively. In some embodiments, each of R¹-R^{3′} are H. In some embodiments, at least one of R¹-R^{3′} is not H. In some embodiments, Y³ is CHR³, and R³ is not H.

In some embodiments of Formula IIA, Y³ is N, and Y¹, and Y² are each CR¹, and CR², respectively. In some embodiments of Formula IIA, Y¹ is N, and Y², and Y³ are each CR², and CR³, respectively. In some embodiments of Formula IIA, Y² is N, and Y¹, and Y³ are each CR¹, and CR³, respectively.

In some embodiments of Formula IIA, R³ is selected from phenyl, pyridyl, alkylene-phenyl, alkylene-pyridyl, and substituted versions thereof.

In some embodiments of Formula IIA, R³ is selected from cycloalkyl, cycloalkenyl, alkylene-cycloalkyl, alkylene-cycloalkenyl, and substituted versions thereof. In some embodiments, R³ is optionally substituted heterocycle, or optionally substituted alkylene-heterocycle. In some embodiments R³ is optionally substituted piperidinyl or optionally substituted piperazinyl. In some embodiments R³ is optionally substituted alkylene-piperidinyl or optionally substituted alkylene-piperazinyl. In some embodiments R³ is optionally substituted cycloalkyl or alkylene-cycloalkyl. In some embodiments R³ is optionally substituted cyclohexyl, or optionally substituted alkylene-cyclohexyl. In some embodiments R³ is optionally substituted cyclopentyl, or optionally substituted alkylene-cyclopentyl. In some embodiments R³ is optionally substituted cyclobutyl, or optionally substituted alkylene-cyclobutyl.

In some embodiments of Formula IIA, Y⁴ is CR⁴. In some embodiments, R⁴ is H.

In some embodiments of Formula IIA, Y⁴ is N.

In some embodiments of Formula IIA, n is 0, such that the piperidine ring does not include any R¹¹ substituents. In some embodiments of Formula IIA, n is 1-9, such that the piperidine ring includes one or more R¹¹ substituents. In some embodiments, n is 1. In some embodiments, n is 2. In some embodiments, n is 3. In some embodiments n is 4. In some embodiments, n is 5. In some embodiments, each R¹¹ is independently selected from C₁₋₃ alkyl, halogen, and CN. In some embodiments, at least one R¹¹ is methyl. In some embodiments, at least one R¹¹ is Cl or F. In some embodiments, at least one R¹¹ is CN.

In some embodiments of Formula IIA, the compound is of the Formula IIIA:

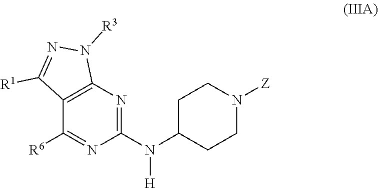

wherein R¹, R³, Y⁴, R⁶ and Z are as defined herein.

In some embodiments of Formula IIIA, R³ is selected from phenyl, pyridyl, alkylene-phenyl, alkylene-pyridyl, and substituted versions thereof.

In some embodiments of Formula IIIA, R³ is selected from cycloalkyl, cycloalkenyl, alkylene-cycloalkyl, alkylene-cycloalkenyl, and substituted versions thereof. In some embodiments of Formula IIIA, R³ is optionally substituted heterocycle, or optionally substituted alkylene-heterocycle. In some embodiments R³ is optionally substituted piperidinyl or optionally substituted piperazinyl. In some embodiments R³ is optionally substituted alkylene-piperidinyl or optionally substituted alkylene-piperazinyl. In some embodiments R³ is optionally substituted cycloalkyl or alkylene-cycloalkyl. In some embodiments R³ is optionally substituted cyclohexyl, or optionally substituted alkylene-cyclohexyl. In some embodiments R³ is optionally substituted cyclopentyl, or optionally substituted alkylene-cyclopentyl. In some embodiments R³ is optionally substituted cyclobutyl, or optionally substituted alkylene-cyclobutyl.

In some embodiments of Formula IIIA, R¹ is H, or optionally substituted (C₁₋₃)alkyl. In some embodiments of Formula IIIA, R⁶ is H.

In some embodiments of Formula α, β, γ, δ, ε, Formula I, or any one of Formula IA-IU, cyclic group A is optionally substituted aryl or optionally substituted heteroaryl. In some embodiments of Formula α, β, γ, δ, ε, Formula I, or any one of Formula IA-IU, A is an optionally substituted aryl. In some embodiments of Formula I, or any one of Formula IA-IJ, A is an optionally substituted heteroaryl. In some embodiments, the heteroaryl is a 5-membered heteroaryl. In some embodiments, the heteroaryl is a 6-membered heteroaryl. In some embodiments, the heteroaryl is pyridyl, pyridazine, pyrimidine or triazine. In some embodiments, A is optionally substituted phenyl. In some embodiments, A is optionally substituted pyridyl (e.g., 2-pyridyl, 3-pyridyl, or 4-pyridyl).

In some embodiments of Formula α, β, γ, δ, ε, Formula I, or any one of Formulae IA-IU, the compound is of Formula IIB:

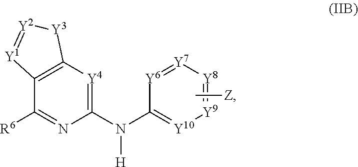

wherein:
Y¹-Y⁴, R⁶ and Z are as defined herein;
Y⁶ to Y¹⁰ are each independently C—Z, CR¹² or N; and
each R¹² is independently selected from H, optionally substituted alkyl, optionally substituted alkoxyl, OH, halogen, and CN.

In some embodiments of Formula IIB, each R¹² is independently selected from H, (C₁₋₅)alkyl, substituted (C₁₋₅)alkyl, (C₁₋₅)alkoxy, substituted (C₁₋₅)alkoxy, halogen, OH, and CN

In some embodiments of Formula IIB, one and only one of Y⁶ to Y¹⁰ is C—Z. In some embodiments of Formula IIB, Y⁸ is C—Z. In some embodiments, Y⁹ is C—Z. In some embodiments, Y⁶ or Y¹⁰ is C—Z.

In some embodiments of Formula IIB, Y⁸ is C—Z, and Y⁶, Y⁷, Y⁹, and Y¹⁰ are each CR¹². In some embodiments, each R¹² is H. In some embodiments, at least one R¹² is not H. In some embodiments, at least one R¹² is selected from optionally substituted C₁₋₃ alkyl, OH, halogen, and CN. In some embodiments, at least one R¹² is methyl. In some embodiments, at least one R¹² is Cl or F. In some embodiments, at least one R¹² is CN.

In some embodiments of Formula IIB, Y⁹ is C—Z, and Y⁶, Y⁷, Y⁸, and Y¹⁰ are each CR¹². In some embodiments, each R¹² is H. In some embodiments, at least one R¹² is not H. In some embodiments, at least one R¹² is selected from optionally substituted C₁₋₃ alkyl, OH, halogen, and CN. In some embodiments, at least one R¹² is methyl. In some embodiments, at least one R¹² is Cl or F. In some embodiments, at least one R¹² is CN.

In some embodiments of Formula IIB, at least one of Y⁶-Y¹⁰ is N. In some embodiments of Formula IIB, one and only one of Y⁶-Y¹¹ is N. In some embodiments of Formula IIB, two of Y⁶-Y¹¹ is N. In some cases, Y⁶ or Y¹⁰ is N. In some cases, both Y⁶ and Y¹⁰ are N. In some cases, Y⁷ or Y⁹ is N. In some cases, both Y⁷ and Y⁹ are N. In some cases, Y⁸ is N.

In some embodiments of the compound of Formula IIB, one or more of Y¹-Y³ is N, or NR³. In some embodiments, Y³ is NR³. In some embodiments, Y² is N. In some embodiments Y¹ is N. In some embodiments of Formula IIB, two or three of Y¹-Y³ is N, or NR³. In some embodiments, Y¹ is N, and Y³ is NR³. In some embodiments, Y² is N, and Y³ is NR³. In some embodiments, Y¹ and Y² are each N.

In some embodiments of Formula IIB, two or more of Y¹-Y³ are CR¹, CR² and CR³R^{3′}, respectively. In some embodiments of Formula IIB, Y¹-Y³ are CR¹, CR² and CR³R^{3′}, respectively. In some embodiments, each of R¹-R^{3′} are H. In some embodiments, at least one of R¹-R^{3′} is not H. In some embodiments, Y³ is CHR³, and R³ is not H.

In some embodiments of Formula IIB, Y⁴ is CR⁵. In some embodiments, R⁴ is H.

In some embodiments of Formula IIB, Y⁴ is N.

In some embodiments of Formula IIB, the compound is of one of Formula (IIIB) to Formula (IIIE):

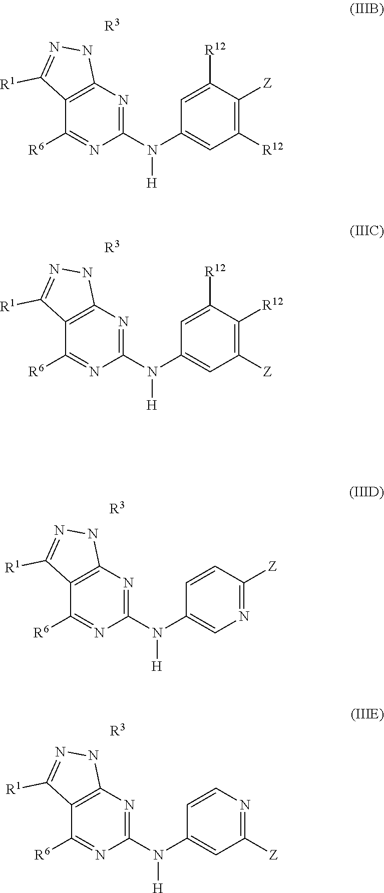

where R¹, R³, R⁶, R¹², and Z are as defined herein.

In some embodiments of Formula IIB, the compound is of one of Formula (IIIF) to Formula (IIIJ):

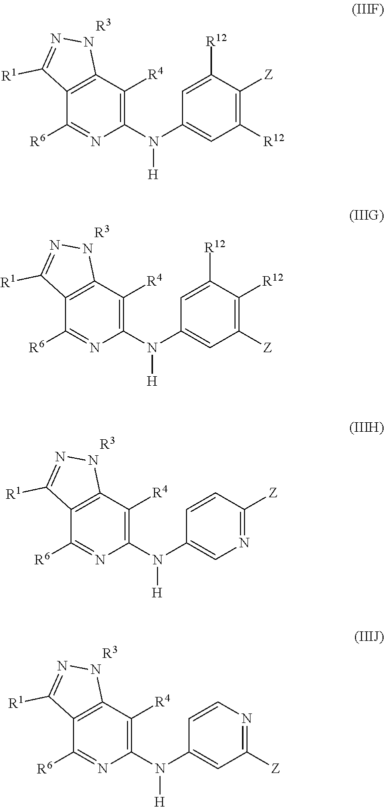

where R¹, R³, R⁴, R⁶, R¹², and Z are as defined herein.

In some embodiments of Formula (I), in R⁵, L¹ and L² are independently absent or (C₁₋₆)alkylene. In some embodiments, L¹ is CH₂, (CH₂)₂, or (CH₂)₃. In some embodiments, L¹ is absent. In some embodiments, L² is CH₂, (CH₂)₂, or (CH₂)₃. In some embodiments of Formula (I), in R⁵, L¹ is absent, and L² is CH₂, (CH₂)₂, or (CH₂)₃.

In some embodiments of any one of Formulae α, β, γ, δ, ε, and I-IIIJ, Z includes a hydrophilic group selected from sulfone, sulfonate, carboxamide, carboxy, hydroxy, hydroxyalkyl, aldehyde, ester, alkoxy, nitrile, halogen, thiolactone, substituted versions thereof, and any combination thereof. In some embodiments, Z includes a sulfone. In some embodiments, Z includes a sulfonate. In some embodiments, Z includes a carboxamide. In some embodiments, Z includes a carboxy. In some embodiments, Z includes a hydroxy or a hydroxyalkyl. In some embodiments, Z includes an aldehyde. In some embodiments, Z includes an ester. In some embodiments, Z includes an alkoxy. In some embodiments, Z includes a nitrile. In some embodiments, Z includes a halogen. In some embodiments, Z includes a thiolactone. In some embodiments, Z includes a carboxamide and a thiolactone.

In some embodiments of any one of Formulae α, β, γ, δ, ε, and I-IIIJ, Z is selected from a sulfone, a sulfonate, carboxy, ester, hydroxy, hydroxyalkyl, aldehyde, an optionally substituted thiolactone, an optionally substituted thioester, and optionally substituted carboxamide. In some embodiments, Z is a sulfone. In some embodiments, Z is a vinylsulfone. In some embodiments, Z is an ynesulfone. In some embodiments, Z is a ketone. In some embodiments, Z is an enone. In some embodiments, Z is an ynone. In some embodiments, Z is an acrylamide. In some embodiments, Z is an acrylate. In some embodiments, Z is a sulfonate. In some embodiments, Z is an optionally substituted carboxamide. In some embodiments, Z is a carboxamide substituted with a thiolactone group. In some embodiments the thiolactone is a γ-thiolactone. In some embodiments, Z is a carboxy group. In some embodiments, Z is a hydroxy or a hydroxyalkyl group. In some embodiments, Z is an aldehyde. In some embodiments, Z is an ester. In some embodiments, Z is a thioester (e.g., —COSR¹² where R¹² is optionally substituted (C₁₋₆)alkyl, optionally substituted aryl, or optionally substituted heterocycle). In some embodiments, Z includes an optionally substituted thiolactone, such as a γ-thiolactone.

In some embodiments of any one of Formulae α, β, γ, δ, ε, and I-IIIJ, L²-Z, or Z is of one of Z1-Z7:

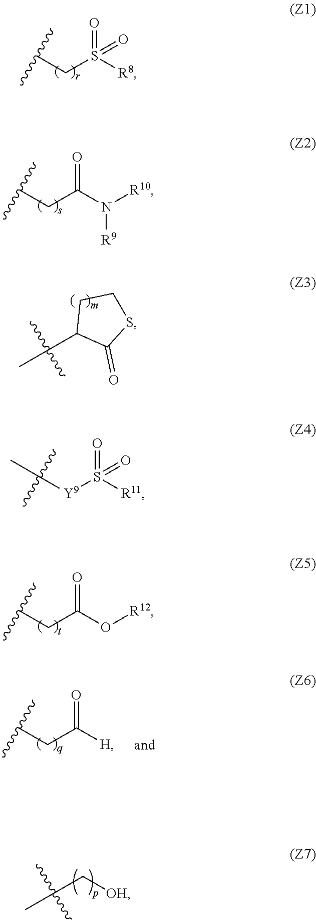

wherein:
R⁸ is (C₁₋₆)alkyl, (C₂₋₆)alkenyl, (C₂₋₆)alkynyl, or a substituted version thereof;
R⁹ is H or optionally substituted (C₁₋₆)alkyl;
R¹⁰ is H, optionally substituted (C₁₋₆)alkyl, a sulfone (e.g., of formula Z1), or a thiolactone (e.g., of formula Z3); and
Y⁹ is absent, —O—, —NR⁹—, —(C₁₋₆)alkylene-O—, —(C₁₋₆)alkylene-NR⁹—;
R¹¹ is halogen, —NR⁹R¹⁰, (C₁₋₆)alkyl, (C₂₋₆)alkenyl, (C₂₋₆)alkynyl, or a substituted version thereof;
R¹² is H, optionally substituted (C₁₋₆)alkyl, optionally substituted aryl, or optionally substituted heterocycle;
m is 0 to 2; and
p, q, r, s and t are independently 0, 1, 2, 3, 4, 5 or 6.

In some embodiments of any one of Formulae α, β, γ, δ, ε, and I-IIIJ, L²-Z, or Z is of Formula Z1:

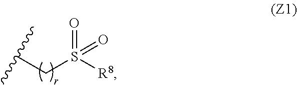

wherein:
R⁸ is optionally substituted alkyl, optionally substituted cycloalkyl, amino, halogen, optionally substituted alkenyl, or optionally substituted alkynyl; and
r is 0, 1, 2, 3, 4, 5 or 6.

In some embodiments of formula Z1, R⁸ is (C₁₋₆)alkyl (e.g., (C₃₋₆)cycloalkyl), (C₂₋₆)alkenyl, (C₂₋₆)alkynyl, or a substituted version thereof.

In some embodiments of formula Z1, R⁸ is optionally substituted alkyl. In some embodiments, R⁸ is NH₂. In some embodiments, R⁸ is NR′R″ wherein R′ and R″ are independently H, or optionally substituted alkyl. In some embodiments, R⁸ is halogen. In some embodiments, R⁸ is optionally substituted alkenyl. In some embodiments, R⁸ is optionally substituted alkynyl.

In some embodiments of any one of Formulae α, β, γ, δ, ε, and I-IIIJ, L²-Z, or Z is of the Formula Z2:

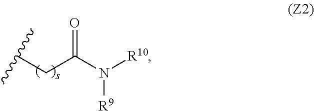

wherein:
R⁹ is H or optionally substituted (C₁₋₆)alkyl;
R¹⁰ is H, optionally substituted (C₁₋₆)alkyl, a sulfone (e.g., of formula Z1), or a thiolactone (e.g., of formula Z3);
s is 0, 1, 2, 3, 4, 5 or 6.

In some embodiments of Formula Z2, R⁹ is H. In some embodiments R⁹ is alkyl. In some embodiments, R¹⁰ is sulfone (e.g., of Formula Z1). In some embodiments, R¹⁰ is thiolactone (e.g., of Formula Z3). In some embodiments, R⁹ is H and R¹⁰ is thiolactone. In some embodiments the thiolatone is a γ-thiolactone.

In some embodiments of any one of Formulae α, β, γ, δ, ε, and I-IIIJ, L²-Z, or Z is of Formula Z3:

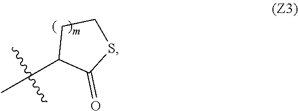

wherein m is 0 to 2. In some embodiments, m is 0. In some embodiments, m is 1. In some embodiments, m is 2.

In some embodiments of any one of Formulae α, β, γ, δ, ε, and I-IIIJ, L²-Z, or Z is of Formula Z4:

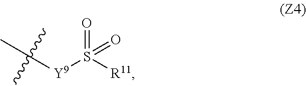

wherein:
Y⁹ is absent, —O—, —NR⁹—, —(C₁₋₆)alkylene-O—, or —(C₁₋₆)alkylene-NR⁹—R⁹ is H or optionally substituted (C₁₋₆)alkyl;
R¹⁰ is H, optionally substituted (C₁₋₆)alkyl, a sulfone (e.g., of formula Z1), or a thiolactone (e.g., of formula Z3); and
R¹¹ is halogen, —NR⁹R¹⁰, alkyl, alkenyl, alkynyl, or a substituted version thereof.

In some embodiments of Z4, R¹¹ is halogen. In some embodiments of Z4, R¹¹ is optionally substituted (C₁₋₆)alkyl. In some embodiments of Z4, R¹¹ is amino. In some embodiments of Z4, R¹¹ is NR⁹R¹⁰. In some embodiments of Z4, R¹¹ is optionally substituted (C₂₋₆)alkenyl. In some embodiments of Z4, R¹¹ is optionally substituted (C₂₋₆)alkynyl.

In some embodiments of Z4, R¹¹ is halogen, —NR⁹R¹⁰, (C₁₋₆)alkyl, (C₂₋₆)alkenyl, (C₂₋₆)alkynyl, or a substituted version thereof

In some embodiments of any one of Formulae α, β, γ, δ, ε, and I-IIIJ, L²-Z, or Z is of Formula Z5:

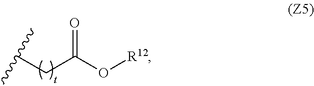

wherein:
R¹² is H, optionally substituted alkyl, optionally substituted aryl, optionally substituted heteroaryl, or optionally substituted heterocycle; and
t is 0, 1, 2, 3, 4, 5 or 6.

In some embodiments of Z5, R¹² is H. In some embodiments of Z5, R¹² is alkyl. In some embodiments of Z5, R¹² is optionally substituted phenyl. In some embodiments of Z5, R¹² is a heterocycle.

In some embodiments of any one of Formulae α, β, γ, δ, ε, and I-IIIJ, L²-Z, or Z is of the Formula Z6:

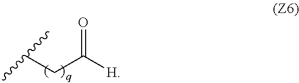

In some embodiments of any one of Formulae α, β, γ, δ, ε, and I-IIIJ, L²-Z, or Z is of the Formula Z7:

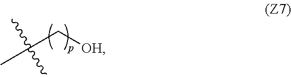

wherein p is 0 to 6. In some embodiments p is 0. In some embodiments, p is 1. In some embodiments, p is 2. In some embodiments, p is 3. In some embodiments, p is 4. In some embodiments, p is 5. In some embodiments, p is 6.

In some embodiments of any one of Formulae α, β, γ, δ, ε, and I-IIIJ, Z is a non-electrophilic hydrophilic group.

In some embodiments, of any one of Formulae α, β, γ, δ, ε, and I-IIIJ, Z is a non-electrophilic hydrophilic group of the formula Z1, wherein R⁸ is optionally substituted alkyl or amino. In some embodiments, Z is Z1, and R⁸ is amino. In some embodiments, Z is Z1 and R⁸ is optionally substituted alkyl. As such, in some embodiments of Formulae α, β, γ, δ, F, and I-IIIJ, Z is an alkylsulfone. In some embodiments, Z is an alkylsulfone of Formula Z1A:

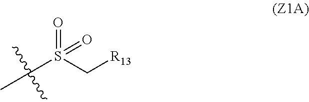

wherein R¹³ is H or optionally substituted C₁-C₆ alkyl. In some embodiments of Z1A, R¹³ is H. In some embodiments of Z1A, R¹³ is C₁-C₃ alkyl.

In some embodiments of any one of Formulae α, β, γ, δ, ε, and I-IIIJ, Z is a non-electrophilic hydrophilic group of Formula Z2, wherein;
R⁹ is H; and
R¹⁰ is of the formula —SO₂R⁸; and
R⁸ is optionally substituted alkyl, or amino.

In some embodiments of any one of Formulae α, β, γ, δ, ε, and I-IIIJ, Z is a carboxamide of Formula Z2, R¹⁰ is —SO₂R⁸, and R⁸ is methyl. In some embodiments, Z is a carboxamide of Formula Z2, R¹⁰ is of the formula —SO₂R⁸, and R⁸ is amino.

In some embodiments of any one of Formulae α, β, γ, δ, ε, and I-IIIJ, Z is a non-electrophilic hydrophilic group of Formula Z7, wherein p is 0, 1 or 2. In some embodiments, p is 0, such that Z7 is —OH. In some embodiments, p is 1, such that Z7 is —CH₂OH. In some embodiments, p is 2, such that Z7 is CH₂CH₂OH.

In some embodiments of any one of Formulae α, β, γ, δ, ε, and I-IIIJ, Z is a non-electrophilic hydrophilic group of formula Z5, where R¹² is H (i.e., Z is —CO₂H).

In some embodiments of any one of Formulae α, β, γ, δ, ε, and I-IIIJ, Z is an electrophilic reactive hydrophilic group.

In some embodiments, of any one of Formulae α, β, γ, δ, ε, and I-IIIJ, Z is an electrophilic reactive hydrophilic group of the formula Z1, wherein R⁸ is optionally substituted alkenyl. Thus, in some embodiments of Formulae α, β, γ, δ, ε, and I-IIIJ, Z is an alkenylsulfone (e.g., a vinylsulfone). In some embodiments, Z is an alkenylsulfone of Formula Z1B:

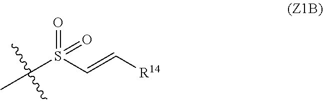

wherein R¹⁴ is H or optionally substituted C₁-C₆ alkyl. In some embodiments of Z1B, R¹⁴ is H. In some embodiments of Z1B, R¹⁴ is optionally substituted C₁-C₃ alkyl.

In some embodiments of any one of Formulae α, β, γ, δ, ε, and I-IIIJ, L²-Z or Z is a carboxamide of Formula Z2, where R¹⁰ is —SO₂R⁸, or thiolactone, and where R⁸ is optionally substituted alkenyl. In some embodiments, Z is a carboxamide of Formula Z2, R¹⁰ is —SO₂R⁸, and R⁸ is substituted alkenyl. In some cases, R⁸ is —CH═CH₂. In some embodiments, Z is a carboxamide of Formula Z2, where R¹⁰ is thiolactone. In some embodiments, Z2 is of Formula Z2A:

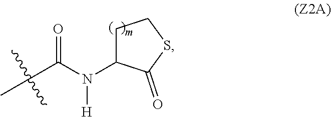

wherein m is 0 to 2. In some embodiments, m is 0. In some embodiments, m is 1. In some embodiments, m is 2.

In some embodiments of any one of Formulae α, β, γ, δ, ε, and I-IIIJ, L²-Z, or Z is a thiolactone of Formula Z3:

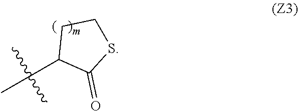

In some embodiments, m is 0. In some embodiments of Z3, m is 1, and Z is γ-thiolactone. In some embodiments, m is 2.

In some embodiments of any one of Formulae α, β, γ, δ, ε, and I-IIIJ, Z is of Formula Z4, where R¹¹ is halogen. In some embodiments, R¹¹ is F.

In some embodiments of any one of Formulae α, β, γ, δ, ε, and I-IIIJ, Z is of Formula Z5, wherein R¹² is optionally substituted alkyl or a heterocycle. In some embodiments, Z is of Formula Z5, where R¹² is a heterocycle. In some embodiments, R¹² is.

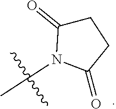 [SMILES: *N1C(=O)CCC1=O]

In some embodiments of any one of Formulae α, β, γ, δ, ε, and I-IIIJ, -L²-Z is of the formula Z6:

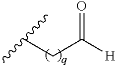

where q is 0, 1, 2, 3, 4, 5 or 6.
In some embodiments, Z is CHO, CH₂CHO, (CH₂)₂CHO, or (CH₂)₃CHO.

In some embodiments of any one of Formulae α, β, γ, δ, ε, and I-IIIJ, Z is of the formula Z7:

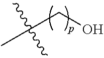

where p is 0, 1, 2, 3, 4, 5 or 6.

In some embodiments, Z is OH, CH₂OH, (CH₂)₂OH, or (CH₂)₃OH.

In some embodiments of any one of Formulae α, β, γ, δ, ε, and I-IIIJ, Z is a moiety that is substantially biologically inert.

In some embodiments of any one of Formulae α, β, γ, δ, ε, and I-IIIJ, Z includes an electrophilic reactive moiety capable of covalently conjugating a biological target.

In some embodiments of Formula I, the compound is of one of Formula (IVA)-(IVC):

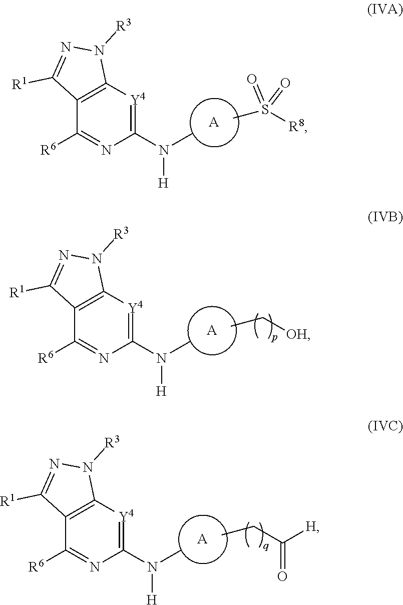

wherein:
R⁸ is optionally substituted (C₁-C₆)alkyl, optionally substituted (C₂-C₆)alkenyl, or optionally substituted (C₂-C₆)alkynyl; and
p is 0, 1, 2 or 3; and
q is 0, 1, 2 or 3.

In some embodiments of Formulae (IVA)-(IVC), A is phenyl, pyridyl (e.g., 3-pyridyl, or 4-pyridyl), 4-piperidinyl, 4-indolyl, 5-indolyl, pyrrolidin-2-one, or a substituted version thereof. In some embodiments of Formulae (IVA)-(IVC), A is substituted phenyl. In some embodiments of Formulae (IVA)-(IVC), A is 3-pyridyl, or 4-pyridyl. In some embodiments of Formulae (IVA)-(IVC), A is optionally substituted 4-piperidinyl.

In some embodiments of any one of Formulae α, β, γ, δ, ε, and (I)-(IVC), R³ is a monocyclic group. In some embodiments of any one of Formulae α, β, γ, δ, ε, and (I)-(IVC), R³ is a bicyclic group. In some embodiments of R³ the bicyclic group is a spirocyclic group. In some embodiments of R³, the bicyclic group is a fused bicyclic group.

In some embodiments of any one of Formulae α, β, γ, δ, ε, and (I)-(IVC), R³ is selected from alkyl, carbocycle, heterocycle, aryl, heteroaryl, alkylene-carbocycle, alkylene-heterocycle, alkylene-aryl, alkylene-heteroaryl, and substituted versions thereof.

In some embodiments of any one of Formulae α, β, γ, δ, ε, and (I)-(IVC), R³ is optionally substituted alkyl.

In some embodiments of any one of Formulae α, β, γ, δ, ε, and (I)-(IVC), R³ is optionally substituted cycloalkyl. In some embodiments of any one of Formulae (I)-(IVC), R³ is optionally substituted cycloalkene.

In some embodiments of any one of Formulae α, β, γ, δ, ε, and (I)-(IVC), R³ is optionally substituted heterocyclyl.

In some embodiments of any one of Formulae α, β, γ, δ, ε, and (I)-(IVC), R³ is optionally substituted aryl.

In some embodiments of any one of Formulae α, β, γ, δ, ε, and (I)-(IVC), R³ is an optionally substituted heteroaryl.

In some embodiments of any one of Formulae α, β, γ, δ, ε, and (I)-(IVC), R³ is of Formula 3A:

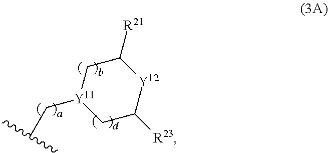

wherein:
Y¹¹ is N or CH;
Y¹² is NR²², C(R^{22′})₂, O or C═O;
a is 0, 1 or 2;
b is 0 or 1;
d is 0 or 1;
R²¹, R²², each R^{22′}, and R²³ are independently selected from H, optionally substituted (C₁₋₅)alkyl, (C₁₋₅)haloalkyl, OH, (C₁₋₅)alkylene-OH, optionally substituted O(C₁₋₅)alkyl, CHO, CN, CO₂R³², CONR³⁰R³¹, NR³⁰R³¹, halogen, and (C₁₋₅)alkylene-X;
R³⁰ and R³¹ are independently H, or (C₁₋₅)alkyl, or R³⁰ and R³¹ are cyclically linked and together with the nitrogen atom to which they are attached provide an optionally substituted heterocyclyl ring;
R³² is H, optionally substituted (C₁₋₅)alkyl, optionally substituted heterocyclyl, or optionally substituted aryl; and
X is halogen.

In some embodiments of Formulae (3A), a is 0. In some embodiments, a is 1. In some embodiments, a is 2. In some embodiments, b is 0. In some embodiments, b is 1. In some embodiments, d is 0. In some embodiments, d is 1. In some embodiments of Formulae (3A), b+d is 1. In some embodiments, b+d is 2. In some embodiments, b+d is 0.

In some embodiments of Formulae (3A), a is 0, and b is 1. In some embodiments, a is 1, and b is 1. In some embodiments, a is 2, and b is 1. In some embodiments, d is 1.

In some embodiments of Formulae (3A), R²¹ is H; and R^{22′}, and R²³ are independently selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₅)alkylene-X, where X is halogen.

In some embodiments of any one of Formulae α, β, γ, δ, ε, and (I)-(IVC), R³ is of Formula 3A:

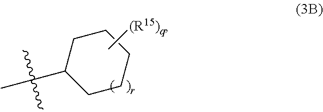

wherein:
R¹⁵ is an optional substituent selected from optionally substituted alkyl, optionally substituted amino, optionally substituted alkoxy, hydroxy, carboxy, halogen, optionally substituted heterocycle, optionally substituted heteroaryl, optionally substituted aryl, optionally substituted cycloalkyl, nitrile, and deuterium;
q is 0 to 11; and
r is 0 or 1.

In some embodiments of Formula 3B, q is 0, such that there are no R⁵ substituents. In some embodiments, q is 1-11, and each R¹⁵ substituent is independently selected from C₁-C₃ alkyl, hydroxy, halogen, nitrile and deuterium. In some embodiments, q is 1 and R¹⁵ is nitrile. In some embodiments, q is 1 and R¹⁵ is hydroxy. In some embodiments of Formula 3A, r is 0. In some embodiments of Formula 3B, r is 1.

In some embodiments, R³ is selected from

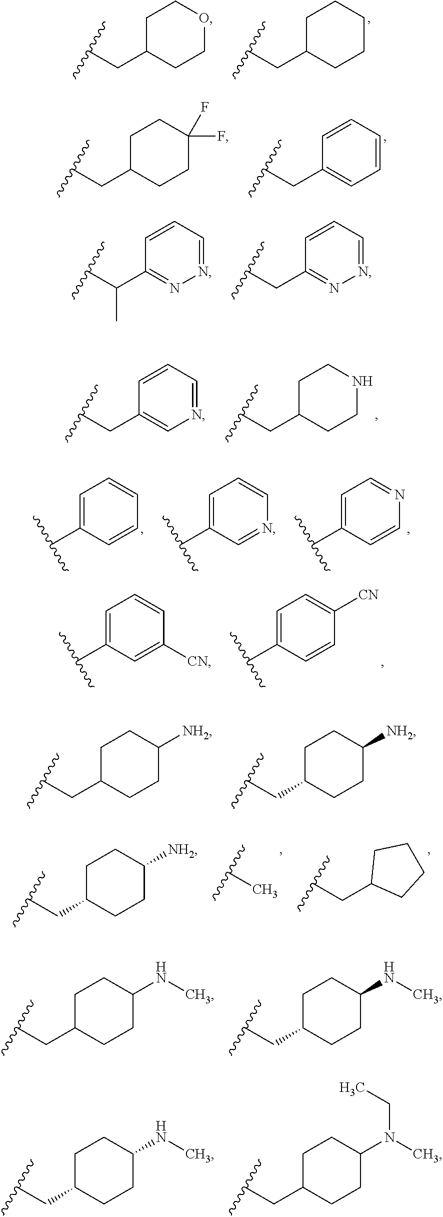

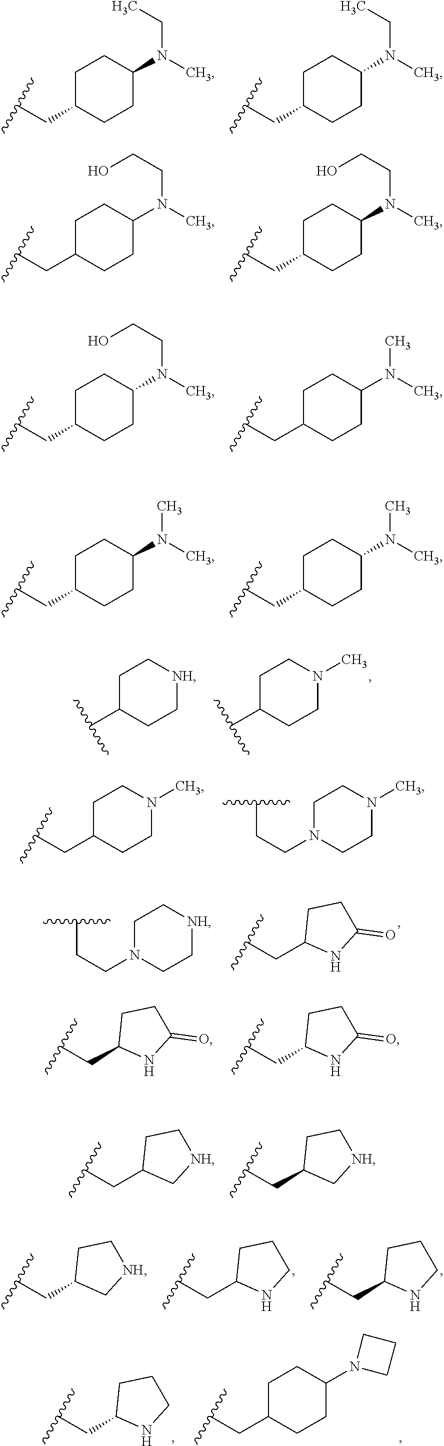

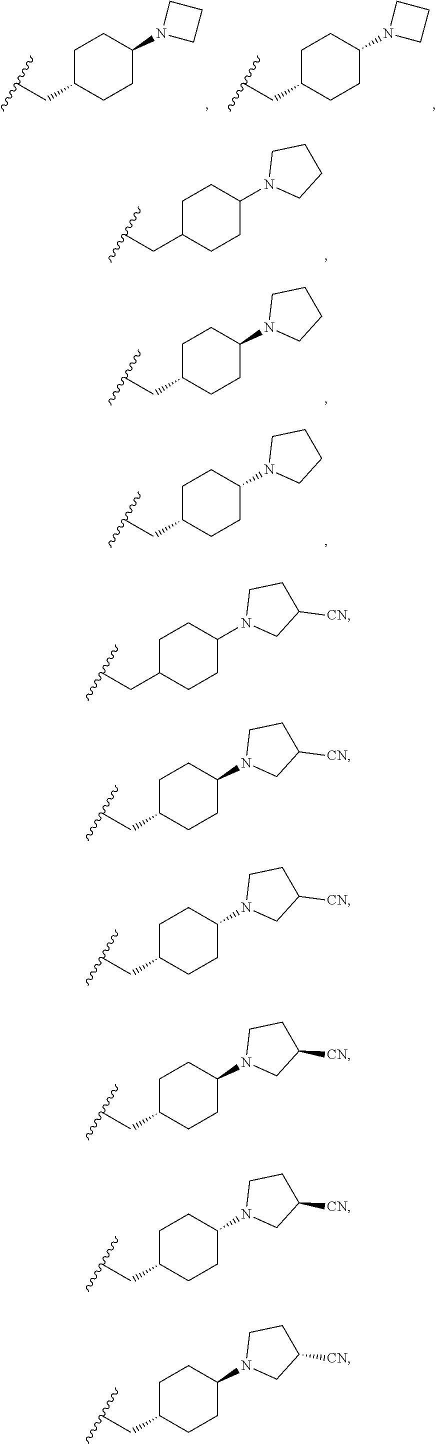

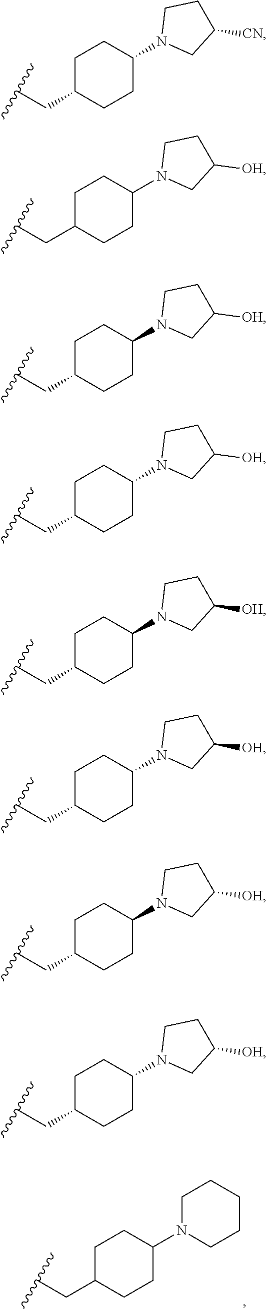

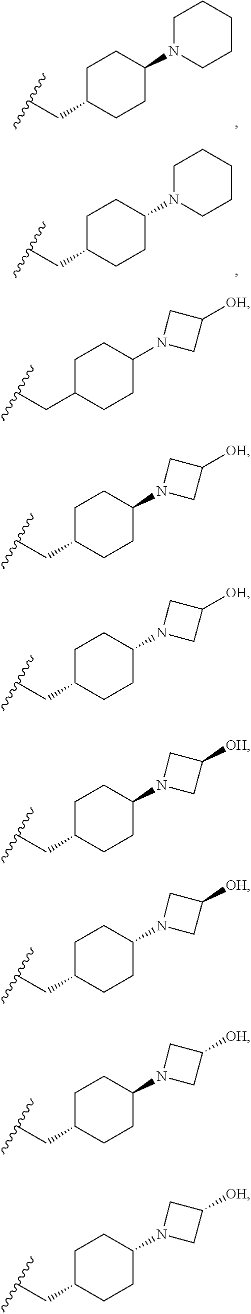

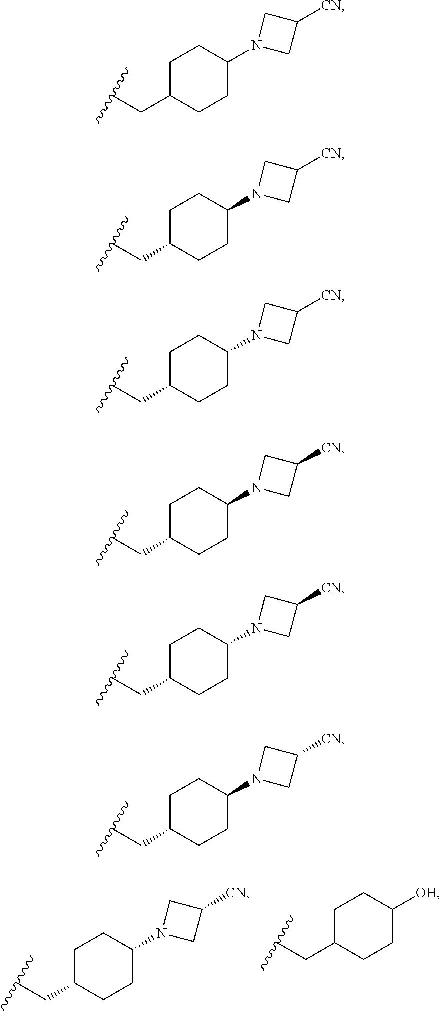

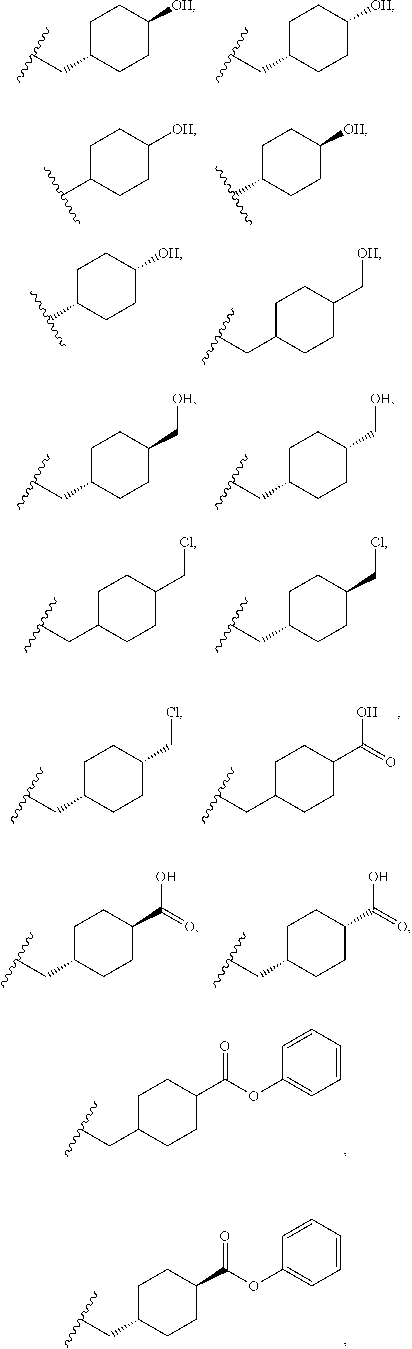

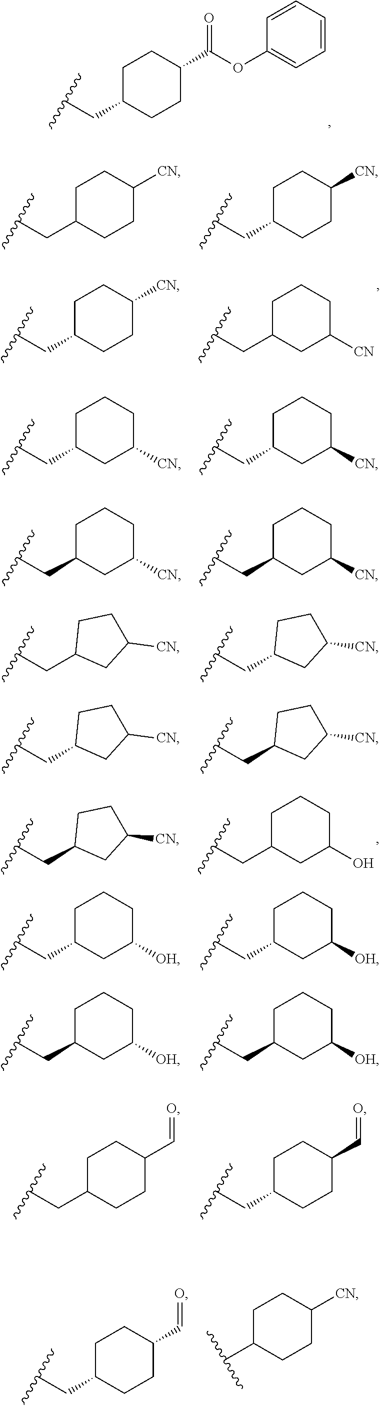

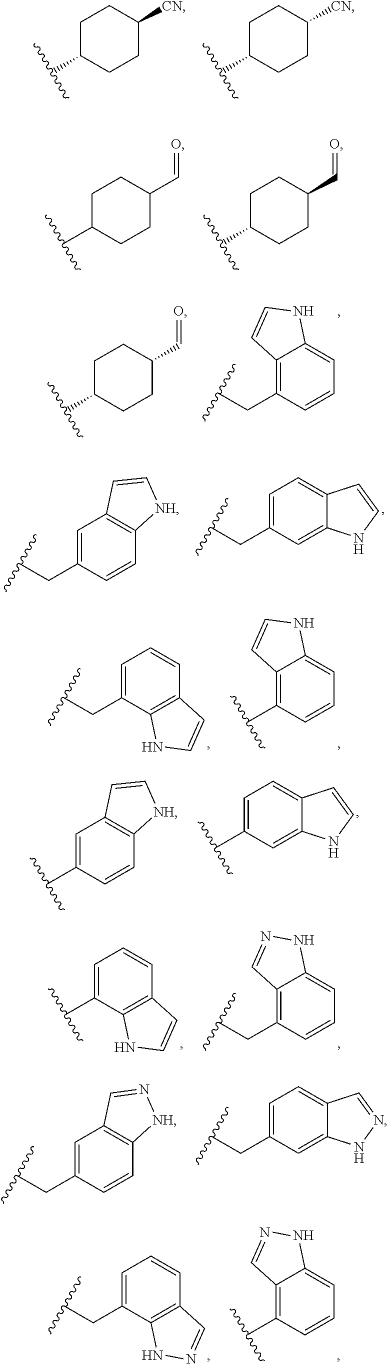

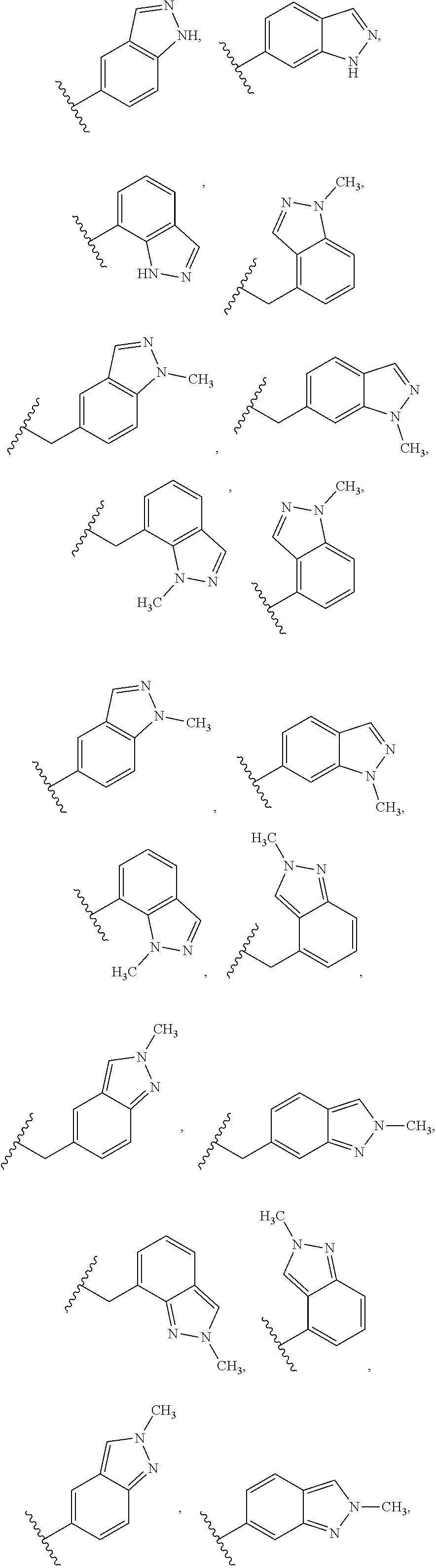

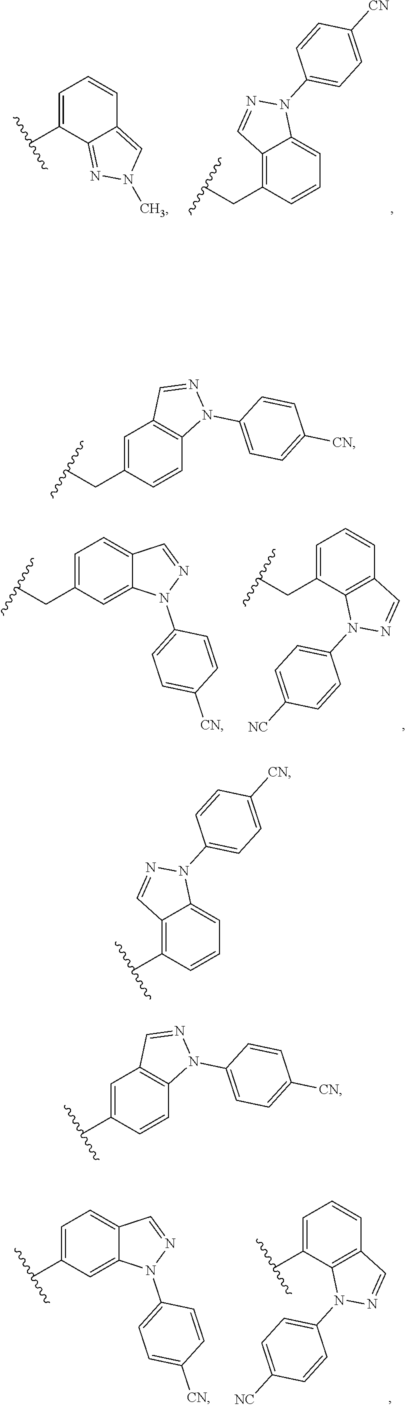

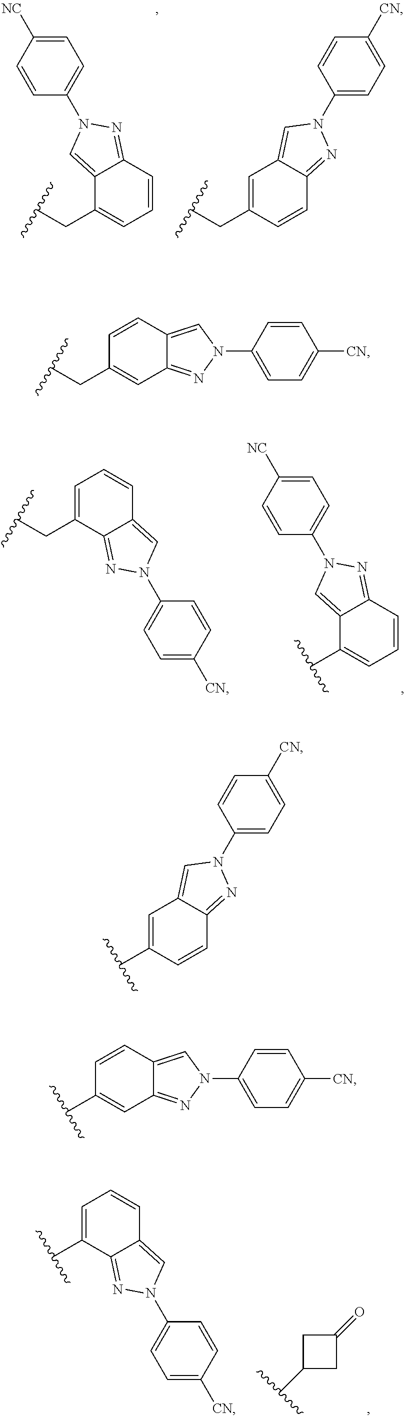

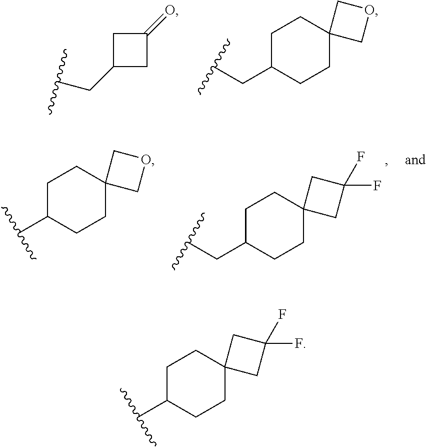 [SMILES: *CC1CC(=O)C1 ; *CC1CCC2(CC1)COC2 ; *C1CCC2(CC1)COC2 ; *CC1CCC2(CC1)CC(F)(F)C2 ; *C1CCC2(CC1)CC(F)(F)C2]

In some embodiments, R³ is

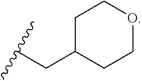 [SMILES: *CC1CCOCC1]

In some embodiments, R³ is

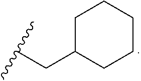 [SMILES: *CC1CCCCC1]

In some embodiments, R³ is

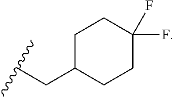 [SMILES: *CC1CCC(F)(F)CC1]

In some embodiments, R³ is

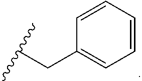 [SMILES: *Cc1ccccc1]

In some embodiments, R³ is

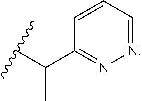 [SMILES: *C(C)c1cccnn1]

In some embodiments, R³ is

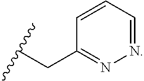 [SMILES: *Cc1cccnn1]

In some embodiments, R³ is

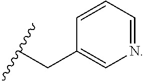 [SMILES: *Cc1cccnc1]

In some embodiments, R³ is

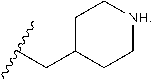 [SMILES: *CC1CCNCC1]

In some embodiments, R³ is

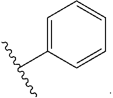 [SMILES: *c1ccccc1]

In some embodiments, R³ is

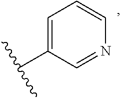 [SMILES: *c1cccnc1]

In some embodiments, R³ is

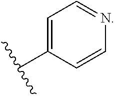 [SMILES: *c1ccncc1]

In some embodiments, R³ is

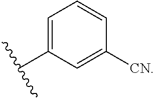 [SMILES: *c1cccc(C#N)c1]

In some embodiments, R³ is

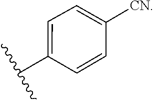 [SMILES: *c1ccc(C#N)cc1]

In some embodiments, R³ is

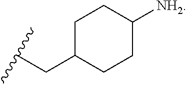 [SMILES: *CC1CCC(N)CC1]

In some embodiments, R³ is

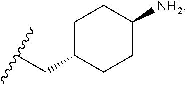 [SMILES: *C[C@H]1CC[C@H](N)CC1]

In some embodiments, R³ is

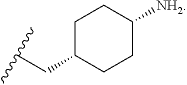 [SMILES: *C[C@H]1CC[C@@H](N)CC1]

In some embodiments, R³ is

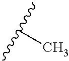

In some embodiments, R³ is

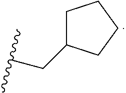 [SMILES: *CC1CCCC1]

In some embodiments, R³ is

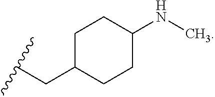 [SMILES: *CC1CCC(NC)CC1]

In some embodiments, R³ is

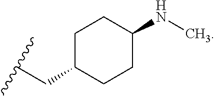 [SMILES: *C[C@H]1CC[C@H](NC)CC1]

In some embodiments, R³ is

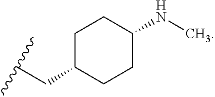 [SMILES: *C[C@H]1CC[C@@H](NC)CC1]

In some embodiments, R³ is

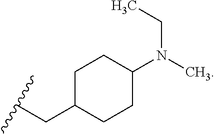 [SMILES: *CC1CCC(N(C)CC)CC1]

In some embodiments, R³ is

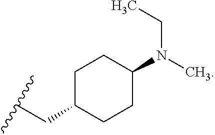 [SMILES: *C[C@H]1CC[C@H](N(C)CC)CC1]

In some embodiments, R³ is

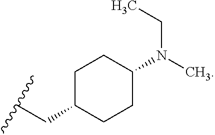 [SMILES: *C[C@H]1CC[C@@H](N(C)CC)CC1]

In some embodiments, R³ is

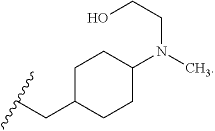 [SMILES: *CC1CCC(N(C)CCO)CC1]

In some embodiments, R³ is

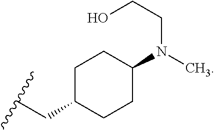 [SMILES: *C[C@H]1CC[C@H](N(C)CCO)CC1]

In some embodiments, R³ is

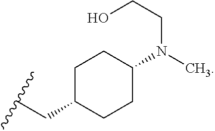 [SMILES: *C[C@H]1CC[C@@H](N(C)CCO)CC1]

In some embodiments, R³ is

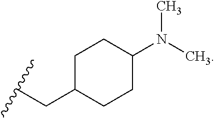 [SMILES: *CC1CCC(N(C)C)CC1]

In some embodiments, R³ is

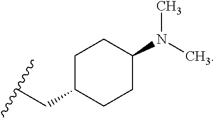 [SMILES: *C[C@H]1CC[C@H](N(C)C)CC1]

In some embodiments, R³ is

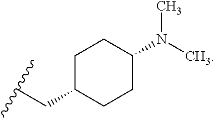 [SMILES: *C[C@H]1CC[C@@H](N(C)C)CC1]

In some embodiments R³ is

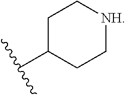 [SMILES: *C1CCNCC1]

In some embodiments, R³ is

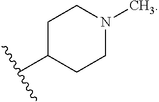 [SMILES: *C1CCN(C)CC1]

In some embodiments, R³ is

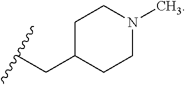 [SMILES: *CC1CCN(C)CC1]

In some embodiments, R³ is

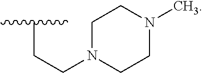 [SMILES: *CCN1CCN(C)CC1]

In some embodiments, R³ is

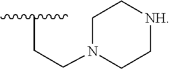 [SMILES: *CCN1CCNCC1]

In some embodiments, R³ is

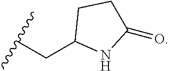 [SMILES: *CC1CCC(=O)N1]

In some embodiments, R³ is

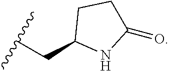 [SMILES: *C[C@H]1CCC(=O)N1]

In some embodiments, R³ is

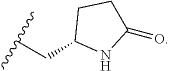 [SMILES: *C[C@@H]1CCC(=O)N1]

In some embodiments, R³ is

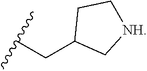 [SMILES: *CC1CCNC1]

In some embodiments, R³ is

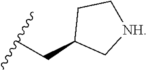 [SMILES: *C[C@H]1CCNC1]

In some embodiments, R³ is

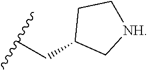 [SMILES: *C[C@@H]1CCNC1]

In some embodiments, R³ is

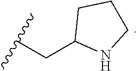 [SMILES: *CC1CCCN1]

In some embodiments, R³ is

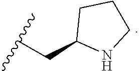 [SMILES: *C[C@H]1CCCN1]

In some embodiments, R³ is

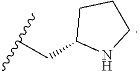 [SMILES: *C[C@@H]1CCCN1]

In some embodiments, R³ is

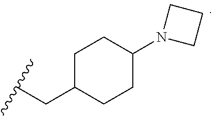 [SMILES: *CC1CCC(N2CCC2)CC1]

In some embodiments, R³ is

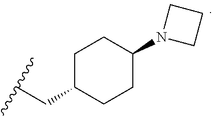 [SMILES: *C[C@H]1CC[C@H](N2CCC2)CC1]

In some embodiments, R³ is

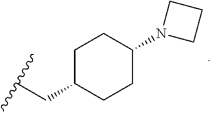 [SMILES: *C[C@H]1CC[C@@H](N2CCC2)CC1]

In some embodiments, R³ is

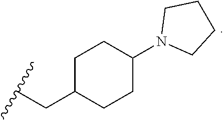 [SMILES: *CC1CCC(N2CCCC2)CC1]

In some embodiments, R³ is

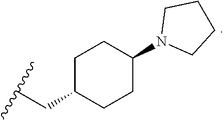 [SMILES: *C[C@H]1CC[C@H](N2CCCC2)CC1]

In some embodiments, R³ is

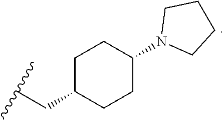 [SMILES: *C[C@H]1CC[C@@H](N2CCCC2)CC1]

In some embodiments, R³ is

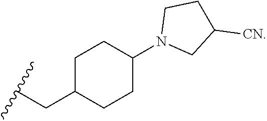 [SMILES: *CC1CCC(N2CCC(C#N)C2)CC1]

In some embodiments, R³ is

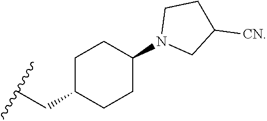 [SMILES: *C[C@H]1CC[C@H](N2CCC(C#N)C2)CC1]

In some embodiments, R³ is

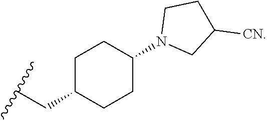 [SMILES: *C[C@H]1CC[C@@H](N2CCC(C#N)C2)CC1]

In some embodiments, R³ is

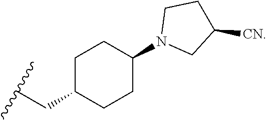 [SMILES: *C[C@H]1CC[C@H](N2CC[C@@H](C#N)C2)CC1]

In some embodiments, R³ is

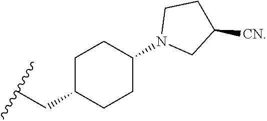 [SMILES: *C[C@H]1CC[C@@H](N2CC[C@@H](C#N)C2)CC1]

In some embodiments, R³ is

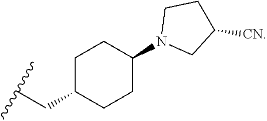 [SMILES: *C[C@H]1CC[C@H](N2CC[C@H](C#N)C2)CC1]

In some embodiments, R³ is

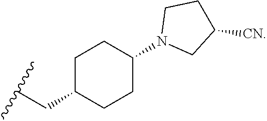 [SMILES: *C[C@H]1CC[C@@H](N2CC[C@H](C#N)C2)CC1]

In some embodiments, R³ is

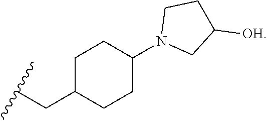 [SMILES: *CC1CCC(N2CCC(O)C2)CC1]

In some embodiments, R³ is

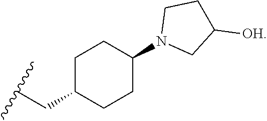 [SMILES: *C[C@H]1CC[C@H](N2CCC(O)C2)CC1]

In some embodiments, R³ is

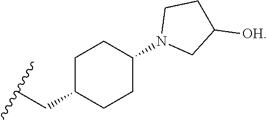 [SMILES: *C[C@H]1CC[C@@H](N2CCC(O)C2)CC1]

In some embodiments, R³ is

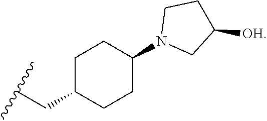 [SMILES: *C[C@H]1CC[C@H](N2CC[C@@H](O)C2)CC1]

In some embodiments, R³ is

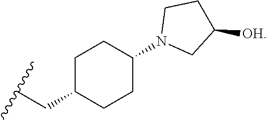 [SMILES: *C[C@H]1CC[C@@H](N2CC[C@@H](O)C2)CC1]

In some embodiments, R³ is

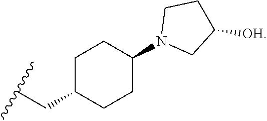 [SMILES: *C[C@H]1CC[C@H](N2CC[C@H](O)C2)CC1]

In some embodiments, R³ is

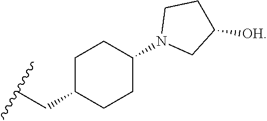 [SMILES: *C[C@H]1CC[C@@H](N2CC[C@H](O)C2)CC1]

In some embodiments, R³ is

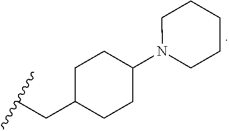 [SMILES: *CC1CCC(N2CCCCC2)CC1]

In some embodiments, R³ is

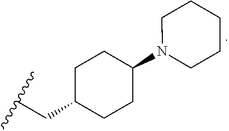 [SMILES: *C[C@H]1CC[C@H](N2CCCCC2)CC1]

In some embodiments, R³ is

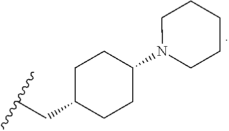 [SMILES: *C[C@H]1CC[C@@H](N2CCCCC2)CC1]

In some embodiments, R³ is

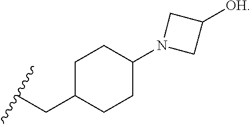 [SMILES: *CC1CCC(N2CC(O)C2)CC1]

In some embodiments, R³ is

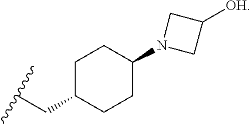 [SMILES: *C[C@H]1CC[C@H](N2CC(O)C2)CC1]

In some embodiments, R³ is

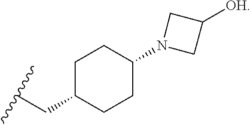 [SMILES: *C[C@H]1CC[C@@H](N2CC(O)C2)CC1]

In some embodiments, R³ is

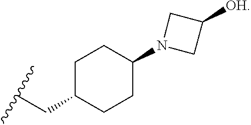

In some embodiments, R³ is

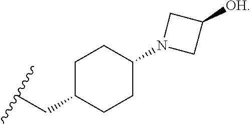

In some embodiments, R³ is

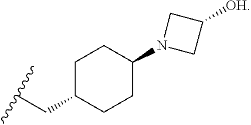

In some embodiments, R³ is

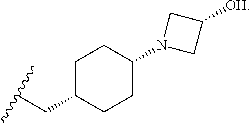

In some embodiments, R³ is

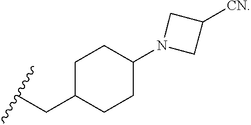 [SMILES: *CC1CCC(N2CC(C#N)C2)CC1]

In some embodiments, R³ is

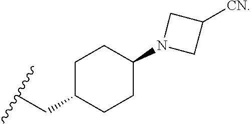 [SMILES: *C[C@H]1CC[C@H](N2CC(C#N)C2)CC1]

In some embodiments, R³ is

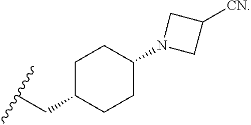 [SMILES: *C[C@H]1CC[C@@H](N2CC(C#N)C2)CC1]

In some embodiments, R³ is

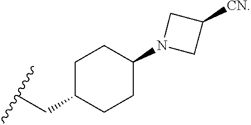

In some embodiments, R³ is

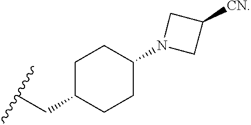

In some embodiments, R³ is

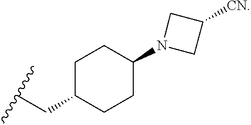

In some embodiments, R³ is

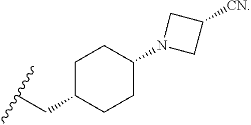

In some embodiments, R³ is

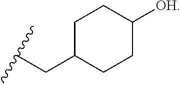 [SMILES: *CC1CCC(O)CC1]

In some embodiments, R³ is

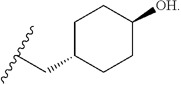 [SMILES: *C[C@H]1CC[C@H](O)CC1]

In some embodiments, R³ is

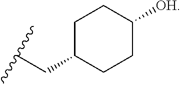 [SMILES: *C[C@H]1CC[C@@H](O)CC1]

In some embodiments, R³ is

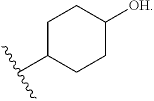 [SMILES: *C1CCC(O)CC1]

In some embodiments, R³ is

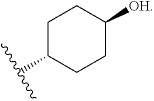 [SMILES: *[C@H]1CC[C@H](O)CC1]

In some embodiments, R³ is

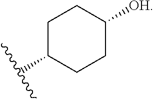 [SMILES: *[C@H]1CC[C@@H](O)CC1]

In some embodiments, R³ is

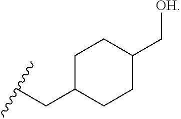 [SMILES: *CC1CCC(CO)CC1]

In some embodiments, R³ is

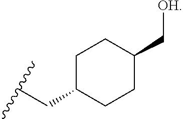 [SMILES: *C[C@H]1CC[C@H](CO)CC1]

In some embodiments, R³ is

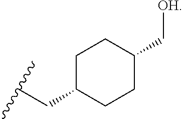 [SMILES: *C[C@H]1CC[C@@H](CO)CC1]

In some embodiments, R³ is

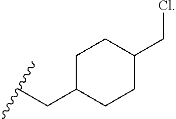 [SMILES: *CC1CCC(CCl)CC1]

In some embodiments, R³ is

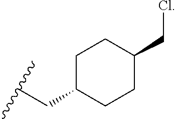 [SMILES: *C[C@H]1CC[C@H](CCl)CC1]

In some embodiments, R³ is

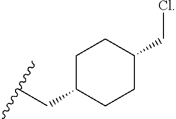 [SMILES: *C[C@H]1CC[C@@H](CCl)CC1]

In some embodiments, R³ is

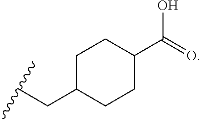 [SMILES: *CC1CCC(C(=O)O)CC1]

In some embodiments, R³ is

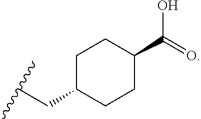 [SMILES: *C[C@H]1CC[C@H](C(=O)O)CC1]

In some embodiments, R³ is

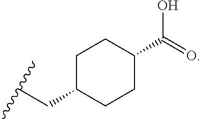 [SMILES: *C[C@H]1CC[C@@H](C(=O)O)CC1]

In some embodiments, R³ is

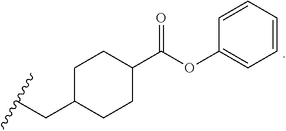 [SMILES: *CC1CCC(C(=O)Oc2ccccc2)CC1]

In some embodiments, R³ is

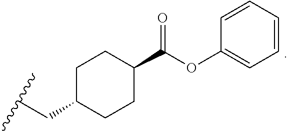 [SMILES: *C[C@H]1CC[C@H](C(=O)Oc2ccccc2)CC1]

In some embodiments, R³ is

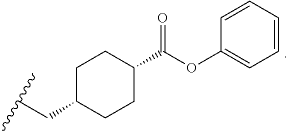 [SMILES: *C[C@H]1CC[C@@H](C(=O)Oc2ccccc2)CC1]

In some embodiments, R³ is

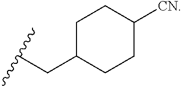 [SMILES: *CC1CCC(C#N)CC1]

In some embodiments, R³ is

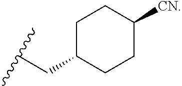 [SMILES: *C[C@H]1CC[C@H](C#N)CC1]

In some embodiments, R³ is

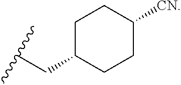 [SMILES: *C[C@H]1CC[C@@H](C#N)CC1]

In some embodiments, R³ is

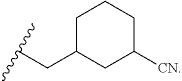 [SMILES: *CC1CCCC(C#N)C1]

In some embodiments, R³ is

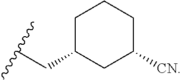 [SMILES: *C[C@@H]1CCC[C@H](C#N)C1]

In some embodiments, R³ is

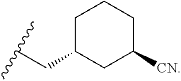 [SMILES: *C[C@@H]1CCC[C@@H](C#N)C1]

In some embodiments, R³ is

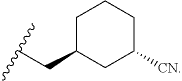 [SMILES: *C[C@H]1CCC[C@H](C#N)C1]

In some embodiments, R³ is

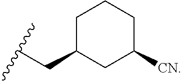 [SMILES: *C[C@H]1CCC[C@@H](C#N)C1]

In some embodiments, R³ is

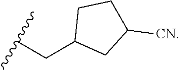 [SMILES: *CC1CCC(C#N)C1]

In some embodiments, R³ is

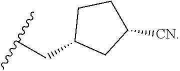 [SMILES: *C[C@@H]1CC[C@H](C#N)C1]

In some embodiments, R³ is

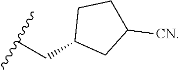 [SMILES: *C[C@@H]1CCC(C#N)C1]

In some embodiments, R³ is

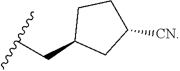 [SMILES: *C[C@H]1CC[C@H](C#N)C1]

In some embodiments, R³ is

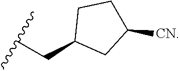 [SMILES: *C[C@H]1CC[C@@H](C#N)C1]

In some embodiments, R³ is

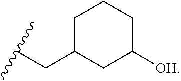 [SMILES: *CC1CCCC(O)C1]

In some embodiments, R³ is

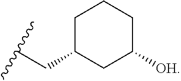 [SMILES: *C[C@@H]1CCC[C@H](O)C1]

In some embodiments, R³ is

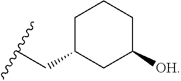 [SMILES: *C[C@@H]1CCC[C@@H](O)C1]

In some embodiments, R³ is

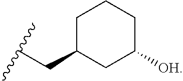 [SMILES: *C[C@H]1CCC[C@H](O)C1]

In some embodiments, R³ is

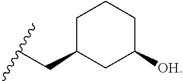 [SMILES: *C[C@H]1CCC[C@@H](O)C1]

In some embodiments, R³ is

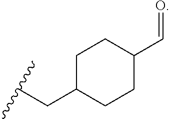 [SMILES: *CC1CCC(C=O)CC1]

In some embodiments, R³ is

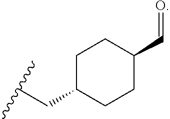 [SMILES: *C[C@H]1CC[C@H](C=O)CC1]

In some embodiments, R³ is

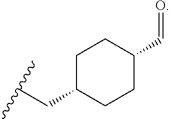 [SMILES: *C[C@H]1CC[C@@H](C=O)CC1]

In some embodiments, R³ is

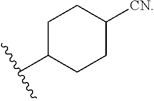 [SMILES: *C1CCC(C#N)CC1]

In some embodiments, R³ is

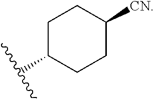 [SMILES: *[C@H]1CC[C@H](C#N)CC1]

In some embodiments, R³ is

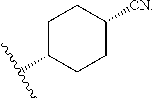 [SMILES: *[C@H]1CC[C@@H](C#N)CC1]

In some embodiments, R³ is

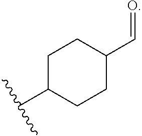 [SMILES: *C1CCC(C=O)CC1]

In some embodiments, R³ is

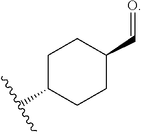 [SMILES: *[C@H]1CC[C@H](C=O)CC1]

In some embodiments, R³ is

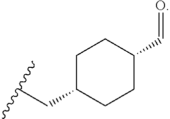 [SMILES: *C[C@H]1CC[C@@H](C=O)CC1]

In some embodiments, R³ is

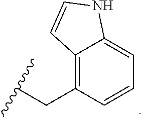 [SMILES: *Cc1cccc2[nH]ccc12]

In some embodiments, R³ is

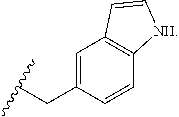 [SMILES: *Cc1ccc2[nH]ccc2c1]

In some embodiments, R³ is

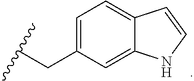 [SMILES: *Cc1ccc2cc[nH]c2c1]

In some embodiments, R³ is

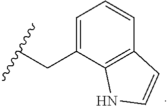 [SMILES: *Cc1cccc2cc[nH]c12]

In some embodiments, R³ is

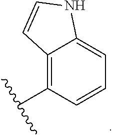 [SMILES: *c1cccc2[nH]ccc12]

In some embodiments, R³ is

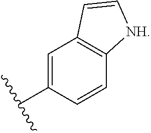 [SMILES: *c1ccc2[nH]ccc2c1]

In some embodiments, R³ is

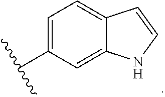 [SMILES: *c1ccc2cc[nH]c2c1]

In some embodiments, R³ is

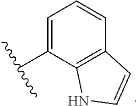 [SMILES: *c1cccc2cc[nH]c12]

In some embodiments, R³ is

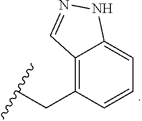 [SMILES: *Cc1cccc2[nH]ncc12]

In some embodiments, R³ is

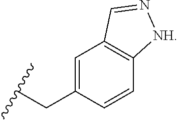 [SMILES: *Cc1ccc2[nH]ncc2c1]

In some embodiments, R³ is

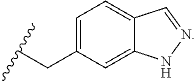 [SMILES: *Cc1ccc2cn[nH]c2c1]

In some embodiments, R³ is

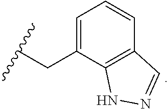 [SMILES: *Cc1cccc2cn[nH]c12]

In some embodiments, R³ is

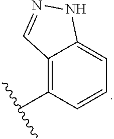 [SMILES: *c1cccc2[nH]ncc12]

In some embodiments, R³ is

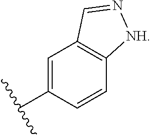 [SMILES: *c1ccc2[nH]ncc2c1]

In some embodiments, R³ is

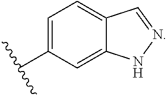 [SMILES: *c1ccc2cn[nH]c2c1]

In some embodiments, R³ is

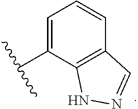 [SMILES: *c1cccc2cn[nH]c12]

In some embodiments, R³ is

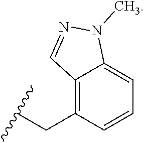 [SMILES: *Cc1cccc2c1cnn2C]

In some embodiments, R³ is

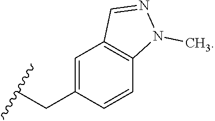 [SMILES: *Cc1ccc2c(cnn2C)c1]

In some embodiments, R³ is

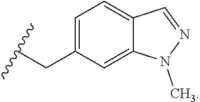 [SMILES: *Cc1ccc2cnn(C)c2c1]

In some embodiments, R³ is

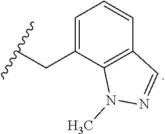 [SMILES: *Cc1cccc2cnn(C)c12]

In some embodiments, R³ is

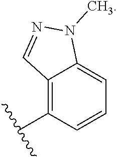 [SMILES: *c1cccc2c1cnn2C]

In some embodiments, R³ is

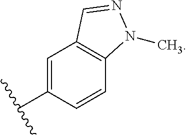 [SMILES: *c1ccc2c(cnn2C)c1]

In some embodiments, R³ is

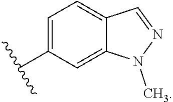 [SMILES: *c1ccc2cnn(C)c2c1]

In some embodiments, R³ is

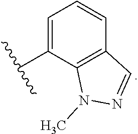 [SMILES: *c1cccc2cnn(C)c12]

In some embodiments, R³ is

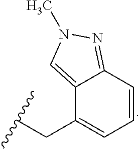 [SMILES: *Cc1cccc2nn(C)cc12]

In some embodiments, R³ is

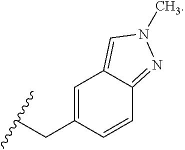 [SMILES: *Cc1ccc2nn(C)cc2c1]

In some embodiments, R³ is

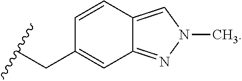 [SMILES: *Cc1ccc2cn(C)nc2c1]

In some embodiments, R³ is

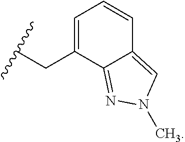 [SMILES: *Cc1cccc2cn(C)nc12]

In some embodiments, R³ is

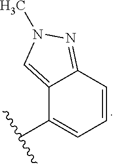 [SMILES: *c1cccc2nn(C)cc12]

In some embodiments, R³ is

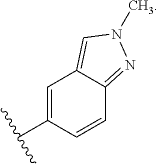 [SMILES: *c1ccc2nn(C)cc2c1]

In some embodiments, R³ is

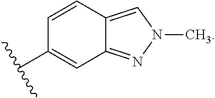 [SMILES: *c1ccc2cn(C)nc2c1]

In some embodiments, R³ is

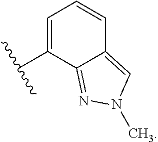 [SMILES: *c1cccc2cn(C)nc12]

In some embodiments, R³ is

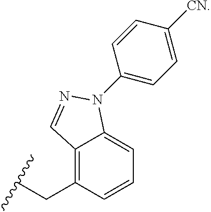 [SMILES: *Cc1cccc2c1cnn2-c1ccc(C#N)cc1]

In some embodiments, R³ is

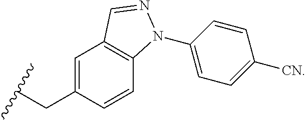 [SMILES: *Cc1ccc2c(cnn2-c2ccc(C#N)cc2)c1]

In some embodiments, R³ is

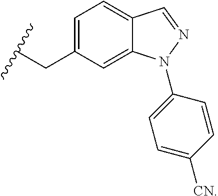 [SMILES: *Cc1ccc2cnn(-c3ccc(C#N)cc3)c2c1]

In some embodiments, R³ is

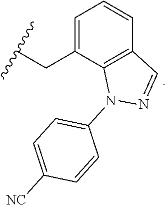 [SMILES: *Cc1cccc2cnn(-c3ccc(C#N)cc3)c12]

In some embodiments, R³ is

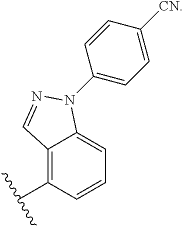 [SMILES: *c1cccc2c1cnn2-c1ccc(C#N)cc1]

In some embodiments, R³ is

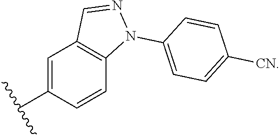 [SMILES: *c1ccc2c(cnn2-c2ccc(C#N)cc2)c1]

In some embodiments, R³ is

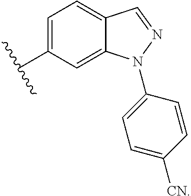 [SMILES: *c1ccc2cnn(-c3ccc(C#N)cc3)c2c1]

In some embodiments, R³ is

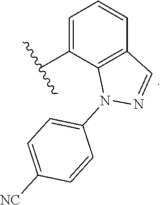 [SMILES: *c1cccc2cnn(-c3ccc(C#N)cc3)c12]

In some embodiments, R³ is

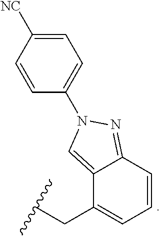 [SMILES: *Cc1cccc2nn(-c3ccc(C#N)cc3)cc12]

In some embodiments, R³ is

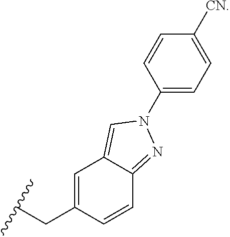 [SMILES: *Cc1ccc2nn(-c3ccc(C#N)cc3)cc2c1]

In some embodiments, R³ is

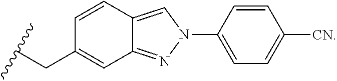 [SMILES: *Cc1ccc2cn(-c3ccc(C#N)cc3)nc2c1]

In some embodiments, R³ is

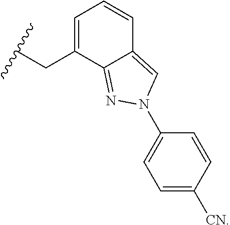 [SMILES: *Cc1cccc2cn(-c3ccc(C#N)cc3)nc12]

In some embodiments, R³ is

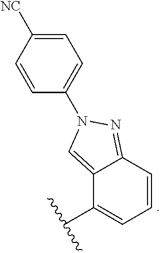 [SMILES: *c1cccc2nn(-c3ccc(C#N)cc3)cc12]

In some embodiments, R³ is

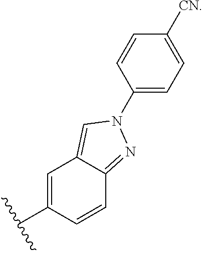 [SMILES: *c1ccc2nn(-c3ccc(C#N)cc3)cc2c1]

In some embodiments, R³ is

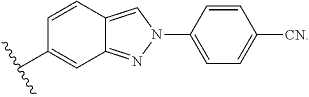 [SMILES: *c1ccc2cn(-c3ccc(C#N)cc3)nc2c1]

In some embodiments, R³ is

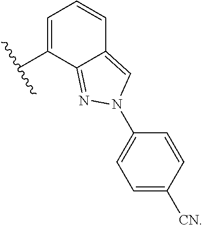 [SMILES: *c1cccc2cn(-c3ccc(C#N)cc3)nc12]

In some embodiments, R³ is

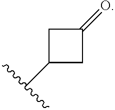 [SMILES: *C1CC(=O)C1]

In some embodiments, R³ is

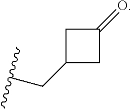 [SMILES: *CC1CC(=O)C1]

In some embodiments, R³ is

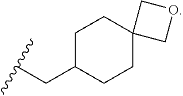 [SMILES: *CC1CCC2(CC1)COC2]

In some embodiments, R³ is

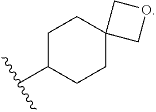 [SMILES: *C1CCC2(CC1)COC2]

In some embodiments, R³ is

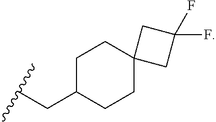 [SMILES: *CC1CCC2(CC1)CC(F)(F)C2]

In some embodiments, R³ is

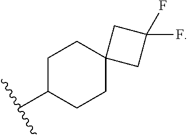 [SMILES: *C1CCC2(CC1)CC(F)(F)C2]

In some embodiments of Formula I, the compound is of one of Formulae (VA)-(VB):

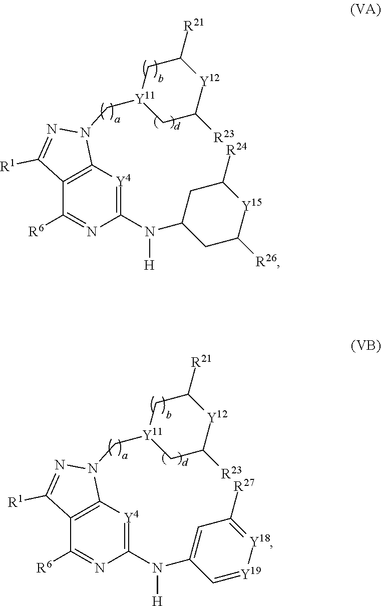

wherein:
Y¹¹ is N or CH;
Y¹² is NR²², C(R^{22′})₂, O or C═O;
Y¹⁵ is NR²⁵ or C(R^{25′})₂;
Y¹⁸ is N or CR²⁸;
Y¹⁹ is N or CR²⁹;
a is 0, 1 or 2;
b is 0 or 1;
d is 0 or 1;
R²¹, R²², each R^{22′}, and R²³ are independently selected from H, (C₁₋₅)alkyl, (C₁₋₅)haloalkyl, OH, (C₁₋₅)alkylene-OH, O(C₁₋₅)alkyl, CHO, CN, CO₂R³², CONR³⁰R³¹, NR³⁰R³¹, halogen, and (C₁₋₅)alkylene-X;
R²⁴, R²⁵, each R^{25′}, and R²⁶ are independently selected from Z, H, OH, (C₁₋₅)alkylene-OH, O(C₁₋₅)alkyl, CHO, CN, CO₂R³², CONR³⁰R³¹, NR³⁰R³¹, halogen, and (C₁₋₅)alkylene-X; and
R²⁷, R²⁸, and R²⁹ are independently selected from Z, H, (C₁₋₅)alkyl, (C₁₋₅)haloalkyl, OH, (C₁₋₅)alkylene-OH, CN, CO₂R³², (C₁₋₅)alkylene-CHO, CHO, CONR³⁰R³¹, NR³⁰R³¹, halogen, and (C₁₋₅)alkylene-X;
R³⁰ and R³¹ are independently H, or (C₁₋₅)alkyl, or R³⁰ and R³¹ are cyclically linked and together with the nitrogen atom to which they are attached provide an optionally substituted heterocyclyl ring;
R³² is H, optionally substituted (C₁₋₅)alkyl, optionally substituted heterocyclyl, or optionally substituted aryl; and
X is halogen.

In some embodiments of Formulae (VA)-(VB), a is 0. In some embodiments, a is 1. In some embodiments, a is 2. In some embodiments, b is 0. In some embodiments, b is 1. In some embodiments, d is 0. In some embodiments, d is 1. In some embodiments of Formulae (VA)-(VB), b+d is 1. In some embodiments, b+d is 2. In some embodiments, b+d is 0.

In some embodiments of Formulae (VA)-(VB), a is 0, and b is 1. In some embodiments, a is 1, and b is 1. In some embodiments, a is 2, and b is 1. In some embodiments, d is 1.

In some embodiments of Formulae (VA)-(VB), R²¹ is H; and R^{22′}, and R²³ are independently selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₅)alkylene-X, where X is halogen.

In some embodiments of Formulae (VA)-(VB), the compound is of one of formula (VIA)-(VIB):

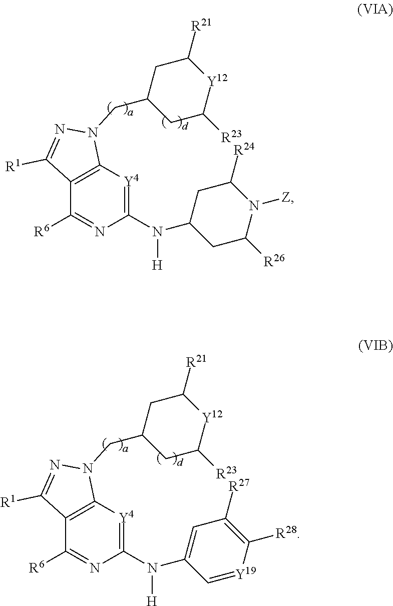

In some embodiments of Formulae (VIA)-(VIB), a is 0. In some embodiments, a is 1. In some embodiments, a is 2. In some embodiments, d is 0. In some embodiments, d is 1.

In some embodiments of Formulae (VIA)-(VIB), a is 0, and d is 1. In some embodiments, a is 1, and d is 1. In some embodiments, a is 2, and d is 1. In some embodiments of Formulae (VIA)-(VIB), a is 0, and d is 0. In some embodiments, a is 1, and d is 0. In some embodiments, a is 2, and d is 0.

In some embodiments of Formulae (VIA)-(VIB), Y¹² is 0. In some embodiments of Formulae (VIA)-(VIB), Y¹² is NR^{22′}. In some embodiments of Formulae (VIA)-(VIB), Y¹² is CHR^{22′}. In some embodiments of Formulae (VIA)-(VIB), R^{22′} is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₅)alkylene-X, where X is halogen.

In some embodiments of Formulae (VIA)-(VIB), R²¹, and R²³ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₅)alkylene-X, where X is halogen. In some embodiments of Formulae (VIA)-(VIB), R²¹ is H.

In some embodiments, the compound is of Formula (VIA), wherein R²⁴ is H; and R²⁶ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X, where X is halogen.

In some embodiments of Formula (VIB), Y¹⁹ is N. In some embodiments of Formula (VIB), Y¹⁹ is CH. In some embodiments of Formula (VIB), Y¹⁹ is CR²⁹. In some embodiments, R²⁹ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X, where X is halogen.

In some embodiments, the compound is of Formula (VIB), wherein: R²⁷ is H or Z; and R²⁸ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X, where X is halogen.

In some embodiments, the compound is of Formula (VIB), wherein: R²⁷ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X, where X is halogen; and R²⁸ is H or Z.

In some embodiments of Formulae (VA)-(VIB), Y⁴ is N. In some embodiments of Formulae (VA)-(VIB), Y⁴ is CR⁴. In some embodiments of Formulae (VA)-(VIB), Y⁴ is CH.

In some embodiments of Formulae (VA)-(VIB), R⁶ is H, CH₃, CF₃, or halogen. In some embodiments of Formulae (VA)-(VIB), R⁶ is H.

In some embodiments of Formulae (VA)-(VIB), R¹ is H, CH₃, CF₃, or halogen. In some embodiments of Formulae (VA)-(VIB), R¹ is H.

In some embodiments of Formulae (VA)-(VIB), the compound includes a group Z or L²-Z. Any convenient Z group described herein can be selected. In some embodiments of Formulae (VA)-(VIB), Z is SO₂R⁸, wherein R⁸ is optionally substituted (C₁-C₆)alkyl, optionally substituted (C₂-C₆)alkenyl, or optionally substituted (C₂-C₆)alkynyl. In some embodiments, R⁸ is (C₁-C₆)alkyl. In some embodiments, R⁸ is methyl. In some embodiments, R⁸ is C₂-C₆ alkenyl. In some embodiments, R⁸ is —CH═CH₂ or —C(CH₃)═CH₂. In some embodiments, R⁸ is C₂-C₆ alkynyl. In some embodiments, R⁸ is —CCH or —CC—CH₃.

In some embodiments of Formulae (VA)-(VIB), the compound includes a Z that is CHO, CH₂CHO, (CH₂)₂CHO, or (CH₂)₃CHO. In some embodiments of Formulae (VA)-(VIB), the compound includes a Z that is Z is OH, CH₂OH, (CH₂)₂OH, or (CH₂)₃OH.

Exemplary CDK (e.g., CDK2) inhibitor compounds of this disclosure are shown in the experimental section and Tables 1 to 3.

In some embodiments, the compound of Formula I is a compound shown in Table 1, or a pharmaceutically acceptable salt thereof. Table 1 illustrates exemplary compounds of Formula (VIA).

TABLE 1
Compounds of Formula (VIA)

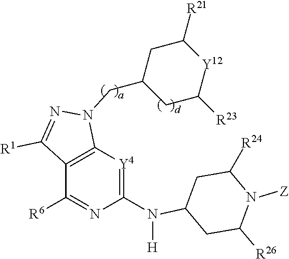

| Cmpd | | | | | | | | | | | |
|---|---|---|---|---|---|---|---|---|---|---|---|
| # | Z | R²⁴ | R²⁶ | Y⁴ | Y¹² | R²¹ | R²³ | a | d | R¹ | R⁶ |
| 1 | SO₂CH═CH₂ | H | H | N | CH₂ | H | H | 1 | 1 | H | H |
| 2 | SO₂CH₃ | H | H | N | CH₂ | H | H | 1 | 1 | H | H |
| 56 | SO₂CH₃ | H | H | N | CHN(CH₃)₂ | H | H | 1 | 1 | H | H |

In some embodiments, the compound of Formula I is a compound shown in Table 2, or a pharmaceutically acceptable salt thereof. Table 2 illustrates exemplary compounds of Formula (VIB).

TABLE 2
Compounds of Formula (VIB)

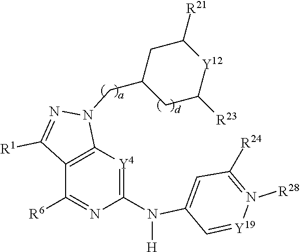

| Cmpd # | Y¹⁹ | R²⁷ | R²⁸ | Y⁴ | Y¹² | R²¹ | R²³ | a | d | R¹ | R⁶ |
|---|---|---|---|---|---|---|---|---|---|---|---|
| 3 | CH | H | SO₂CH₃ | N | O | H | H | 0 | 1 | H | H |
| 6 | CH | H | SO₂CH₃ | N | CHNH₂ | H | H | 1 | 1 | H | H |
| 7 | CH | H | SO₂CH₃ | N | NH | H | H | 1 | 1 | H | H |
| 8 | CH | H | SO₂CH═CH₂ | N | CH₂ | H | H | 1 | 1 | H | H |
| 11 | CH | H | SO₂CH₃ | N | CHCH₂OH | H | H | 1 | 1 | H | H |
| 12 | CH | H | SO₂CH₃ | N | CH₂ | H | H | 1 | 1 | H | H |
| 13 | CH | H | SO₂CH₃ | N | CH₂ | H | H | 1 | 0 | H | H |
| 14 | CH | H | SO₂CH₃ | N | NH | H | H | 0 | 1 | H | H |
| 16 | CH | H | SO₂CH₃ | N | CHCO₂H | H | H | 1 | 1 | H | H |
| 17 | CH | H | SO₂CH₃ | N | CHCH₂OH | H | H | 1 | 1 | H | H |
| 18 | CH | H | SO₂CH₃ | N | CHC(O)O(4- | H | H | 1 | 1 | H | H |
| | | | | | fluorophenyl) | | | | | | |
| 19 | CH | H | SO₂CH₃ | N | CH₂ | H | H | 1 | 1 | CH₃ | H |
| 20 | CH | CH₂OH | H | N | CH₂ | H | H | 1 | 1 | H | H |
| 22 | CH | H | (CH₂)₂OH | N | CH₂ | H | H | 1 | 1 | H | H |
| 23 | CH | CH₃ | SO₂CH═CH₂ | N | CH₂ | H | H | 1 | 1 | H | H |
| 24 | CH | H | CH₂OH | N | CH₂ | H | H | 1 | 1 | H | H |
| 25 | CH | H | CHO | N | CH₂ | H | H | 1 | 1 | H | H |
| 26 | CH | CHO | H | N | CH₂ | H | H | 1 | 1 | H | H |
| 27 | CH | H | SO₂CH₃ | N | CHCHO | H | H | 1 | 1 | H | H |
| 28 | CH | H | SO₂CH₃ | N | CHCN | H | H | 1 | 1 | H | H |
| 30 | CH | H | SO₂C(CH₃)═CH₂ | N | CH₂ | H | H | 1 | 1 | H | H |
| 31 | N | C1 | SO₂CH═CH₂ | N | CH₂ | H | H | 1 | 1 | H | H |
| 32/33 | CH | H | SO₂CH₃ | N | CHCN | H | H | 0 | 1 | H | H |
| 34 | N | H | SO₂CH═CH₂ | N | CH₂ | H | H | 1 | 1 | H | H |
| 35 | CH | (CH₂)₂OH | H | N | CH₂ | H | H | 1 | 1 | H | H |
| 36 | CH | F | SO₂CH═CH₂ | N | CH₂ | H | H | 1 | 1 | H | H |
| 37 | CF | F | SO₂CH═CH₂ | N | CH₂ | H | H | 1 | 1 | H | H |
| 38 | CH | H | COC═CH | N | CH₂ | H | H | 1 | 1 | H | H |
| 40 | CH | H | SO₂CH₃ | N | CHN(CH₃)₂ | H | H | 1 | 1 | H | H |
| 41 | CH | H | SO₂CH₃ | N | oxetane | H | H | 0 | 1 | H | H |
| 42 | CH | CF₃ | SO₂CH═CH₂ | N | CH₂ | H | H | 1 | 1 | H | H |
| 43 | CH | H | SO₂CC—CH₃ | N | CH₂ | H | H | 1 | 1 | H | H |
| 44 | CH | H | COCH═CH₂ | N | CH₂ | H | H | 1 | 1 | H | H |
| 45 | CH | H | SO₂CH₃ | N | CHNHCH₃ | H | H | 1 | 1 | H | H |
| 46 | CH | H | SO₂CH₃ | N | CH₂ | CN | H | 0 | 1 | H | H |
| 47 | CH | H | SO₂CH₃ | N | CH₂ | CN | H | 0 | 0 | H | H |
| 48/49 | CH | H | SO₂CH₃ | N | CHCH₂Cl | H | H | 1 | 1 | H | H |
| 50 | CH | H | SO₂CH₃ | N | CH₂ | OH | H | 0 | 0 | H | H |
| 51 | CH | H | SO₂CH₃ | N | CH₂ | OH | H | 0 | 1 | H | H |
| 52 | CH | H | SO₂CH₃ | N | CH₂ | CN | H | 0 | 1 | H | H |
| 53 | CH | H | SO₂CH₃ | N | C═O | H | H | 0 | 1 | H | H |
| 54 | CH | H | SO₂CH₃ | N | CHOH | H | H | 0 | 1 | H | H |
| 55 | CH | CH₂OH | OH | N | CHOH | H | H | 0 | 1 | H | H |
| 58/59 | CH | H | SO₂CH₃ | N | CH(N- | H | H | 1 | 1 | H | H |
| | | | | | pyrrolidinyl) | | | | | | |
| 60/61 | CH | H | SO₂CH₃ | N | CH(N- | H | H | 1 | 1 | H | H |
| | | | | | azetidinyl) | | | | | | |
| 63 | CH | H | SO₂CH₃ | N | O | H | H | 1 | 1 | H | H |
| 64/66 | CH | H | SO₂CH₃ | N | CH(3-HO-N- | H | H | 1 | 1 | H | H |
| | | | | | azeidinyl) | | | | | | |
| 65/67 | CH | H | SO₂CH₃ | N | CH(3- | H | H | 1 | 1 | H | H |
| | | | | | cyano-N- | | | | | | |
| | | | | | azeidinyl) | | | | | | |
| 69/70 | CH | H | SO₂CH₃ | N | CH((S)-3- | H | H | 1 | 1 | H | H |
| | | | | | cyano-N- | | | | | | |
| | | | | | pyrrolidinyl) | | | | | | |
| 71/72 | CH | H | SO₂CH₃ | N | CH((R)-3- | H | H | 1 | 1 | H | H |
| | | | | | cyano-N- | | | | | | |
| | | | | | pyrrolidinyl) | | | | | | |
| 73 | CH | H | SO₂CH₃ | N | CH((R)-3- | H | H | 1 | 1 | H | H |
| | | | | | HO-N- | | | | | | |
| | | | | | pyrrolidinyl) | | | | | | |
| 74 | CH | H | SO₂CH₃ | N | CH((S)-3- | H | H | 1 | 1 | H | H |
| | | | | | HO-N- | | | | | | |
| | | | | | pyrrolidinyl) | | | | | | |
| 75 | CH | H | SO₂CH₃ | N | CH(N- | H | H | 1 | 1 | H | H |
| | | | | | methyl, N- | | | | | | |
| | | | | | ethyl) | | | | | | |
| 79 | CH | CF₃ | SO₂CH₃ | N | CH₂ | H | H | 1 | 1 | H | H |
| 80 | CH | F | SO₂CH₃ | N | CH₂ | H | H | 1 | 1 | H | H |
| 84 | CH | H | SO₂CH₃ | N | CH(N- | H | H | 1 | 1 | H | H |
| | | | | | methyl, N- | | | | | | |
| | | | | | ethanol) | | | | | | |

In certain embodiments, the compound is described by the structure of one of the compounds of Tables 1-9. It is understood that any of the compounds shown in Tables 1-9 may be present in a salt form. In some cases, the salt form of the compound is a pharmaceutically acceptable salt.

“Pharmaceutically acceptable salt” includes both acid and base addition salts. Compounds included in the present compositions, that are acidic in nature are capable of forming base salts with various pharmacologically acceptable cations Salts formed with free carboxyl groups can also be derived from inorganic bases such as, for example, sodium, potassium, lithium, ammonium, calcium, magnesium, iron, zinc, copper, manganese, aluminum salts, and the like. Pharmaceutically acceptable salts include the acid addition salts (e.g., formed with the free amino groups of the compound) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, and the like. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above.

The term “solvate” refers to a crystalline solid that contains one or more molecules of solvent among the crystalline assembly (stoichiometrically or nonstoichiometrically). For example, a “hydrate” is a special case solvate where the solvent molecule associated with the crystalline assembly is a water molecule. It is understood that any of the compounds shown in Tables 1-9 may be present as solvates or hydrates. Such compounds may be useful as, for example, analytical tools, probes in biological assays, or therapeutic agents.

The term “prodrug” refers to an agent which is converted into the drug in vivo by some physiological or chemical process (e.g., a prodrug on being brought to the physiological pH is converted to the desired drug form). It is understood that a prodrug of a compound of the present disclosure can have different pharmacokinetic properties (e.g., bioavailability), from pharmacodynamically active form of the compound. It is understood that any of the compounds shown in Tables 1-9 may be present in a prodrug form.

The present disclosure includes compounds (e.g., as described herein, such as Tables 1-9) with at least one desired isotopic substitution of an atom, at an amount above the natural abundance of the isotope, i.e., isotopically enriched atoms. Isotopes are atoms having the same atomic number but different mass numbers, i.e., the same number of protons but a different number of neutrons. For example, compounds having the present structures except for the replacement of hydrogen by deuterium (²H) or tritium (³H), or the replacement of a carbon by a ¹³C- or ¹⁴C-carbon atom are within the scope of this disclosure. Such compounds may be useful as, for example, analytical tools, probes in biological assays, or therapeutic agents.

Examples of isotopes that can be incorporated into compounds of the invention include isotopes of hydrogen, carbon, nitrogen, oxygen, phosphorous, fluorine, chlorine and iodine such as ²H, ³H, ¹¹C, ¹³C, ¹⁴C, ¹⁵N, ¹⁸F ³¹P, ³²P, ³⁵S, ³⁶Cl, and ¹²⁵I respectively. In one non-limiting embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in treatment of patients. In particular, an ¹⁸F labeled compound may be particularly desirable for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In some embodiments, the substitution of a hydrogen atom for a deuterium atom can be provided in any compound of Formulas α, β, γ, δ, ε, I, IA-IU, and IIA-VIB, such as a compound of Tables 1-4. In one non-limiting embodiment, the substitution of a hydrogen atom for a deuterium atom occurs within one or more groups selected from any of R¹, R², R³, R⁴, R⁵, R⁶, R⁷, R⁸, R⁹, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R, R′, and R″ etc. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle the unsubstituted carbons may be deuterated.

### 3.2 Pharmaceutical Compositions

Also provided herein is a pharmaceutical composition comprising the subject CDK2 inhibitor compounds and a pharmaceutically acceptable carrier or excipient.

A “pharmaceutical composition” is meant to encompass a composition suitable for administration to a subject, such as a mammal, especially a human. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

In general, the compositions of the disclosure will include and be administered in a therapeutically effective amount by the desired mode of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

The terms “effective amount,” “pharmaceutically effective amount,” or “therapeutically effective amount” as used herein mean a sufficient amount of the composition to provide the desired utility when administered to a subject having a particular condition. The term “therapeutically effective amount” therefore refers to an amount of therapeutic cells or a composition having therapeutic cells that is sufficient to promote a particular effect when administered to a subject in need of treatment. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. “Pharmaceutically acceptable carrier or excipient” can encompass substances referred to as pharmaceutically acceptable diluents, pharmaceutically acceptable additives, and pharmaceutically acceptable carriers.

A pharmaceutically acceptable carrier, diluent or excipient for therapeutic use are well known in the pharmaceutical art, and are described, for example, in Remington's Pharmaceutical Sciences, 18th Edition (Easton, Pa.: Mack Publishing Company, 1990), and include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Carriers include excipients must be of sufficiently high purity and sufficiently low toxicity to render them suitable for administration to the patient being treated. The carrier can be inert or it can possess pharmaceutical benefits of its own. The amount of carrier employed in conjunction with the disclosed compound is sufficient to provide a practical quantity of material for administration per unit dose of the compound, as described in more detail herein.

Polymorphs and/or solvates of compounds of the present disclosure can be administered alone or as an active ingredient of a formulation. Thus, the present disclosure also includes pharmaceutical compositions of polymorphs and solvates of the invention, containing, for example, one or more pharmaceutically acceptable carriers.

Numerous standard references are available that describe procedures for preparing various formulations suitable for administering the compounds according to the invention. Examples of potential formulations and preparations are contained, for example, in the Handbook of Pharmaceutical Excipients, American Pharmaceutical Association (current edition); Pharmaceutical Dosage Forms: Tablets (Lieberman, Lachman and Schwartz, editors) current edition, published by Marcel Dekker, Inc., as well as Remington's Pharmaceutical Sciences (Arthur Osol, editor), 1553-1593 (current edition).

Administration of the polymorphs and solvates of the present disclosure may be accomplished according to patient needs, for example, orally, nasally, parenterally (subcutaneously, intravenously, intramuscularly, intrasternally and by infusion) by inhalation, rectally, vaginally, topically and by ocular administration.

Various solid oral dosage forms can be used for administering the polymorphs and solvates of the compounds of the present disclosure, including such solid forms as tablets, gelcaps, capsules, caplets, granules, lozenges and bulk powders. The polymorphs and solvates of compounds of the present disclosure can be administered alone or combined with various pharmaceutically acceptable carriers, diluents (such as sucrose, mannitol, lactose, starches) and excipients known in the art, including but not limited to suspending agents, solubilizers, buffering agents, binders, disintegrants, preservatives, colorants, flavorants, lubricants and the like. Time release capsules, tablets and gels are also advantageous in administering the compounds of the present disclosure.

Various liquid oral dosage forms can also be used for administering the polymorphs and solvates of compounds of the present disclosure, including aqueous and non-aqueous solutions, emulsions, suspensions, syrups, and elixirs. Such dosage forms can also contain suitable inert diluents known in the art such as water and suitable excipients known in the art such as preservatives, wetting agents, sweeteners, flavorants, as well as agents for emulsifying and/or suspending the compounds of the invention. The polymorphs and solvates of compounds of the present disclosure may be injected, for example, intravenously, in the form of an isotonic sterile solution. Other preparations are also possible.

Suppositories for rectal administration of the polymorphs and solvates of compounds of the present disclosure can be prepared by mixing the compound with a suitable excipient such as cocoa butter, salicylates and polyethylene glycols. Formulations for vaginal administration can be in the form of a pessary, tampon, cream, gel, past foam, or spray formula containing, in addition to the active ingredient, such suitable carriers as are known in the art.

For topical administration, a pharmaceutical composition can be in the form of creams, ointments, liniments, lotions, emulsions, suspensions, gels, solutions, pastes, powders, sprays, and drops suitable for administration to the skin, eye, ear, or nose. Topical administration may also involve transdermal administration via means such as transdermal patches.

Aerosol formulations suitable for administering via inhalation also can be made. For example, for treatment of disorders of the respiratory tract, the compounds according to the present disclosure can be administered by inhalation in the form of a powder (e.g., micronized) or in the form of atomized solutions or suspensions. The aerosol formulation can be placed into a pressurized acceptable propellant.

### 3.3 Methods of Use

Also provided herein are methods for using the subject CDK2 inhibitor compounds and pharmaceutical compositions in research and as therapeutics.

a. Methods of Inhibiting CDK2

As illustrated in the working examples the subject compounds can inhibit cyclin-dependent kinase 2 (CDK2). By inhibiting a CDK2 it is meant that the activity of the enzyme is decreased by 10% or more, such as 20% or more, 30% or more, 40% or more, 50% or more, 60% or more, 70% or more, 80% or more, 90% or more, 95% or more (e.g., relative to a control in any convenient in vitro inhibition assay). In some cases, inhibiting a CDK2 means decreasing the activity of the enzyme by a factor of 2 or more, such as 3 or more, 5 or more, 10 or more, 100 or more, or 1000 or more, relative to its normal activity (e.g., relative to a control as measured by any convenient assay).

In some cases, the method is a method of inhibiting CDK2 in a sample. The term “sample” as used herein relates to a material or mixture of materials, typically, although not necessarily, in fluid form, containing one or more components of interest.

Accordingly, in some embodiments there is provided a method of inhibiting cyclin-dependent kinase 2 (CDK2), the method comprising contacting a biological sample containing CDK2 with an effective amount of a subject compound or a pharmaceutical composition, as described herein, to inhibit CDK2.

In some embodiments, there is provided a method of inhibiting CDK2, the method comprising contacting a sample with a CDK2 inhibitor to inhibit activity of CDK2. In some cases, the sample is a cellular sample.

In certain embodiments the CDK2 inhibitor is an inhibitor as defined herein. In some embodiments, the CDK2 inhibitor is an inhibitor according to any one of formulas I, IIA, IIB, IIIA, IIIB, IVA, IVB, IVC, IVD, VA, VB or VI. In some cases, the CDK2 inhibitor is any compound illustrated in Tables 1-9, or a pharmaceutically acceptable salt thereof. In some cases, the CDK2 inhibitor is any one of compounds 1-84, or a pharmaceutically acceptable salt thereof.

In some embodiments the CDK2 inhibitor is a covalent inhibitor. In some embodiments, there is provided a method of inhibiting CDK2, the method comprising contacting a sample with a CDK2 inhibitor that covalently binds to CDK2 to inhibit CDK2. In some embodiments, the CDK2 inhibitor covalently binds to a lysine residue of CDK2 to inhibit CDK2.

In some embodiments the CDK2 inhibitor is a classic inhibitor. In some embodiments, there is provided a method of inhibiting CDK2, the method comprising contacting a sample with a CDK2 inhibitor that associates with CDK2 to inhibit CDK2.

In some embodiments the CDK2 inhibitor is a covalent reversible inhibitor. In some embodiments, there is provided a method of inhibiting CDK2, the method comprising contacting a sample with a CDK2 inhibitor that reversibly covalently binds to CDK2 to inhibit CDK2.

In some embodiments, the subject compounds have a CDK2 inhibition profile that reflects activity against additional enzymes. In some embodiments, the subject compounds specifically inhibit CDK2 without undesired inhibition of one or more other enzymes. In some embodiments, the subject compounds specifically inhibit CDK2 without undesired inhibition of CDK1.

In some embodiments, the subject compounds inhibit CDK2, as determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

As summarized above, aspects of the disclosure include methods of inhibiting CDK2. A subject compound (e.g., as described herein) may inhibit at activity of CDK2 in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10^{″6} M or less (e.g., 1×10^{″6} M or less, 1×10^{″7} M or less, 1×10^{″8} M or less, 1×10⁻⁹ M or less, 1×10¹⁰ M or less, or 1×10¹¹ M or less).

The protocols that may be employed in determining CDK2 activity are numerous, and include but are not limited to cell-free assays, e.g., binding assays; assays using purified enzymes, cellular assays in which a cellular phenotype is measured, e.g., gene expression assays; and in vivo assays that involve a particular animal (which, in certain embodiments may be an animal model for a condition related to the target pathogen).

In some embodiments, the subject method is an in vitro method that includes contacting a sample with a subject compound that specifically inhibits CDK2. In certain embodiments, the sample is suspected of containing CDK2 and the subject method further includes evaluating whether the compound inhibits CDK2.

In certain embodiments, the subject compound is a modified compound that includes a label, e.g., a fluorescent label, and the subject method further includes detecting the label, if present, in the sample, e.g., using optical detection.

In certain embodiments, the compound is modified with a support or with affinity groups that bind to a support (e.g., biotin), such that any sample that does not bind to the compound may be removed (e.g., by washing). The specifically bound CDK2, if present, may then be detected using any convenient means, such as, using the binding of a labeled target specific probe, or using a fluorescent protein reactive reagent.

In another embodiment of the subject method, the sample is known to contain CDK2.

In some embodiments, the method is a method of reducing cancer cell proliferation, where the method includes contacting the cell with an effective amount of a subject CDK2 inhibitor compound (e.g., as described herein) to reduce cancer cell proliferation. The method can be performed in combination with a chemotherapeutic agent (e.g., as described herein). The cancer cells can be in vitro or in vivo. In certain instances, the method includes contacting the cell with a CDK2 inhibitor compound (e.g., as described herein) and contacting the cell with a chemotherapeutic agent. Any convenient cancer cells can be targeted.

In some embodiments, there is provided a method of inhibiting CDK2 in a subject, comprising administering to the subject a compound or a pharmaceutical composition, as described herein, to inhibit CDK2 in the subject.

b. Methods of Treatment

Compounds of the present disclosure can inhibit a CDK, such as CDK2, or CDK4, and therefore are useful for treating diseases wherein the underlying pathology is, wholly or partially, mediated by the CDK, such as CDK2 or CDK4. Accordingly, provided herein is a method of treating a disease or disorder associated with CDK2 in a subject, comprising administering to the subject suffering from a disease or disorder associated with CDK2 a therapeutically effective amount of a compound, or a pharmaceutical composition as described herein.

Such diseases or disorders associated with CDK2 include cancer and other diseases of proliferation disorders. In some embodiments, the present disclosure provides treatment of an individual or a patient in vivo using a subject compound (e.g., a compound of Formula (I) or a salt or stereoisomer thereof) such that growth of cancerous tumors is inhibited. A subject CDK2 inhibitor compound can be used to inhibit the growth of cancerous tumors with aberrations that activate the CDK2 kinase activity. These include, but are not limited to, disease (e.g., cancers) that are characterized by amplification or overexpression of CCNE1.

In certain embodiments the CDK2 inhibitor is an inhibitor as defined herein. In some embodiments, the CDK2 inhibitor is an inhibitor according to any one of formulas described herein (e.g., Formula I-VIIB). In some cases, the CDK2 inhibitor is a compound illustrated in any of Tables 1-9, or a pharmaceutically acceptable salt thereof. In some cases, the CDK2 inhibitor is any one of compounds 1-84, or an isotopically labelled version thereof, or a stereoisomer thereof, or a prodrug thereof, and/or a pharmaceutically acceptable salt thereof.

Accordingly, in some embodiments of the methods, the patient has been previously determined to have an amplification of the cyclin E1 (CCNE1) gene and/or an expression level of CCNE1 in a biological sample obtained from the human subject that is higher than a control expression level of CCNE1. Alternatively, a subject CDK2 inhibitor compound can be used in conjunction with other agents or standard cancer treatments.

In one embodiment, the present disclosure provides a method for inhibiting growth of tumor cells in vitro. The method includes contacting the tumor cells in vitro with a subject compound. In another embodiment, the present disclosure provides a method for inhibiting growth of tumor cells with CCNE1 amplification and overexpression in an individual or a patient. The method includes administering to the individual or patient in need thereof a therapeutically effective amount of a subject compound.

In some embodiments, provided herein is a method of inhibiting CDK2, comprising contacting the CDK2 with a subject CDK2 inhibitor compound. In some embodiments, provided herein is a method of inhibiting CDK2 in a patient, comprising administering to the patient a compound of Formula (I) or any of the Formulae as described herein, a compound as recited in any of the claims and described herein, or a salt thereof.

In some embodiments, provided herein is a method for treating cancer. The method includes administering to a patient (in need thereof), a therapeutically effective amount of a subject CDK2 inhibitor compound. In another embodiment, the cancer is characterized by amplification or overexpression of CCNE1.

In some embodiments, provided herein is a method of treating a disease or disorder associated with CDK2 in a patient, comprising administering to the patient a therapeutically effective amount of a subject CDK2 inhibitor compound. In some embodiments, the disease or disorder associated with CDK2 is associated with an amplification of the cyclin E1 (CCNE1) gene and/or overexpression of CCNE1. In some embodiments, the disease or disorder associated with CDK2 is N-myc amplified neuroblastoma cells, K-Ras mutant lung cancers, and cancers with FBXW7 mutation and CCNE1 overexpression.

### 3.4 Utility

The subject methods and compositions, e.g., as described above, can be used in any application where CDK2 inhibition is desired. Applications of interest include both research and therapeutic applications. Applications of interest include, but are not limited to: research applications, diagnostic applications and therapeutic applications.

In some instances, the application of interest is a therapeutic application, for example, in the treatment of a disease. For example, the compositions and methods of the present application may be used to deliver a subject CDK2 inhibitor to a cell to treat a disease or condition where the underlying pathology is, wholly or partially, mediated by CDK2. As one nonlimiting example, compositions of the present application may be used in the treatment of diseases or disorders associated with CDK2 such as cancer and other diseases with proliferation disorder.

### 3.5 Definitions

Unless defined otherwise, all technical and scientific terms used herein have the same meaning as is commonly understood by one of skill in the art to which this invention belongs.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Compounds are described using standard nomenclature. The compounds in any of the formulas described herein may be in the form of a racemate, enantiomer, mixture of enantiomers, diastereomer, mixture of diastereomers, tautomer, N-oxide, isomer; such as rotamer, as if each is specifically described unless specifically excluded by context.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

A dash (“-”) that is not between two letters or symbols is used to indicate a point of attachment for a substituent. For example, —(C═O)NH₂ is attached through carbon of the carbonyl (C═O) group.

“Aliphatic” refers to a saturated or unsaturated, straight, branched, or cyclic hydrocarbon. “Aliphatic” is intended herein to include, but is not limited to, alkyl, alkenyl, alkynyl, cycloalkyl, cycloalkenyl, and cycloalkynyl moieties, and thus incorporates each of these definitions. In one embodiment, “aliphatic” is used to indicate those aliphatic groups having 1-20 carbon atoms. The aliphatic chain can be, for example, mono-unsaturated, di-unsaturated, tri-unsaturated, or polyunsaturated, or alkynyl. Unsaturated aliphatic groups can be in a cis or trans configuration. In one embodiment, the aliphatic group contains from 1 to about 12 carbon atoms, more generally from 1 to about 6 carbon atoms or from 1 to about 4 carbon atoms. In one embodiment, the aliphatic group contains from 1 to about 8 carbon atoms. In certain embodiments, the aliphatic group is C₁-C₂, C₁-C₃, C₁-C₄, C₁-C₅ or C₁-C₆. The specified ranges as used herein indicate an aliphatic group having each member of the range described as an independent species. For example, the term C₁-C₆ aliphatic as used herein indicates a straight or branched alkyl, alkenyl, or alkynyl group having from 1, 2, 3, 4, 5, or 6 carbon atoms and is intended to mean that each of these is described as an independent species. For example, the term C₁-C₄ aliphatic as used herein indicates a straight or branched alkyl, alkenyl, or alkynyl group having from 1, 2, 3, or 4 carbon atoms and is intended to mean that each of these is described as an independent species. In one embodiment, the aliphatic group is substituted with one or more functional groups that results in the formation of a stable moiety.

“Alkyl” is a branched or straight chain saturated aliphatic hydrocarbon group. In one non-limiting embodiment, the alkyl group contains from 1 to about 12 carbon atoms, more generally from 1 to about 6 carbon atoms or from 1 to about 4 carbon atoms. In one non-limiting embodiment, the alkyl contains from 1 to about 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₆ alkyl as used herein indicates a straight or branched alkyl group having from 1, 2, 3, 4, 5, or 6 carbon atoms and is intended to mean that each of these is described as an independent species and therefore each subset is considered separately disclosed. For example, the term C₁-C₄ alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In an alternative embodiment, the alkyl group is optionally substituted. The term “alkyl” also encompasses cycloalkyl or carbocyclic groups. For example, when a term is used that includes “alk” then “cycloalkyl” or “carbocyclic” can be considered part of the definition, unless unambiguously excluded by the context. For example and without limitation, the terms alkyl, alkoxy, haloalkyl, etc. can all be considered to include the cyclic forms of alkyl, unless unambiguously excluded by context.

“Alkenyl” is a linear or branched aliphatic hydrocarbon groups having one or more carbon-carbon double bonds that may occur at a stable point along the chain. The specified ranges as used herein indicate an alkenyl group having each member of the range described as an independent species, as described above for the alkyl moiety. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. In an alternative embodiment, the alkenyl group is optionally substituted. The term “Alkenyl” also encompasses cycloalkyl or carbocyclic groups possessing at least one point of unsaturation.

“Alkynyl” is a branched or straight chain aliphatic hydrocarbon group having one or more carbon-carbon triple bonds that may occur at any stable point along the chain. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. In an alternative embodiment, the alkynyl group is optionally substituted. The term “Alkynyl” also encompasses cycloalkyl or carbocyclic groups possessing at least one triple bond.

“Alkylene” is a bivalent saturated hydrocarbon. Alkylenes, for example, can be a 1, 2, 3, 4, 5, 6, 7 to 8 carbon moiety, 1 to 6 carbon moiety, or an indicated number of carbon atoms, for example C₁-C₂alkylene, C₁-C₃alkylene, C₁-C₄alkylene, C₁-C₆alkylene, or C₁-C₆alkylene.

“Alkenylene” is a bivalent hydrocarbon having at least one carbon-carbon double bond. Alkenylenes, for example, can be a 2 to 8 carbon moiety, 2 to 6 carbon moiety, or an indicated number of carbon atoms, for example C₂-C₄alkenylene.

“Alkynylene” is a bivalent hydrocarbon having at least one carbon-carbon triple bond. Alkynylenes, for example, can be a 2 to 8 carbon moiety, 2 to 6 carbon moiety, or an indicated number of carbon atoms, for example C₂-C₄alkynylene.

The term “amino” refers to the group —NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

“Chain” indicates a linear chain to which all other chains, long or short or both, may be regarded as being pendant. Where two or more chains could equally be considered to be the main chain, “chain” refers to the one which leads to the simplest representation of the molecule.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

“Halo” and “halogen” refers to fluorine, chlorine, bromine or iodine.

“Haloalkyl” is a branched or straight-chain alkyl groups substituted with 1 or more halo atoms described above, up to the maximum allowable number of halogen atoms. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include but are not limited to, trifluoromethyl and pentafluoroethyl.

“Haloalkoxy” indicates a haloalkyl group as defined herein attached through an oxygen bridge (oxygen of an alcohol radical).

The term “heteroaliphatic” refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In one embodiment, the only heteroatom is nitrogen. In one embodiment, the only heteroatom is oxygen. In one embodiment, the only heteroatom is sulfur. “Heteroaliphatic” is intended herein to include, but is not limited to, heteroalkyl, heteroalkenyl, heteroalkynyl, heterocycloalkyl, heterocycloalkenyl, and heterocycloalkynyl moieties. In one embodiment, “heteroaliphatic” is used to indicate a heteroaliphatic group (cyclic, acyclic, substituted, unsubstituted, branched or unbranched) having 1-20 carbon atoms. In one embodiment, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

“Heterocycloalkyl” is an alkyl group as defined herein substituted with a heterocyclo group as defined herein.

“Arylalkyl” is an alkyl group as defined herein substituted with an aryl group as defined herein.

“Heteroarylalkyl” is an alkyl group as defined herein substituted with a heteroaryl group as defined herein.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. The one or more fused carbocyclyl or heterocyclyl groups can be 4 to 7 or 5 to 7-membered saturated or partially unsaturated carbocyclyl or heterocyclyl groups that optionally contain 1, 2, or 3 heteroatoms independently selected from nitrogen, oxygen, phosphorus, sulfur, silicon and boron, to form, for example, a 3,4-methylenedioxyphenyl group. In one non-limiting embodiment, aryl groups are pendant. An example of a pendant ring is a phenyl group substituted with a phenyl group. In an alternative embodiment, the aryl group is optionally substituted as described above. In certain embodiments, the aryl group is an unsubstituted C₆₋₁₄ aryl. In certain embodiments, the aryl group is a substituted C₆₋₁₄ aryl. An aryl group may be optionally substituted with one or more functional groups that include but are not limited to, halo, hydroxy, nitro, amino, cyano, haloalkyl, aryl, heteroaryl, and heterocyclo.

The term “heterocyclyl” (or “heterocyclo”) includes saturated, and partially saturated heteroatom-containing ring radicals, where the heteroatoms may be selected from nitrogen, sulfur and oxygen. Heterocyclic rings comprise monocyclic 3-8 membered rings, as well as 5-16 membered bicyclic ring systems (which can include bridged fused and spiro-fused bicyclic ring systems). It does not include rings containing —O—O—. —O—S— or —S—S— portions. Said “heterocyclyl” group may be optionally substituted, for example, with 1, 2, 3, 4 or more substituents that include but are not limited to, hydroxyl, Boc, halo, haloalkyl, cyano, alkyl, aralkyl, oxo, alkoxy, and amino. Examples of saturated heterocyclo groups include saturated 3- to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include but are not limited to, dihydrothienyl, dihydropyranyl, dihydrofuryl, and dihydrothiazolyl. Examples of partially saturated and saturated heterocyclo groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl.

Heterocyclo groups also include radicals where heterocyclic radicals are fused/condensed with aryl or heteroaryl radicals: such as unsaturated condensed heterocyclic group containing 1 to 5 nitrogen atoms, for example, indoline, isoindoline, unsaturated condensed heterocyclic group containing 1 to 2 oxygen atoms and 1 to 3 nitrogen atoms, unsaturated condensed heterocyclic group containing 1 to 2 sulfur atoms and 1 to 3 nitrogen atoms, and saturated, partially unsaturated and unsaturated condensed heterocyclic group containing 1 to 2 oxygen or sulfur atoms.

The term “heteroaryl” denotes aryl ring systems that contain one or more heteroatoms selected from O, N and S, wherein the ring nitrogen and sulfur atom(s) are optionally oxidized, and nitrogen atom(s) are optionally quarternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, IH-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

As used herein, the terms “may,” “optional,” “optionally,” or “may optionally” mean that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present.

The term “optionally substituted” denotes the substitution of a group herein by a moiety including, but not limited to, C₁-C₁₀ alkyl, C₂-C₁₀ alkenyl, C₂-C₁₀ alkynyl, C₃-C₁₂ cycloalkyl, C₃-C₁₂ cycloalkenyl, C₁-C₁₂ heterocycloalkyl, C₃-C₁₂ heterocycloalkenyl, C₁-C₁₀ alkoxy, aryl, aryloxy, heteroaryl, heteroaryloxy, amino, C₁-C₁₀ alkylamino, C₁-C₁₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, azido, acyl, thioacyl, acyloxy, carboxyl, and carboxylic ester.

In one alternative embodiment any suitable group may be present on a “substituted” or “optionally substituted” position if indicated that forms a stable molecule and meets the desired purpose of the invention and includes, but is not limited to, e.g., halogen (which can independently be F, Cl, Br or I); cyano; hydroxyl; nitro; azido; alkanoyl (such as a C₂-C₆ alkanoyl group); carboxamide; alkyl, cycloalkyl, alkenyl, alkynyl, alkoxy, aryloxy such as phenoxy; thioalkyl including those having one or more thioether linkages; alkylsulfinyl; alkylsulfonyl groups including those having one or more sulfonyl linkages; aminoalkyl groups including groups having more than one N atoms; aryl (e.g., phenyl, biphenyl, naphthyl, or the like, each ring either substituted or unsubstituted); arylalkyl having for example, 1 to 3 separate or fused rings and from 6 to about 14 or 18 ring carbon atoms, with benzyl being an exemplary arylalkyl group; arylalkoxy, for example, having 1 to 3 separate or fused rings with benzyloxy being an exemplary arylalkoxy group; or a saturated or partially unsaturated heterocycle having 1 to 3 separate or fused rings with one or more N, O or S atoms, or a heteroaryl having 1 to 3 separate or fused rings with one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such groups may be further substituted, e.g. with hydroxy, alkyl, alkoxy, halogen and amino. In certain embodiments “optionally substituted” includes one or more substituents independently selected from halogen, hydroxyl, amino, cyano, —CHO, —COH, —CONH₂, alkyl including C₁-C₆alkyl, alkenyl including C₂-C₆alkenyl, alkynyl including C₂-C₆alkynyl, —C₁-C₆alkoxy, alkanoyl including C₂-C₆alkanoyl, C₁-C₆alkylester, (mono- and di-C₁-C₆alkylamino)C₀-C₂alkyl, haloalkyl including C₁-C₆haloalkyl, hydoxyC₁-C₆alkyl, ester, carbamate, urea, sulfonamide, —C₁-C₆alkyl(heterocyclo), C₁-C₆alkyl(heteroaryl), —C₁-C₆alkyl(C₃-C₇cycloalkyl), O—C₁-C₆alkyl(C₃-C₇cycloalkyl), B(OH)₂, phosphate, phosphonate and haloalkoxy including C₁-C₆haloalkoxy.

When the term “substituted” appears prior or after a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined herein.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “hydroxyalkyl” refers to the group HO-(alkyl)-.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound.

A compound of this disclosure may form a solvate with a solvent (including water). Therefore, in one non-limiting embodiment, the present disclosure includes a solvated form of the compound. The term “solvate” refers to a molecular complex of a compound (including a salt thereof) with one or more solvent molecules. Non-limiting examples of solvents are water, ethanol, isopropanol, dimethyl sulfoxide, acetone and other common organic solvents. The term “hydrate” refers to a molecular complex comprising a compound and water. Pharmaceutically acceptable solvates in accordance with the invention include those wherein the solvent may be isotopically substituted, e.g. D₂O, d₆-acetone, d₆-DMSO. A solvate can be in a liquid or solid form.

Salts, solvates, hydrates, and prodrug forms of a compound are of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

The terms “individual,” “subject”, “patient” and “host” are used interchangeably herein and refer to any subject for whom diagnosis, treatment or therapy is desired. In some aspects, the subject is a mammal. In some aspects, the subject is a human being. In some aspects, the subject is a patient. In some aspects, the subject is a human patient. In some aspects, the subject can have or is suspected of having a disorder or health condition associated with a gene-of-interest (GOI). In some aspects, the subject is a human who is diagnosed with a risk of disorder or health condition associated with a GOI at the time of diagnosis or later. In some cases, the diagnosis with a risk of disorder or health condition associated with a GOI can be determined based on the presence of one or more mutations in the endogenous GOI or genomic sequence near the GOI in the genome that may affect the expression of GOI.

The term “treatment” used to refer to a disease or condition means that at least an amelioration of the symptoms associated with the condition afflicting an individual is achieved, where amelioration is used in a broad sense to refer to at least a reduction in the magnitude of a parameter, e.g., a symptom, associated with the condition (e.g., hemophilia A) being treated. As such, treatment also includes situations where the pathological condition, or at least symptoms associated therewith, are completely inhibited, e.g., prevented from happening, or eliminated entirely such that the host no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression; (ii) inhibition, that is, arresting the development or further development of clinical symptoms, e.g., mitigating or completely inhibiting an active disease.

Definitions of other terms and concepts appear throughout the detailed description.

### Enumerated Clauses

Additional embodiments of this disclosure include those of following clauses

Clause 1. A compound of formula (I):

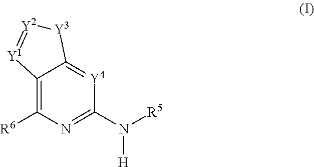

or a pharmaceutically acceptable salt, solvate or prodrug thereof,
wherein:
Y¹ is N or CR¹, and
Y² is N or CR², or
—Y¹═Y²— is an optionally substituted, fused 5-membered or 6-membered ring B:

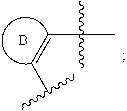

Y³ is NR³ or CR³R^{3′};
Y⁴ is N or CR⁴;
R¹ is H, (C₁₋₅)alkyl, substituted (C₁₋₅)alkyl, (C₁₋₅)alkoxyl, substituted (C₁₋₅)alkoxyl, haloalkyl, halogen, OH, or CN;
R² is H, (C₁₋₅)alkyl, substituted (C₁₋₅)alkyl, (C₁₋₅)alkoxyl, substituted (C₁₋₅)alkoxyl, haloalkyl, halogen, OH, or CN;
R³ is selected from H, alkyl, carbocycle, heterocycle, aryl, heteroaryl, alkylene-carbocycle, alkylene-heterocycle, alkylene-aryl, alkylene-heteroaryl, and substituted versions thereof; and
R^{3′} is H, or (C₁₋₅)alkyl;
R⁴ is H, (C₁₋₅)alkyl, substituted (C₁₋₅)alkyl, haloalkyl, halogen, or CN;
R⁵ is

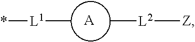

wherein:
   A is a cyclic group selected from carbocyclyl, heterocyclyl, aryl, heteroaryl, and substituted versions thereof;
   Z is an optional hydrophilic group;
   L¹ and L² are optional linkers; and
      * represents the point of attachment of R⁵ to the nitrogen atom; and
R⁶ is H, (C₁₋₅)alkyl, substituted (C₁₋₅)alkyl, (C₁₋₅)alkoxyl, substituted (C₁₋₅)alkoxyl, haloalkyl, halogen, OH, or CN.

Clause 2. The compound of clause 1, wherein one or more of Y¹ to Y³ is N, or NR³.

Clause 3. The compound of clause 1 or 2, wherein two or three of Y¹ to Y³ are N, or NR³.

Clause 4. The compound of clause 2 or 3, wherein Y³ is NR³.

Clause 5. The compound of any one of clauses 1 to 4, wherein Y⁴ is N.

Clause 6. The compound of any one of clauses 1 to 4, wherein Y⁴ is CR⁴.

Clause 7. The compound of any one of clauses 1 to 6, wherein the compound is of one of Formula (IA) to Formula (IH):

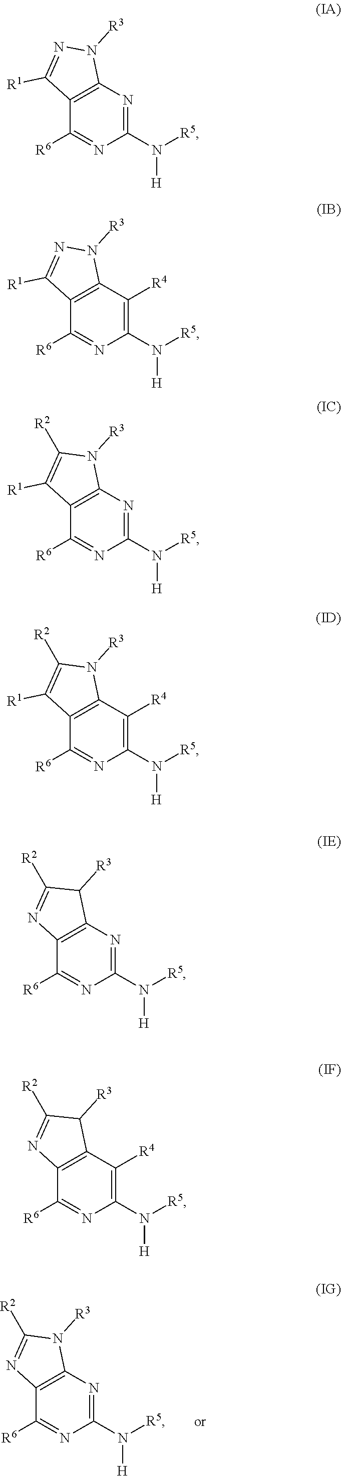

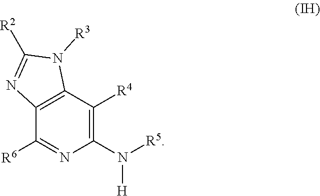

Clause 8. The compound of clause 1, wherein —Y¹═Y²— is the optionally substituted fused 5-membered or 6-membered ring B, and the compound is of Formula (IJ′), or Formula (IK′):

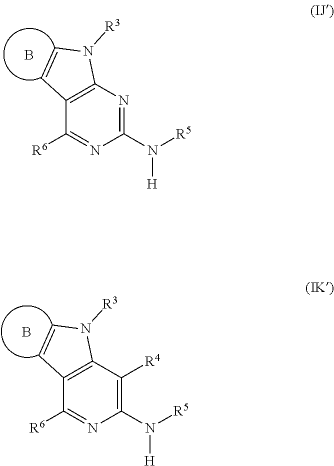

wherein: the fused 5-membered or 6-membered ring B is selected from cycloalkenyl, aryl, heterocycle, heteroaryl, and substituted versions thereof.

Clause 9. The compound of clause 7, wherein the compound is of Formula (IA) or Formula (IB), wherein R⁴ is H.

Clause 10. The compound of clause 7, wherein the compound is of Formula (IC) or Formula (ID), wherein R⁴ is H.

Clause 11. The compound of clause 7, wherein the compound is of Formula (IG) or Formula (IH), wherein R⁴ is H.

Clause 12. The compound of any one of clauses 1 to 11, wherein A is selected from cycloalkyl, cycloalkenyl, heterocyclyl, aryl, heteroaryl, and substituted versions thereof.

Clause 13. The compound of clause 12, wherein A is optionally substituted heterocyclyl.

Clause 14. The compound of clause 13, wherein the compound is of formula (IIA):

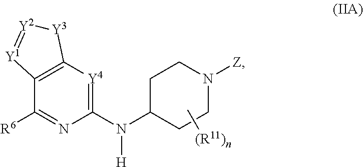

wherein,
each R¹¹ is independently selected from (C₁₋₅)alkyl, substituted (C₁₋₅)alkyl, (C₁₋₅)alkoxy, substituted (C₁₋₅)alkoxy, halogen, OH, and CN; and
n is 0 to 9.

Clause 15. The compound of clause 14, wherein the compound is of the Formula (IIIA):

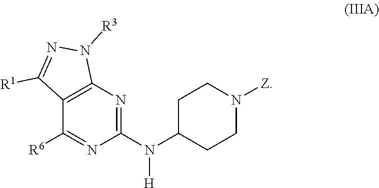

Clause 16. The compound of clause 12, wherein A is optionally substituted aryl or optionally substituted heteroaryl.

Clause 17. The compound of clause 16, wherein A is optionally substituted phenyl or optionally substituted pyridyl.

Clause 18. The compound of clause 16, wherein the compound is of Formula (IIB):

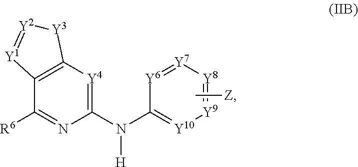

wherein:
Y⁶ to Y¹⁰ are each independently C—Z, CR¹² or N; and
each R¹² is independently selected from H, (C₁₋₅)alkyl, substituted (C₁₋₅)alkyl, (C₁₋₅)alkoxy, substituted (C₁₋₅)alkoxy, halogen, OH, and CN.

Clause 19. The compound of clause 18, wherein one and only one of Y⁶ to Y¹⁰ is C—Z.

Clause 20. The compound of clause 18, wherein Y⁷ is C—Z.

Clause 21. The compound of clause 18, wherein Y⁸ is C—Z.

Clause 22. The compound of any one of clauses 18 to 21, wherein one or two of Y⁶ to Y¹⁰ is N.

Clause 23. The compound of any one of clauses 18 to 22, wherein four of Y⁶ to Y¹⁰ are CR¹².

Clause 24. The compound of clause 22 or 23, wherein each R¹² is H.

Clause 25. The compound of any one of clauses 18 to 24, wherein the compound is of one of Formula (IIIB) to Formula (IIIJ):

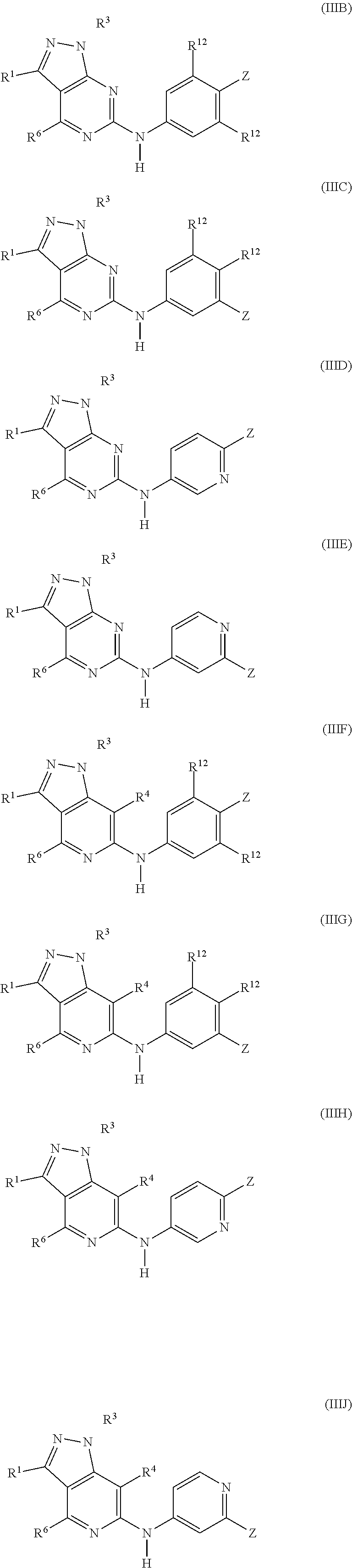

wherein:
R¹² is H, (C₁₋₅)alkyl, substituted (C₁₋₅)alkyl, (C₁₋₅)alkoxy, substituted (C₁₋₅)alkoxy, halogen, OH, or CN.

Clause 26. The compound of any one of clauses 1 to 25, wherein Z comprises a group selected from sulfone, sulfonate, carboxamide, carboxy, hydroxy, hydroxyalkyl, aldehyde, ester, alkoxy, nitrile, halogen, thiolactone, and any combination thereof.

Clause 27. The compound of any one of clauses 1 to 26, wherein L¹ and L² are independently absent or (C₁₋₆)alkylene (e.g., CH₂, (CH₂)₂, or (CH₂)₃).

Clause 28. The compound of any one of clauses 1 to 27, wherein -L²-Z or Z is of one of Z1-Z7:

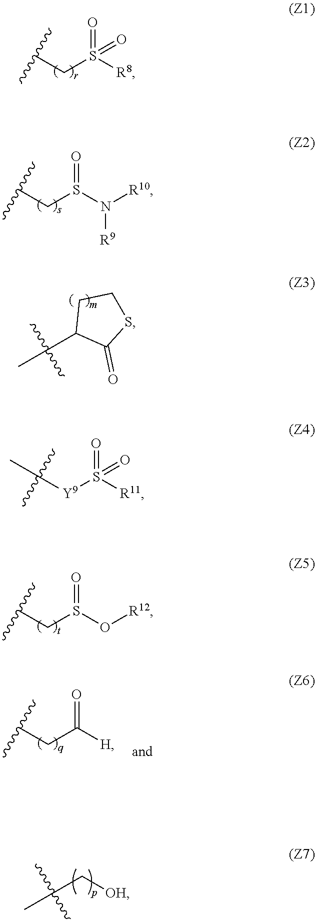

wherein:
R⁸ is (C₁₋₆)alkyl (e.g., (C₃₋₆)cycloalkyl), (C₂₋₆)alkenyl, (C₂₋₆)alkynyl, or a substituted version thereof;
R⁹ is H or optionally substituted (C₁₋₆)alkyl;
R¹⁰ is H, optionally substituted (C₁₋₆)alkyl, a sulfone (e.g., of formula Z1), or a thiolactone (e.g., of formula Z3); and
Y⁹ is absent, —O—, —NR⁹—, —(C₁₋₆)alkylene-O—, —(C₁₋₆)alkylene-NR⁹—;
R¹¹ is halogen, —NR⁹R¹⁰, (C₁₋₆)alkyl, (C₂₋₆)alkenyl, (C₂₋₆)alkynyl, or a substituted version thereof;
R¹² is H, optionally substituted (C₁₋₆)alkyl, optionally substituted aryl, or optionally substituted heterocycle;
m is 0 to 2; and
p, q, r, s and t are independently 0, 1, 2, 3, 4, 5 or 6.

Clause 29. The compound of clause 28, wherein the -L²-Z or Z is of formula Z1.

Clause 30. The compound of clause 29, wherein R⁸ is (C₁-C₆)alkyl.

Clause 31. The compound of clause 30, wherein R⁸ is methyl.

Clause 32. The compound of clause 28, wherein R⁸ is C₂-C₆ alkenyl.

Clause 33. The compound of clause 33, wherein R⁸ is —CH═CH₂ or —C(CH₃)═CH₂.

Clause 34. The compound of clause 28, wherein R⁸ is C₂-C₆ alkynyl.

Clause 35. The compound of clause 34, wherein R⁸ is —CCH or —CC—CH₃.

Clause 36. The compound of clause 28, wherein -L²-Z or Z is of the formula Z2.

Clause 37. The compound of clause 36, wherein -L²-Z or Z is of Formula (Z2A):

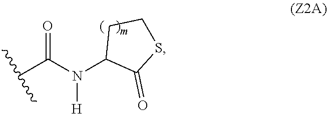

wherein m is 0 to 2.

Clause 38. The compound of clause 37, wherein m is 1.

Clause 39. The compound of clause 37 or 38, wherein R⁹ is H.

Clause 40. The compound of clause 28, wherein -L²-Z or Z is of the formula Z3.

Clause 41. The compound of clause 40, wherein m is 1.

Clause 42. The compound of clause 28, wherein -L²-Z or Z is of the formula Z6.

Clause 43. The compound of clause 42, wherein Z is CHO, CH₂CHO, (CH₂)₂CHO, or (CH₂)₃CHO.

Clause 44. The compound of clause 28, wherein -L²-Z or Z is of the formula Z7.

Clause 45. The compound of clause 44, wherein -L²-Z or Z is OH, CH₂OH, (CH₂)₂OH, or (CH₂)₃OH.

Clause 46. The compound of any one of clauses 1 to 28, wherein Z is a moiety that is substantially biologically inert.

Clause 47. The compound of any one of clauses 1 to 28, wherein Z comprises an electrophilic reactive moiety capable of covalently conjugating a biological target.

Clause 48. The compound of clause 28, wherein the compound is of one of formulae (IVA)-(IVC):

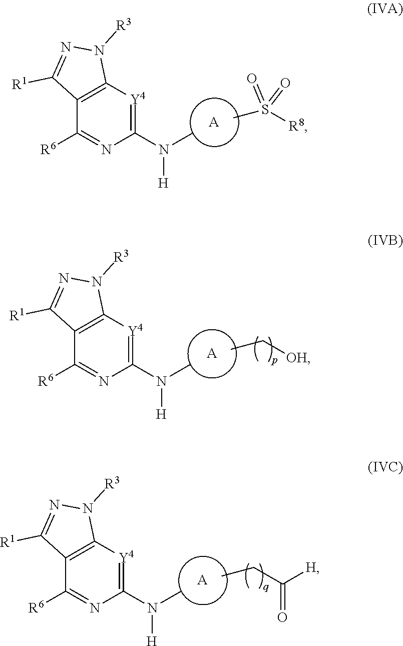

wherein:
R⁸ is optionally substituted (C₁-C₆)alkyl, optionally substituted (C₂-C₆)alkenyl, or optionally substituted (C₂-C₆)alkenyl; and
p is 0, 1, 2 or 3; and
q is 0, 1, 2 or 3.

Clause 49. The compound of clause 48, wherein A is phenyl, 3-pyridyl, 4-pyridyl, 4-piperidinyl, 4-indolyl, 5-indolyl, pyrrolidin-2-one, or a substituted version thereof.

Clause 50. The compound of clause 28, wherein the compound is of one of formulae (VA)-(VC):

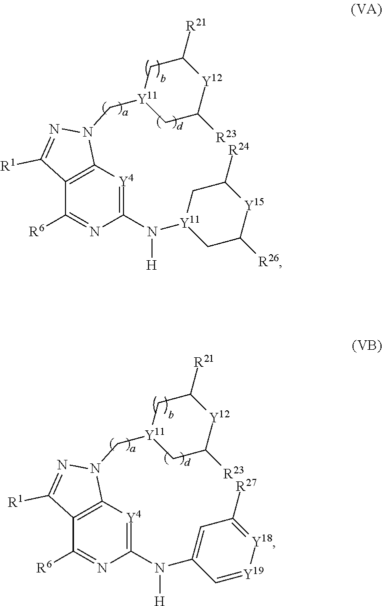

wherein:
Y¹¹ is N or CH;
Y¹² is NR²², C(R^{22′})₂, O or C═O;
Y¹⁵ is NR²⁵ or C(R^{25′})₂;
Y¹⁸ is N or CR²⁸;
Y¹⁹ is N or CR²⁹;
a is 0, 1 or 2;
b is 0 or 1;
d is 0 or 1;
R²¹, R²², each R^{22′}, and R²³ are independently selected from H, (C₁₋₅)alkyl, (C₁₋₅)haloalkyl, OH, (C₁₋₅)alkylene-OH, O(C₁₋₅)alkyl, CHO, CN, CO₂R³², CONR³⁰R³¹, NR³⁰R³¹, halogen, and (C₁₋₅)alkylene-X;
R²⁴, R²⁵, each R^{25′}, and R²⁶ are independently selected from Z, H, OH, (C₁₋₅)alkylene-OH, O(C₁₋₅)alkyl, CHO, CN, CO₂R³², CONR³⁰R³¹, NR³⁰R³¹, halogen, and (C₁₋₅)alkylene-X; and
R²⁷, R²⁸, and R²⁹ are independently selected from Z, H, (C₁₋₅)alkyl, (C₁₋₅)haloalkyl, OH, (C₁₋₅)alkylene-OH, CN, CO₂R³², (C₁₋₅)alkylene-CHO, CHO, CONR³⁰R³¹, NR³⁰R³¹, halogen, and (C₁₋₅)alkylene-X;
R³⁰ and R³¹ are independently H, or (C₁₋₅)alkyl, or R³⁰ and R³¹ are cyclically linked and together with the nitrogen atom to which they are attached provide an optionally substituted heterocyclyl ring;
R³² is H, optionally substituted (C₁₋₅)alkyl, optionally substituted heterocyclyl, or optionally substituted aryl; and
X is halogen.

Clause 51. The compound of clause 50, wherein the compound is of one of Formula (VIA)-(VIB):

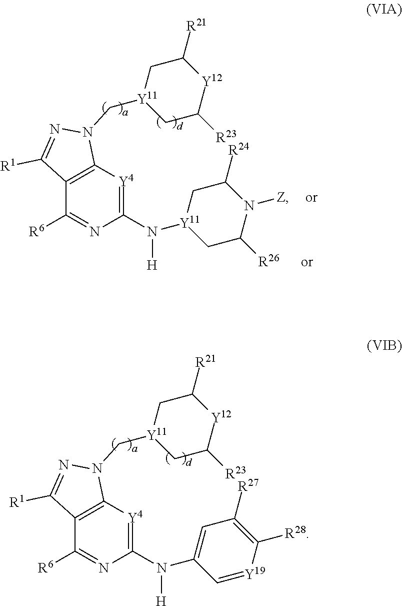

Clause 52. The compound of clause 50 or 51, wherein a is 0.

Clause 53. The compound of clause 50 or 51, wherein a is 1.

Clause 54. The compound of any one of clauses 50 to 53, wherein d is 0.

Clause 55. The compound of any one of clauses 50 to 53, wherein d is 1.

Clause 56. The compound of any one of clauses 50 to 55, wherein:
R²¹ is H; and
R^{22′}, and R²³ are independently selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₅)alkylene-X.

Clause 57. The compound of any one of clauses 50 to 56, wherein Y¹⁹ is N.

Clause 58. The compound of any one of clauses 50 to 56, wherein Y¹⁹ is CH.

Clause 59. The compound of any one of clauses 50 to 58, wherein the compound is of Formula (VIA), wherein:
R²⁴ is H; and
R²⁶ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X.

Clause 60. The compound of any one of clauses 50 to 58, wherein the compound is of Formula (VIB), wherein:
R²⁷ is H or Z; and
R²⁸ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X.

Clause 61. The compound of any one of clauses 50 to 58, wherein the compound is of Formula (VIB), wherein:
R²⁷ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X; and
R²⁸ is H or Z.

Clause 62. The compound of any one of clauses 50 to 61, wherein Y⁴ is N.

Clause 63. The compound of any one of clauses 50 to 61, wherein Y⁴ is CR⁴ or CH.

Clause 64. The compound of any one of clauses 50 to 63, wherein R⁶ is H, CH₃, CF₃, or halogen.

Clause 65. The compound of clause 64, wherein R⁶ is H.

Clause 66. The compound of any one of clauses 1 to 65, wherein R¹ is H, CH₃, CF₃, or halogen.

Clause 67. The compound of clause 66, wherein R¹ is H.

Clause 68. The compound of any one of clauses 50 to 67, wherein the Z is SO₂R⁸, wherein R⁸ is optionally substituted (C₁-C₆)alkyl, optionally substituted (C₂-C₆)alkenyl, or optionally substituted (C₂-C₆)alkynyl.

Clause 69. The compound of clause 68, wherein R⁸ is (C₁-C₆)alkyl.

Clause 70. The compound of clause 69, wherein R⁸ is methyl.

Clause 71. The compound of clause 68, wherein R⁸ is C₂-C₆ alkenyl.

Clause 72. The compound of clause 71, wherein R⁸ is —CH═CH₂ or —C(CH₃)═CH₂.

Clause 73. The compound of clause 68, wherein R⁸ is C₂-C₆ alkynyl.

Clause 74. The compound of clause 73, wherein R⁸ is —CCH or —CC—CH₃.

Clause 75. The compound of any one of clauses 50 to 67, wherein Z is CHO, CH₂CHO, (CH₂)₂CHO, or (CH₂)₃CHO.

Clause 76. The compound of any one of clauses 50 to 67, wherein Z is OH, CH₂OH, (CH₂)₂OH, or (CH₂)₃OH.

Clause 77. The compound of any one of clauses 1-76, wherein the compound is selected from Tables 1-9, or a pharmaceutically acceptable salt thereof.

Clause 78. A pharmaceutical composition comprising a compound of any one of clauses 1 to 77 and a pharmaceutically acceptable excipient.

Clause 79. A method of inhibiting cyclin-dependent kinase 2 (CDK2), the method comprising contacting a biological sample containing CDK2 with an effective amount of a compound according to any one of clauses 1 to 77, or a pharmaceutical composition according to clause 53 to inhibit CDK2.

Clause 80. A method of inhibiting CDK2 in a subject, comprising administering to the subject a compound of any one of clauses 1 to 77, or a pharmaceutical composition according to clause 78 to inhibit CDK2 in the subject.

Clause 81. A method of treating a disease or disorder associated with CDK2 in a subject, comprising administering to the subject suffering from a disease or disorder associated with CDK2 a therapeutically effective amount of a compound of any one of clauses 1 to 77, or a pharmaceutical composition according to clause 78.

The following example(s) is/are offered by way of illustration and not by way of limitation.

### 5. EXAMPLES

The following examples are put forth so as to provide those of ordinary skill in the art with a complete disclosure and description of how to make and use the compounds of the present disclosure, and are not intended to limit the scope of what the inventors regard as their disclosure nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### Methods of Preparation

Synthetic schemes for preparing intermediates and compounds of the disclosure are described below. The illustrative schemes described are not intended to limit the methods available to prepare the compounds or imply a specific ordering of steps is required, nor are any of the variable groups (e.g., R groups) intended to limit any of the claims described herein. Unless specified otherwise, compounds were prepared as racemic mixtures. Chirally pure materials could be obtained by chiral preparative HPLC separation of the racemic mixtures or by employing homochiral starting materials.

Abbreviations used herein are defined as follows:

| Me | methyl | mg | milligram, milligrams |
|---|---|---|---|
| Et | ethyl | g | gram, grams |
| Pr | propyl | L | liter, liters |
| iPr | isopropyl | mL | milliliter, milliliters |
| Bu | butyl | μL | microliter, microliters |
| tBu | tert-butyl | N | normal |
| Ph | phenyl | M | molar |
| ACN | acetonitrile | mmol | millimole, millimoles |
| DCM | dichloromethane | μmol | micromole, |
| DMF | dimethylformamide | rt | micromoles |
| DMSO | dimethylsulfoxide | RT | room temperature |
| EtOAc | ethyl acetate | min | retention time |
| IPA | isopropanol | h | minutes |
| MeOH | methanol | atm | hours |
| MTBE | methyl tert-butyl ether | psi | atmosphere |
| NMP | N-methylpyrrolidine | conc. | pounds per square |
| PE | petroleum ether | sat | inch |
| THF | tetrahydrofuran | aq | concentrated |
| TFE | 2,2,2-trifluoroethanol | eq | saturated |
| AcOH | acetic acid | MS | aqueous |
| mCPBA | meta-chloroperoxybenzoic | ESI | equivalents |
| | acid | | mass spectrometry |
| DIPEA | diisopropylethylamine | LCMS | electrospray |
| DMAP | 4-dimethylaminopyridine | | ionization mass |
| DMP | Dess-Martin | HPLC | liquid chromatography |
| | periodinane | prep | mass |
| DIAD | Diisopropyl | SFC | high pressure liquid |
| | azodicarboxylate | | preparative |
| EDC | 1-ethyl-3-(3- | TLC | supercritical fluid |
| | diimethylaminopropyl) | | chromatography |
| | carbodiimide | UV | thin layer |
| HOBt | 1-hydroxybenzotriazole | NMR | chromatography |
| LiOH | lithium hydroxide | | ultraviolet |
| MeI | methyl iodide | ¹H | nuclear magnetic |
| Oxone ® | potassium | δ | resonance |
| | peroxymonosulfate | | proton |
| Py | pyridine | bs | delta |
| TFA | trifluoroacetic acid | S | broad singlet |
| TfOH | trifluoromethanesulfonic | d | singlet |
| | acid | | doublet |
| TMSC1 | trimethylsilyl chloride | t | triplet |
| TBSCI | tert-butyldimethylsilyl | q | quartet |
| | chloride | m | multiplet |
| TEA | triethylamine | br | broad |
| 1 × | once | ppm | parts per million |
| 2 × | twice | MHz | megahertz |
| 3 × | thrice | ° C. | degrees Celsius |

The synthetic schemes for the preparation of intermediates and compounds of the present invention are detailed below. The schemes are illustrative, and the intermediates and compounds may be synthesized by many methods known to those skilled in the art of organic chemistry, not limited to those described. The R groups within the schemes are illustrative and the order of the steps is suggested, and neither are intended to limit any of claims. General procedures for representative intermediates and compounds are described and are prepared as racemic mixtures unless specified. Chirally pure intermediates or compounds can be obtained by techniques known to a person who is skilled in the art. For example, using preparative chiral HPLC or the use of chirally pure starting materials.

Scheme 1 illustrates an approach to prepare substituted pyrazolopyrimidines. The disubstituted pyrazolopyrimidine core can be prepared by N-alkylation of chloropyrazolopyrimidine with an alkyl halide or mesylate or similar followed by nucleophilic aromatic substitution with the requisite amine. Boc deprotection reveals the pyrrolidine amine for derivatization to final compounds.

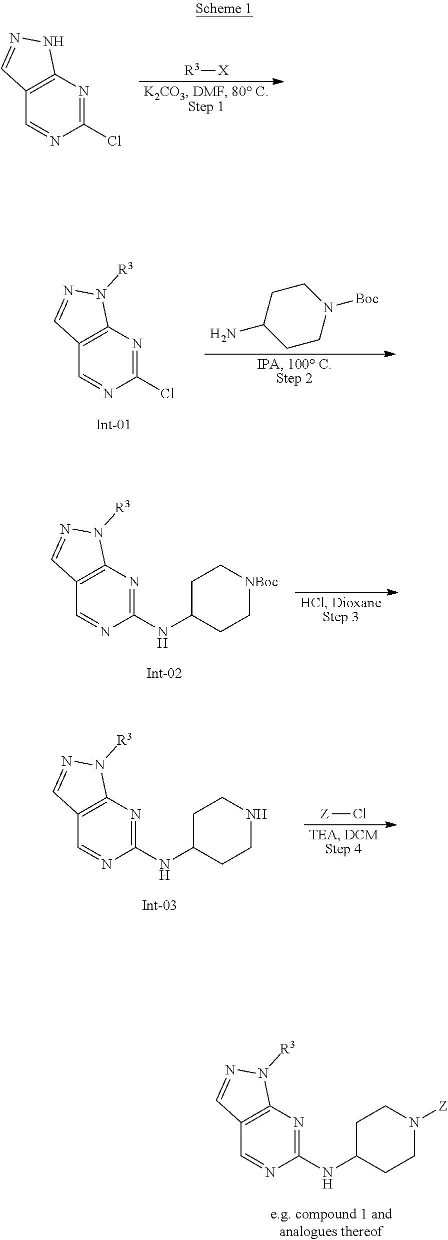

Scheme 2 illustrates an approach to prepare compounds containing the methyl phenyl sulfone. Nucleophilic aromatic substitution of the pyrazolopyrimidine core followed by N-alkylation yields a variety compounds represented in Table 1. Alternatively, Buchwald-Hartwig cross-coupling with intermediate 04 will yield compounds represented in Table 3 or Suzuki-Miyaura coupling will yield compounds similar to 68 and 83.

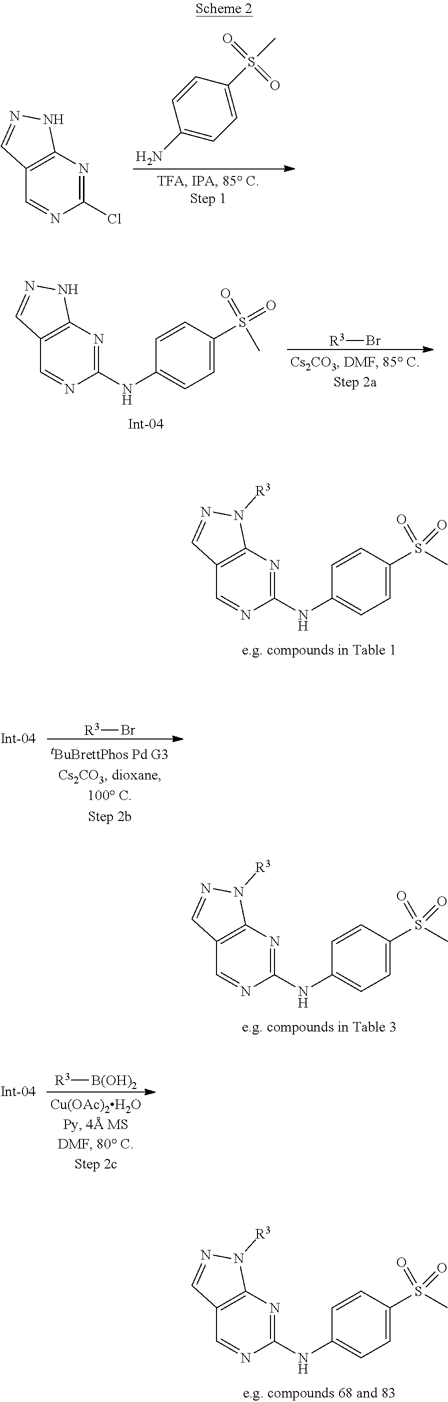

Furthermore, the methylcyclohexanone can be further derivatized by reductive amination with an amine to yield compounds represented in Table 2.

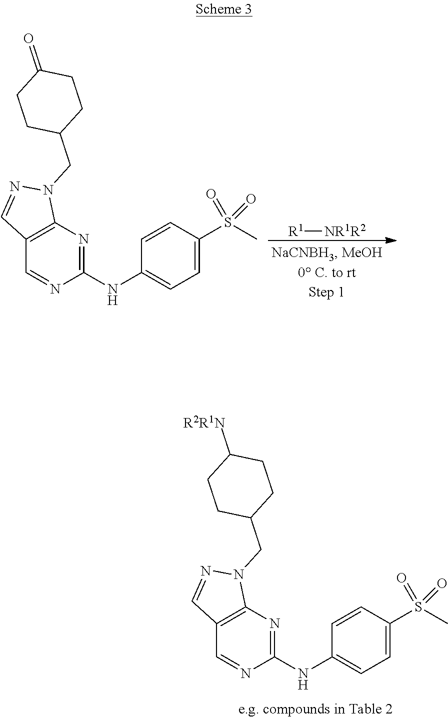

Alternatively, the coupling steps can be reversed as illustrated in Scheme 4. The Ni substituent can be installed first by Mitsunobu reaction of chloropyrazolopyrimidine with an alcohol followed by Buchwald-Hartwig cross-coupling with the requisite amine to yield compounds similar to 77.

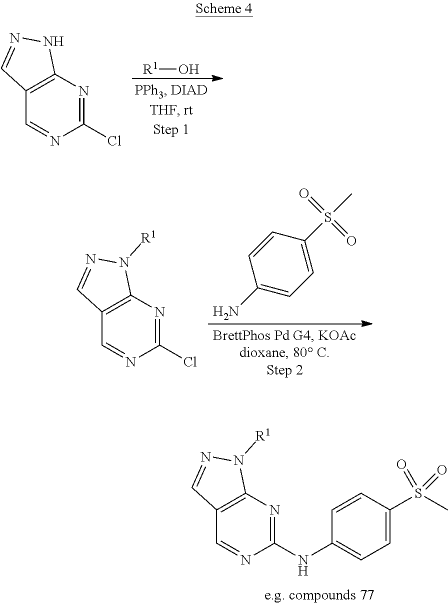

Scheme 5 illustrates an approach to prepare compounds containing the vinyl phenyl sulfone similar to compound 8. The disubstituted pyrazolopyrimidine core can be prepared by N-alkylation of chloropyrazolopyrimidine with an alkyl halide or similar followed by nucleophilic aromatic substitution with the requisite amine. Oxidation of the thiol can be achieved using an oxidant known by those skilled in the art, for example mCPBA or Oxone®. The alcohol can be converted to a leaving group and elimination to the alkene using a base to give the vinyl phenyl sulfone.

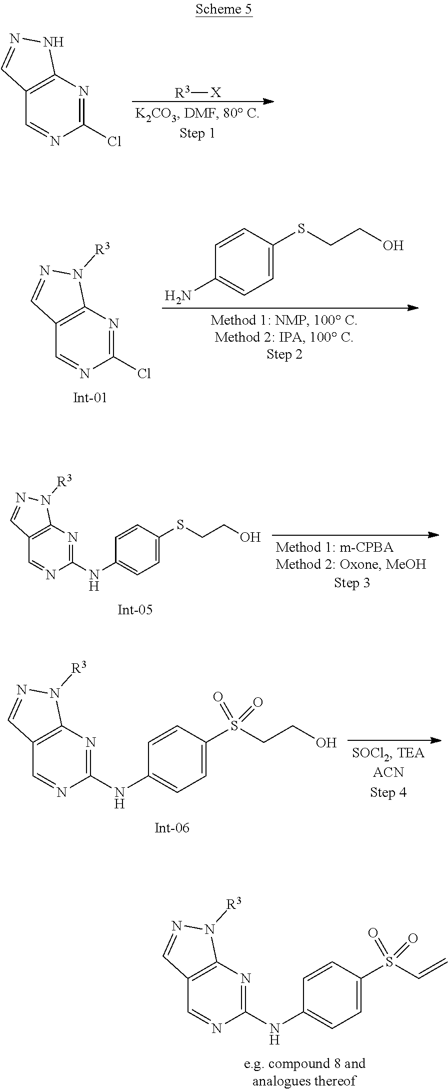

Scheme 6 illustrates an approach to prepare compounds with alternative C6 groups such as substituted aromatic groups represented in Table 4, alternative vinyl sulfones represented in Table 6 or alternative electrophiles such as compounds represented in Table 5 or 7.

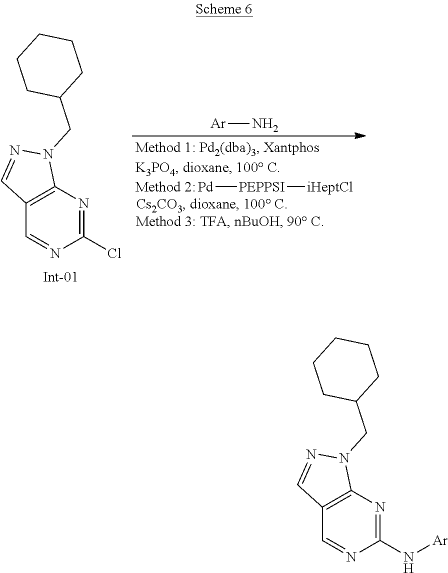

Reactions were performed under nitrogen atmosphere and progress of the reaction was monitored by TLC and LCMS unless stated otherwise. Intermediates and compounds were purified by normal or reverse phase chromatography using the following instruments and methods. Normal phase chromatography was carried out using Biotage Isolera (100-200 mesh size silica gel) using gradients of ethyl acetate in hexanes or methanol in dichloromethane as the eluent. Reverse phase chromatography was carried out using Biotage Isolera (C18 column) using gradients of aq NH₄HCO₃ and ACN. Reverse phase prep HPLC was carried out using Shimadzu LC; Prominence-I series instruments with UV detection (Maxplot). Mobile Phase: A: 0.1% formic acid in Water, B: ACN; flow rate: 2.0 mL/min; Column: X-Select CSH C18 (4.6×150 mm), 5 μm.

NMR data was acquired in Bruker Avance Neo nano bay 400 MHz NMR Spectrometer. Chemical shifts are reported in ppm relative to dimethyl sulfoxide (δ 2.50), methanol (δ 3.31), chloroform (δ 7.26) or other solvent as indicated in NMR spectral data. Small amounts (1-5 mg) of samples were dissolved in appropriate deuterated solvents (0.6 mL).

Purity of intermediates and compounds were determined by LCMS and NMR spectroscopy using the following instruments and methods:

Instrument names: Shimadzu LC2020 Nexera Series; Shimadzu MS2020 N-Series; Agilent 1290.

Method A: Mobile Phase: A: 0.1% formic acid in water, B: ACN; Column: ZORBAX ECLIPSE PLUS C18 (50×2.1 mm), 1.8 μm.

Method B: Mobile Phase: A: 10 mM NH₄HCO₃ in water, B: ACN; Column: Phenomenex Kinetex EVO C18 (50×3.0 mm), 2.5 μm.

Method C: Mobile Phase: A: 0.1% TFA in water, B: ACN; Column: XBRIGDE C8 (4.6×50 mm), 3.5 μm.

Method D: Mobile Phase: A: 10 mM NH₄HCO₃ in water, B: ACN; Column: Phenomenex Kinetex EVOC18 (3.0×50 mm), 2.6 μm.

### Synthesis of Intermediate 03:

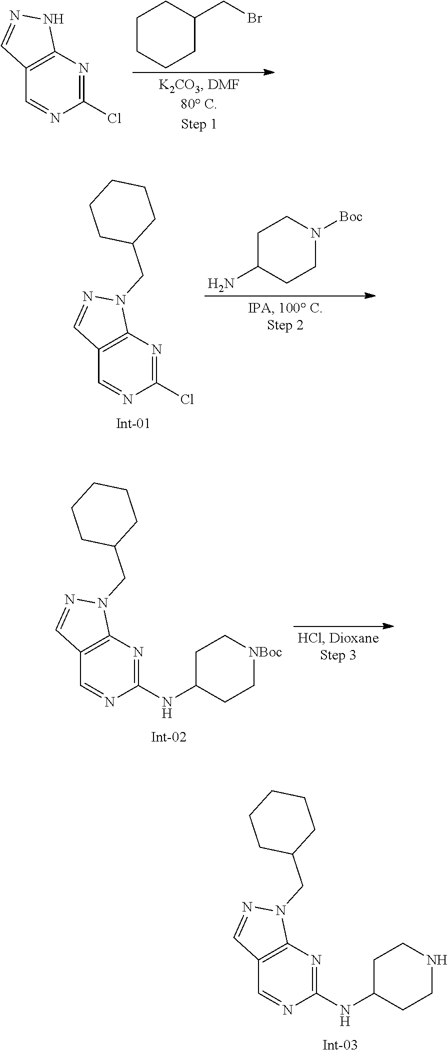 [SMILES: Clc1ncc2cn[nH]c2n1 ; BrCC1CCCCC1 ; Clc1ncc2cnn(CC3CCCCC3)c2n1 ; CC(C)(C)OC(=O)N1CCC(N)CC1 ; CC(C)(C)OC(=O)N1CCC(Nc2ncc3cnn(CC4CCCCC4)c3n2)CC1 ; c1nc(NC2CCNCC2)nc2c1cnn2CC1CCCCC1]

### Intermediate 01: 6-chloro-1-(cyclohexylmethyl)-1H-pyrazolo[3,4-d]pyrimidine

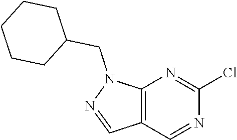 [SMILES: Clc1ncc2cnn(CC3CCCCC3)c2n1]

To a solution of 6-chloro-1H-pyrazolo[3,4-d]pyrimidine (1.00 g, 6.47 mmol) in DMF (10 mL) were added K₂CO₃ (2.24 g, 16.2 mmol) and (bromomethyl)cyclohexane (1.38 g, 7.76 mmol) at rt, under nitrogen atmosphere. The reaction mixture was stirred at 80° C. for 4 h. After completion, the reaction was quenched with ice-cold water (50 mL) and the aqueous phase was extracted with EtOAc (3×50 mL). The combined organic layers were washed with brine (50 mL), dried over anhydrous Na₂SO₄, filtered and concentrated in vacuo to afford a crude mixture, which was purified by Biotage Isolera (60% EtOAc in hexanes) to obtain the desired product, 6-chloro-1-(cyclohexylmethyl)-1H-pyrazolo[3,4-d]pyrimidine Int-01 (450 mg, 1.65 mmol, 25.5% yield) as an off-white solid. LCMS (Method D): 98%, RT: 1.74 min, m/z: 251.0 (M+H)⁺.

### Intermediate 02: tert-butyl-4-((1-(cyclohexylmethyl)-1H-pyrazolo[3,4-d]pyrimidin-6-yl)amino)piperidine-1-carboxylate

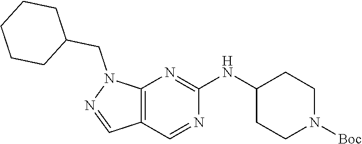 [SMILES: CC(C)(C)OC(=O)N1CCC(Nc2ncc3cnn(CC4CCCCC4)c3n2)CC1]

To a solution of 6-chloro-1-(cyclohexylmethyl)-1H-pyrazolo[3,4-d]pyrimidine Int-01 (300 mg, 1.20 mmol) in IPA (2 mL) was added tert-butyl 4-aminopiperidine-1-carboxylate (719 mg, 3.59 mmol) at rt and reaction mixture was heated at 100° C. for 16 h. After completion, solid precipitate was filtered, washed with MTBE and dried to afford crude compound as a brown solid. Crude compound was purified by Biotage Isolera (60% EtOAc in hexanes) to obtain the desired product, tert-butyl 4-((1-(cyclohexylmethyl)-1H-pyrazolo[3,4-d]pyrimidin-6-yl)amino)piperidine-1-carboxylate Int-02 (472 mg, 1.14 mmol, 95.1% yield) as a brown liquid. LCMS (Method D): 95.7%, RT: 2.39 min, m/z: 415.2 (M+H)⁺.

### Intermediate 03: 1-(cyclohexylmethyl)-N-(piperidin-4-yl)-1H-pyrazolo[3,4-d]pyrimidin-6-amine

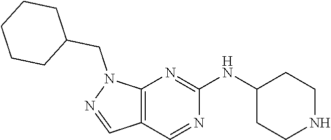 [SMILES: c1nc(NC2CCNCC2)nc2c1cnn2CC1CCCCC1]

To a solution of tert-butyl 4-((1-(cyclohexylmethyl)-1H-pyrazolo[3,4-d]pyrimidin-6-yl)amino)piperidine-1-carboxylate Int-02 (540 mg, 1.303 mmol) in 1,4-dioxane (4 mL) was added 4 M HCl in 1,4-dioxane (0.724 mL, 20.84 mmol) at 0° C. The reaction mixture was stirred at rt for 3 h. After completion, solvent was removed in vacuo. The crude solid was triturated with MTBE and dried to afford the desired product, 1-(cyclohexylmethyl)-N-(piperidin-4-yl)-1H-pyrazolo[3,4-d]pyrimidin-6-amine hydrochloride Int-03 (350 mg, 0.657 mmol, 50.4% yield) as an off-white solid. LCMS (Method D): 59.9%, RT: 1.54 min, m/z: 315.1 (M+H)⁺.

### Compound 1: 1-(cyclohexylmethyl)-N-(1-(vinylsulfonyl)piperidin-4-yl)-1H-pyrazolo[3,4-d]pyrimidin-6-amine

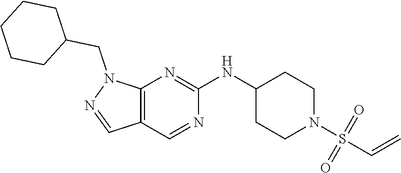 [SMILES: C=CS(=O)(=O)N1CCC(Nc2ncc3cnn(CC4CCCCC4)c3n2)CC1]

To a solution of 1-(cyclohexylmethyl)-N-(piperidin-4-yl)-1H-pyrazolo[3,4-d]pyrimidin-6-amine Int-03 (200 mg, 0.636 mmol) in DCM (20 mL) were added TEA (0.355 mL, 2.54 mmol) and 2-chloroethane-1-sulfonyl chloride (0.073 mL, 0.70 mmol) at 0° C. The reaction mixture was stirred at rt for 1 h. After completion, the reaction mixture was quenched with water (30 mL) and extracted with DCM (3×30 mL). The combined organic layers were washed with brine (30 mL), dried over anhydrous Na₂SO₄, filtered and concentrated in vacuo to afford crude compound as a brown solid, which was purified by prep HPLC and pure fractions were lyophilized to afford the desired compound, 1-(cyclohexylmethyl)-N-(1-(vinylsulfonyl)piperidin-4-yl)-1H-pyrazolo[3,4-d]pyrimidin-6-amine (22.0 mg, 0.053 mmol, 8.3% yield) as an off-white solid. ¹H NMR (400 MHz, DMSO-d₆): δ 8.80 (s, 1H), 7.94 (s, 1H), 7.48 (s, 1H), 6.83 (dd, J=16.5, 10.0 Hz, 1H), 6.21-6.03 (m, 2H), 4.03 (d, J=7.0 Hz, 2H), 3.95-3.80 (m, 1H), 3.60-45 (m, 2H), 2.90-2.75 (m, 2H), 2.07-1.86 (m, 3H), 1.76-1.44 (m, 7H), 1.26-1.09 (m, 3H), 0.98 (m, 2H). LCMS (Method D): 99.2%, RT: 2.03 min, m/z: 405.1 (M+H)⁺.

### Compound 2: 1-(cyclohexylmethyl)-N-(1-(methylsulfonyl)piperidin-4-yl)-1H-pyrazolo[3,4-d]pyrimidin-6-amine

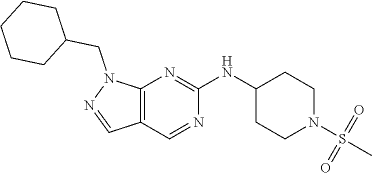 [SMILES: CS(=O)(=O)N1CCC(Nc2ncc3cnn(CC4CCCCC4)c3n2)CC1]

To a solution of 1-(cyclohexylmethyl)-N-(piperidin-4-yl)-1H-pyrazolo[3,4-d]pyrimidin-6-amine Int-03 (60 mg, 0.191 mmol) in DCM (5 mL) were added TEA (0.080 mL, 0.572 mmol) and MeSO₂Cl (0.015 mL, 0.191 mmol) at 0° C. The reaction mixture was stirred at rt for 30 min. After completion, the reaction mixture was diluted with water (30 mL) and extracted with DCM (3×30 mL). The combined organic layers were washed with brine (30 mL), dried over anhydrous Na₂SO₄, filtered and concentrated in vacuo to afford crude compound as a brown solid, which was purified by prep HPLC and pure fractions were lyophilized to afford the desired compound, 1-(cyclohexylmethyl)-N-(1-(methylsulfonyl)piperidin-4-yl)-1H-pyrazolo[3,4-d]pyrimidin-6-amine (8 mg, 0.019 mmol, 10.1% yield) as an off-white solid. ¹H NMR (400 MHz, DMSO-d₆): δ 8.81 (s, 1H), 7.94 (s, 1H), 7.48 (s, 1H), 4.18-3.75 (m, 3H), 3.60-3.50 (m, 2H), 2.89 (m, 5H), 2.07-1.76 (m, 3H), 1.70-1.45 (m, 7H), 1.25-1.05 (m, 3H), 1.03-0.90 (m, 2H). LCMS (Method D): 99.8% RT: 1.86 min, m/z: 393.1 (M+H)⁺.

### General Procedure for the Syntheses of Compounds 3-7

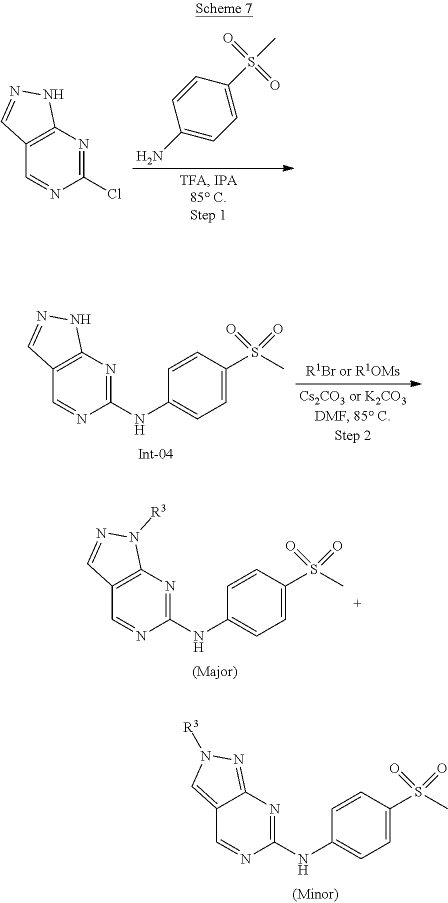

### Intermediate 04: N-(4-(methylsulfonyl)phenyl)-1H-pyrazolo[3,4-d]pyrimidin-6-amine

To a solution of 6-chloro-1H-pyrazolo[3,4-d]pyrimidine (1.00 g, 6.47 mmol) and 4-(methylsulfonyl)aniline (997 mg, 5.82 mmol) in IPA (10 mL) was added TFA (0.248 mL, 3.24 mmol) and the reaction mixture was heated at 85° C. for 16 h. After completion, the reaction mixture was diluted with EtOAc (30 mL) and solid precipitated was filtered, washed with 10% IPA in EtOAc (50 mL) to obtain the desired product, N-(4-(methylsulfonyl)phenyl)-1H-pyrazolo[3,4-d]pyrimidin-6-amine Int-04 (1.40 g, 4.83 mmol, 74.6% yield) as a pale yellow solid. LCMS (Method A): 99.8%, RT: 1.66 min, m/z: 291.0 (M+H)⁺.

### Compound 3: N-(4-(methylsulfonyl)phenyl)-1-(tetrahydro-2H-pyran-4-yl)-1H-pyrazolo[3,4-d]pyrimidin-6-amine

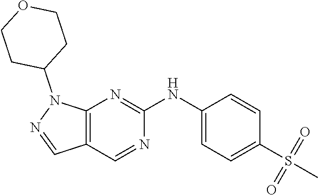 [SMILES: CS(=O)(=O)c1ccc(Nc2ncc3cnn(C4CCOCC4)c3n2)cc1]

To a solution of N-(4-(methylsulfonyl)phenyl)-1H-pyrazolo[3,4-d]pyrimidin-6-amine Int-04 (100 mg, 0.346 mmol) in DMF (5 mL) were added tetrahydro-2H-pyran-4-yl methanesulfonate (93 mg, 0.518 mmol) and Cs₂CO₃ (338 mg, 1.037 mmol) at rt. The resultant reaction mixture was heated at 85° C. for 16 h. After completion, the reaction mixture was diluted with water (30 mL) and extracted with DCM (2×30 mL). The combined organic layers were washed with brine (20 mL), dried over anhydrous Na₂SO₄, filtered and concentrated in vacuo to afford crude mixture, which was purified by prep HPLC and pure fractions were lyophilized to obtain the desired product, N-(4-(methylsulfonyl)phenyl)-1-(tetrahydro-2H-pyran-4-yl)-1H-pyrazolo[3,4-d]pyrimidin-6-amine (25 mg, 0.067 mmol, 19.3% yield) as an off-white solid (peak-2 from prep HPLC). ¹H NMR (400 MHz, DMSO-d₆): δ 10.46 (bs, 1H), 9.09 (s, 1H), 8.18 (s, 1H), 8.17-8.08 (m, 2H), 7.91-7.83 (m, 2H), 5.00-4.84 (m, 1H), 4.10-4.00 (m, 2H), 3.69-3.55 (m, 2H), 3.17 (s, 3H), 2.23-2.14 (m, 2H), 1.95-1.85 (m, 2H). LCMS (Method A): 99.8%, RT: 1.97 min, m/z: 374.0 [M+H]⁺.

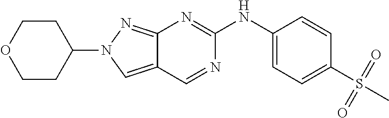 [SMILES: CS(=O)(=O)c1ccc(Nc2ncc3cn(C4CCOCC4)nc3n2)cc1]

The other regio-isomer, N-(4-(methylsulfonyl)phenyl)-2-(tetrahydro-2H-pyran-4-yl)-2H-pyrazolo[3,4-d]pyrimidin-6-amine (2.3 mg, 6.12 μmol, 1.8% yield) was also isolated as an off-white solid (peak-1 from prep HPLC). ¹H NMR (400 MHz, DMSO-d₆): δ 10.23 (s, 1H), 9.24 (s, 1H), 8.61 (s, 1H), 8.19 (d, J=8.8 Hz, 2H), 7.85 (d, J=8.8 Hz, 2H), 4.76-4.66 (m, 1H), 4.05-3.98 (m, 2H), 3.60-3.43 (m, 2H), 3.16 (s, 3H), 2.15-2.07 (m, 4H). LCMS (Method A): 99.1%, RT: 1.80 min, m/z: 374.2 [M+H]⁺.

### Compound 4: 1-benzyl-N-(4-(methylsulfonyl)phenyl)-1H-pyrazolo[3,4-d]pyrimidin-6-amine

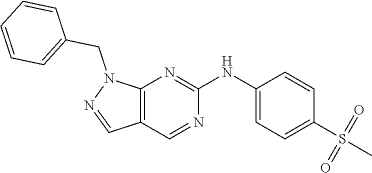 [SMILES: CS(=O)(=O)c1ccc(Nc2ncc3cnn(Cc4ccccc4)c3n2)cc1]

To a solution of N-(4-(methylsulfonyl)phenyl)-1H-pyrazolo[3,4-d]pyrimidin-6-amine Int-04 (200 mg, 0.691 mmol) and (bromomethyl)benzene (118 mg, 0.691 mmol) in DMF (10 mL) was added Cs₂CO₃ (676 mg, 2.07 mmol) at rt. The reaction mixture was heated at 80° C. for 16 h. After completion, the reaction mixture was diluted with water (20 mL) and filtered to obtain a crude solid, which was purified by prep HPLC and pure fractions were lyophilized to obtain the desired product, 1-benzyl-N-(4-(methylsulfonyl)phenyl)-1H-pyrazolo[3,4-d]pyrimidin-6-amine (44 mg, 0.112 mmol, 16.2% yield) as an off-white solid (peak-2 from prep HPLC). ¹H NMR (400 MHz, DMSO-d₆): δ 10.49 (bs, 1H), 9.11 (s, 1H), 8.17-8.08 (m, 3H), 7.90-7.81 (m, 2H), 7.43-7.20 (m, 5H), 5.60 (s, 2H), 3.17 (s, 3H). LCMS (Method A): 96.22% RT: 2.38 min, m/z: 380.2 [M+H]⁺.

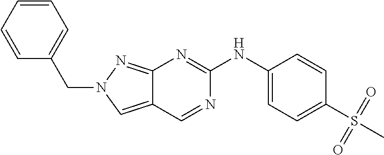 [SMILES: CS(=O)(=O)c1ccc(Nc2ncc3cn(Cc4ccccc4)nc3n2)cc1]

The minor regio-isomer, 2-benzyl-N-(4-(methylsulfonyl)phenyl)-2H-pyrazolo[3,4-d]pyrimidin-6-amine (27 mg, 0.071 mmol, 10.3% yield) was isolated as an off-white solid (peak-1 from prep HPLC). ¹H NMR (400 MHz, DMSO-d₆): δ 10.25 (s, 1H), 9.26 (s, 1H), 8.69 (s, 1H), 8.18-8.167 (m, 2H), 7.85-7.82 (m, 2H), 7.41-7.30 (m, 5H), 5.59 (s, 2H), 3.15 (s, 3H). LCMS (Method A): 99.3%, RT: 2.13 min, m/z: 380.0 [M+H]⁺.

### Compound 5: N-(4-(methylsulfonyl)phenyl)-1-(pyridine-3-ylmethyl)-1H-pyrazolo[3,4-d]pyrimidin-6-amine

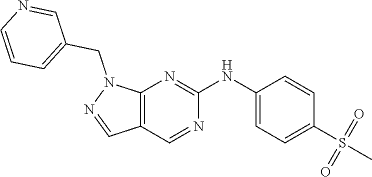 [SMILES: CS(=O)(=O)c1ccc(Nc2ncc3cnn(Cc4cccnc4)c3n2)cc1]

To a solution of N-(4-(methylsulfonyl)phenyl)-1H-pyrazolo[3,4-d]pyrimidin-6-amine Int-04 (150 mg, 0.518 mmol) in DMF (8 mL) were added Cs₂CO₃ (507 mg, 1.555 mmol) and 3-(bromomethyl)pyridine hydrobromide (131 mg, 0.518 mmol) at rt. The reaction mixture was stirred at 80° C. for 16 h. After completion, reaction mixture was quenched with water (30 mL) and extracted with EtOAc (3×50 mL). The combined organic layers were dried over anhydrous Na₂SO₄, and concentrated in vacuo to get crude product, which was purified by prep HPLC and pure fractions were lyophilized to obtain the desired product, N-(4-(methylsulfonyl)phenyl)-1-(pyridine-3-ylmethyl)-1H-pyrazolo[3,4-d]pyrimidin-6-amine (6 mg, 0.016 mmol, 3.01% yield) as an off-white solid. ¹H NMR (400 MHz, DMSO-d₆): δ 10.52 (bs, 1H), 9.11 (s, 1H), 8.64 (d, J=1.6 Hz, 1H), 8.51 (dd, J=4.8, 1.7 Hz, 1H), 8.21 (s, 1H), 8.17-8.10 (m, 2H), 7.92-7.80 (m, 2H), 7.72-7.69 (m, 1H), 7.40-7.37 (m, 1H), 5.66 (s, 2H), 3.17 (s, 3H). LCMS (Method A): 98.6%, RT: 1.63 min, m/z: 381.2 (M+H)⁺.

### Compound 6: 1-(((1S,4S)-4-aminocyclohexyl)methyl)-N-(4-(methylsulfonyl)phenyl)-1H-pyrazolo[3,4-d]pyrimidin-6-amine

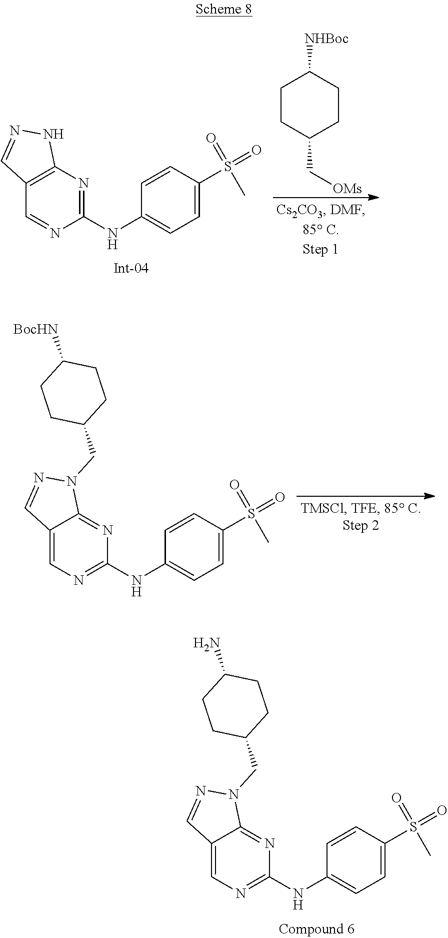

### Step 1: tert-butyl ((1S,4S)-4-((6-((4-(methylsulfonyl)phenyl)amino)-1H-pyrazolo[3,4-d]pyrimidin-1-yl)methyl)cyclohexyl)carbamate

To a solution of N-(4-(methylsulfonyl)phenyl)-1H-pyrazolo[3,4-d]pyrimidin-6-amine Int-04 (150 mg, 0.518 mmol) in DMF (8 mL) were added Cs₂CO₃ (507 mg, 1.56 mmol) and cis-4-((tert-butoxycarbonyl)amino)cyclohexyl)methyl methanesulfonate (159 mg, 0.518 mmol) at rt. The reaction mixture was heated at 80° C. for 16 h. After completion, the reaction mixture was quenched with water (20 mL) and extracted with EtOAc (3×20 mL). The combined organic layers were dried over anhydrous Na₂SO₄ and concentrated in vacuo to afford crude product, which was purified by prep HPLC and pure fractions were lyophilized to obtain the desired product, tert-butyl ((1S,4S)-4-((6-((4-(methylsulfonyl)phenyl)amino)-1H-pyrazolo[3,4-d]pyrimidin-1-yl)methyl)cyclohexyl)carbamate (200 mg, 0.400 mmol, 77.2% yield) as an off-white solid. LCMS (Method A): 22.9%, RT: 2.51 min, m/z: 499.4 [M−H]⁻.

### Step 2. Compound 6: 1-(((1S,4S)-4-aminocyclohexyl)methyl)-N-(4-(methylsulfonyl)phenyl)-1H-pyrazolo[3,4-d]pyrimidin-6-amine

To a solution of tert-butyl ((1S,4S)-4-((6-((4-(methylsulfonyl)phenyl)amino)-1H-pyrazolo[3,4-d]pyrimidin-1-yl)methyl)cyclohexyl)carbamate (60.0 mg, 0.120 mmol) in TFE (10 mL) was added TMSCl (13 mg, 0.120 mmol) at 0° C. The reaction mixture was stirred at rt for 30 min. After completion, the reaction mixture was concentrated in vacuo to afford the desired product, 1-(((1S,4S)-4-aminocyclohexyl)methyl)-N-(4-(methylsulfonyl)phenyl)-1H-pyrazolo[3,4-d]pyrimidin-6-amine hydrochloride (41 mg, 0.092 mmol, 76.6% yield) as a white solid. ¹H NMR (400 MHz, DMSO-d₆): δ 10.49 (s, 1H), 9.09 (s, 1H), 8.18-8.11 (m, 5H), 7.97-7.87 (m, 2H), 4.40-4.31 (m, 2H), 3.18 (s, 4H), 2.20-2.15 (m, 1H), 1.87-1.73 (m, 2H), 1.69-1.63 (m, 2H), 1.62-1.43 (m, 4H). LCMS (Method A): 98.2%, RT: 1.58 min, m/z: 401.3 [M+H]⁺.

### Compound 7: 1-(((1S,4S)-4-aminocyclohexyl)methyl)-N-(4-(methylsulfonyl)phenyl)-1H-pyrazolo[3,4-d]pyrimidin-6-amine

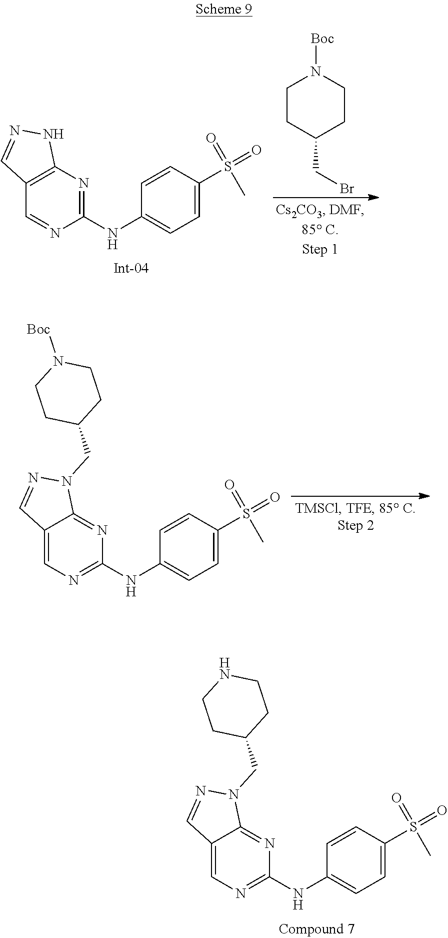

### Step 1: tert-butyl 4-((6-((4-(methylsulfonyl)phenyl)amino)-1H-pyrazolo[3,4-d]pyrimidin-1-yl)methyl)piperidine-1-carboxylate

To a solution of N-(4-(methylsulfonyl)phenyl)-1H-pyrazolo[3,4-d]pyrimidin-6-amine (150 mg, 0.518 mmol) in DMF (8 mL) were added Cs₂CO₃ (507 mg, 1.555 mmol) and tert-butyl 4-(bromomethyl)piperidine-1-carboxylate (144 mg, 0.518 mmol) at rt. The reaction mixture was stirred at 80° C. for 16 h. After completion, the reaction mixture was quenched with water (30 mL) and extracted with EtOAc (3×50 mL). The combined organic layers were dried over anhydrous Na₂SO₄ and concentrated in vacuo to get crude product, which was purified by prep HPLC and pure fractions were lyophilized to obtain the desired product, tert-butyl 4-((6-((4-(methylsulfonyl)phenyl)amino)-1H-pyrazolo[3,4-d]pyrimidin-1-yl)methyl)piperidine-1-carboxylate (100 mg, 0.205 mmol, 39.5% yield) as an off-white solid (Peak 2 from prep HPLC). ¹H NMR (400 MHz, DMSO-d₆): δ 10.46 (s, 1H), 9.09 (s, 1H), 8.20-8.12 (m, 3H), 7.87 (d, J=8.8 Hz, 2H), 4.27 (d, J=6.9 Hz, 2H), 4.10-4.01 (m, 2H), 3.16 (s, 3H), 2.80-2.62 (m, 2H), 2.19-2.09 (m, 1H), 1.59-1.51 (m, 2H), 1.37 (s, 9H), 1.20-1.15 (m, 2H). LCMS (Method A): 99.9%, RT: 2.44 min, m/z: 387.2 [(M-Boc)+H]⁺.

The other regio-isomer, tert-butyl 4-((6-((4-(methylsulfonyl)phenyl)amino)-2H-pyrazolo[3,4-d]pyrimidin-2-yl)methyl)piperidine-1-carboxylate (50 mg, 0.102 mmol, 19.72% yield) was also isolated as an off-white solid (peak-1 from prep HPLC). ¹H NMR (400 MHz, DMSO-d₆): δ 10.24 (s, 1H), 9.25 (s, 1H), 8.53 (s, 1H), 8.19 (d, J=8.8 Hz, 2H), 7.85 (d, J=8.8 Hz, 2H), 4.28 (d, J=7.2 Hz, 2H), 4.09-4.00 (m, 2H), 3.16 (s, 3H), 2.80-2.62 (m, 2H), 2.29-2.20 (m, 1H), 1.51-1.41 (m, 2H), 1.39 (s, 9H), 1.21-1.10 (m, 2H). LCMS (Method A): 99.6%, RT: 2.29 min, m/z: 431.0 [(M-C₄H₈)+H)]⁺, 387.2 [(M-Boc)+H]⁺

### Step 2. Compound 7: N-(4-(methylsulfonyl)phenyl)-1-(piperidin-4-ylmethyl)-1H-pyrazolo[3,4-d]pyrimidin-6-amine hydrochloride

To a solution of tert-butyl 4-((6-((4-(methylsulfonyl)phenyl)amino)-1H-pyrazolo[3,4-d]pyrimidin-1-yl)methyl)piperidine-1-carboxylate (60.0 mg, 0.123 mmol) in TFE (10 mL) was added TMSCl (13 mg, 0.123 mmol) at 0° C. The reaction mixture was stirred at rt for 30 min. After completion, the reaction mixture was concentrated in vacuo to obtain the desired product, N-(4-(methylsulfonyl)phenyl)-1-(piperidin-4-ylmethyl)-1H-pyrazolo[3,4-d]pyrimidin-6-amine hydrochloride (45.0 mg, 0.106 mmol, 86.6% yield) as a white solid. ¹H NMR (400 MHz, DMSO-d₆): δ 10.51 (s, 1H), 9.11 (s, 1H), 8.98-8.82 (m, 1H), 8.75-8.60 (m, 1H), 8.29-8.09 (m, 3H), 7.99-7.81 (m, 2H), 4.31 (d, J=6.8 Hz, 2H), 3.30-3.19 (m, 2H), 3.18 (s, 3H), 2.95-2.75 (m, 2H), 2.29-2.18 (m, 1H), 1.80-1.65 (m, 2H), 1.62-1.42 (m, 2H). LCMS (Method A): 99.9%, RT: 1.51 min, m/z: 387.2 [M+H]⁺.

### Compound 8: 1-(cyclohexylmethyl)-N-(4-(vinylsulfonyl)phenyl)-1H-pyrazolo[3,4-d]pyrimidin-6-amine

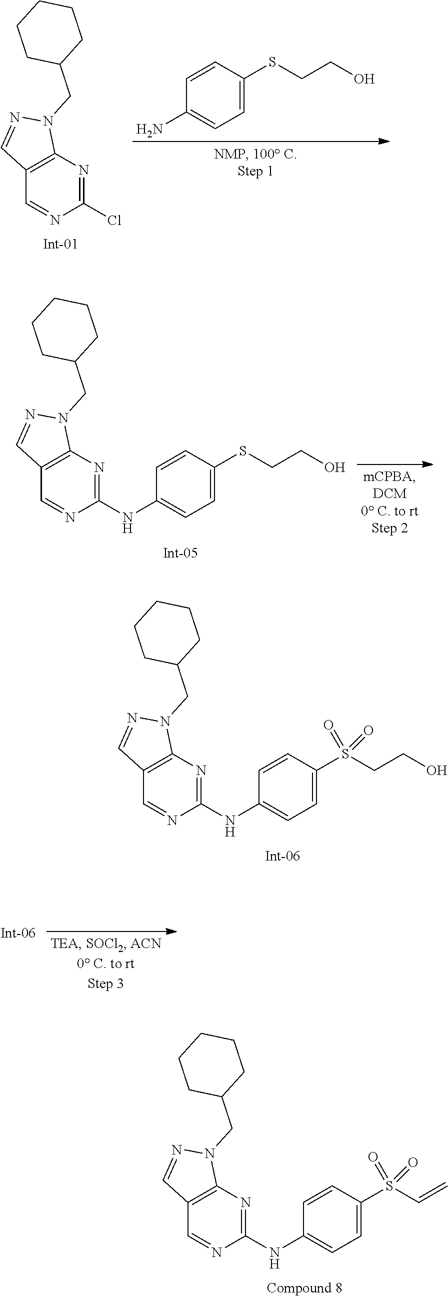 [SMILES: Clc1ncc2cnn(CC3CCCCC3)c2n1 ; Nc1ccc(SCCO)cc1 ; OCCSc1ccc(Nc2ncc3cnn(CC4CCCCC4)c3n2)cc1 ; O=S(=O)(CCO)c1ccc(Nc2ncc3cnn(CC4CCCCC4)c3n2)cc1 ; C=CS(=O)(=O)c1ccc(Nc2ncc3cnn(CC4CCCCC4)c3n2)cc1]

### Step 1. Intermediate 05: 2-((4-((1-(cyclohexylmethyl)-1H-pyrazolo[3,4-d]pyrimidin-6-yl)amino)phenyl)thio)ethan-1-ol

To a solution of 6-chloro-1-(cyclohexylmethyl)-1H-pyrazolo[3,4-d]pyrimidine Int-01 (481 mg, 1.920 mmol) in NMP (1.0 mL) was added 2-((4-aminophenyl)thio)ethan-1-ol (250 mg, 1.477 mmol) at rt and the resultant solution was heated at 100° C. for 16 h. After completion, solid precipitate was filtered, washed with MTBE (10 mL) and dried to afford the desired product, 2-((4-((1-(cyclohexylmethyl)-1H-pyrazolo[3,4-d]pyrimidin-6-yl)amino)phenyl)thio)ethan-1-ol Int-05 (550 mg, 1.420 mmol, 73.9% yield) as a light green solid. LCMS (Method A): 99.8%, RT: 1.99 min, m/z: 384.1 (M+H)⁺.

### Step 2. Intermediate 06: 2-((4-((1-(cyclohexylmethyl)-1H-pyrazolo[3,4-d]pyrimidin-6-yl)amino)phenyl)sulfonyl)ethan-1-ol

To a solution of 2-((4-((1-(cyclohexylmethyl)-1H-pyrazolo[3,4-d]pyrimidin-6-yl)amino)phenyl)thio)ethan-1-ol Int-05 (240 mg, 0.626 mmol) in DCM (5 mL) was added mCPBA (498 mg, 1.88 mmol) at 0° C. The reaction mixture was stirred at rt for 1.5 h. After completion, the reaction mixture was quenched with saturated solution of aqueous NaHCO₃ (50 mL) and extracted with EtOAc (3×30 mL). The combined organic layers were washed with brine (30 mL), dried over anhydrous Na₂SO₄, filtered, and concentrated in vacuo to afford crude compound, which was purified by Biotage Isolera (100% EtOAc) to obtain the desired product, 2-((4-((1-(cyclohexylmethyl)-1H-pyrazolo[3,4-d]pyrimidin-6-yl)amino)phenyl)sulfonyl)ethan-1-ol Int-06 (145 mg, 0.314 mmol, 50.2% yield) as an off-white solid. LCMS (Method A): 98.5%, RT: 1.90 min, m/z: 416.3 (M+H)⁺.

### Step 3. Compound 8: 1-(cyclohexylmethyl)-N-(4-(vinylsulfonyl)phenyl)-1H-pyrazolo[3,4-d]pyrimidin-6-amine

To a solution of 2-((4-((1-(cyclohexylmethyl)-1H-pyrazolo[3,4-d]pyrimidin-6-yl)amino)phenyl)sulfonyl)ethan-1-ol Int-06 (80.0 mg, 0.193 mmol) in ACN (5 mL) were added TEA (0.268 mL, 1.925 mmol) and SOCl₂ (0.028 mL, 0.385 mmol) at 0° C. The reaction mixture was stirred at rt for 1 h. After completion, the reaction mixture was concentrated in vacuo to get crude compound as brown solid, which was purified by prep HPLC and pure fractions were lyophilized to afford the desired compound, 1-(cyclohexylmethyl)-N-(4-(vinylsulfonyl)phenyl)-1H-pyrazolo[3,4-d]pyrimidin-6-amine (18 mg, 0.046 mmol, 23.8% yield) as an off-white solid.

¹H NMR (400 MHz, DMSO-d₆): δ 10.48 (s, 1H), 9.08 (s, 1H), 8.21-8.13 (m, 3H), 7.85-7.77 (m, 2H), 7.08 (dd, J=16.4, 9.9 Hz, 1H), 6.27 (d, J=16.4 Hz, 1H), 6.13 (d, J=9.8 Hz, 1H), 4.21 (d, J=7.0 Hz, 2H), 1.99-1.96 (m, 1H), 1.66 (m, 2H), 1.58 (m, 3H), 1.26-1.13 (m, 2H), 1.05 (m, 3H). LCMS (Method D): 99.3%, RT: 2.07 min, m/z: 398.0 (M+H)⁺.

### Compound 9: N-(4-(vinylsulfonyl)phenyl)-1H-pyrazolo[3,4-d]pyrimidin-6-amine

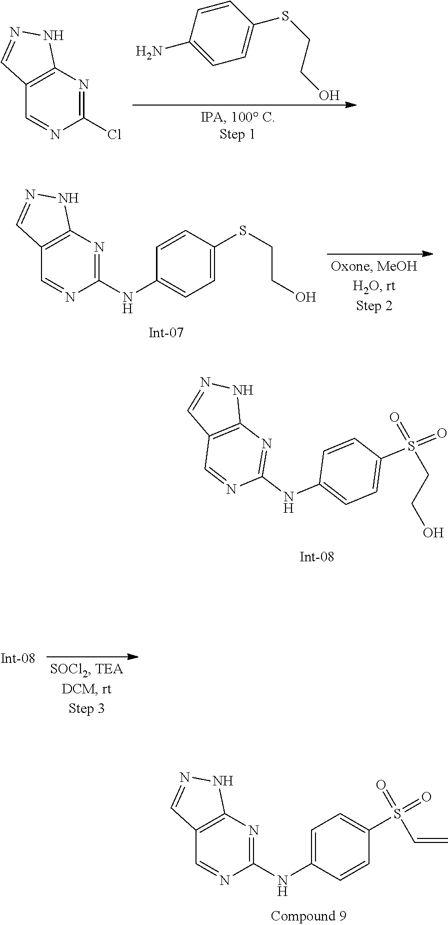 [SMILES: Clc1ncc2cn[nH]c2n1 ; Nc1ccc(SCCO)cc1 ; OCCSc1ccc(Nc2ncc3cn[nH]c3n2)cc1 ; O=S(=O)(CCO)c1ccc(Nc2ncc3cn[nH]c3n2)cc1 ; C=CS(=O)(=O)c1ccc(Nc2ncc3cn[nH]c3n2)cc1]

### Step 1. Intermediate 07: 2-((4-((1H-pyrazolo[3,4-d]pyrimidin-6-yl)amino)phenyl)thio)ethan-1-ol

To a solution of 6-chloro-1H-pyrazolo[3,4-d]pyrimidine (416 mg, 2.69 mmol) in IPA (3 mL) was added 2-((4-aminophenyl)thio)ethan-1-ol (350 mg, 2.07 mmol) at rt. The resultant solution was heated at 100° C. for 16 h. After completion, solid precipitate obtained was filtered, washed with MTBE (10 mL) and dried to afford the desired product, 2-((4-((1H-pyrazolo[3,4-d]pyrimidin-6-yl)amino)phenyl)thio)ethan-1-ol Int-07 (550 mg, 1.86 mmol, 90% yield) as a pale yellow solid. LCMS (Method D): 97.6%, RT: 1.32 min, m/z: 288.0 [M+H]⁺.

### Step 2. Intermediate 08: 2-((4-((1H-pyrazolo[3,4-d]pyrimidin-6-yl)amino)phenyl)sulfonyl)ethan-1-ol

To a solution of 2-((4-((1H-pyrazolo[3,4-d]pyrimidin-6-yl)amino)phenyl)tho)ethan-1-ol (430 mg, 1.50 mmol) in H₂O (5 mL) and MeOH (5 mL) was added Oxone® (2.76 g, 4.49 mmol) at 0° C. The reaction mixture was stirred at rt for 1 h. After completion, the reaction mixture was quenched with saturated aqueous solution of NaHCO₃ (30 mL) and extracted with DCM (3×30 mL). The combined organic layers were washed with brine (30 mL), dried over anhydrous Na₂SO₄, filtered and concentrated in vacuo to afford crude product, which was purified by Biotage Isolera (80% EtOAc in hexanes) to obtain the desired product, 2-((4-((1H-pyrazolo[3,4-d]pyrimidin-6-yl)amino)phenyl)sulfonyl)ethan-1-ol Int-08 (185 mg, 0.556 mmol, 37.2% yield) as an off-white solid. LCMS (Method A): 96.6%, RT: 1.22 min, m/z: 320.2 [M+H]⁺.

### Step 3. Compound 9: N-(4-(vinylsulfonyl)phenyl)-1H-pyrazolo[3,4-d]pyrimidin-6-amine

To a solution of 2-((4-((1H-pyrazolo[3,4-d]pyrimidin-6-yl)amino)phenyl)sulfonyl)ethan-1-ol (85 mg, 0.266 mmol) in ACN (5 mL) were added TEA (0.371 mL, 2.66 mmol) and SOCl₂ (0.039 mL, 0.532 mmol) at 0° C. The reaction mixture was stirred at rt for 1 h. After completion, the reaction mixture was concentrated in vacuo to afford crude product, which was purified by prep HPLC and pure fractions were lyophilized to obtain the desired product, N-(4-(vinylsulfonyl)phenyl)-1H-pyrazolo[3,4-d]pyrimidin-6-amine (2 mg, 6.11 μmol, 2.2% yield) as an off-white solid. ¹H NMR (400 MHz, DMSO-d₆): δ 10.40 (bs, 1H), 9.10 (s, 2H), 8.20-8.02 (m, 3H), 7.86-7.69 (m, 2H), 7.07 (dd, J=16.4, 9.9 Hz, 1H), 6.26 (d, J=16.5 Hz, 1H), 6.13 (d, J=9.8 Hz, 1H). LCMS (Method D): 98.8%, RT: 1.38 min, m/z: 302.0 (M+H)⁺.

### Compound 10: 1-methyl-N-(4-(vinylsulfonyl)phenyl)-1H-pyrazolo[3,4-d]pyrimidin-6-amine

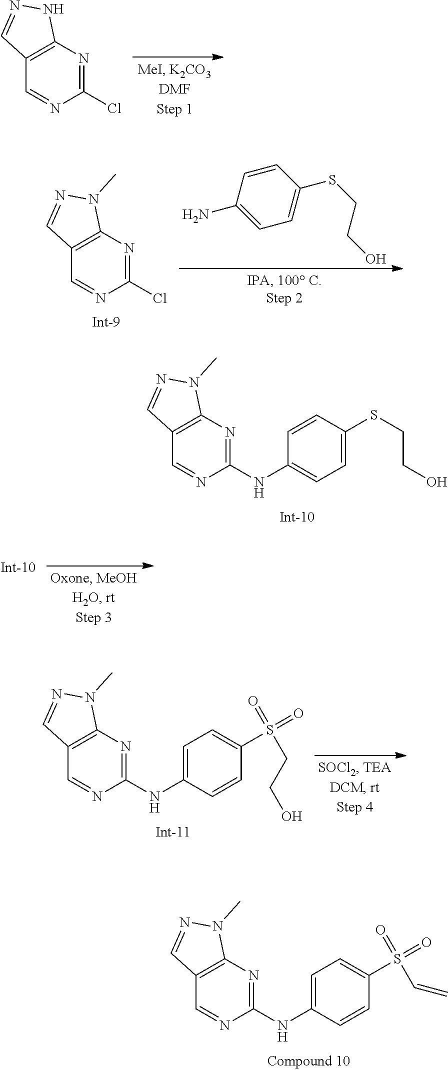 [SMILES: Clc1ncc2cn[nH]c2n1 ; Cn1ncc2cnc(Cl)nc21 ; Nc1ccc(SCCO)cc1 ; Cn1ncc2cnc(Nc3ccc(SCCO)cc3)nc21 ; Cn1ncc2cnc(Nc3ccc(S(=O)(=O)CCO)cc3)nc21 ; C=CS(=O)(=O)c1ccc(Nc2ncc3cnn(C)c3n2)cc1]

### Step 1. Intermediate 09: 6-chloro-1-methyl-1H-pyrazolo[3,4-d]pyrimidine

To a solution of 6-chloro-1H-pyrazolo[3,4-d]pyrimidine (500 mg, 3.24 mmol) in DMF (5 mL) were added K₂CO₃ (1.12 g, 8.09 mmol) and Mel (0.607 mL, 9.71 mmol) at 0° C. The reaction mixture was stirred at rt for 4 h. After completion, the reaction mixture was quenched with ice-cold water (10 mL) and extracted with EtOAc (3×30 mL). The combined organic layers were washed with brine (30 mL), dried over anhydrous Na₂SO₄, filtered and concentrated in vacuo to afford crude as a brown solid, which was purified by Biotage Isolera (60% EtOAc in hexanes) to obtain the desired product, 6-chloro-1-methyl-1H-pyrazolo[3,4-d]pyrimidine Int-09 (210 mg, 1.23 mmol, 38.1% yield) as an off-white solid. LCMS (Method D): 99.9%, RT: 1.14 min, m/z: 169.0 (M+H)⁺.

### Step 2. Intermediate 10: 2-((4-((1-methyl-1H-pyrazolo[3,4-d]pyrimidin-6-yl)amino)phenyl)thio)ethan-1-ol

To a solution of 6-chloro-1-methyl-1H-pyrazolo[3,4-d]pyrimidine Int-09 (104 mg, 0.615 mmol) in IPA (3 mL) was added 2-((4-aminophenyl)thio)ethan-1-ol (80.0 mg, 0.473 mmol) at rt. The resultant reaction mixture was heated at 100° C. for 16 h. After completion, solid precipitate was filtered, washed with MTBE (10 mL) and then dried to afford the desired product, 2-((4-((1-methyl-1H-pyrazolo[3,4-d]pyrimidin-6-yl)amino)phenyl)thio)ethan-1-ol Int-10 (140 mg, 0.498 mmol, 98% yield) as a yellow solid. LCMS (Method D): 99.2%, RT: 1.46 min, m/z: 302.0 (M+H)⁺.

### Step 3. Intermediate 11: 2-((4-((1-methyl-1H-pyrazolo[3,4-d]pyrimidin-6-yl)amino)phenyl)sulfonyl)ethan-1-ol

To a solution of 2-((4-((1-methyl-1H-pyrazolo[3,4-d]pyrimidin-6-yl)amino)phenyl)thio)ethan-1-ol Int-10 (300 mg, 0.995 mmol) in water (3 mL) and MeOH (3 mL) was added Oxone® (2.45 g, 3.98 mmol) at 0° C. The reaction mixture was stirred at rt for 1 h. After completion, the reaction mixture was quenched with saturated aqueous solution of NaHCO₃ (20 mL) and extracted with DCM (3×30 mL). The combined organic layers were washed with brine (30 mL), dried over anhydrous Na₂SO₄, filtered and concentrated in vacuo to afford crude compound as a brown solid, which was purified by Biotage Isolera 80% EtOAc in hexanes) to obtain the desired product, 2-((4-((1-methyl-1H-pyrazolo[3,4-d]pyrimidin-6-yl)amino)phenyl)sulfonyl)ethan-1-ol Int-11 (300 mg, 0.801 mmol, 80% yield) as an off-white solid. LCMS (Method A): 88.0%, RT: 1.28 min, m/z: 334.2 (M+H)⁺.

### Step 4. Compound 10: 1-methyl-N-(4-(vinylsulfonyl)phenyl)-1H-pyrazolo[3,4-d]pyrimidin-6-amine

To a solution of 2-((4-((1-methyl-1H-pyrazolo[3,4-d]pyrimidin-6-yl)amino)phenyl)sulfonyl)ethan-1-ol Int-11 (90 mg, 0.270 mmol)) in ACN (3 mL) were added TEA (0.376 mL, 2.70 mmol) and SOCl₂ (0.039 mL, 0.540 mmol) at 0° C. The reaction mixture was stirred at rt for 1 h. After completion, the reaction mixture was concentrated in vacuo to get crude compound as a brown solid, which was purified by prep HPLC and pure fractions were lyophilized to obtain the desired product, 1-methyl-N-(4-(vinylsulfonyl)phenyl)-1H-pyrazolo[3,4-d]pyrimidin-6-amine (26 mg, 0.081 mmol, 29.9% yield) as an off-white solid. ¹H NMR (400 MHz, DMSO-d₆): δ 10.50 (bs, 1H), 9.09 (s, 1H), 8.25-8.06 (m, 3H), 7.87-7.72 (m, 2H), 7.07 (dd, J=16.4, 9.8 Hz, 1H), 6.27 (d, J=16.4 Hz, 1H), 6.13 (d, J=9.8 Hz, 1H), 3.98 (s, 3H). LCMS (Method D): 99.6%, RT: 1.52 min, m/z: 315.9 (M+H)⁺.

### Compound 11: ((1r,4r)-4-((6-((4-(methylsulfonyl)phenyl)amino)-1H-pyrazolo[3,4-d]pyrimidin-1-yl)methyl)cyclohexyl)methanol

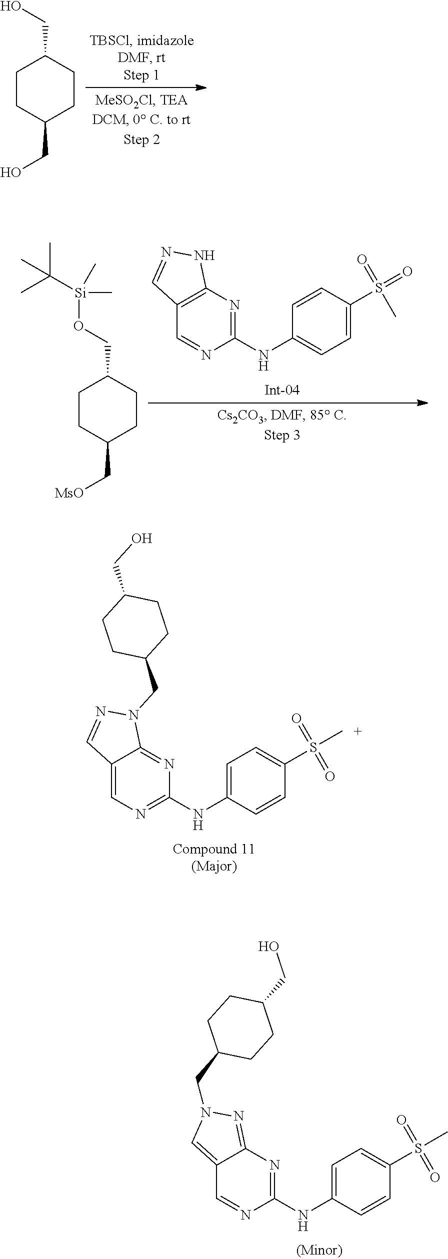

### Step 1: ((1r,4r)-4-(((tert-butyldimethylsilyl)oxy)methyl)cyclohexyl)methanol

To a stirred solution ((1r,4r)-cyclohexane-1,4-diyl)dimethanol (500 mg, 3.47 mmol) in DMF (5 mL) were added imidazole (142 mg, 2.08 mmol) and TBSCl (261 mg, 1.734 mmol) at rt. The reaction mixture was stirred at rt for 24 h. After completion, the reaction mixture was diluted with water (100 mL) and extracted with EtOAc (2×100 mL). The combined organic layers were washed with brine (50 mL), dried over anhydrous Na₂SO₄, filtered, and concentrated in vacuo to afford a crude liquid, which was purified by Biotage Isolera (5% MeOH in DCM) to obtain the desired product, ((1r,4r)-4-(((tert-butyldimethylsilyl)oxy)methyl)cyclohexyl)methanol (190 mg, 0.735 mmol, 21.2% yield) as a colorless liquid.

### Step 2: ((1r,4r)-4-(((tert-butyldimethylsilyl)oxy)methyl)cyclohexyl)methyl methanesulfonate

To a stirred solution of ((1r,4r)-4-(((tert-butyldimethylsilyl)oxy)methyl)cyclohexyl)methanol (190 mg, 0.735 mmol) in DCM (5 mL) were added TEA (0.205 mL, 1.47 mmol) and MeSO₂Cl (0.086 mL, 1.10 mmol) at 0° C. The reaction mixture was stirred at rt for 1 h. After completion, the reaction mixture was diluted with water (70 mL) and extracted with DCM (2×100 mL). The combined organic layers were washed with brine (50 mL), dried over anhydrous Na₂SO₄, filtered and concentrated in vacuo to afford the desired product, ((1r,4r)-4-(((tert-butyldimethylsilyl)oxy)methyl)cyclohexyl)methyl methanesulfonate (230 mg, crude), which was used without further purification.

### Step 3. Compound 11: ((1r,4r)-4-((6-((4-(methylsulfonyl)phenyl)amino)-1H-pyrazolo[3,4-d]pyrimidin-1-yl)methyl)cyclohexyl)methanol

To a solution of N-(4-(methylsulfonyl)phenyl)-1H-pyrazolo[3,4-d]pyrimidin-6-amine (120 mg, 0.415 mmol) in DMF (8 mL) were added Cs₂CO₃ (405 mg, 1.24 mmol) and ((1r,4r)-4-(((tert-butyldimethylsilyl)oxy)methyl)cyclohexyl)methyl methanesulfonate (209 mg, 0.622 mmol) at rt. The reaction mixture was stirred at 85° C. for 16 h. After completion, the reaction mixture was diluted with water (100 mL) and extracted with EtOAc (2×100 mL). The combined organic layers were washed with brine (100 mL), dried over anhydrous Na₂SO₄, filtered, and concentrated in vacuo to afford a crude solid, which was purified by reverse phase chromatography using Biotage Isolera (30% aq NH₄HCO₃ in ACN) and pure fractions were lyophilized to afford the desired compound, ((1r,4r)-4-((6-((4-(methylsulfonyl)phenyl)amino)-1H-pyrazolo[3,4-d]pyrimidin-1-yl)methyl)cyclohexyl)methanol (8 mg, 0.012 mmol, 2.8% yield) as a white solid (peak-2).

¹H NMR (400 MHz, DMSO-d₆): δ 10.45 (s, 1H), 9.08 (s, 1H), 8.19-8.11 (m, 3H), 7.87 (d, J=9.2 Hz, 2H), 4.34 (t, J=5.2 Hz, 1H), 4.21 (d, J=6.8 Hz, 2H), 3.19-3.18 (m, 5H), 1.99-1.88 (m, 1H), 1.78-1.70 (m, 2H), 1.67-1.57 (m, 2H), 1.39-1.27 (m, 1H), 1.18-0.96 (m, 2H), 0.90-0.78 (m, 2H). LCMS (Method A): 98.5%, RT: 2.12 min, m/z: 416.1 [M+H]⁺.

### Compound 16: (1s,4s)-4-((6-((4-(methylsulfonyl)phenyl)amino)-1H-pyrazolo[3,4-d]pyrimidin-1-yl)methyl)cyclohexane-1-carboxylic acid

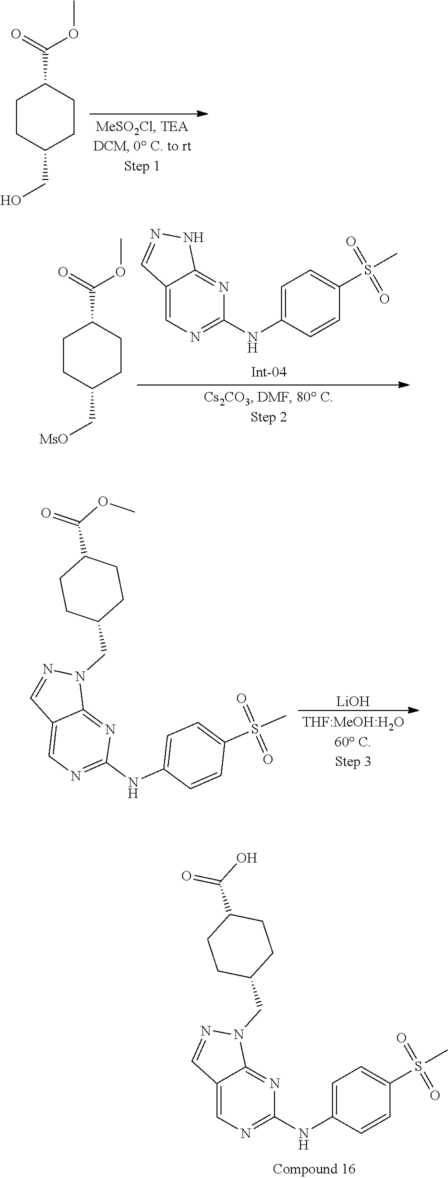

### Step 1: Methyl (1s,4s)-4-(((methylsulfonyl)oxy)methyl)cyclohexane-1-carboxylate

To a stirred solution of methyl (1s,4s)-4-(hydroxymethyl)cyclohexane-1-carboxylate (100 mg, 0.581 mmol) in DCM (10 mL) were added TEA (59 mg, 0.581 mmol) and MeSO₂Cl (67 mg, 0.58 mmol) added at 0° C. The reaction mixture was stirred at rt for 16 h. After completion, the reaction mixture was diluted with water (50 mL) and extracted with DCM (3×50 mL). The combined organic layers were washed with brine (50 mL), dried over anhydrous Na₂SO₄, filtered and concentrated in vacuo to afford the desired product, methyl (1s,4s)-4-(((methylsulfonyl)oxy)methyl)cyclohexane-1-carboxylate as a crude liquid, which was used without further purification.

### Step 2: Methyl (1s,4s)-4-((6-((4-(methylsulfonyl)phenyl)amino)-1H-pyrazolo[3,4-d]pyrimidin-1-yl)methyl)cyclohexane-1-carboxylate

To a stirred solution of N-(4-(methylsulfonyl)phenyl)-1H-pyrazolo[3,4-d]pyrimidin-6-amine (100 mg, 0.346 mmol) in DMF (8 mL) were added Cs₂CO₃ (338 mg, 1.04 mmol) and methyl (1s,4s)-4-(((methylsulfonyl)oxy)methyl)cyclohexane-1-carboxylate (87 mg, 0.35 mmol) at rt. The reaction mixture was stirred at 80° C. for 16 h. After completion, the reaction mixture was diluted with water (100 mL) and extracted with EtOAc (3×100 mL). The combined organic layers were washed with brine (100 mL), dried over anhydrous Na₂SO₄, filtered and concentrated in vacuo to afford a crude mixture of two regioisomers (100 mg), which was used without further purification. LCMS (Method A): 29.4% and 67.6%, RT: 2.17 and 2.35 min respectively, m/z: 444.2 [M+H]⁺.

### Step 3. Compound 16: (1s,4s)-4-((6-((4-(methylsulfonyl)phenyl)amino)-1H-pyrazolo[3,4-d]pyrimidin-1-yl)methyl)cyclohexane-1-carboxylic acid

To a stirred solution of methyl (1s,4s)-4-((6-((4-(methylsulfonyl)phenyl)amino)-1H-pyrazolo[3,4-d]pyrimidin-1-yl)methyl)cyclohexane-1-carboxylate (100 mg, 0.225 mmol) in THE (0.5 mL) and MeOH (0.2 mL) were added LiOH (5 mg, 0.225 mmol) and water (0.3 mL) at rt. The reaction mixture was stirred at 60° C. for 2 h. After completion, the reaction mixture was concentrated in vacuo and the residue was taken in water (5 mL) and acidified using aqueous citric acid. The aqueous layer was extracted with DCM (20 mL) and washed with water (20 mL) and brine (20 mL). The organic layer was dried over anhydrous Na₂SO₄, filtered, and concentrated in vacuo to afford crude product, which was purified by prep HPLC and pure fractions were lyophilized to obtain the desired compound, (1s,4s)-4-((6-((4-(methylsulfonyl)phenyl)amino)-1H-pyrazolo[3,4-d]pyrimidin-1-yl)methyl)cyclohexane-1-carboxylic acid (29 mg, 0.064 mmol, 28.5% yield) as a white solid. ¹H NMR (400 MHz, DMSO-d₆): δ 11.58 (bs, 1H), 10.43 (s, 1H), 9.08 (s, 1H), 8.17 (s, 1H), 8.14 (d, J=8.8 Hz, 2H), 7.86 (d, J=8.8 Hz, 2H), 4.26 (d, J=7.2 Hz, 2H), 3.17 (s, 3H), 2.54-2.44 (m, 2H), 2.20-2.11 (m, 1H), 1.98-1.91 (m, 2H), 1.59-1.40 (m, 3H), 1.38-1.26 (m, 2H). LCMS (Method A): 90.0%, RT: 2.17 min, m/z: 430.0 [M+H]⁺.

~20% of other regioisomer was also observed in LCMS of crude sample, but not isolated. Compounds with R³ variables

TABLE 2
The following compounds were prepared in a manner like those exemplified in
Compounds 3-7, 11, and 16. Compounds are racemic or achiral unless specified otherwise.

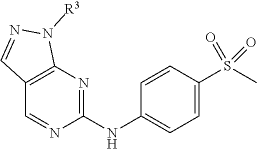

| | | | | Name |
|---|---|---|---|---|
| Ex No. | R³ | [M + H]⁺ | LCMS RT | ¹H NMR (400 MHZ, DMSO-d₆) δ |
| 12 | 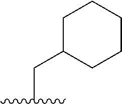 [SMILES: *CC1CCCCC1] | 386.0 | 2.59 | 1-(cyclohexylmethyl)-N-(4-(methylsulfonyl)phenyl)- 1H-pyrazolo[3,4-d]pyrimidin-6-amine 10.45 (s, 1H), 9.08 (s, 1H), 8.20-8.11 (m, 3H), 7.87 (d, J = 8.8 Hz, 2H), 4.21 (d, J = 7.0 Hz, 2H), 3.18 (s, 3H), 2.08-1.92 (m, 1H), 1.70-1.51 (m, 5H), 1.22- 0.99 (m, 5H). |
| 13 | 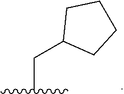 [SMILES: *CC1CCCC1] | 372.2 | 2.43 | 1-(cyclopentylmethyl)-N-(4-(methylsulfonyl)phenyl)- 1H-pyrazolo[3,4-d]pyrimidin-6-amine 10.46 (s, 1H), 9.09 (s, 1H), 8.21-8.11 (m, 3H), 7.86 (d, J = 8.8 Hz, 2H), 4.28 (d, J = 7.4 Hz, 2H), 3.18 (s, 3H), 2.58-2.48 (m, 1H), 1.71-1.60 (m, 4H), 1.58- 1.49 (m, 2H), 1.41-1.35 (m, 2H). |
| 14 | 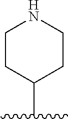 [SMILES: *C1CCNCC1] | 373.2 | 1.49 | N-(4-(methylsulfonyl)phenyl)-1-(piperidin-4-yl)-1H- pyrazolo[3,4-d]pyrimidin-6-amine 10.41 (s, 1H), 9.08 (s, 1H), 8.19-8.11 (m, 3H), 7.87 (d, J = 8.8 Hz, 2H), 4.73-4.66 (m, 1H), 3.35-3.30 (m, 1H), 3.17 (s, 3H), 3.16-3.05 (m, 2H), 2.74-2.63 (m, 2H), 2.12-2.01 (m, 2H), 1.90-1.81 (m, 2H). |
| 15 | 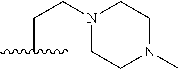 [SMILES: *CCN1CCN(C)CC1] | 416.2 | 1.50 | 1-(2-(4-methylpiperazin-1-yl)ethyl)-N-(4- (methylsulfonyl)phenyl)-1H-pyrazolo[3,4- d]pyrimidin-6-amine (formate salt) 10.44 (s, 1H), 9.07 (s, 1H), 8.20-8.11 (m, 3H), 7.85 (d, J = 8.8 Hz, 2H), 4.47 (t, J = 6.4 Hz, 2H), 3.17 (s, |
| | | | | 3H), 2.80 (t, J = 6.4 Hz, 2H), 2.51-2.40 (m, 5H), 2.22- |
| | | | | 2.11 (m, 3H), 2.07 (s, 3H). |
| 17 | 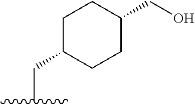 [SMILES: *C[C@H]1CC[C@@H](CO)CC1] | 416.2 | 2.08 | ((1s,4s)-4-((6-((4-(methylsulfonyl)phenyl)amino)-1H- pyrazolo[3,4-d]pyrimidin-1-yl)methyl)cyclohexyl) 10.46 (s, 1H), 9.08 (s, 1H), 8.21-8.12 (m, 3H), 7.86 (d, J = 8.4 Hz, 2H), 4.41-4.32 (m, 3H), 3.39-3.31 (m, 2H), 3.17 (s, 3H), 2.30-2.21 (m, 1H), 1.51-1.29 (m, 9H). |
| 28 | 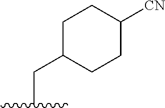 [SMILES: *CC1CCC(C#N)CC1] | 411.2 | 2.25 | 4-((6-((4-(methylsulfonyl)phenyl)amino)-1H- pyrazolo[3,4-d]pyrimidin-1-yl)methyl)cyclohexane-1- carbonitrile 10.46 (s, 1H), 9.10 (s, 1H), 8.18 (s, 1H), 8.16 (d, J = 8.8 Hz, 2H), 7.86 (d, J = 8.8 Hz, 2H), 4.28 (d, J = 7.2 Hz, 2H), 3.16 (s, 3H), 3.13-3.07 (m, 1H), 2.11-2.01 (m, 1H), 1.91-1.82 (m, 2H), 1.65-1.51 (m, 4H), 1.40- |
| | | | | 1.26 (m, 2H). |
| | Single isomer, relative | | | |
| | stereochemistry | | | |
| | unknown | | | |
| 29 | 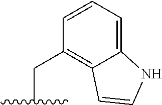 [SMILES: *Cc1cccc2[nH]ccc12] | 419.1 | 1.81 | 1-((1H-indol-4-yl)methyl)-N-(4- (methylsulfonyl)phenyl)-1H-pyrazolo[3,4- d]pyrimidin-6-amine 11.18 (s, 1H), 10.47 (s, 1H), 9.09 (s, 1H), 8.17 (s, 1H), 8.12 (d, J = 8.8 Hz, 2H), 7.83 (d, J = 8.8 Hz, 2H), 7.41 7.30 (m, 2H), 7.13-7.05 (m, 1H), 6.97 (d, J = 7.2 Hz, 1H), 6.49 (s, 1H), 5.81 (s, 2H), 3.17 (s, 3H). |
| 32 | 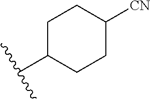 [SMILES: *C1CCC(C#N)CC1] | 397.2 | 2.17 | 4-(6-((4-(methylsulfonyl)phenyl)amino)-1H- pyrazolo[3,4-d]pyrimidin-1-yl)cyclohexane-1- carbonitrile 10.46 (s, 1H), 9.09 (s, 1H), 8.18 (s, 1H), 8.15 (d, J = 8.8 Hz, 2H), 7.89 (d, J = 8.8 Hz, 2H), 4.74-4.69 (m, 1H), 3.32-3.28 (m, 1H), 3.15 (s, 3H), 2.30-2.18 (m, 2H), 2.13-1.80 (m, 6H). |
| | Single isomer, relative | | | |
| | stereochemistry | | | |
| | unknown | | | |
| 33 | 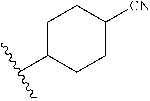 [SMILES: *C1CCC(C#N)CC1] | 397.2 | 2.20 | 4-(6-((4-(methylsulfonyl)phenyl)amino)-1H- pyrazolo[3,4-d]pyrimidin-1-yl)cyclohexane-1- carbonitrile 10.46 (s, 1H), 9.07 (s, 1H), 8.19-8.10 (m, 3H), 7.98- 7.86 (m, 2H), 4.82-4.62 (m, 1H), 3.17 (s, 3H), 2.90 2.85 (m, 1H), 2.29-2.14 (m, 2H), 2.10-1.98 (m, 4H), |
| | | | | 1.92-1.80 (m, 2H). |
| | Single isomer, relative | | | |
| | stereochemistry | | | |
| | unknown | | | |
| 39 | 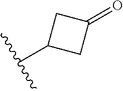 [SMILES: *C1CC(=O)C1] | 358.2 | 1.42 | 3-(6-((4-(methylsulfonyl)phenyl)amino)-1H- pyrazolo[3,4-d]pyrimidin-1-yl)cyclobutan-1-one 10.52 (s, 1H), 9.11 (s, 1H), 8.25 (s, 1H), 8.15 (d, J = 9.2 Hz, 2H), 7.86 (d, J = 8.8 Hz, 2H), 5.70-5.55 (m, 1H), 3.74 (d, J = 7.0 Hz, 4H), 3.17 (s, 3H). |
| 41 | 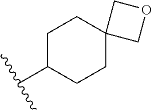 [SMILES: *C1CCC2(CC1)COC2] | 414.0 | 2.05 | N-(4-(methylsulfonyl)phenyl)-1-(2- oxaspiro[3.5]nonan-7-yl)-1H-pyrazolo[3,4- d]pyrimidin-6-amine 10.44 (s, 1H), 9.07 (s, 1H), 8.17-8.12 (m, 3H), 7.93 7.81 (m, 2H), 4.65-4.57 (m, 1H), 4.47 (s, 2H), 4.32 (s, 2H), 3.17 (s, 3H), 2.28-2.20 (m, 2H), 2.00-1.87 (m, 4H), 1.78-1.68 (m, 2H). |
| 45 | 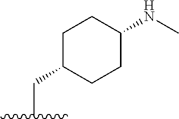 [SMILES: *C[C@H]1CC[C@@H](NC)CC1] | 415.3 | 1.30 | 1-(((1s,4s)-4-(methylamino)cyclohexyl)methyl)-N-(4- (methylsulfonyl)phenyl)-1H-pyrazolo[3,4- d]pyrimidin-6-amine 10.43 (s, 1H), 9.08 (s, 1H), 8.20-8.12 (m, 3H), 7.86 (d, J = 8.8 Hz, 2H), 4.26 (d, J = 7.2 Hz, 2H), 3.40 3.33 (m, 1H), 3.17 (s, 3H), 2.24 (s, 3H), 2.15-2.06 (m, 1H), 1.65-1.55 (m, 2H), 1.52-1.38 (m, 5H), 1.25 1.36 (m, 2H). |
| 46 | 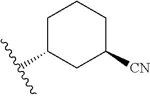 [SMILES: *[C@@H]1CCC[C@@H](C#N)C1] | 397.3 | 2.58 | (1R,3R)-3-(6-((4-(methylsulfonyl)phenyl)amino)-1H- pyrazolo[3,4-d]pyrimidin-1-yl)cyclohexane-1- carbonitrile 10.51 (s, 1H), 9.10 (s, 1H), 8.23-8.11 (m, 3H), 7.84 (d, J = 8.8 Hz, 2H), 4.98-4.90 (m, 1H), 3.51 (s, 1H), 3.15 (s, 3H), 2.20-2.13 (m, 2H), 2.10-2.00 (m, 2H), |
| | | | | 1.99-1.88 (m, 2H), 1.75-1.66 (m, 2H). |
| 47 | 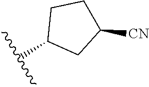 [SMILES: *[C@@H]1CC[C@@H](C#N)C1] | 383.1 | 2.22 | (1R,3R)-3-(6-((4-(methylsulfonyl)phenyl)amino)-1H- pyrazolo[3,4-d]pyrimidin-1-yl)cyclopentane-1- carbonitrile 10.45 (s, 1H), 9.09 (s, 1H), 8.18 (s, 1H), 8.14 (d, J = 8.8 Hz, 2H), 7.88 (d, J = 8.8 Hz, 2H), 5.46-5.42 (m, 1H), 3.42-3.46 (m, 1H), 3.17 (s, 3H), 2.33-2.48 (m, 4H), |
| | | | | 1.96-2.15 (m, 2H). |
| 50 | 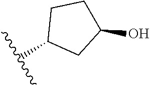 [SMILES: *[C@@H]1CC[C@@H](O)C1] | 374.3 | 2.44 | (1R,3R)-3-(6-((4-(methylsulfonyl)phenyl)amino)-1H- pyrazolo[3,4-d]pyrimidin-1-yl)cyclopentan-1-o1 10.43 (s, 1H), 9.07 (s, 1H), 8.22-8.09 (m, 3H), 7.87 (d, J = 8.8 Hz, 2H), 5.49-5.38 (m, 1H), 4.76 (d, J = 3.6 Hz, 1H), 4.48-4.40 (m, 1H), 3.17 (s, 3H), 2.36 2.19 (m, 2H), 2.19-1.93 (m, 3H), 1.71-1.62 (m, 1H). |
| 51 | 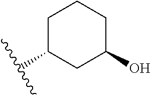 [SMILES: *[C@@H]1CCC[C@@H](O)C1] | 388.3 | 2.03 | (1R,3R)-3-(6-((4-(methylsulfonyl)phenyl)amino)-1H- pyrazolo[3,4-d]pyrimidin-1-yl)cyclohexan-1-ol 10.44 (s, 1H), 9.08 (s, 1H), 8.18-8.07 (m, 3H), 7.85 (d, J = 9.2 Hz, 2H), 5.15-5.00 (m, 1H), 4.72 (d, J = 2.8 Hz, 1H), 4.22-4.14 (m, 1H), 3.18 (s, 3H), 2.21- 2.07 (m, 1H), 2.02-1.78 (m, 4H), 1.73-1.61 (m, 2H), |
| | | | | 1.58-1.46 (m, 1H). |
| 52 | 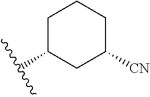 [SMILES: *[C@@H]1CCC[C@H](C#N)C1] | 397.0 | 2.28 | (1S,3R)-3-(6-((4-(methylsulfonyl)phenyl)amino)-1H- pyrazolo[3,4-d]pyrimidin-1-yl)cyclohexane-1- carbonitrile 10.46 (s, 1H), 9.09 (s, 1H), 8.18 (s, 1H), 8.15 (d, J = 9.2 Hz, 2H), 7.88 (d, J = 8.8 Hz, 2H), 4.78-4.65 (m, 1H), 3.17-3.12 (m, 4H), 2.40-2.29 (m, 1H), 2.29-2.20 |
| | | | | (m, 1H), 2.19-2.05 (m, 1H), 2.01-1.85 (m, 3H), 1.71 |
| | | | | 1.60 (m, 2H). |
| 53 | 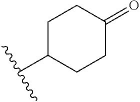 [SMILES: *C1CCC(=O)CC1] | 386.4 | 2.02 | 4-(6-((4-(methylsulfonyl)phenyl)amino)-1H- pyrazolo[3,4-d]pyrimidin-1-yl)cyclohexan-1-one 10.49 (s, 1H), 9.11 (s, 1H), 8.23-8.13 (m, 3H), 7.93 7.85 (m, 2H), 5.30-5.14 (m, 1H), 3.17 (s, 3H), 2.80- 2.65 (m, 2H), 2.49-2.38 (m, 4H), 2.35-2.22 (m, 2H). |
| 57 | 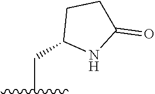 [SMILES: *C[C@@H]1CCC(=O)N1] | 387.2 | 1.35 | (S)-5-((6-((4-(methylsulfonyl)phenyl)amino)-1H- pyrazolo[3,4-d]pyrimidin-1-yl)methyl)pyrrolidin-2- one 10.47 (s, 1H), 9.09 (s, 1H), 8.21-8.11 (m, 3H), 7.93 7.85 (m, 2H), 7.85 (s, 1H), 4.50-4.27 (m, 2H), 4.08 4.05 (m, 1H), 3.17 (s, 3H), 2.19-2.03 (m, 3H), 2.02 |
| | | | | 1.88 (m, 1H). |
| 62 | 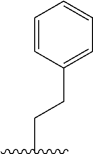 [SMILES: *CCc1ccccc1] | 394.2 | 2.33 | N-(4-(methylsulfonyl)phenyl)-1-phenethyl-1H- pyrazolo[3,4-d]pyrimidin-6-amine 10.37 (s, 1H), 9.03 (s, 1H), 8.16 (s, 1H), 8.05 (d, J = 8.8 Hz, 2H), 7.85 (d, J = 8.8 Hz, 2H), 7.23-7.06 (m, 5H), 4.60 (t, J = 7.0 Hz, 2H), 3.23-3.18 (m, 5H). |
| 63 | 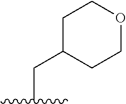 [SMILES: *CC1CCOCC1] | 388.2 | 1.99 | N-(4-(methylsulfonyl)phenyl)-1-((tetrahydro-2H- pyran-4-yl)methyl)-1H-pyrazolo[3,4-d]pyrimidin-6- amine 10.47 (s, 1H), 9.09 (s, 1H), 8.25-8.12 (m, 3H), 7.94 7.83 (m, 2H), 4.28 (d, J = 7.2 Hz, 2H), 3.93-3.73 (m, 2H), 3.32-3.23 (m, 2H), 3.18 (s, 3H), 2.24-2.19 (m, 1H), 1.48-1.44 (m, 2H), 1.40-1.29 (m, 2H). |

### Compound 18: 4-Fluorophenyl (1s,4s)-4-((6-((4-(methylsulfonyl)phenyl)amino)-1H-pyrazolo[3,4-d]pyrimidin-1-yl)methyl)cyclohexane-1-carboxylate

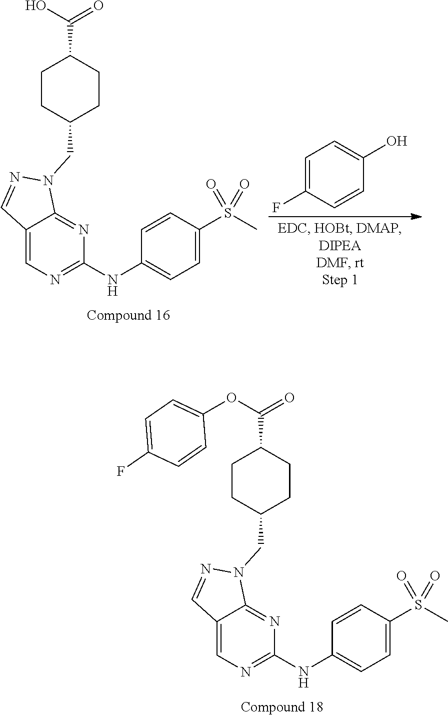

To a stirred solution of Compound 16 (1s,4s)-4-((6-((4-(methylsulfonyl)phenyl)amino)-1H-pyrazolo[3,4-d]pyrimidin-1-yl)methyl)cyclohexane-1-carboxylic acid (70 mg, 0.163 mmol) and 4-fluorophenol (18 mg, 0.163 mmol) in DMF (1 mL) were added EDC (47 mg, 0.244 mmol), HOBt (37 mg, 0.244 mmol), DMAP (2 mg, 0.016 mmol), and DIPEA (105 mg, 0.815 mmol) at 0° C. The reaction mixture was stirred at rt for 16 h. After completion, the reaction mixture diluted with water (10 mL) and extracted with EtOAc (3×20 mL). The combined organic layers were washed with brine (20 mL), dried over anhydrous Na₂SO₄, filtered, and concentrated in vacuo to afford crude product, which was purified by prep HPLC and pure fractions were lyophilized to obtain the desired compound, 4-fluorophenyl (1s,4s)-4-((6-((4-(methylsulfonyl)phenyl)amino)-1H-pyrazolo[3,4-d]pyrimidin-1-yl)methyl)cyclohexane-1-carboxylate (19 mg, 0.035 mmol, 21.8% yield) as an off-white solid. ¹H NMR (400 MHz, DMSO-d₆): δ 10.47 (s, 1H), 9.09 (s, 1H), 8.21-8.10 (m, 3H), 7.85 (d, J=8.8 Hz, 2H), 7.31-7.20 (m, 2H), 7.16-7.07 (m, 2H), 4.32 (d, J=7.2 Hz, 2H), 3.10 (s, 3H), 2.90-2.82 (m, 1H), 2.25-2.14 (m, 1H), 2.10-2.02 (m, 2H), 1.75-1.62 (m, 2H), 1.60-1.52 (m, 2H), 1.41-1.32 (m, 2H). LCMS (Method A): 99.3%, RT: 2.69 min, m/z: 524.2 [M+H]⁺.

### Compound 27: (1s,4s)-4-((6-((4-(methylsulfonyl)phenyl)amino)-1H-pyrazolo[3,4-d]pyrimidin-1-yl)methyl)cyclohexane-1-carbaldehyde

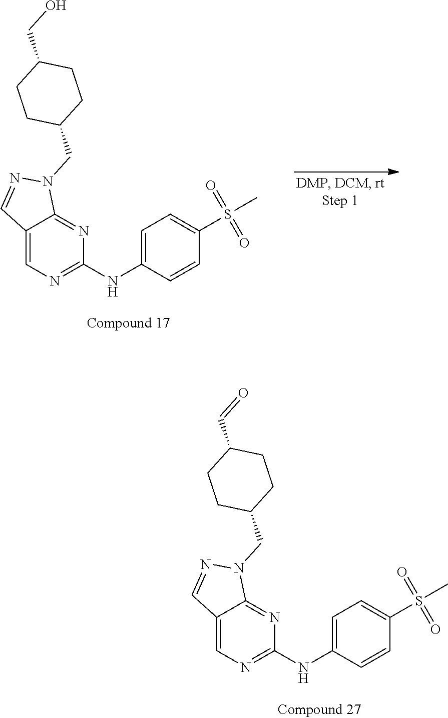 [SMILES: CS(=O)(=O)c1ccc(Nc2ncc3cnn(C[C@H]4CC[C@@H](CO)CC4)c3n2)cc1 ; CS(=O)(=O)c1ccc(Nc2ncc3cnn(C[C@H]4CC[C@@H](C=O)CC4)c3n2)cc1]

To a stirred solution of Compound 17 (1s,4s)-4-((6-((4-(methylsulfonyl)phenyl)amino)-1H-pyrazolo[3,4-d]pyrimidin-1-yl)methyl)cyclohexyl)methanol (50 mg, 0.120 mmol) in DCM (8 mL) was added DMP (77 mg, 0.181 mmol) at rt. The reaction mixture was stirred at rt for 16 h. After completion, the reaction mixture was diluted with water (10 mL) and extracted with EtOAc (2×20 mL). The combined organic layers were washed with brine (10 mL), dried over anhydrous Na₂SO₄, filtered, and concentrated in vacuo to afford crude product, which was purified by reverse phase chromatography using Biotage Isolera (35% aq NaHCO₃ in ACN) and fractions were lyophilized to afford the desired compound, (1s,4s)-4-((6-((4-(methylsulfonyl)phenyl)amino)-1H-pyrazolo[3,4-d]pyrimidin-1-yl)methyl)cyclohexane-1-carbaldehyde (4 mg, 9.29 μmol, 7.7% yield) as a white solid. ¹H NMR (400 MHz, DMSO-d₆): δ 10.43 (s, 1H), 9.63 (s, 1H), 9.08 (s, 1H), 8.20-8.11 (m, 3H), 7.86 (d, J=8.8 Hz, 2H), 4.22 (d, J=7.2 Hz, 2H), 3.17 (s, 3H), 2.59-2.45 (m, 1H), 2.18-1.91 (m, 3H), 1.61-1.44 (m, 4H), 1.19-1.08 (m, 2H). LCMS (Method A): 98.1%, RT: 2.31 min, m/z: 414.2 [M+H]⁺.

### Compound 40: 1-(((1s,4s)-4-(dimethylamino)cyclohexyl)methyl)-N-(4-(methylsulfonyl)phenyl)-1H-pyrazolo[3,4-d]pyrimidin-6-amine formate

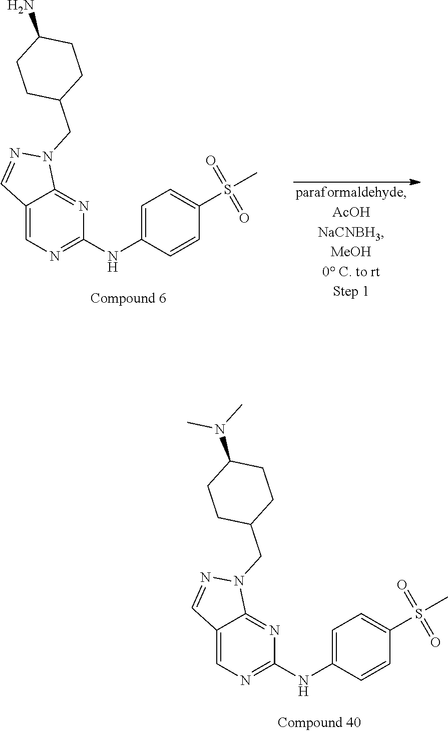

To a stirred solution of Compound 6 1-(((1s,4s)-4-aminocyclohexyl)methyl)-N-(4-(methylsulfonyl)phenyl)-1H-pyrazolo[3,4-d]pyrimidin-6-amine (50 mg, 0.125 mmol) in MeOH (2 mL) were added AcOH (38 mg, 0.624 mmol) and paraformaldehyde (30 mg) at rt. The reaction mixture was stirred at rt for 1 h and NaCNBH₃ (17 mg, 0.262 mmol) was added at 0° C. The resulting reaction mixture was stirred at rt for 16 h. After completion, the reaction mixture was evaporated in vacuo to give crude product, which was purified by prep HPLC and pure fractions were lyophilized to obtain the desired compound, 1-(((1s,4s)-4-(dimethylamino)cyclohexyl)methyl)-N-(4-(methylsulfonyl)phenyl)-1H-pyrazolo[3,4-d]pyrimidin-6-amine (28 mg, 0.065 mmol, 51.9% yield) as a light brown solid. ¹H NMR (400 MHz, DMSO-d₆): δ 10.47 (s, 1H), 9.08 (s, 1H), 8.20-8.11 (m, 3H), 7.85 (d, J=8.8 Hz, 2H), 4.31 (d, J=7.6 Hz, 2H), 3.16 (s, 3H), 2.22-2.10 (m, 8H), 1.67-1.80 (m, 2H), 1.47-1.20 (m, 6H). LCMS (Method A): 99.2%, RT: 1.65 min, m/z: 429.2 [M+H]⁺.

### Compound 48: 1-(((1r,4r)-4-(chloromethyl)cyclohexyl)methyl)-N-(4-(methylsulfonyl)phenyl)-1H-pyrazolo[3,4-d]pyrimidin-6-amine

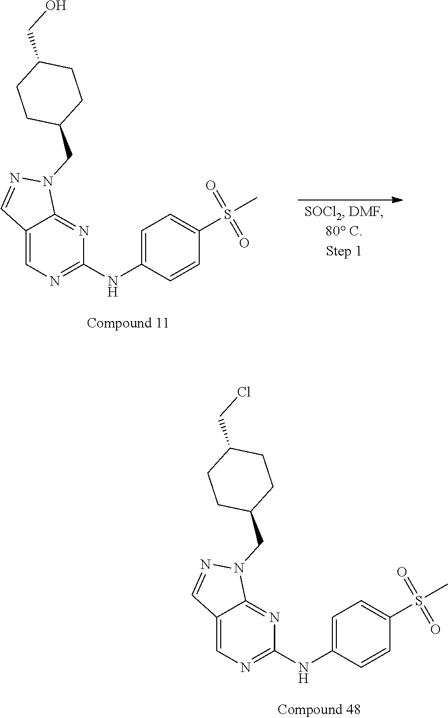 [SMILES: CS(=O)(=O)c1ccc(Nc2ncc3cnn(C[C@H]4CC[C@H](CO)CC4)c3n2)cc1 ; CS(=O)(=O)c1ccc(Nc2ncc3cnn(C[C@H]4CC[C@H](CCl)CC4)c3n2)cc1]

To a stirred solution of Compound 11 (1r,4r)-4-((6-((4-(methylsulfonyl)phenyl)amino)-1H-pyrazolo[3,4-d]pyrimidin-1-yl)methyl)cyclohexyl)methanol (50 mg, 0.120 mmol) in DMF (2 mL) was added SOCl₂ (29 mg, 0.241 mmol) at rt. The reaction mixture was stirred at 80° C. for 16 h and progress of the reaction was monitored by TLC and ¹H NMR. After completion, the reaction mixture was concentrated in vacuo to give crude product, which was purified by prep HPLC and pure fractions were lyophilized to obtain the desired compound, 1-(((1r,4r)-4-(chloromethyl)cyclohexyl)methyl)-N-(4-(methylsulfonyl)phenyl)-1H-pyrazolo[3,4-d]pyrimidin-6-amine (21 mg, 0.047 mmol, 39.6% yield) as off-white solid. ¹H NMR (400 MHz, DMSO-d₆): δ 10.45 (s, 1H), 9.09 (s, 1H), 8.22-8.12 (m, 3H), 7.93-7.82 (m, 2H), 4.22 (d, J=7.2 Hz, 2H), 3.48 (d, J=6.0 Hz, 2H), 3.17 (s, 3H), 2.61-2.50 (m, 1H), 2.00-1.89 (m, 1H), 1.76-1.83 (m, 2H), 1.69-1.49 (m, 2H), 1.19-1.08 (m, 2H), 1.05-0.92 (m, 2H). LCMS (Method A): 93.9%, RT: 2.73 min, m/z: 434.1 [M+H]⁺.

### Compound 49: 1-(((1s,4s)-4-(chloromethyl)cyclohexyl)methyl)-N-(4-(methylsulfonyl)phenyl)-1H-pyrazolo[3,4-d]pyrimidin-6-amine

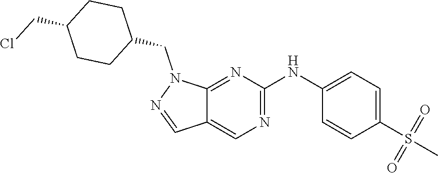

Compound 49 was prepared from Compound 17 in a manner like that exemplified in Compound 48. ¹H NMR (400 MHz, DMSO-d₆): δ 10.45 (s, 1H), 9.08 (s, 1H), 8.25-8.05 (m, 3H), 7.87 (d, J=8.8 Hz, 2H), 4.35 (d, J=7.6 Hz, 2H), 3.63 (d, J=6.8 Hz, 2H), 3.16 (s, 3H), 2.30-2.21 (m, 1H), 1.80-1.71 (m, 1H), 1.61-1.32 (m, 8H). LCMS (Method A): 99.5%, RT: 2.68 min, m/z: 434.4 [M+H]⁺

### Compound 54: (1r,4r)-4-(6-((4-(methylsulfonyl)phenyl)amino)-1H-pyrazolo[3,4-d]pyrimidin-1-yl)cyclohexan-1-ol

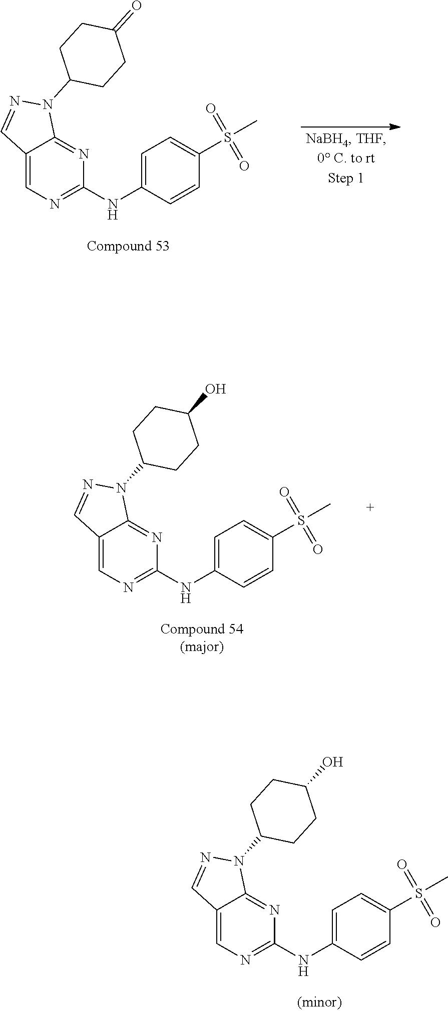

To a stirred solution of Compound 53 4-(6-((4-(methylsulfonyl)phenyl)amino)-1H-pyrazolo[3,4-d]pyrimidin-1-yl)cyclohexan-1-one (100 mg, 0.259 mmol) in THE (2 mL) was added NaBH₄ (9.8 mg, 0.259 mmol) at 0° C. The reaction mixture was stirred at rt for 1 h. After completion, the reaction mixture was diluted with water (10 mL) and extracted with DCM (3×20 mL). The combined organic layers were washed with brine (20 mL), dried over anhydrous Na₂SO₄, filtered, and concentrated in vacuo to afford crude product, which was purified by SFC purification and pure fractions were in vacuo to afford the desired product, (1r,4r)-4-(6-((4-(methylsulfonyl)phenyl)amino)-1H-pyrazolo[3,4-d]pyrimidin-1-yl)cyclohexan-1-ol (19 mg, 0.047 mmol, 18.2% yield), as a white solid (peak-2 from SFC).

¹H NMR (400 MHz, DMSO-d₆): δ 10.44 (s, 1H), 9.07 (s, 1H), 8.16-8.13 (m, 3H), 7.92-7.81 (m, 2H), 4.88-4.59 (m, 2H), 3.63-3.54 (m, 1H), 3.18 (s, 3H), 2.13-1.89 (m, 6H), 1.49-1.41 (m, 2H). LCMS (Method B): 98.9%, RT: 1.40 min, m/z: 388.3 [M+H]⁺.

The other regio-isomer, (1s,4s)-4-(6-((4-(methylsulfonyl)phenyl)amino)-1H-pyrazolo[3,4-d]pyrimidin-1-yl)cyclohexan-1-ol (2 mg, 5.16 μmol, 1.99% yield) was also isolated as a white solid (peak-1 from SFC). ¹H NMR (400 MHz, DMSO-d₆): δ 10.42 (s, 1H), 9.07 (s, 1H), 8.20-8.12 (m, 3H), 7.86 (d, J=8.8 Hz, 2H), 4.68-4.59 (m, 2H), 3.94-3.90 (m, 1H), 3.16 (s, 3H), 2.57-2.50 (m, 2H), 1.86-1.84 (m, 2H), 1.70-1.68 (m, 4H). LCMS (Method A): 97.8%, RT: 1.94 min, m/z: 388.2 [M+H]⁺.

### Compound 58 (peak 1) and 59 (peak 2): N-(4-(methylsulfonyl)phenyl)-1-((4-(pyrrolidin-1-yl)cyclohexyl)methyl)-1H-pyrazolo[3,4-d]pyrimidin-6-amine

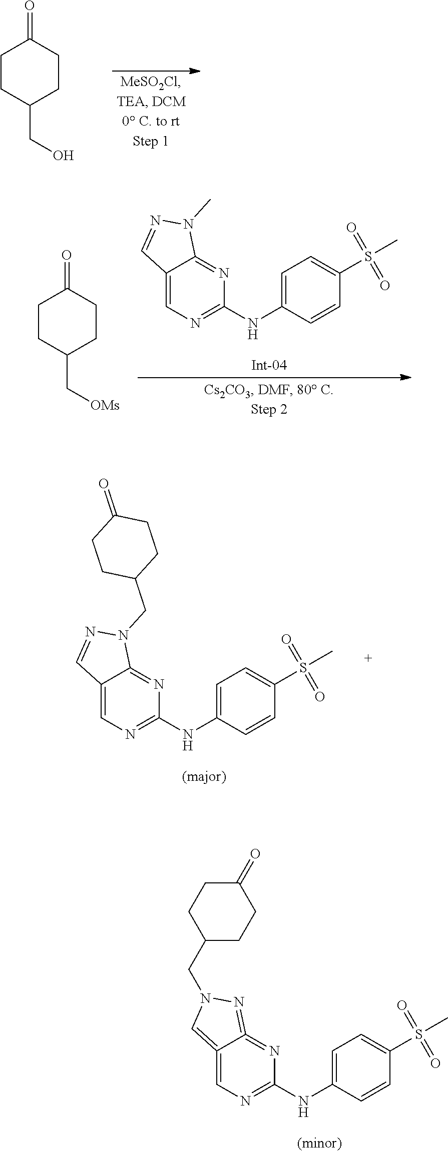

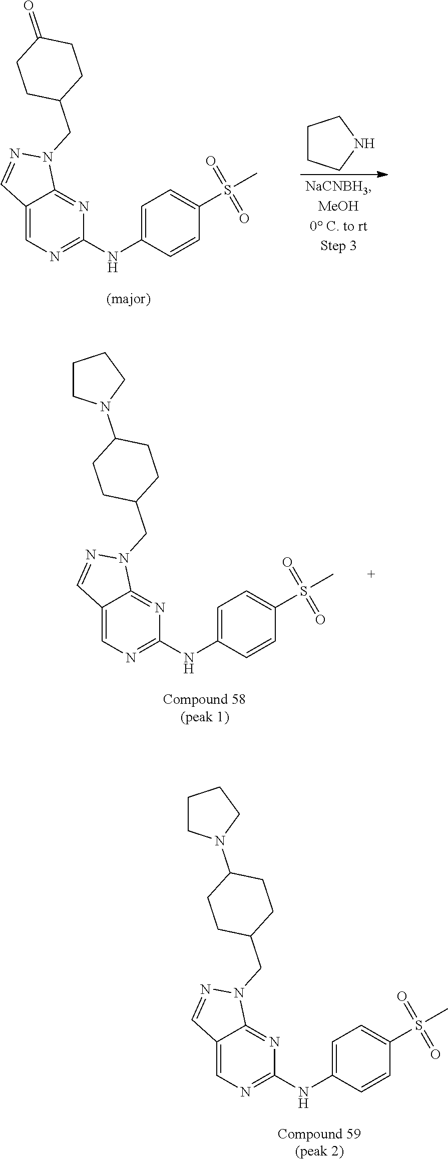 [SMILES: CS(=O)(=O)c1ccc(Nc2ncc3cnn(CC4CCC(=O)CC4)c3n2)cc1 ; C1CCNC1 ; CS(=O)(=O)c1ccc(Nc2ncc3cnn(CC4CCC(N5CCCC5)CC4)c3n2)cc1 ; CS(=O)(=O)c1ccc(Nc2ncc3cnn(CC4CCC(N5CCCC5)CC4)c3n2)cc1]

### Step 1: (4-oxocyclohexyl)methyl methanesulfonate

To a stirred solution of 4-(hydroxymethyl)cyclohexan-1-one (2 g, 15.60 mmol) in DCM (50 mL) were added TEA (4.35 mL, 31.2 mmol) and MeSO₂Cl (1.328 mL, 17.16 mmol) at 0° C. The reaction mixture was stirred at rt for 1 h and progress of the reaction was monitored by TLC. After completion, the reaction mixture was diluted with water (100 mL) and extracted with DCM (3×100 mL). The combined organic layers were washed with brine (100 mL), dried over anhydrous Na₂SO₄, filtered and concentrated in vacuo to obtain the desired compound, (4-oxocyclohexyl)methyl methanesulfonate (2.9 g, 14.1 mmol, 90% yield), as a pale yellow liquid, which was used without further purification.

### Step 2: 4-((6-((4-(methylsulfonyl)phenyl)amino)-1H-pyrazolo[3,4-d]pyrimidin-1-yl)methyl)cyclohexan-1-one

To a stirred solution of (4-oxocyclohexyl)methyl methanesulfonate (2.9 g, 14.06 mmol) in DMF (40 mL) were added Cs₂CO₃ (13.74 g, 42.2 mmol) and N-(4-(methylsulfonyl)phenyl)-1H-pyrazolo[3,4-d]pyrimidin-6-amine (4.07 g, 14.06 mmol) at rt. The reaction mixture was stirred at 80° C. for 16 h. After completion, the reaction mixture was diluted with water (100 mL) and extracted with EtOAc (3×100 mL). The combined organic layers were washed with brine (100 mL), dried over anhydrous Na₂SO₄, filtered and concentrated in vacuo to afford crude product, which was purified by Biotage Isolera (100-200 silica gel, using 75% EtOAc in hexanes as an eluent) to obtain the desired product, 4-((6-((4-(methylsulfonyl)phenyl)amino)-1H-pyrazolo[3,4-d]pyrimidin-1-yl)methyl)cyclohexan-1-one (2.3 g, 5.55 mmol, 39.5% yield) as an off-white solid (peak-1). LCMS (Method A): 96.4%, RT: 1.59 min, m/z: 400.3 [M+H]⁺.

The other regio-isomer, 4-((6-((4-(methylsulfonyl)phenyl)amino)-2H-pyrazolo[3,4-d]pyrimidin-2-yl)methyl)cyclohexan-1-one (100 mg, 0.250 mmol, 1.78% yield) was also isolated as an off-white solid (peak-2). LCMS (Method A): 94.5%, RT: 1.43 min, m/z: 400.3 [M+H]⁺.

### Step 3. Compound 58 (peak 1) and Compound 59 (peak 2): N-(4-(methylsulfonyl)phenyl)-1-((4-(pyrrolidin-1-yl)cyclohexyl)methyl)-1H-pyrazolo[3,4-d]pyrimidin-6-amine

To a stirred solution of 4-((6-((4-(methylsulfonyl)phenyl)amino)-1H-pyrazolo[3,4-d]pyrimidin-1-yl)methyl)cyclohexan-1-one (200 mg, 0.501 mmol, peak-1) in MeOH (5 mL) was added pyrrolidine (43 mg, 0.601 mmol) at rt. After stirring the reaction mixture for 1 h, NaCNBH₃ (47 mg, 0.751 mmol) was added at 0° C. The reaction mixture was stirred at rt for 16 h. After completion, the reaction mixture was diluted with water (20 mL) and extracted with EtOAc (3×20 mL). The combined organic layers were washed with brine (20 mL), dried over anhydrous Na₂SO₄, filtered, and concentrated in vacuo to give crude product, which was purified by SFC purification and pure fractions were concentrated in vacuo to give the desired compound, Compound 58, N-(4-(methylsulfonyl)phenyl)-1-((4-(pyrrolidin-1-yl)cyclohexyl)methyl)-1H-pyrazolo[3,4-d]pyrimidin-6-amine (26 mg, 0.054 mmol, 10.9% yield) as an off-white solid (peak-1 from SFC). ¹H NMR (400 MHz, DMSO-d₆): δ 10.46 (s, 1H), 9.09 (s, 1H), 8.27-8.03 (m, 3H), 7.84 (d, J=8.8 Hz, 2H), 4.26 (d, J=7.2 Hz, 2H), 3.16 (s, 3H), 2.50-2.40 (m, 4H), 2.15-2.05 (m, 2H), 1.83-1.62 (m, 6H), 1.58-1.26 (m, 6H).

LCMS (Method A): 99.8%, RT: 1.28 min, m/z: 455.3 [M+H]⁺.

The other diastereomer, Compound 59, N-(4-(methylsulfonyl)phenyl)-1-((4-(pyrrolidin-1-yl)cyclohexyl)methyl)-1H-pyrazolo[3,4-d]pyrimidin-6-amine (19 mg, 0.041 mmol, 8.1% yield) was also isolated as an off-white solid (peak-2 from SFC). ¹H NMR (400 MHz, DMSO-d₆): δ 10.45 (s, 1H), 9.08 (s, 1H), 8.17-8.15 (m, 3H), 7.87 (d, J=9.2 Hz, 2H), 4.22 (d, J=6.8 Hz, 2H), 3.18 (s, 3H), 2.55-2.44 (s, 4H), 2.02-1.85 (m, 4H), 1.65-1.55 (m, 6H), 1.12-1.09 (m, 4H). LCMS (Method A): 98.9%, RT: 1.31 min, m/z: 455.3 [M+H]⁺.

TABLE 3
The following compounds were prepared in a manner like those exemplified in
Compounds 58-59. Compounds are racemic or achiral unless specified otherwise.

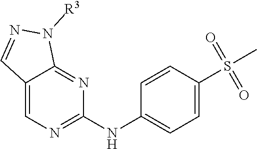

| | | | | NAME |
|---|---|---|---|---|
| Ex No. | R³ | [M + H]⁺ | LCMS RT | ¹H NMR (400 MHz, DMSO-d₆) δ |
| 60 | 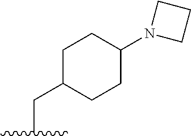 [SMILES: *CC1CCC(N2CCC2)CC1] | 441.2 | 1.24 | 1-((4-(azetidin-1-yl)cyclohexyl)methyl)-N-(4- (methylsulfonyl)phenyl)-1H-pyrazolo[3,4- d]pyrimidin-6-amine 10.45 (s, 1H), 9.08 (s, 1H), 8.24-8.05 (m, 3H), 7.88 (d, J = 8.8 Hz, 2H), 4.23 (d, J = 7.6 Hz, 2H), 3.17 (s, 3H), 3.04 (t, J = 6.8 Hz, 4H), 2.18-2.10 (m, 1H), 2.08-2.01 (m, 1H), 1.97-1.91 (m, 2H), 1.55- 1.35 (m, 4H), 1.35-1.26 (m, 4H). |
| | Single isomer, relative | | | |
| | stereochemistry unknown | | | |
| 61 | 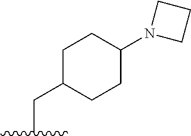 [SMILES: *CC1CCC(N2CCC2)CC1] | 441.2 | 1.34 | 1-((4-(azetidin-1-yl)cyclohexyl)methyl)-N-(4- (methylsulfonyl)phenyl)-1H-pyrazolo[3,4- d]pyrimidin-6-amine 10.45 (s, 1H), 9.08 (s, 1H), 8.23-8.15 (m, 3H), 7.87 (d, J = 8.8 Hz, 2H), 4.22 (d, J = 6.8 Hz, 2H), 3.18 (s, 3H), 3.12 (t, J = 6.9 Hz, 4H), 2.06-1.79 (m, 4H), 1.72-1.69 (m, 2H), 1.60-1.58 (m, 2H), 1.10- 1.00 (m, 2H), 0.87-0.79 (m, 2H). |
| | Single isomer, relative | | | |
| | stereochemistry unknown | | | |
| 64 | 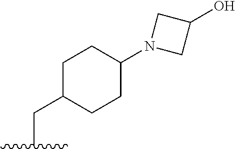 [SMILES: *CC1CCC(N2CC(O)C2)CC1] | 457.2 | 1.24 | 1-(4-((6-((4-(methylsulfonyl)phenyl)amino)-1H- pyrazolo[3,4-d]pyrimidin-1- yl)methyl)cyclohexyl)azetidin-3-ol 10.45 (s, 1H), 9.08 (s, 1H), 8.21-8.06 (m, 3H), 7.87 (d, J = 8.8 Hz, 2H), 5.18 (d, J = 6.4 Hz, 1H), 4.23- 4.14 (m, 3H), 3.54-3.41 (m, 2H), 3.17 (s, 3H), 2.59-2.55 (m, 2H), 2.11-2.06 (m, 2H), 1.51-1.48 (m, 2H), 1.40-1.26 (m, 6H). |
| | Single isomer, relative | | | |
| | stereochemistry unknown | | | |
| 65 | 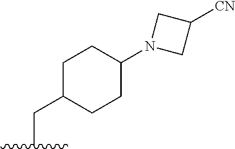 [SMILES: *CC1CCC(N2CC(C#N)C2)CC1] | 466 | 1.27 | 1-(4-((6-((4-(methylsulfonyl)phenyl)amino)-1H- pyrazolo[3,4-d]pyrimidin-1- yl)methyl)cyclohexyl)azetidine-3-carbonitrile 10.45 (s, 1H), 9.08 (s, 1H), 8.16-8.14 (m, 3H), 7.87 (d, J = 8.8 Hz, 2H), 4.23 (d, J = 7.2 Hz, 2H), 3.44- 3.39 (m, 3H), 3.18-3.13 (m, 5H), 2.28-2.20 (m, 1H), 2.08-2.06 (m, 1H), 1.50-1.40 (m, 2H), 1.40- 1.19 (m, 6H). |
| | Single isomer, relative | | | |
| | stereochemistry unknown | | | |
| 66 | 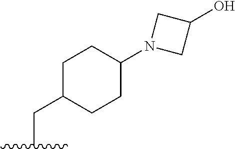 [SMILES: *CC1CCC(N2CC(O)C2)CC1] | 457.2 | 1.24 | 1-(4-((6-((4-(methylsulfonyl)phenyl)amino)-1H- pyrazolo[3,4-d]pyrimidin-1- yl)methyl)cyclohexyl)azetidin-3-ol 10.45 (s, 1H), 9.08 (s, 1H), 8.26-8.07 (m, 3H), 7.87 (d, J = 8.8 Hz, 2H), 5.17 (d, J = 6.8 Hz, 1H), 4.21 (d, J = 7.2 Hz, 2H), 4.10-4.05 (m, 1H), 3.42-3.39 (m, 2H), 3.18 (s, 3H), 2.61-2.58 (m, 2H), 1.89- 1.81 (m, 2H), 1.75-1.65 (m, 2H), 1.65-1.55 (m, 2H), 1.03-1.01 (m, 2H), 0.86-0.78 (m, 2H). |
| | Single isomer, relative | | | |
| | stereochemistry unknown | | | |
| 67 | 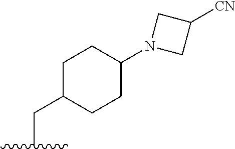 [SMILES: *CC1CCC(N2CC(C#N)C2)CC1] | 466 | 1.27 | 1-(4-((6-((4-(methylsulfonyl)phenyl)amino)-1H- pyrazolo[3,4-d]pyrimidin-1- yl)methyl)cyclohexyl)azetidine-3-carbonitrile 10.45 (s, 1H), 9.08 (s, 1H), 8.16-8.14 (m, 3H), 7.87 (d, J = 8.8 Hz, 2H), 4.22 (d, J = 6.8 Hz, 2H), 3.36- 3.32 (m, 3H), 3.18-3.16 (m, 5H), 2.03-1.80 (m, 2H), 1.78-1.47 (m, 4H), 1.11-1.01 (m, 2H), 0.87- 0.78 (m, 2H). |
| | Single isomer, relative | | | |
| | stereochemistry unknown | | | |
| 69 | 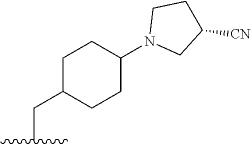 [SMILES: *CC1CCC(N2CC[C@H](C#N)C2)CC1] | 480.4 | 1.74 | (S)-1-(4-((6-((4-(methylsulfonyl)phenyl)amino)- 1H-pyrazolo[3,4-d]pyrimidin-1- yl)methyl)cyclohexyl)pyrrolidine-3-carbonitrile 10.46 (s, 1H), 9.09 (s, 1H), 8.17-8.15 (m, 3H), 7.90-7.78 (m, 2H), 4.26 (d, J = 7.5 Hz, 2H), 3.24-3.25 (m, 1H), 3.17 (s, 3H), 2.85-2.75 (m, 1H), 2.70-2.62 (m, 2H), 2.19-2.17 (m, 3H), 2.04-1.90 (m, 1H), 1.80-1.70 (m, 2H), 1.54- 1.27 (m, 7H). |
| | Single isomer, relative | | | |
| | stereochemistry unknown | | | |
| 70 | 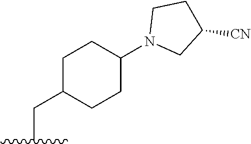 [SMILES: *CC1CCC(N2CC[C@H](C#N)C2)CC1] | 480.4 | 1.57 | (S)-1-(4-((6-((4-(methylsulfonyl)phenyl)amino)- 1H-pyrazolo[3,4-d]pyrimidin-1- yl)methyl)cyclohexyl)pyrrolidine-3-carbonitrile 10.45 (s, 1H), 9.09 (s, 1H), 8.20-8.12 (m, 3H), 7.87 (d, J = 8.8 Hz, 2H), 4.22 (d, J = 7.2 Hz, 2H), 3.20 3.15 (m, 4H), 2.80-2.68 (m, 3H), 2.55-2.46 (m, 1H), 2.18-2.04 (m, 2H), 1.92-1.80 (m, 4H), 1.65- 1.52 (m, 2H), 1.12-1.08 (m, 4H). |
| | Single isomer, relative | | | |
| | stereochemistry unknown | | | |
| 71 | 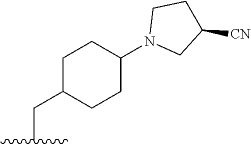 [SMILES: *CC1CCC(N2CC[C@@H](C#N)C2)CC1] | 480.4 | 1.35 | (R)-1-(4-((6-((4-(methylsulfonyl)phenyl)amino)- 1H-pyrazolo[3,4-d]pyrimidin-1- yl)methyl)cyclohexyl)pyrrolidine-3-carbonitrile 10.46 (s, 1H), 9.09 (s, 1H), 8.17-8.15 (m, 3H), 7.85 (d, J = 8.8 Hz, 2H), 4.26 (d, J = 7.5 Hz, 2H), 3.28- 3.25 (m, 1H), 3.17 (s, 3H), 2.79 (t, J = 8.0 Hz, 1H), 2.70-2.60 (m, 3H), 2.25-2.10 (m, 3H), 2.02- 1.87 (m, 1H), 1.76-1.73 (m, 2H), 1.56-1.29 (m, 6H) |
| | Single isomer, relative | | | |
| | stereochemistry unknown | | | |
| 72 | 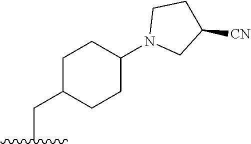 [SMILES: *CC1CCC(N2CC[C@@H](C#N)C2)CC1] | 480.4 | 1.37 | (R)-1-(4-((6-((4-(methylsulfonyl)phenyl)amino)- 1H-pyrazolo[3,4-d]pyrimidin-1- yl)methyl)cyclohexyl)pyrrolidine-3-carbonitrile 10.45 (s, 1H), 9.08 (s, 1H), 8.21-8.12 (m, 3H), 7.87 (d, J = 9.2 Hz, 2H), 4.22 (d, J = 6.9 Hz, 2H), 3.19 3.15 (m, 4H), 2.80-2.60 (m, 3H), 2.48-2.43 (m, 1H), 2.18-1.99 (m, 2H), 1.97-1.81 (m, 4H), 1.64- 1.62 (m, 2H), 1.16-1.08 (m, 4H). |
| | Single isomer, relative | | | |
| | stereochemistry unknown | | | |
| 73 | 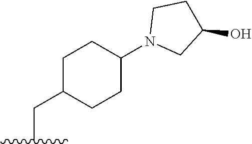 [SMILES: *CC1CCC(N2CC[C@@H](O)C2)CC1] | 471.3 | 1.26 | (R)-1-(4-((6-((4-(methylsulfonyl)phenyl)amino)- 1H-pyrazolo[3,4-d]pyrimidin-1- yl)methyl)cyclohexyl)pyrrolidin-3-ol 10.45 (s, 1H), 9.08 (s, 1H), 8.17-8.15 (m, 3H), 7.87 (d, J = 8.8 Hz, 2H), 4.59 (d, J = 4.6 Hz, 1H), 4.21 (d, J = 6.9 Hz, 2H), 4.16-4.05 (m, 1H), 3.18 (s, 3H), 2.71-2.67 (m, 1H), 2.61-2.56 (m, 1H), 2.50- 2.40 (m, 1H), 2.39-2.20 (m, 1H), 1.93-1.85 (m, 5H), 1.61-1.59 (m, 2H), 1.51-1.40 (m, 1H), 1.15- |
| | | | | 1.11 (m, 4H). |
| | Single isomer, relative | | | |
| | stereochemistry unknown | | | |
| 74 | 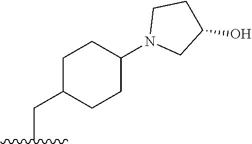 [SMILES: *CC1CCC(N2CC[C@H](O)C2)CC1] | 471.2 | 1.26 | (S)-1-(4-((6-((4-(methylsulfonyl)phenyl)amino)- 1H-pyrazolo[3,4-d]pyrimidin-1- yl)methyl)cyclohexyl)pyrrolidin-3-ol 10.45 (s, 1H), 9.09 (s, 1H), 8.21-8.07 (m, 3H), 7.85 (d, J = 8.8 Hz, 2H), 4.61 (d, J = 4.0 Hz, 1H), 4.32- 4.15 (m, 3H), 3.17 (s, 3H), 2.78-2.74 (m, 1H), 2.47- 2.45 (m, 2H), 2.34-2.33 (m, 1H), 2.20-2.05 (m, 2H), 2.01-1.96 (m, 1H), 1.80-1.65 (m, 2H), 1.60- 1.50 (m, 1H), 1.46-1.35 (m, 4H), 1.36-1.25 (m, 2H). |
| | Single isomer, relative | | | |
| | stereochemistry unknown | | | |
| 75 | 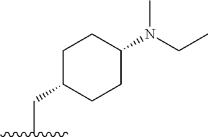 [SMILES: *C[C@H]1CC[C@@H](N(C)CC)CC1] | 443.2 | 1.52 | 1-(((1s,4s)-4- (ethyl(methyl)amino)cyclohexyl)methyl)-N-(4- (methylsulfonyl)phenyl)-1H-pyrazolo[3,4- d]pyrimidin-6-amine 10.48 (s, 1H), 9.09 (s, 1H), 8.22-8.12 (m, 3H), 7.84 (d, J = 8.8 Hz, 2H), 4.35 (d, J = 7.7 Hz, 2H), 3.16 (s, 3H), 2.51 (s, 3H), 2.33-2.20 (m, 4H), 1.72- 1.70 (m, 2H), 1.59-1.26 (m, 6H), 1.00-0.90 (m, 3H). |
| 78 | 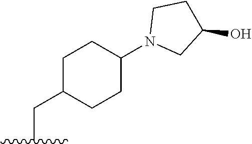 [SMILES: *CC1CCC(N2CC[C@@H](O)C2)CC1] | 471.3 | 1.27 | (R)-1-(4-((6-((4-(methylsulfonyl)phenyl)amino)- 1H-pyrazolo[3,4-d]pyrimidin-1- yl)methyl)cyclohexyl)pyrrolidin-3-ol 10.46 (s, 1H), 9.09 (s, 1H), 8.23-8.07 (m, 3H), 7.85 (d, J = 8.8 Hz, 2H), 4.68-4.60 (m, 1H), 4.34-4.07 (m, 3H), 3.17 (s, 3H), 2.78-2.74 (m, 1H), 2.46 (s, 1H), 2.32-2.22 (m, 1H), 2.20-2.08 (m, 2H), 2.08- 1.96 (m, 1H), 1.78-1.62 (m, 2H), 1.63-1.40 (m, 5H), 1.36-1.32 (m, 2H), 1.05-1.04 (m, 1H). |
| | Single isomer, relative | | | |
| | stereochemistry unknown | | | |
| 84 | 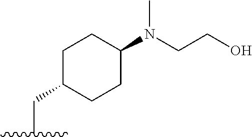 [SMILES: *C[C@H]1CC[C@H](N(C)CCO)CC1] | 459.4 | 1.38 | 2-(methyl((1r,4r)-4-((6-((4- (methylsulfonyl)phenyl)amino)-1H-pyrazolo[3,4- d\pyrimidin-1-yl)methyl)cyclohexyl)amino)ethan- 1-ol 10.45 (bs, 1H), 9.09 (s, 1H), 8.21-8.11 (m, 3H), 7.87 (d, J = 9.2 Hz, 2H), 4.20 (d, J = 6.9 Hz, 2H), 3.95-3.85 (m, 1H), 3.41-3.32 (m, 2H), 3.18 (s, 3H), 2.42 (t, J = 6.6 Hz, 2H), 2.31 (s, 1H), 2.15 (s, 3H), 1.95-1.85 (m, 1H), 1.75-1.60 (m, 4H), 1.24- |
| | | | | 1.04 (m, 4H). |

### Compound 76: 1-(1H-indazol-6-yl)-N-(4-(methylsulfonyl)phenyl)-1H-pyrazolo[3,4-d]pyrimidin-6-amine

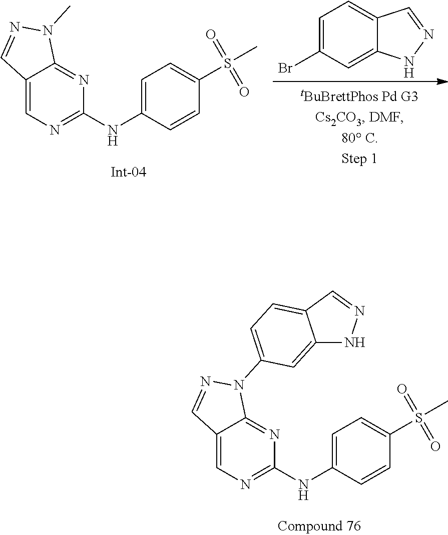

To a stirred solution of N-(4-(methylsulfonyl)phenyl)-1H-pyrazolo[3,4-d]pyrimidin-6-amine (500 mg, 1.73 mmol) and 6-bromo-1H-indazole (341 mg, 1.73 mmol) in dioxane (5 mL) was added cesium carbonate (1.689 g, 5.18 mmol) at rt and the reaction mixture was degassed for 10 mins. ^{t}BuBrettPhos Pd G3 (148 mg, 0.173 mmol) was added at rt and the reaction mixture was stirred at 100° C. for 16 h. After completion, the reaction mixture was filtered through Celite® bed, and the filtrate was diluted with water (20 mL) and extracted with EtOAc (3×30 mL). The combined organic layers were dried over anhydrous Na₂SO₄, filtered, and concentrated in vacuo to afford crude product, which was purified by prep HPLC and pure fractions were lyophilized to obtain the desired compound, 1-(1H-indazol-6-yl)-N-(4-(methylsulfonyl)phenyl)-1H-pyrazolo[3,4-d]pyrimidin-6-amine (7 mg, 0.017 mmol, 0.97% yield) as an off-white solid. ¹H NMR (400 MHz, DMSO-d₆): 313.41 (s, 1H), 10.64 (s, 1H), 9.23 (s, 1H), 8.48 (s, 1H), 8.30 (s, 1H), 8.22-8.13 (m, 3H), 8.05-7.90 (m, 4H), 3.19 (s, 3H).

LCMS (Method A): 97.1%, RT: 1.66 min, m/z: 406.0 [M+H]⁺.

TABLE 4
The following compounds were prepared in a manner like those exemplified in
Compound 76. Compounds are racemic or achiral unless specified otherwise.

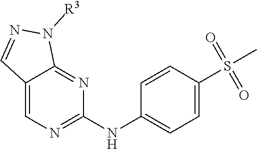

| Ex No. | R³ | [M + H]⁺ | LCMS RT | ¹H NMR (400 MHz, DMSO-d₆) δ |
|---|---|---|---|---|
| 81 | 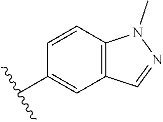 [SMILES: *c1ccc2c(cnn2C)c1] | 420.2 | 1.77 | 1-(1-methyl-1H-indazol-5-yl)-N-(4- (methylsulfonyl)phenyl)-1H-pyrazolo[3,4- d]pyrimidin-6-amine 10.59 (s, 1H), 9.22 (s, 1H), 8.48-8.44 (m, 2H), 8.24 (s, 1H), 8.18-8.14 (m, 3H), 7.93-7.86 (m, 3H), 4.15 (s, 3H), 3.19 (s, 3H). |
| 82 | 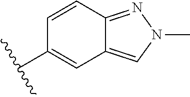 [SMILES: *c1ccc2nn(C)cc2c1] | 420.2 | 1.65 | 1-(2-methyl-2H-indazol-5-yl)-N-(4- (methylsulfonyl)phenyl)-1H-pyrazolo[3,4- d]pyrimidin-6-amine 10.58 (s, 1H), 9.21 (s, 1H), 8.53 (s, 1H), 8.43 (s, 1H), 8.39 (dd, J = 2.1, 0.8 Hz, 1H), 8.14 (dd, J = 6.8, 2.0 Hz, 2H), 8.01 (dd, J = 9.2, 2.1 Hz, 1H), |
| | | | | 7.92-7.83 (m, 3H), 4.25 (s, 3H), 3.19 (s, 3H). |

### Compound 68 and 83: 4-(6-((4-(methylsulfonyl)phenyl)amino)-1H-pyrazolo[3,4-d]pyrimidin-1-yl)benzonitrile and 4-(6-((4-(methylsulfonyl)phenyl)amino)-2H-pyrazolo[3,4-d]pyrimidin-2-yl)benzonitrile

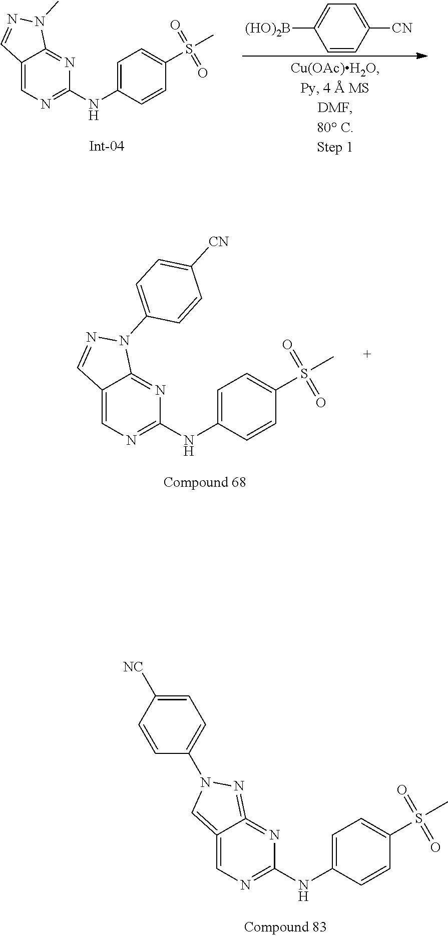

To a stirred solution of N-(4-(methylsulfonyl)phenyl)-1H-pyrazolo[3,4-d]pyrimidin-6-amine (500 mg, 1.73 mmol) and (4-cyanophenyl)boronic acid (305 mg, 2.07 mmol) in DMF (5 mL) were added copper(II) acetate monohydrate (690 mg, 3.46 mmol), pyridine (0.28 mL, 3.46 mmol) and 4 Å molecular sieves (300 mg, 1.73 mmol) at rt. The reaction mixture was stirred at 80° C. for 16 h. After completion, the reaction mixture was filtered through Celite® bed, and the filtrate was concentrated in vacuo to give the crude product, which was purified by prep HPLC and pure fractions were lyophilized to obtain the desired compound, Compound 68 4-(6-((4-(methylsulfonyl)phenyl)amino)-1H-pyrazolo[3,4-d]pyrimidin-1-yl)benzonitrile (7 mg, 0.017 mmol, 1.0% yield) as an off-white solid (peak-2 from HPLC). ¹H NMR (400 MHz, DMSO-d₆): 310.72 (s, 1H), 9.23 (s, 1H), 8.53 (s, 1H), 8.48 (d, J=8.8 Hz, 2H), 8.15 (d, J=8.8 Hz, 2H), 8.09 (d, J=8.8 Hz, 2H), 7.95 (d, J=8.4 Hz, 2H), 3.20 (s, 3H). LCMS (Method A): 99.9%, RT: 2.05 min, m/z: 391.3 [M+H]⁺.

The other regio-isomer, Compound 83 4-(6-((4-(methylsulfonyl)phenyl)amino)-2H-pyrazolo[3,4-d]pyrimidin-2-yl)benzonitrile (5 mg, 0.012 mmol, 1.31% yield) was also isolated as an off-white solid (peak-1 from HPLC). ¹H NMR (400 MHz, DMSO-d₆): 310.60 (s, 1H), 9.17 (s, 1H), 8.47-8.43 (m, 3H), 8.10-8.04 (m, 4H), 7.92 (d, J=8.8 Hz, 2H), 3.17 (s, 3H). LCMS (Method B): 99.3%, RT: 1.75 min, m/z: 391.3 [M+H]⁺.

### Compound 77: N-(4-(methylsulfonyl)phenyl)-1-(1-(pyridazin-3-yl)ethyl)-1H-pyrazolo[4,3-d]pyridin-6-amine

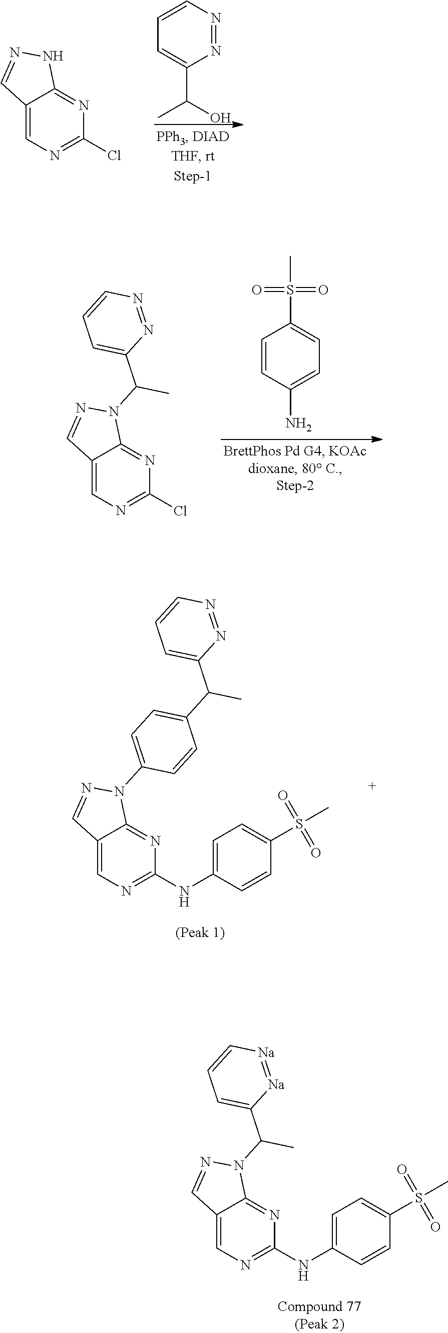

### Step 1: 6-chloro-1-(1-(pyridazin-3-yl)ethyl)-1H-pyrazolo[4,3-c]pyridine

To a stirred solution of 6-chloro-1H-pyrazolo[3,4-d]pyrimidine (240 mg, 1.55 mmol) in THF (5 mL) were added 1-(pyridazin-3-yl)ethan-1-ol (212 mg, 1.71 mmol), DIAD (610 μL, 3.11 mmol) and triphenylphosphine (815 mg, 3.11 mmol) at rt. The reaction was stirred at rt for 1 h. After completion, the reaction mixture was diluted with water (20 mL) and extracted with EtOAc (3×20 mL). The combined organic layers were washed with brine (20 mL), dried over anhydrous Na₂SO₄, filtered, and concentrated in vacuo to afford crude product, which was purified by Biotage Isolera (35% EtOAc in hexanes) to obtain the desired product, 6-chloro-1-(1-(pyridazin-3-yl)ethyl)-1H-pyrazolo[4,3-c]pyridine (230 mg, 0.886 mmol, 57.1% yield) as a light brown solid. LCMS (Method A): 99.3%, RT: 1.37 min, m/z: 260.2 [M+H]⁺.

### Step 2. Compound 77: N-(4-(methylsulfonyl)phenyl)-1-(1-(pyridazin-3-yl)ethyl)-1H-pyrazolo[4,3-c]pyridin-6-amine

To a stirred solution of 6-chloro-1-(1-(pyridazin-3-yl)ethyl)-1H-pyrazolo[4,3-c]pyridine (100 mg, 0.385 mmol) in 1,4-dioxane (2 mL) were added 4-(methylsulfonyl)aniline (73 mg, 0.424 mmol) and KOAc (113 mg, 1.16 mmol) at rt. The reaction mixture was degassed for 10 min and BrettPhos Pd G4 (18 mg, 0.019 mmol) was added at rt. The resulting reaction mixture was stirred at 80° C. for 16 h. The reaction mixture was diluted with water (20 mL) and extracted with EtOAc (3×20 mL). The combined organic layers were washed with brine (20 mL), dried over anhydrous Na₂SO₄, filtered, and concentrated in vacuo to afford crude product, which was purified by chiral SFC purification and pure fractions were concentrated in vacuo to afford the desired compound, N-(4-(methylsulfonyl)phenyl)-1-(1-(pyridazin-3-yl)ethyl)-1H-pyrazolo[4,3-c]pyridin-6-amine (10 mg, 0.026 mmol, 6.8% yield) as a white solid (peak-2 from SFC). ¹H NMR (400 MHz, DMSO-d₆): 310.48 (s, 1H), 9.18 (dd, J=4.9, 1.6 Hz, 1H), 9.12 (s, 1H), 8.24 (s, 1H), 8.02 (d, J=8.8 Hz, 2H), 7.82 (m, J=8.8 Hz, 2H), 7.68 (dd, J=8.6, 4.9 Hz, 1H), 7.54 (dd, J=8.6, 1.6 Hz, 1H), 6.42 (q, J=7.1 Hz, 1H), 3.17 (s, 3H), 2.05 (d, J=7.0 Hz, 3H). LCMS (Method A): 99.4%, RT: 1.60 min, m/z: 396.2 [M+H]⁺.

The other enantiomer, N-(4-(methylsulfonyl)phenyl)-1-(1-(pyridazin-3-yl)ethyl)-1H-pyrazolo[3,4-d]pyrimidin-6-amine (8 mg, 0.020 mmol, 5.1% yield) was also isolated as a white solid (peak-1 from SFC). ¹H NMR (400 MHz, DMSO-d₆): 310.48 (s, 1H), 9.18 (dd, J=5.2, 1.6 Hz, 1H), 9.12 (s, 1H), 8.24 (s, 1H), 8.25 (d, J=8.8 Hz, 2H), 7.23 (d, J=8.8 Hz, 2H), 7.67 (dd, J=8.8, 5.2 Hz, 1H), 7.54 (dd, J=8.4, 1.2 Hz, 1H), 6.41 (q, J=6.8 Hz, 1H), 3.17 (s, 3H), 2.04 (d, J=7.2 Hz, 3H). LCMS (Method A): 99.8%, RT: 1.60 min, m/z: 396.2 [M+H]⁺.

Compounds with R⁵ Variables

### General Procedure for the Synthesis of Compounds 20, 22, 24, 35, 55, 79 and 80

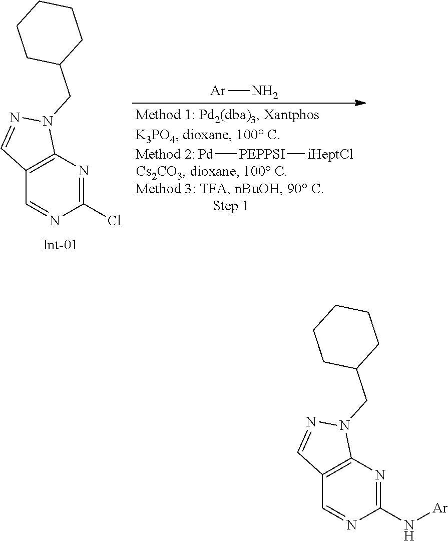

### Method 1

To a stirred solution of Int-01 (1 mmol) and arylamine (1 mmol) in dioxane (5 mL) were added K₃PO₄ (3 mmol) and Xantphos (0.1 mmol) at rt. The reaction mixture was degassed for 10 minutes. Pd₂(dba)₃ (0.05 mmol) was added, and the resulting reaction mixture was stirred at 100° C. After completion, the reaction mixture was filtered through Celite® bed and washed with EtOAc (10 mL). The filtrate was concentrated in vacuo and the residue was purified by Biotage Isolera to obtain the desired product.

### Method 2

To a stirred solution of arylamine (1 mmol) and Int-01 (1.2 mmol) in dioxane (4 mL) was added Cs₂CO₃ (3 mmol) at rt. The reaction mixture was degassed for 5 min and Pd-PEPPSI-iHeptCl (0.05 mmol) was added. The reaction mixture was heated at 120° C. After completion, the reaction mixture was filtered through Celite® bed and washed with EtOAc (10 mL). The filtrate was concentrated in vacuo and the residue was purified by Biotage Isolera to obtain the desired product.

### Method 3

To a solution of arylamine (1 mmol) and Int-01 (1.2 mmol) in nBuOH (3 mL) was added TFA (3 mmol) at rt. The reaction mixture was stirred at 90° C. After completion, the reaction mixture was diluted with water (10 mL) and extracted with EtOAc (2×20 mL). The combined organic layers were dried over anhydrous Na₂SO₄, filtered, and concentrated in vacuo to give crude product, which was purified by Biotage Isolera to obtain the desired product.

TABLE 5
The following compounds were prepared in a manner like those exemplified in
above general procedure. Compounds are racemic or achiral unless specified otherwise.

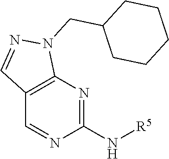

| | | | | NAME |
|---|---|---|---|---|
| Ex No. | R⁵ | [M + H]⁺ | LCMS RT | ¹H NMR (400 MHz, DMSO-d₆) δ |
| 20 | 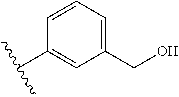 [SMILES: *c1cccc(CO)c1] | 338.2 | 2.48 | (3-((1-(cyclohexylmethyl)-1H-pyrazolo[3,4- d)pyrimidin-6-yl)amino)phenyl)methanol 9.87 (s, 1H), 8.98 (s, 1H), 8.07 (s, 1H), 8.05 (s, 1H), 7.66-7.64 (m, 1H), 7.26 (t, J = 7.8 Hz, 1H), 6.95 (d, J = 7.6 Hz, 1H), 5.16 (t, J = 5.7 Hz, 1H), 4.52 (d, J = 5.6 Hz, 2H), 4.16 (d, J = 7.1 Hz, 2H), 2.01-1.94 (m, |
| | | | | 1H), 1.67-1.55 (m, 5H), 1.24-1.09 (m, 5H). |
| 22 | 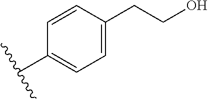 [SMILES: *c1ccc(CCO)cc1] | 352.0 | 2.54 | 2-(4-((1-(cyclohexylmethyl)-1H-pyrazolo[3,4- d]pyrimidin-6-yl)amino)phenyl)ethan-1-ol 9.78 (s, 1H), 8.96 (s, 1H), 8.05 (s, 1H), 7.78 (d, J = 8.4 Hz, 2H), 7.16 (d, J = 8.4 Hz, 2H)), 4.61 (t, J = 5.2 Hz 1H), 4.14 (d, J = 7.2 Hz, 2H), 3.61-3.57 (m, 2H), 2.69 (t, J = 7.2 Hz, 2H), 2.00-1.95 (m, 1H), 1.68 1.51 (m, 5H), 1.21-1.00 (m, 5H). |
| 24 | 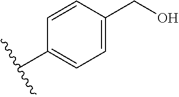 [SMILES: *c1ccc(CO)cc1] | 338.2 | 1.99 | (4-((1-(cyclohexylmethyl)-1H-pyrazolo[3,4- d]pyrimidin-6-yl)amino)phenyl)methanol 9.74 (s, 1H), 8.94 (s, 1H), 8.04 (s, 1H), 7.80 (d, J = 8.2 Hz, 2H), 7.25 (d, J = 8.3 Hz, 2H), 5.26 (t, J = 5.7 Hz, 1H), 4.45 (d, J = 5.6 Hz, 2H), 4.12 (d, J = 7.0 Hz, 2H), 2.00-1.95 (m, 1H), 1.67-1.57 (m, 5H), 1.23- |
| | | | | 0.95 (m, 5H). |
| 35 | 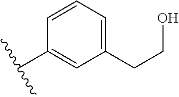 [SMILES: *c1cccc(CCO)c1] | 352.3 | 1.96 | 2-(3-((1-(cyclohexylmethyl)-1H-pyrazolo[3,4- d]pyrimidin-6-yl)amino)phenyl)ethan-1-o1 9.82 (s, 1H), 8.97 (s, 1H), 8.06 (s, 1H), 7.94 (s, 1H), 7.64-7.53 (m, 1H), 7.21 (t, J = 7.8 Hz, 1H), 6.84 (d, J = 8.4 Hz, 1H), 4.65 (t, J = 5.2 Hz, 1H), 4.16 (d, J = 7.1 Hz, 2H), 3.68-3.63 (m, 2H), 2.74 (t, J = 7.2 Hz, |
| | | | | 2H), 2.07-1.95 (m, 1H), 1.75-1.50 (m, 5H), 1.24- |
| | | | | 1.02 (m, 5H). |
| 55 | 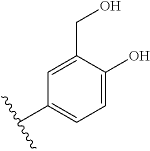 [SMILES: *c1ccc(O)c(CO)c1] | 354.2 | 2.45 | 4-((1-(cyclohexylmethyl)-1H-pyrazolo[3,4- d]pyrimidin-6-yl)amino)-2-(hydroxymethyl)phenol 9.55 (s, 1H), 8.99 (s, 1H), 8.89 (s, 1H), 8.00 (s, 1H), 7.93 (bs, 1H), 7.44 (dd, J = 8.8, 2.8 Hz, 1H), 6.71 (d, J = 8.8 Hz, 1H), 4.94 (t, J = 5.5 Hz, 1H), 4.50 (d, J = 5.4 Hz, 2H), 4.12 (d, J = 7.1 Hz, 2H), 1.98-1.94 (m, 1H), 1.67-1.52 (m, 5H), 1.19-1.08 (m, 5H). |
| 79 | 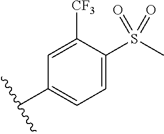 [SMILES: *c1ccc(S(C)(=O)=O)c(C(F)(F)F)c1] | 454.2 | 2.24 | 1-(cyclohexylmethyl)-N-(4-(methylsulfonyl)-3- (trifluoromethyl)phenyl)-1H-pyrazolo[3,4- d]pyrimidin-6-amine 10.82 (s, 1H), 9.13 (s, 1H), 8.94 (d, J = 1.6 Hz, 1H), 8.24-8.16 (m, 3H), 4.22 (d, J = 7.2 Hz, 2H), 3.26 (s, 3H), 2.00-1.91 (m, 1H), 1.67-1.54 (m, 5H), 1.24-0.98 (m, 5H). |
| 80 | 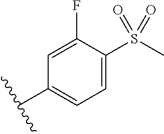 [SMILES: *c1ccc(S(C)(=O)=O)c(F)c1] | 404.1 | 2.43 | 1-(cyclohexylmethyl)-N-(3-fluoro-4- (methylsulfonyl)phenyl)-1H-pyrazolo[3,4- d]pyrimidin-6-amine 10.68 (s, 1H), 9.12 (s, 1H), 8.25-8.19 (m, 2H), 7.80- 7.78 (m, 2H), 4.22 (d, J = 7.0 Hz, 2H), 3.28 (s, 3H), 2.02-1.97 (m, 1H), 1.69-1.57 (m, 5H), 1.12-1.03 (m, 5H). |

### General Procedure for the Synthesis of Compounds 25-26

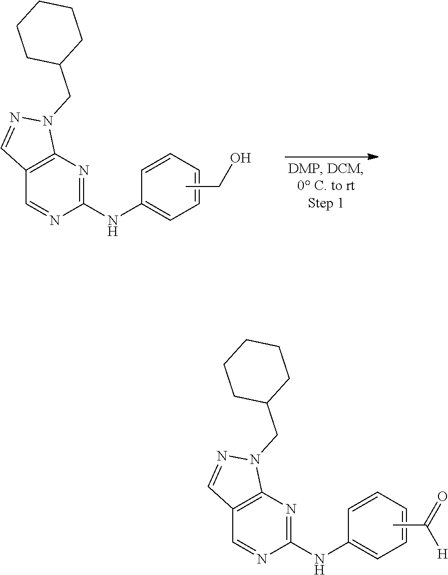

To a stirred solution of benzylic alcohol (1 mmol) in DCM (20 mL) was added DMP (1.5 mmol) at 0° C. The reaction mixture was stirred at rt. After completion, the reaction mixture was diluted with aqueous sodium bicarbonate solution (20 mL) and extracted with DCM (2×30 mL). The combined organic layers were dried over anhydrous Na₂SO₄, filtered, and concentrated in vacuo to afford crude product, which was purified by Biotage Isolera to obtain the corresponding aldehyde.

TABLE 6
The following compounds were prepared in a manner like those exemplified in above
general procedure. Compounds are racemic or achiral unless specified otherwise.

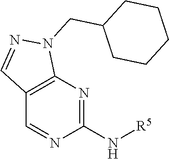

| | | | | NAME |
|---|---|---|---|---|
| Ex No. | R⁵ | [M + H]⁺ | LCMS RT | ¹H NMR (400 MHz, DMSO-d₆) δ |
| 25 | 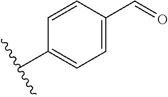 [SMILES: *c1ccc(C=O)cc1] | 336.1 | 2.30 | 4-((1-(cyclohexylmethyl)-1H-pyrazolo[3,4-d]pyrimidin- 6-yl)amino)benzaldehyde 10.47 (s, 1H), 9.87 (s, 1H), 9.09 (s, 1H), 8.16 (s, 1H), 8.14 (d, J = 8.8 Hz, 2H), 7.89 (d, J = 8.8 Hz, 2H), 4.22 (d, J = 7.2 Hz, 2H), 2.09-1.99 (m, 1H), 1.68-1.60 (m, 5H), 1.17-1.06 (m, 5H). |
| 26 | 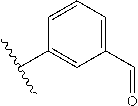 [SMILES: *c1cccc(C=O)c1] | 336.2 | 2.81 | 3-((1-(cyclohexylmethyl)-1H-pyrazolo[3,4-d]pyrimidin- 6-yl)amino)benzaldehyde 10.23 (s, 1H), 10.02 (s, 1H), 9.04 (s, 1H), 8.77 (s, 1H), 8.12 (s, 1H), 8.03-8.00 (m, 1H), 7.57-7.55 (m, 2H), 4.21 (d, J = 7.2 Hz, 2H), 2.03-1.97 (m, 1H), 1.69-1.56 (m, 5H), 1.22-1.03 (m, 5H). |

### General Procedure for the Synthesis of Compounds 21, 23, 30, 31, 34, 36, 37 and 42

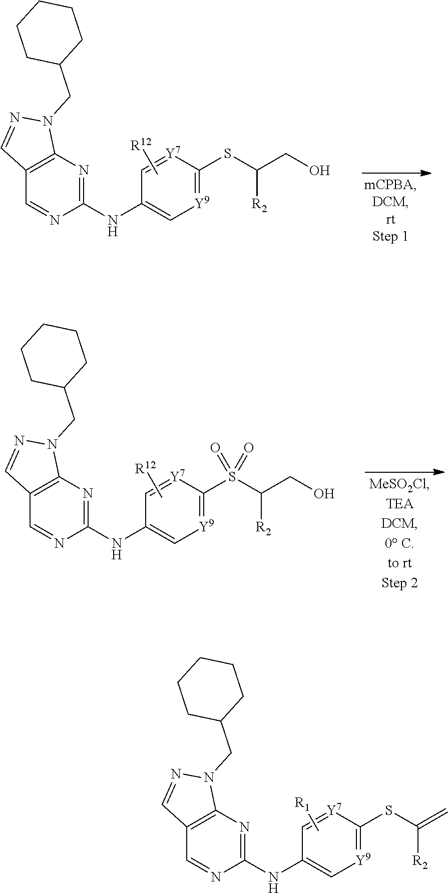

### Step 1:

To a stirred solution of sulfide (1 mmol) in DCM (30 mL) was added mCPBA (4 mmol) at 0° C. and the reaction mixture was stirred at rt. After completion, the reaction mixture was diluted with aqueous sodium bicarbonate solution (20 mL) and extracted with DCM (2×30 mL). The combined organic layers were dried over anhydrous Na₂SO₄, filtered, and concentrated in vacuo to afford crude product, which was purified by Biotage Isolera to obtain the corresponding β-hydroxy sulfone.

### Step 2:

To a stirred solution of β-hydroxy sulfone (1 mmol) in DCM (5 mL) was added MeSO₂Cl (4 mmol) dropwise at 0° C. followed by addition of TEA (10 mmol). The reaction mixture was stirred at 0° C. After completion, the reaction mixture was diluted with water (10 mL) and extracted with EtOAc (2×20 mL). The combined organic layers were washed with brine (10 mL), dried over anhydrous Na₂SO₄, filtered, and concentrated in vacuo to afford crude product, which was purified by Biotage Isolera to afford the corresponding vinyl sulfone.

TABLE 7
The following compounds were prepared in a manner similar to those exemplified in
above general procedure. Compounds are racemic or achiral unless specified otherwise.

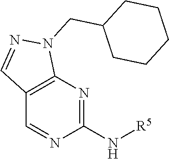

| | | | | NAME |
|---|---|---|---|---|
| Ex No. | R⁵ | [M + H]⁺ | LCMS RT | ¹H NMR (400 MHz, DMSO-d₆) δ |
| 21 | 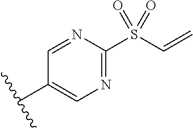 [SMILES: *c1cnc(S(=O)(=O)C=C)nc1] | 400.2 | 2.59 | 1-(cyclohexylmethyl)-N-(2-(vinylsulfonyl)pyrimidin- 5-yl)-1H-pyrazolo[3,4-d]pyrimidin-6-amine 10.80 (s, 1H), 9.50 (s, 2H), 9.16 (s, 1H), 8.23 (s, 1H), 7.29 (dd, J = 16.8, 10.0 Hz, 1H), 6.55-6.37 (m, 2H), 4.23 (d, J = 6.9 Hz, 2H), 2.04-1.96 (m, 1H), 1.67- 1.54 (m, 5H), 1.30-1.02 (m, 5H). |
| 23 | 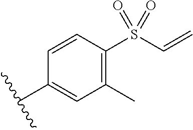 [SMILES: *c1ccc(S(=O)(=O)C=C)c(C)c1] | 412.0 | 2.85 | 1-(cyclohexylmethyl)-N-(3-methyl-4-(vinylsulfonyl) phenyl)-1H-pyrazolo[3,4-d]pyrimidin-6-amine 10.40 (s, 1H), 9.08 (s, 1H), 8.16 (s, 1H), 8.06 (s, 1H), 7.96 (dd, J = 8.8, 2.0 Hz, 1H), 7.87 (d, J = 8.8 Hz, 1H), 7.04 (dd, J = 16.4, 10.0 Hz, 1H), 6.29 (d, J = 16.4 Hz, 1H), 6.19 (d, J = 10.0 Hz, 1H), 4.21 (d, J = 7.6 Hz, 2H), 2.51 (s, 3H), 2.02-1.96 (m, 1H), 1.62-1.52 (m, 5H), 1.25-1.02 (m, 5H). |
| 30 | 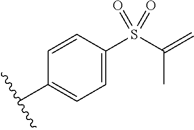 [SMILES: *c1ccc(S(=O)(=O)C(=C)C)cc1] | 412.3 | 2.14 | 1-(cyclohexylmethyl)-N-(4-(prop-1-en-2-ylsulfonyl) phenyl)-1H-pyrazolo[3,4-d]pyrimidin-6-amine 10.50 (s, 1H), 9.09 (s, 1H), 8.18 (d, J = 8.8 Hz, 2H), 8.16 (s, 1H), 7.79 (d, J = 8.8 Hz, 2H), 6.11 (s, 1H), 5.88 (s, 1H), 4.22 (d, J = 7.0 Hz, 2H), 2.05-1.93 (m, 1H), 1.92 (s, 3H), 1.66-1.54 (m, 5H), 1.25-0.91 (m, 5H). |
| 31 | 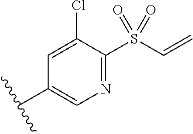 [SMILES: *c1cnc(S(=O)(=O)C=C)c(Cl)c1] | 433.0 | 2.72 | N-(5-chloro-6-(vinylsulfonyl)pyridin-3-yl)-1- (cyclohexylmethyl)-1H-pyrazolo[3,4-d]pyrimidin-6- amine 10.86 (s, 1H), 9.16 (s, 1H), 8.93 (s, 2H), 8.23 (s, 1H), 7.30 (dd, J = 16.4, 10.0 Hz, 1H), 6.44 (d, J = 16.8 Hz, 1H), 6.39 (d, J = 10.0 Hz, 1H), 4.22 (d, J = 7.0 Hz, 2H), 2.03-1.97 (m, 1H), 1.74-1.54 (m, 5H), 1.27-0.98 (m, 5H). |
| 34 | 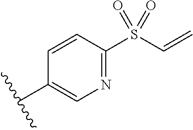 [SMILES: *c1ccc(S(=O)(=O)C=C)nc1] | 399.2 | 2.54 | 1-(cyclohexylmethyl)-N-(6-(vinylsulfonyl)pyridin-3- yl)-1H-pyrazolo[3,4-d]pyrimidin-6-amine 10.67 (s, 1H), 9.17 (s, 1H), 9.16 (s, 1H), 8.69 (dd, J = 8.8, 2.6 Hz, 1H), 8.20 (s, 1H), 8.09 (d, J = 8.8 Hz, 1H), 7.14 (dd, J = 16.4, 10.0 Hz, 1H), 6.39 (d, J = 16.4 Hz, 1H), 6.25 (d, J = 10.0 Hz, 1H), 4.22 (d, J = 7.0 Hz, 2H), 2.00-1.89 (m, 1H), 1.70-1.40 (m, 5H), 1.26-0.97 (m, 5H). |
| 36 | 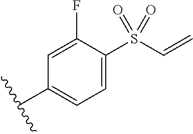 [SMILES: *c1ccc(S(=O)(=O)C=C)c(F)c1] | 416.2 | 2.76 | 1-(cyclohexylmethyl)-N-(3-fluoro-4-(vinylsulfonyl) phenyl)-1H-pyrazolo[3,4-d]pyrimidin-6-amine 10.70 (s, 1H), 9.12 (s, 1H), 8.22 (s, 1H), 8.19 (s, 1H), 7.80 (bs, 2H), 7.12 (dd, J = 16.4, 9.8 Hz, 1H), 6.37 (d, J = 16.4 Hz, 1H), 6.26 (d, J = 9.8 Hz, 1H), 4.21 (d, J = 6.8 Hz, 2H), 2.10-1.87 (m, 1H), 1.77-1.44 (m, 5H), 1.22-0.90 (m, 5H). |
| 37 | 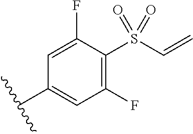 [SMILES: *c1cc(F)c(S(=O)(=O)C=C)c(F)c1] | 434.0 | 2.79 | 1-(cyclohexylmethyl)-N-(3,5-difluoro-4- (vinylsulfonyl) phenyl)-1H-pyrazolo[3,4-d]pyrimidin- 6-amine 10.84 (s, 1H), 9.15 (s, 1H), 8.22 (s, 1H), 7.84-7.81 (m, 2H), 7.25 (dd, J = 16.4, 9.6 Hz, 1H), 6.39 (d, J = 16.4 Hz, 1H), 6.29 (d, J = 9.6 Hz, 1H), 4.21 (d, J = 7.0 Hz, 2H), 2.13-1.90 (m, 1H), 1.77-1.55 (m, 5H), 1.30- 0.93 (m, 5H). |
| 42 | 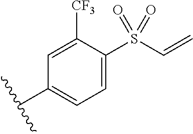 [SMILES: *c1ccc(S(=O)(=O)C=C)c(C(F)(F)F)c1] | 466.0 | 2.96 | 1-(cyclohexylmethyl)-N-(3-(trifluoromethyl)-4-(vinyl sulfonyl)phenyl)-1H-pyrazolo[3,4-d]pyrimidin-6- amine 10.86 (s, 1H), 9.14 (s, 1H), 8.94 (s, 1H), 8.25-8.16 (m, 3H), 7.10 (dd, J = 16.4, 10.0 Hz, 1H), 6.29 (d, J = 16.4 Hz, 1H), 6.22 (d, J = 9.6 Hz, 1H), 4.22 (d, J = 7.2 Hz, 2H), 2.01-1.92 (m, 1H), 1.70-1.53 (m, 5H), 1.30- 1.01 (m, 5H). |

### General Procedure for Synthesis of Compounds 38 and 44

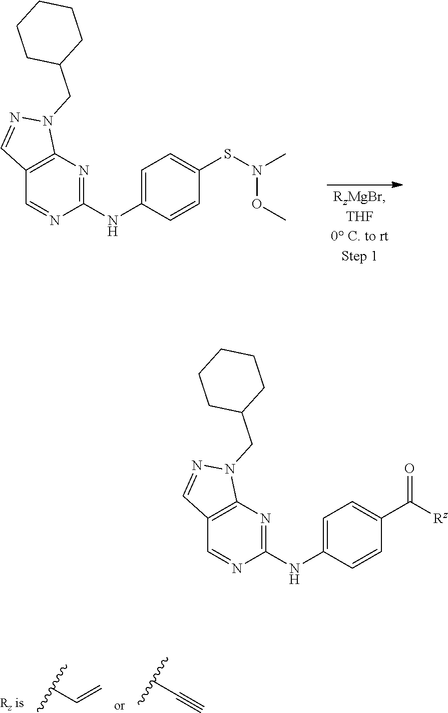

To a stirred solution of Weinreb amide (1 mmol) in THE (10 mL) was added Grignard reagent (5 mmol) dropwise at 0° C. The reaction mixture was stirred at rt. After completion, the reaction mixture was cooled to 0° C., quenched with aqueous ammonium chloride solution (10 mL) and extracted with EtOAc (2×20 mL). The combined organic layers were washed with brine (15 mL), dried over anhydrous Na₂SO₄, filtered, and concentrated in vacuo to afford crude product, which was purified by Biotage Isolera to afford the corresponding ketone.

TABLE 8
The following compounds were prepared in a manner like those exemplified in above
general procedure. Compounds are racemic or achiral unless specified otherwise.

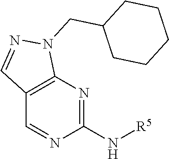

| | | | | NAME |
|---|---|---|---|---|
| Ex No. | R⁵ | [M + H]⁺ | LCMS RT | ¹H NMR (400 MHz, DMSO-d₆) δ |
| 38 | 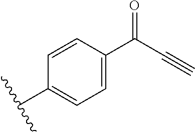 [SMILES: *c1ccc(C(=O)C#C)cc1] | 360.0 | 2.91 | 1-(4-((1-(cyclohexylmethyl)-1H-pyrazolo[3,4- d]pyrimidin-6-yl)amino)phenyl)prop-2-yn-1-one 10.55 (s, 1H), 9.10 (s, 1H), 8.19-8.11 (m, 3H), 8.09- 8.04 (m, 2H), 4.98 (s, 1H), 4.23 (d, J = 7.0 Hz, 2H), 2.01-1.95 (m, 1H), 1.66-1.55 (m, 5H), 1.23-1.03 (m, 5H). |
| 44 | 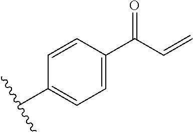 [SMILES: *c1ccc(C(=O)C=C)cc1] | 362.3 | 3.04 | 1-(4-((1-(cyclohexylmethyl)-1H-pyrazolo[3,4- d]pyrimidin-6-yl)amino)phenyl)prop-2-en-1-one 10.41 (s, 1H), 9.08 (s, 1H), 8.15 (s, 1H), 8.11-8.03 (m, 4H), 7.48 (dd, J = 17.2, 10.0 Hz, 1H), 6.34 (d, J = 16.8 Hz, 1H), 5.94 (d, J = 10.0 Hz, 1H), 4.21 (d, J = 7.0 Hz, 2H), 2.02-1.96 (m, 1H), 1.73-1.46 (m, 5H), 1.29-0.94 (m, 5H). |

### Compound 43: 1-(cyclohexylmethyl)-N-(4-(prop-1-yn-1-ylsulfonyl)phenyl)-1H-pyrazolo[3,4-d]pyrimidin-6-amine

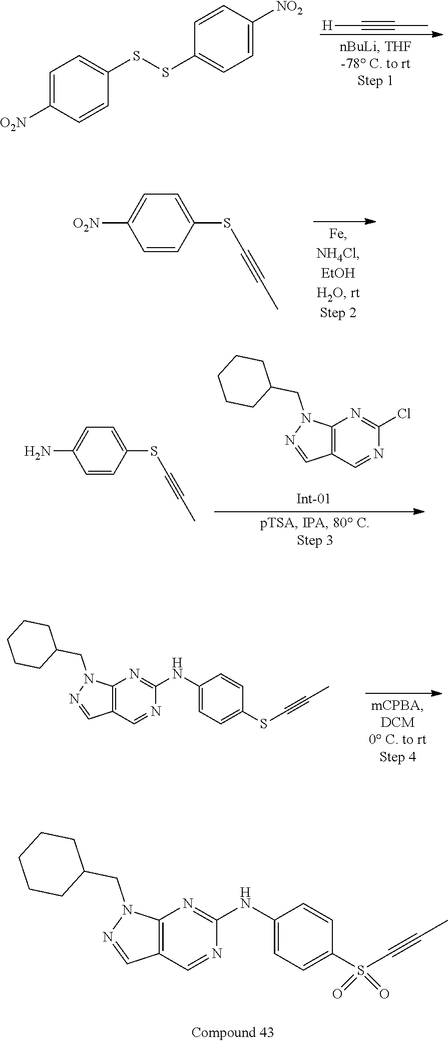

### Step 1: (4-nitrophenyl)(prop-1-yn-1-yl)sulfane

To a stirred solution of prop-1-yne (37.4 mL, 37.4 mmol, 1M solution in THF) was added nBuLi (15 mL, 37.4 mmol, 2.5 M solution in THF) dropwise at −78° C. After stirring the reaction mixture at the same temperature for 1 h, a solution of 1,2-bis(4-nitrophenyl)disulfane (11.5 g, 37.4 mmol) in THF (40 mL) was added at the same temperature. The reaction mixture was brought to rt and stirred for 2 h. After completion, the reaction mixture was cooled to −78° C. and quenched with aq NH₄Cl (100 mL) slowly. The reaction mixture was warmed to rt and extracted with EtOAc (3×100 mL). The combined organic layers were washed with brine (50 mL), dried over anhydrous Na₂SO₄, filtered, and concentrated in vacuo to afford the desired product, (4-nitrophenyl)(prop-1-yn-1-yl)sulfane (4.1 g, 20.84 mmol, 56.7% yield) as a yellow solid.

LCMS (Method B): 98.2%, RT: 2.06 min, m/z: 387.2 [2M+H]⁺.

### Step 2: 4-(prop-1-yn-1-ylthio)aniline

To a stirred solution of (4-nitrophenyl)(prop-1-yn-1-yl)sulfane (4.1 g, 21.2 mmol) in EtOH (40 mL) and water (40 mL) were added NH₄Cl (11.4 g, 212 mmol) and iron powder (11.9 g, 212 mmol) portionwise. The reaction mixture was stirred at rt for 16 h. After completion, the reaction mixture was filtered through Celite® bed and washed with EtOAc (50 mL), and the filtrate was extracted with EtOAc (3×30 mL). The combined organic layers were washed with brine (20 mL), dried over anhydrous Na₂SO₄, filtered and the filtrate was concentrated in vacuo to crude product, which was purified by Biotage Isolera (15% EtOAc in hexanes) to obtain the desired product, 4-(prop-1-yn-1-ylthio)aniline (2.9 g, 17.4 mmol, 82.1% yield) as a yellow solid.

LCMS (Method B): 98.1%, RT: 1.73 min, m/z: 164.2 [M+H]⁺.

### Step 3: 1-(cyclohexylmethyl)-N-(4-(prop-1-yn-1-ylthio)phenyl)-1H-pyrazolo[3,4-d]pyrimidin-6-amine

To a stirred solution of 6-chloro-1-(cyclohexylmethyl)-1H-pyrazolo[3,4-d]pyrimidine (800 mg, 3.19 mmol) in IPA (10 mL) were added 4-(prop-1-yn-1-ylthio)aniline (1.04 g, 6.38 mmol) and p-toluenesulfonic acid monohydrate (61 mg, 0.319 mmol). The reaction mixture was stirred at 80° C. for 16 h. After completion, the reaction mixture was quenched with ice-cold water (15 mL) and extracted with EtOAc (3×20 mL). The combined organic layers were washed with brine (20 mL), dried over anhydrous Na₂SO₄, filtered and concentrated in vacuo to give crude product, which was purified by Biotage Isolera (30% EtOAc in hexanes) to obtain the desired product, 1-(cyclohexylmethyl)-N-(4-(prop-1-yn-1-ylthio)phenyl)-1H-pyrazolo[3,4-d]pyrimidin-6-amine (754 mg, 1.568 mmol, 49.2% yield) as an off-white solid.

LCMS (Method A): 78.5%, RT: 3.61 min, m/z: 378.2 [M+H]⁺.

### Step 4. Compound 43: 1-(cyclohexylmethyl)-N-(4-(prop-1-yn-1-ylsulfonyl)phenyl)-1H-pyrazolo[3,4-d]pyrimidin-6-amine

To a stirred solution of 1-(cyclohexylmethyl)-N-(4-(prop-1-yn-1-ylthio)phenyl)-1H-pyrazolo[3,4-d]pyrimidin-6-amine (350 mg, 0.927 mmol) in DCM (5 mL) was added mCPBA (571 mg, 2.32 mmol) at 0° C. The reaction mixture was stirred at rt for 2 h. After completion, the reaction mixture was quenched with aqueous sodium bicarbonate solution (20 mL) and extracted with DCM (2×30 mL). The combined organic layers were dried over anhydrous Na₂SO₄, filtered, and concentrated in vacuo to afford crude product, which was purified by Biotage Isolera (30% EtOAc in hexanes) to obtain the desired product, 1-(cyclohexylmethyl)-N-(4-(prop-1-yn-1-ylsulfonyl)phenyl)-1H-pyrazolo[3,4-d]pyrimidin-6-amine (25 mg, 0.061 mmol, 6.6% yield) as a white solid. ¹H NMR (400 MHz, DMSO-d₆): δ 10.58 (s, 1H), 9.10 (s, 1H), 8.22-8.15 (m, 3H), 7.94-7.91 (m, 2H), 4.23 (d, J=7.0 Hz, 2H), 2.13 (s, 3H), 2.04-1.98 (m, 1H), 1.68-1.55 (m, 5H), 1.28-0.95 (m, 5H). LCMS (Method A): 99.6%, RT: 2.86 min, m/z: 410.2 [M+H]⁺.

### Compound 19: 1-(cyclohexylmethyl)-3-methyl-N-(4-(methylsulfonyl)phenyl)-1H-pyrazolo[3,4-d]pyrimidin-6-amine

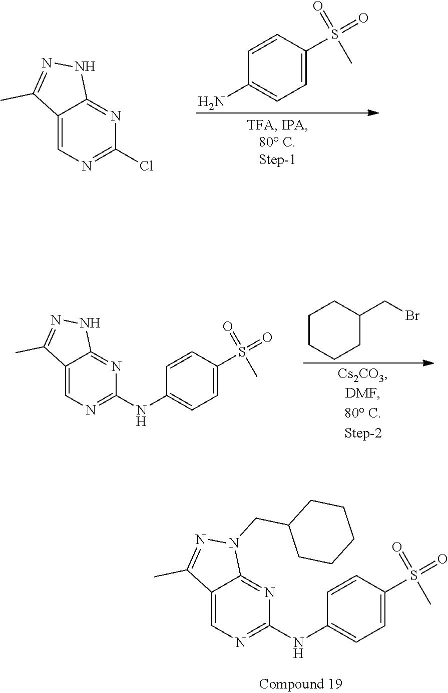

### Step 1: 3-methyl-N-(4-(methylsulfonyl)phenyl)-1H-pyrazolo[3,4-d]pyrimidin-6-amine

To a stirred solution of 6-chloro-3-methyl-1H-pyrazolo[3,4-d]pyrimidine (100 mg, 0.593 mmol) and 4-(methylsulfonyl)aniline (102 mg, 0.593 mmol) in IPA (5 mL) was added TFA (15 mg, 0.130 mmol) at rt. The reaction mixture stirred at 80° C. for 16 h. After completion, the reaction mixture was concentrated in vacuo, diluted with water (10 mL), and extracted with EtOAc (3×10 mL). The combined organic layers were dried over anhydrous Na₂SO₄, filtered, and concentrated in vacuo to afford crude product, which was purified by Biotage Isolera (40% EtOAc in hexanes) to obtain the desired product, 3-methyl-N-(4-(methylsulfonyl)phenyl)-1H-pyrazolo[3,4-d]pyrimidin-6-amine (95 mg, 0.310 mmol, 52.3% yield) as an off-white solid.

LCMS (Method A): 99.8%, RT: 1.81 min, m/z: 304.0 (M+H)⁺.

### Step 2. Compound 19: 1-(cyclohexylmethyl)-3-methyl-N-(4-(methylsulfonyl)phenyl)-1H-pyrazolo[3,4-d]pyrimidin-6-amine

To a stirred solution of 3-methyl-N-(4-(methylsulfonyl)phenyl)-1H-pyrazolo[3,4-d]pyrimidin-6-amine (95 mg, 0.313 mmol) in DMF (5 mL) were added Cs₂CO₃ (306 mg, 0.940 mmol) and (bromomethyl)cyclohexane (83 mg, 0.470 mmol) at rt. The reaction mixture was stirred at 90° C. for 16 h. After completion of the reaction, the reaction mixture was concentrated in vacuo to give the crude compound, which was purified by prep HPLC and pure fractions were lyophilized to give 1-(cyclohexylmethyl)-3-methyl-N-(4-(methylsulfonyl)phenyl)-1H-pyrazolo[3,4-d]pyrimidin-6-amine (6 mg, 0.014 mmol, 4.6% yield), as a white solid. ¹H NMR (400 MHz, DMSO-d₆): δ 10.40 (s, 1H), 9.05 (s, 1H), 8.15 (d, J=8.8 Hz, 2H), 7.86 (d, J=8.8 Hz, 2H), 4.12 (d, J=7.0 Hz, 2H), 3.17 (s, 3H), 2.49 (s, 3H), 2.01-1.90 (m, 1H), 1.71-1.52 (m, 5H), 1.25-1.00 (m, 5H). LCMS (Method B): 97.7% RT: 2.60 min, m/z: 400.1 (M+H)⁺.

### Compound 56: 1-(((1s,4s)-4-(dimethylamino)cyclohexyl)methyl)-N-(1-(methylsulfonyl)piperidin-4-yl)-1H-pyrazolo[3,4-d]pyrimidin-6-amine

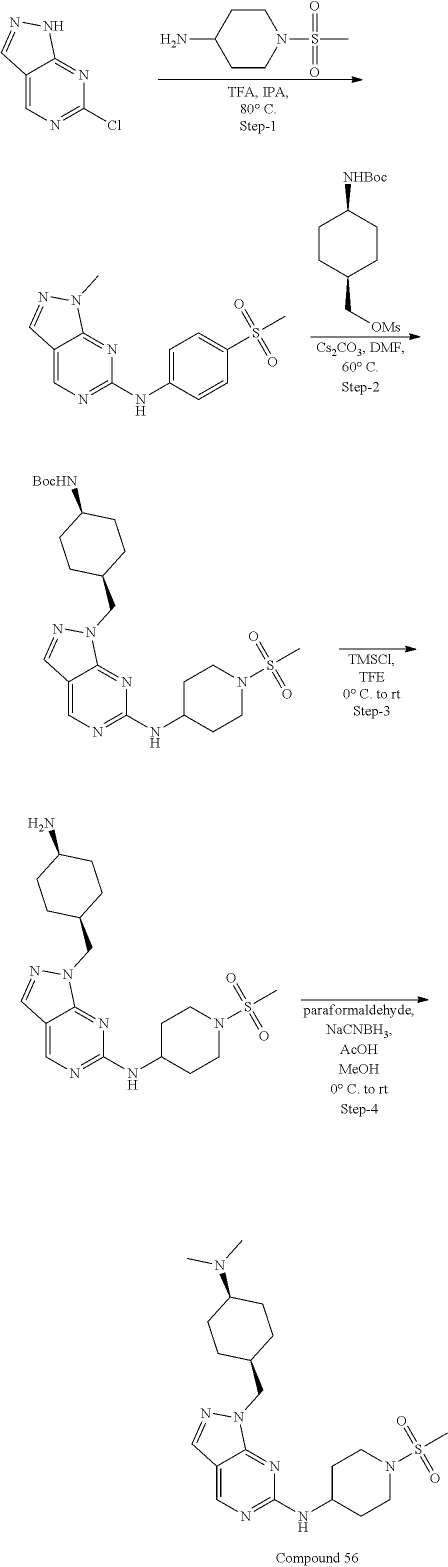

### Step 1: N-(1-(methylsulfonyl)piperidin-4-yl)-1H-pyrazolo[3,4-d]pyrimidin-6-amine

To a stirred solution of 6-chloro-1H-pyrazolo[3,4-d]pyrimidine (3 g, 19.4 mmol) in IPA (3 mL) were added 1-(methylsulfonyl)piperidin-4-amine (5.19 g, 29.1 mmol) and TFA (22.1 g, 194 mmol) at rt. The reaction mixture was stirred at 80° C. for 16 h. After completion, the solid precipitated was filtered and was washed with MTBE (50 mL) and dried in vacuo to afford the desired product, N-(1-(methylsulfonyl)piperidin-4-yl)-1H-pyrazolo[3,4-d]pyrimidin-6-amine (2.5 g, 2.26 mmol, 11.7% yield) as an off white solid, which was used without further purification.

### Step 2: tert-butyl ((1s,4s)-4-((6-((1-(methylsulfonyl)piperidin-4-yl)amino)-1H-pyrazolo[3,4-d]pyrimidin-1-yl)methyl)cyclohexyl)carbamate

To a stirred solution of N-(1-(methylsulfonyl)piperidin-4-yl)-1H-pyrazolo[3,4-d]pyrimidin-6-amine (400 mg, 1.35 mmol) in DMF (3 mL) were added Cs₂CO₃ (1.32 g, 4.05 mmol) and ((1s,4s)-4-((tert-butoxycarbonyl)amino)cyclohexyl)methyl methanesulfonate (602 mg, 1.96 mmol, prepared from corresponding alcohol) at rt. The reaction mixture was stirred at 60° C. for 16 h and progress of reaction was monitored by TLC and LCMS. After completion, the reaction mixture was quenched with ice-cold water (20 mL) and extracted with EtOAc (3×20 mL). The combined organic layers were washed with brine (20 mL), dried over anhydrous Na₂SO₄, filtered and concentrated in vacuo to give the desired compound, tert-butyl ((1s,4s)-4-((6-((1-(methylsulfonyl)piperidin-4-yl)amino)-1H-pyrazolo[3,4-d]pyrimidin-1-yl)methyl)cyclohexyl)carbamate (500 mg, 0.865 mmol, 64.1% yield) as an off-white solid. LCMS (Method A): 87.8%, RT: 2.37 min, m/z: 508.2 (M+H)⁺.

### Step 3: 1-(((1s,4s)-4-aminocyclohexyl)methyl)-N-(1-(methylsulfonyl)piperidin-4-yl)-1H-pyrazolo[3,4-d]pyrimidin-6-amine

To a stirred solution of tert-butyl ((1s,4s)-4-((6-((1-(methylsulfonyl)piperidin-4-yl)amino)-1H-pyrazolo[3,4-d]pyrimidin-1-yl)methyl)cyclohexyl)carbamate (500 mg, 0.985 mmol) in TFE (3 mL) was added TMSCl (0.38 mL, 2.95 mmol) dropwise at 0° C. The reaction mixture stirred at rt for 2 h. After completion, the reaction mixture was concentrated in vacuo to give a sticky solid, which was triturated with MTBE (30 mL) and dried in vacuo to afford the desired compound, 1-(((1s,4s)-4-aminocyclohexyl)methyl)-N-(1-(methylsulfonyl)piperidin-4-yl)-1H-pyrazolo[3,4-d]pyrimidin-6-amine (300 mg, 0.632 mmol, 64.2% yield) as a yellow solid.

LCMS (Method A): 85.9%, RT: 1.11 min, m/z: 408.2 (M+H)⁺.

### Step 4. Compound 56: 1-(((1s,4s)-4-(dimethylamino)cyclohexyl)methyl)-N-(1-(methylsulfonyl)piperidin-4-yl)-1H-pyrazolo[3,4-d]pyrimidin-6-amine

A solution of 1-(((1s,4s)-4-aminocyclohexyl)methyl)-N-(1-(methylsulfonyl)piperidin-4-yl)-1H-pyrazolo[3,4-d]pyrimidin-6-amine (100 mg, 0.245 mmol), AcOH (2 mg, 0.025 mmol) and paraformaldehyde (60 mg) in MeOH (3 mL) was stirred at rt for 1 h. NaCNBH₃ (32 mg, 0.515 mmol) was added at 0° C. The resulting reaction mixture was stirred at rt for 16 h. After completion, the reaction mixture was concentrated in vacuo, quenched with ice-cold water (10 mL) and extracted with EtOAc (2×20 mL). The combined organic layers were washed with brine (10 mL), dried over anhydrous Na₂SO₄, filtered, and concentrated in vacuo to afford crude product, which was purified by prep HPLC and pure fractions were lyophilized to give the desired compound, 1-(((1s,4s)-4-(dimethylamino)cyclohexyl)methyl)-N-(1-(methylsulfonyl)piperidin-4-yl)-1H-pyrazolo[3,4-d]pyrimidin-6-amine (15 mg, 0.034 mmol, 14% yield) as an off-white solid. ¹H NMR (400 MHz, DMSO-d₆): δ 8.80 (s, 1H), 7.95 (s, 1H), 7.51 (bs, 1H), 4.20-3.90 (m, 3H), 3.57-3.54 (m, 2H), 2.91-2.86 (m, 5H), 2.20-1.95 (m, 10H), 1.75-1.57 (m, 4H), 1.45-1.30 (m, 6H). LCMS (Method A): 99.4%, RT: 1.17 min, m/z: 436.4 (M+H)⁺.

The following table shows the structures of exemplary compounds which are prepared according to the methods described above.

TABLE 9
Exemplary Compound Structures

| Cmpd | | |
|---|---|---|
| # | Structure | Name |
| 1 | 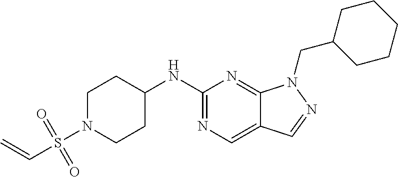 [SMILES: C=CS(=O)(=O)N1CCC(Nc2ncc3cnn(CC4CCCCC4)c3n2)CC1] | 1-(cyclohexylmethyl)-N-(1- (vinylsulfonyl)piperidin-4- yl)-1H-pyrazolo[3,4- d]pyrimidin-6-amine |
| 2 | 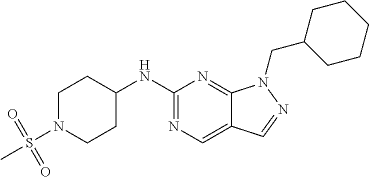 [SMILES: CS(=O)(=O)N1CCC(Nc2ncc3cnn(CC4CCCCC4)c3n2)CC1] | 1-(cyclohexylmethyl)-N-(1- (methylsulfonyl)piperidin-4- yl)-1H-pyrazolo[3,4- d]pyrimidin-6-amine |
| 3 | 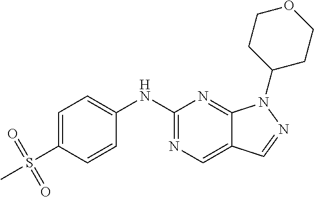 [SMILES: CS(=O)(=O)c1ccc(Nc2ncc3cnn(C4CCOCC4)c3n2)cc1] | N-(4- (methylsulfonyl)phenyl)-1- (tetrahydro-2H-pyran-4-yl)- 1H-pyrazolo[3,4- d]pyrimidin-6-amine |
| 4 | 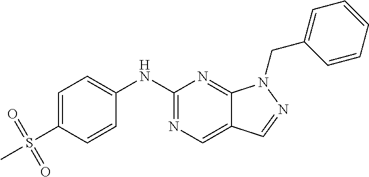 [SMILES: CS(=O)(=O)c1ccc(Nc2ncc3cnn(Cc4ccccc4)c3n2)cc1] | 1-benzyl-N-(4- (methylsulfonyl)phenyl)-1H- pyrazolo[3,4-d]pyrimidin-6- amine |
| 5 | 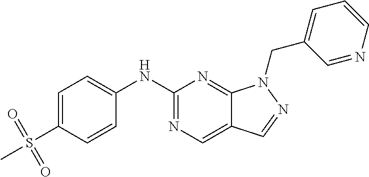 [SMILES: CS(=O)(=O)c1ccc(Nc2ncc3cnn(Cc4cccnc4)c3n2)cc1] | N-(4- (methylsulfonyl)phenyl)-1- (pyridine-3-ylmethyl)-1H- pyrazolo[3,4-d]pyrimidin-6- amine |
| 6 | | 1-(((1S,4S)-4- |
| | | aminocyclohexyl)methyl)-N- |
| | | (4-(methylsulfonyl)phenyl)- |
| | | 1H-pyrazolo[3,4- |
| | | d]pyrimidin-6-amine |
| 7 | | 1-(((15,4S)-4- |
| | | aminocyclohexyl)methyl)-N- |
| | | (4-(methylsulfonyl)phenyl)- |
| | | 1H-pyrazolo[3,4- |
| | | d]pyrimidin-6-amine |
| 8 | 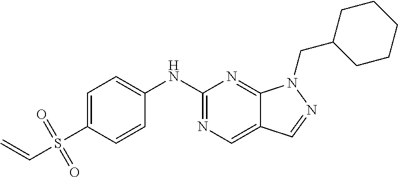 [SMILES: C=CS(=O)(=O)c1ccc(Nc2ncc3cnn(CC4CCCCC4)c3n2)cc1] | 1-(cyclohexylmethyl)-N-(4- (vinylsulfonyl)phenyl)-1H- pyrazolo[3,4-d]pyrimidin-6- amine |
| 9 | 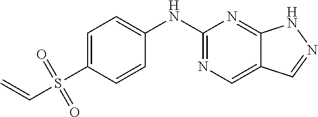 [SMILES: C=CS(=O)(=O)c1ccc(Nc2ncc3cn[nH]c3n2)cc1] | N-(4-(vinylsulfonyl)phenyl)- 1H-pyrazolo[3,4- d]pyrimidin-6-amine |
| 10 | 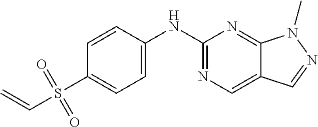 [SMILES: C=CS(=O)(=O)c1ccc(Nc2ncc3cnn(C)c3n2)cc1] | 1-methyl-N-(4- (vinylsulfonyl)phenyl)-1H- pyrazolo[3,4-d]pyrimidin-6- amine |
| 11 | 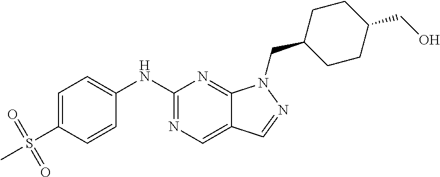 [SMILES: CS(=O)(=O)c1ccc(Nc2ncc3cnn(C[C@H]4CC[C@H](CO)CC4)c3n2)cc1] | ((1r,4r)-4-((6-((4- (methylsulfonyl)phenyl) amino)-1H-pyrazolo[3,4- d]pyrimidin-1- yl)methyl)cyclohexyl) methanol |
| 12 | 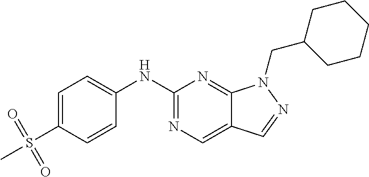 [SMILES: CS(=O)(=O)c1ccc(Nc2ncc3cnn(CC4CCCCC4)c3n2)cc1] | 1-(cyclohexylmethyl)-N-(4- (methylsulfonyl)phenyl)-1H- pyrazolo[3,4-d]pyrimidin-6- amine |
| 13 | 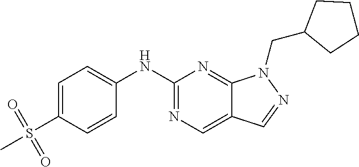 [SMILES: CS(=O)(=O)c1ccc(Nc2ncc3cnn(CC4CCCC4)c3n2)cc1] | 1-(cyclopentylmethyl)-N-(4- (methylsulfonyl)phenyl)-1H- pyrazolo[3,4-d]pyrimidin-6- amine |
| 14 | 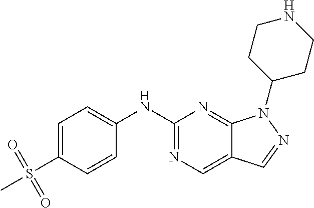 [SMILES: CS(=O)(=O)c1ccc(Nc2ncc3cnn(C4CCNCC4)c3n2)cc1] | N-(4- (methylsulfonyl)phenyl)-1- (piperidin-4-yl)-1H- pyrazolo[3,4-d]pyrimidin-6- amine |
| 15 | 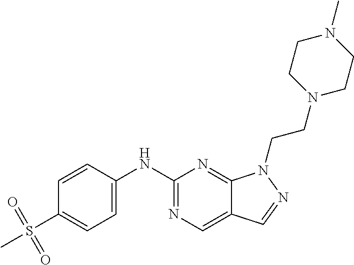 [SMILES: CN1CCN(CCn2ncc3cnc(Nc4ccc(S(C)(=O)=O)cc4)nc32)CC1] | 1-(2-(4-methylpiperazin-1- yl)ethyl)-N-(4- (methylsulfonyl)phenyl)-1H- pyrazolo[3,4-d]pyrimidin-6- amine |
| 16 | 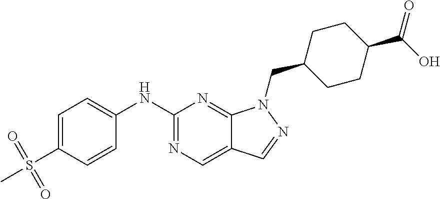 [SMILES: CS(=O)(=O)c1ccc(Nc2ncc3cnn(C[C@H]4CC[C@@H](C(=O)O)CC4)c3n2)cc1] | (1S,4S)-4-((6-((4- (methylsulfonyl)phenyl) amino)-1H-pyrazolo[3,4- d]pyrimidin-1- yl)methyl)cyclohexane-1- carboxylic acid |
| 17 | 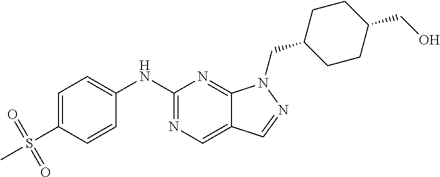 [SMILES: CS(=O)(=O)c1ccc(Nc2ncc3cnn(C[C@H]4CC[C@@H](CO)CC4)c3n2)cc1] | ((1S,4S)-4-((6-((4- (methylsulfonyl)phenyl) amino)-1H-pyrazolo[3,4- d]pyrimidin-1- yl)methyl)cyclohexyl) methanol |
| 18 | 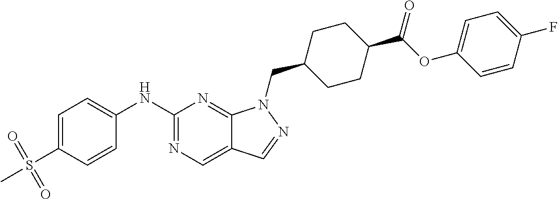 [SMILES: CS(=O)(=O)c1ccc(Nc2ncc3cnn(C[C@H]4CC[C@@H](C(=O)Oc5ccc(F)cc5)CC4)c3n2)cc1] | 4-fluorophenyl (1S,4S)-4- ((6-((4- (methylsulfonyl)phenyl) amino)-1H-pyrazolo[3,4- d]pyrimidin-1- yl)methyl)cyclohexane-1- carboxylate |
| 19 | 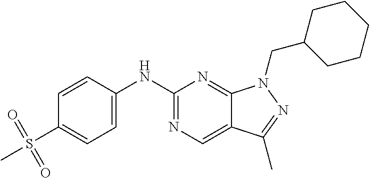 [SMILES: Cc1nn(CC2CCCCC2)c2nc(Nc3ccc(S(C)(=O)=O)cc3)ncc12] | 1-(cyclohexylmethyl)-3- methyl-N-(4- (methylsulfonyl)phenyl)-1H- pyrazolo[3,4-d]pyrimidin-6- amine |
| 20 | 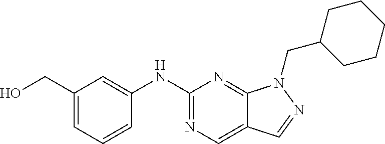 [SMILES: OCc1cccc(Nc2ncc3cnn(CC4CCCCC4)c3n2)c1] | (3-((1-(cyclohexylmethyl)- 1H-pyrazolo[3,4- d]pyrimidin-6- yl)amino)phenyl)methanol |
| 21 | 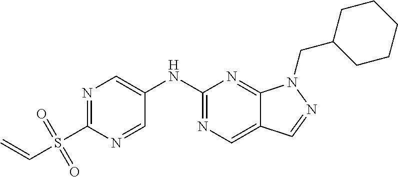 [SMILES: C=CS(=O)(=O)c1ncc(Nc2ncc3cnn(CC4CCCCC4)c3n2)cn1] | 1-(cyclohexylmethyl)-N-(2- (vinylsulfonyl)pyrimidin-5- yl)-1H-pyrazolo[3,4- d]pyrimidin-6-amine |
| 22 | 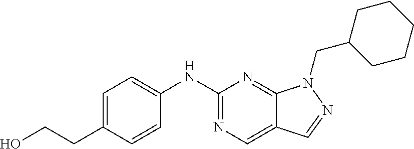 [SMILES: OCCc1ccc(Nc2ncc3cnn(CC4CCCCC4)c3n2)cc1] | 2-(4-((1-(cyclohexylmethyl)- 1H-pyrazolo[3,4- d]pyrimidin-6- yl)amino)phenyl)ethan-1-ol |
| 23 | 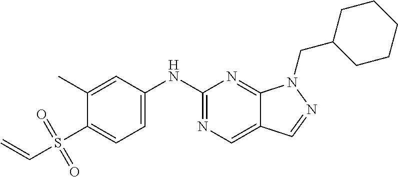 [SMILES: C=CS(=O)(=O)c1ccc(Nc2ncc3cnn(CC4CCCCC4)c3n2)cc1C] | 1-(cyclohexylmethyl)-N-(3- methyl-4- (vinylsulfonyl)phenyl)-1H- pyrazolo[3,4-d]pyrimidin-6- amine |
| 24 | 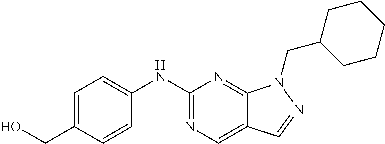 [SMILES: OCc1ccc(Nc2ncc3cnn(CC4CCCCC4)c3n2)cc1] | (4-((1-(cyclohexylmethyl)- 1H-pyrazolo[3,4- d]pyrimidin-6- yl)amino)phenyl)methanol |
| 25 | 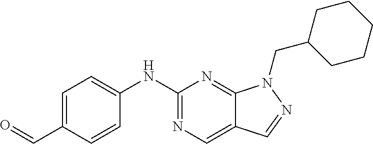 [SMILES: O=Cc1ccc(Nc2ncc3cnn(CC4CCCCC4)c3n2)cc1] | 4-((1-(cyclohexylmethyl)- 1H-pyrazolo[3,4- d]pyrimidin-6- yl)amino)benzaldehyde |
| 26 | 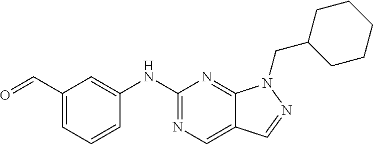 [SMILES: O=Cc1cccc(Nc2ncc3cnn(CC4CCCCC4)c3n2)c1] | 3-((1-(cyclohexylmethyl)- 1H-pyrazolo[3,4- d]pyrimidin-6- yl)amino)benzaldehyde |
| 27 | 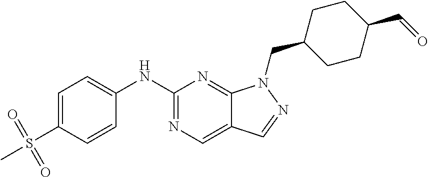 [SMILES: CS(=O)(=O)c1ccc(Nc2ncc3cnn(C[C@H]4CC[C@@H](C=O)CC4)c3n2)cc1] | (1s,4s)-4-((6-((4- (methylsulfonyl)phenyl) amino)-1H-pyrazolo[3,4- d]pyrimidin-1- yl)methyl)cyclohexane-1- carbaldehyde |
| 28 | 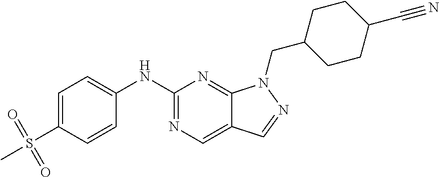 [SMILES: CS(=O)(=O)c1ccc(Nc2ncc3cnn(CC4CCC(C#N)CC4)c3n2)cc1] | 4-((6-((4- (methylsulfonyl)phenyl) amino)-1H-pyrazolo[3,4- d]pyrimidin-1- yl)methyl)cyclohexane-1- carbonitrile (stereoisomer A) |
| 29 | 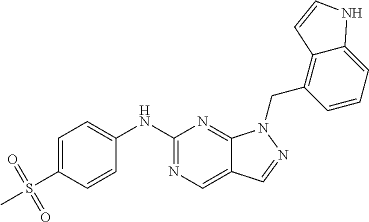 [SMILES: CS(=O)(=O)c1ccc(Nc2ncc3cnn(Cc4cccc5[nH]ccc45)c3n2)cc1] | 1-((1H-indol-4-yl)methyl)- N-(4- (methylsulfonyl)phenyl)-1H- pyrazolo[3,4-d]pyrimidin-6- amine |
| 30 | 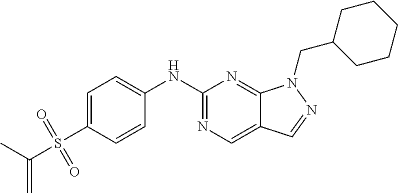 [SMILES: C=C(C)S(=O)(=O)c1ccc(Nc2ncc3cnn(CC4CCCCC4)c3n2)cc1] | 1-(cyclohexylmethyl)-N-(4- (prop-1-en-2- ylsulfonyl)phenyl)-1H- pyrazolo[3,4-d]pyrimidin-6- amine |
| 31 | 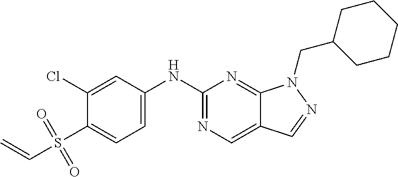 [SMILES: C=CS(=O)(=O)c1ccc(Nc2ncc3cnn(CC4CCCCC4)c3n2)cc1Cl] | N-(5-chloro-6- (vinylsulfonyl)pyridin-3-yl)- 1-(cyclohexylmethyl)-1H- pyrazolo[3,4-d]pyrimidin-6- amine |
| 32 | 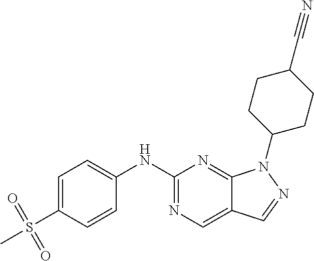 [SMILES: CS(=O)(=O)c1ccc(Nc2ncc3cnn(C4CCC(C#N)CC4)c3n2)cc1] | 4-(6-((4- (methylsulfonyl)phenyl) amino)-1H-pyrazolo[3,4- d]pyrimidin-1- yl)cyclohexane-1- carbonitrile (stereoisomer A) |
| 33 | 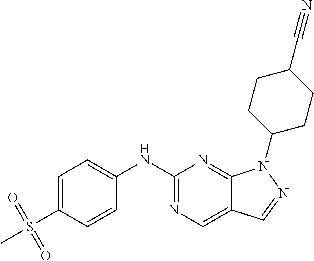 [SMILES: CS(=O)(=O)c1ccc(Nc2ncc3cnn(C4CCC(C#N)CC4)c3n2)cc1] | 4-(6-((4- (methylsulfonyl)phenyl) amino)-1H-pyrazolo[3,4- d]pyrimidin-1- yl)cyclohexane-1- carbonitrile (stereoisomer B) |
| 34 | 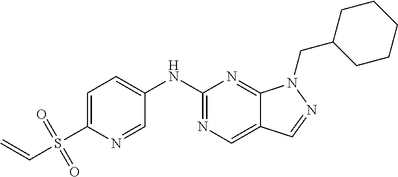 [SMILES: C=CS(=O)(=O)c1ccc(Nc2ncc3cnn(CC4CCCCC4)c3n2)cn1] | 1-(cyclohexylmethyl)-N-(6- (vinylsulfonyl)pyridin-3-yl)- 1H-pyrazolo[3,4- d]pyrimidin-6-amine |
| 35 | 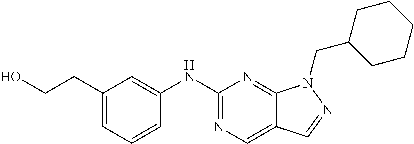 [SMILES: OCCc1cccc(Nc2ncc3cnn(CC4CCCCC4)c3n2)c1] | 2-(3-((1-(cyclohexylmethyl)- 1H-pyrazolo[3,4- d]pyrimidin-6- yl)amino)phenyl)ethan-1-ol |
| 36 | 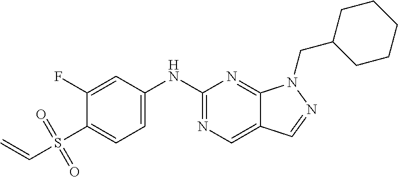 [SMILES: C=CS(=O)(=O)c1ccc(Nc2ncc3cnn(CC4CCCCC4)c3n2)cc1F] | 1-(cyclohexylmethyl)-N-(3- fluoro-4- (vinylsulfonyl)phenyl)-1H- pyrazolo[3,4-d]pyrimidin-6- amine |
| 37 | 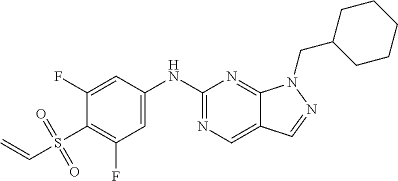 [SMILES: C=CS(=O)(=O)c1c(F)cc(Nc2ncc3cnn(CC4CCCCC4)c3n2)cc1F] | 1-(cyclohexylmethyl)-N- (3,5-difluoro-4- (vinylsulfonyl)phenyl)-1H- pyrazolo[3,4-d]pyrimidin-6- amine |
| 38 | 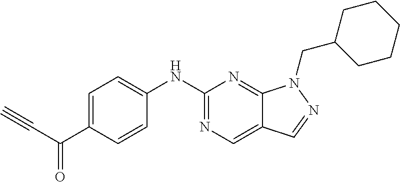 [SMILES: C#CC(=O)c1ccc(Nc2ncc3cnn(CC4CCCCC4)c3n2)cc1] | 1-(4-((1-(cyclohexylmethyl)- 1H-pyrazolo[3,4- d]pyrimidin-6- yl)amino)phenyl)prop-2-yn- 1-one |
| 39 | 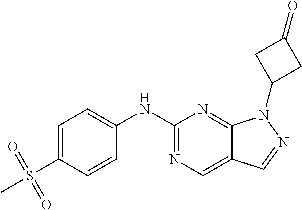 [SMILES: CS(=O)(=O)c1ccc(Nc2ncc3cnn(C4CC(=O)C4)c3n2)cc1] | 3-(6-((4- (methylsulfonyl)phenyl) amino)-1H-pyrazolo[3,4- d]pyrimidin-1- yl)cyclobutan-1-one |
| 40 | 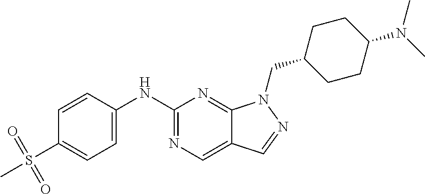 | 1-(((1S,4S)-4- (dimethylamino)cyclohexyl) methyl)-N-(4- (methylsulfonyl)phenyl)-1H- pyrazolo[3,4-d]pyrimidin-6- amine |
| 41 | 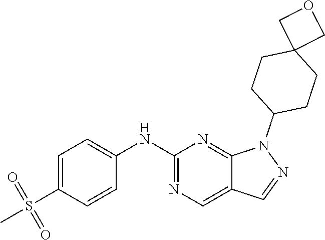 [SMILES: CS(=O)(=O)c1ccc(Nc2ncc3cnn(C4CCC5(CC4)COC5)c3n2)cc1] | N-(4- (methylsulfonyl)phenyl)-1- (2-oxaspiro[3.5]nonan-7-yl)- 1H-pyrazolo[3,4- d]pyrimidin-6-amine |
| 42 | 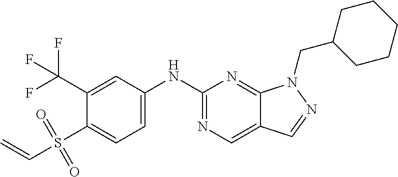 [SMILES: C=CS(=O)(=O)c1ccc(Nc2ncc3cnn(CC4CCCCC4)c3n2)cc1C(F)(F)F] | 1-(cyclohexylmethyl)-N-(3- (trifluoromethyl)-4- (vinylsulfonyl)phenyl)-1H- pyrazolo[3,4-d]pyrimidin-6- amine |
| 43 | 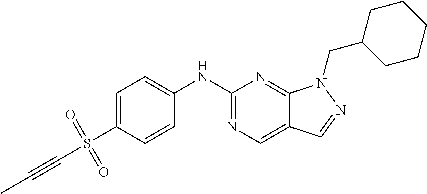 [SMILES: CC#CS(=O)(=O)c1ccc(Nc2ncc3cnn(CC4CCCCC4)c3n2)cc1] | 1-(cyclohexylmethyl)-N-(4- (prop-1-yn-1- ylsulfonyl)phenyl)-1H- pyrazolo[3,4-d]pyrimidin-6- amine |
| 44 | 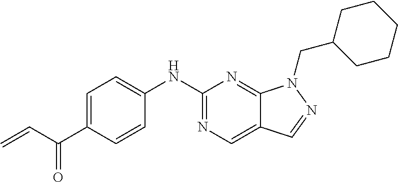 [SMILES: C=CC(=O)c1ccc(Nc2ncc3cnn(CC4CCCCC4)c3n2)cc1] | 1-(4-((1-(cyclohexylmethyl)- 1H-pyrazolo[3,4- d]pyrimidin-6- yl)amino)phenyl)prop-2-en- 1-one |
| 45 | 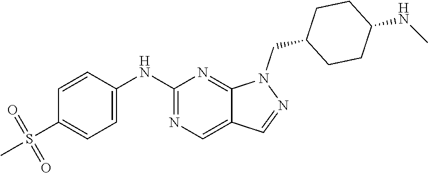 | 1-(((1s,4s)-4- (methylamino)cyclohexyl) methyl)-N-(4- (methylsulfonyl)phenyl)-1H- pyrazolo[3,4-d]pyrimidin-6- amine |
| 46 | 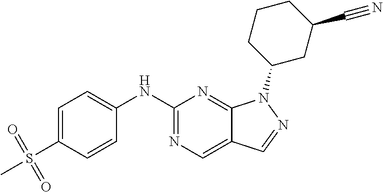 [SMILES: CS(=O)(=O)c1ccc(Nc2ncc3cnn([C@@H]4CCC[C@@H](C#N)C4)c3n2)cc1] | (1R,3R)-3-(6-((4- (methylsulfonyl)phenyl) amino)-1H-pyrazolo[3,4- d]pyrimidin-1- yl)cyclohexane-1- carbonitrile |
| 47 | 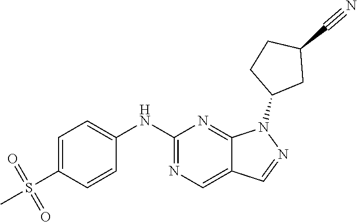 [SMILES: CS(=O)(=O)c1ccc(Nc2ncc3cnn([C@@H]4CC[C@@H](C#N)C4)c3n2)cc1] | (1R,3R)-3-(6-((4- (methylsulfonyl)phenyl) amino)-1H-pyrazolo[3,4- d]pyrimidin-1- yl)cyclopentane-1- carbonitrile |
| 48 | 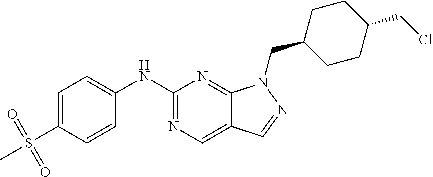 [SMILES: CS(=O)(=O)c1ccc(Nc2ncc3cnn(C[C@H]4CC[C@H](CCl)CC4)c3n2)cc1] | 1-(((1R,4R)-4- (chloromethyl)cyclohexyl) methyl)-N-(4- (methylsulfonyl)phenyl)-1H- pyrazolo[3,4-d]pyrimidin-6- amine |
| 49 | 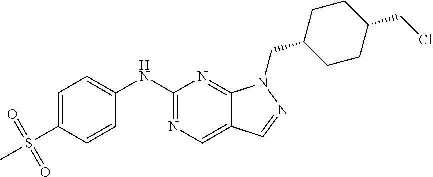 [SMILES: CS(=O)(=O)c1ccc(Nc2ncc3cnn(C[C@H]4CC[C@@H](CCl)CC4)c3n2)cc1] | 1-(((1S,4S)-4- (chloromethyl)cyclohexyl) methyl)-N-(4- (methylsulfonyl)phenyl)-1H- pyrazolo[3,4-d]pyrimidin-6- amine |
| 50 | 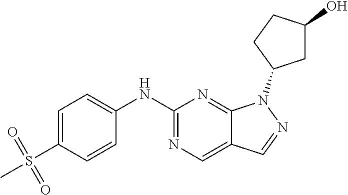 [SMILES: CS(=O)(=O)c1ccc(Nc2ncc3cnn([C@@H]4CC[C@@H](O)C4)c3n2)cc1] | (1R,3R)-3-(6-((4- (methylsulfonyl)phenyl) amino)-1H-pyrazolo[3,4- d]pyrimidin-1- yl)cyclopentan-1-ol |
| 51 | 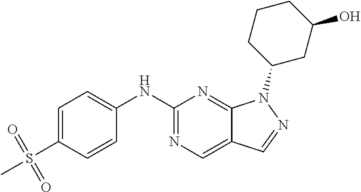 [SMILES: CS(=O)(=O)c1ccc(Nc2ncc3cnn([C@@H]4CCC[C@@H](O)C4)c3n2)cc1] | (1R,3R)-3-(6-((4- (methylsulfonyl)phenyl)ami no)-1H-pyrazolo[3,4- d]pyrimidin-1- yl)cyclohexan-1-ol |
| 52 | 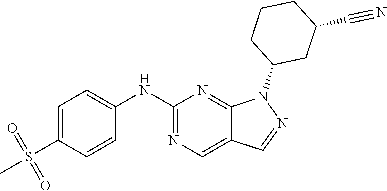 [SMILES: CS(=O)(=O)c1ccc(Nc2ncc3cnn([C@@H]4CCC[C@H](C#N)C4)c3n2)cc1] | (1S,3R)-3-(6-((4- (methylsulfonyl)phenyl) amino)-1H-pyrazolo[3,4- d]pyrimidin-1- yl)cyclohexane-1- carbonitrile |
| 53 | 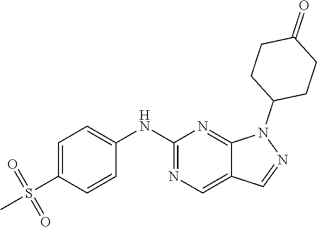 [SMILES: CS(=O)(=O)c1ccc(Nc2ncc3cnn(C4CCC(=O)CC4)c3n2)cc1] | 4-(6-((4- (methylsulfonyl)phenyl) amino)-1H-pyrazolo[3,4- d]pyrimidin-1- yl)cyclohexan-1-one |
| 54 | 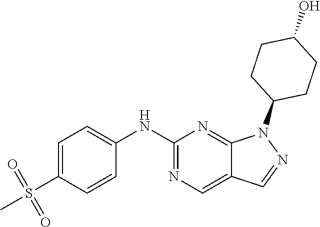 [SMILES: CS(=O)(=O)c1ccc(Nc2ncc3cnn([C@H]4CC[C@H](O)CC4)c3n2)cc1] | (1R,4R)-4-(6-((4- (methylsulfonyl)phenyl)ami no)-1H-pyrazolo[3,4- d]pyrimidin-1- yl)cyclohexan-1-ol |
| 55 | 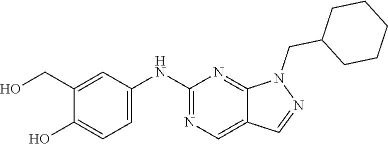 [SMILES: OCc1cc(Nc2ncc3cnn(CC4CCCCC4)c3n2)ccc1O] | 4-((1-(cyclohexylmethyl)- 1H-pyrazolo[3,4- d]pyrimidin-6-yl)amino)-2- (hydroxymethyl)phenol |
| 56 | 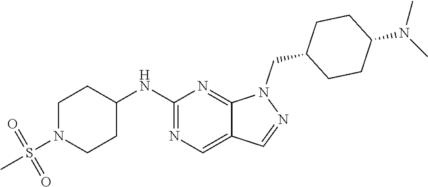 | 1-(((1S,4S)-4- (dimethylamino)cyclohexyl) methyl)-N-(1- (methylsulfonyl)piperidin-4- yl)-1H-pyrazolo[3,4- d]pyrimidin-6-amine |
| 57 | 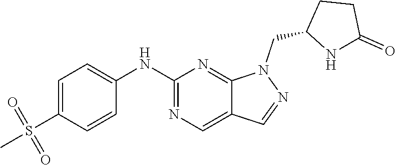 [SMILES: CS(=O)(=O)c1ccc(Nc2ncc3cnn(C[C@@H]4CCC(=O)N4)c3n2)cc1] | (S)-5-((6-((4- (methylsulfonyl)phenyl) amino)-1H-pyrazolo[3,4- d]pyrimidin-1- yl)methyl)pyrrolidin-2-one |
| 58 | 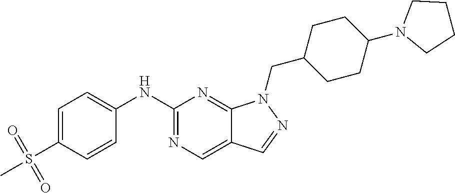 [SMILES: CS(=O)(=O)c1ccc(Nc2ncc3cnn(CC4CCC(N5CCCC5)CC4)c3n2)cc1] | N-(4- (methylsulfonyl)phenyl)-1- ((4-(pyrrolidin-1- yl)cyclohexyl)methyl)-1H- pyrazolo[3,4-d]pyrimidin-6- amine (stereoisomer A) |
| 59 | 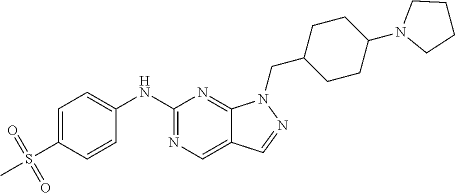 [SMILES: CS(=O)(=O)c1ccc(Nc2ncc3cnn(CC4CCC(N5CCCC5)CC4)c3n2)cc1] | N-(4- (methylsulfonyl)phenyl)-1- ((4-(pyrrolidin-1- yl)cyclohexyl)methyl)-1H- pyrazolo[3,4-d]pyrimidin-6- amine (stereoisomer B) |
| 60 | 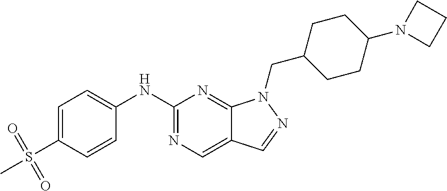 [SMILES: CS(=O)(=O)c1ccc(Nc2ncc3cnn(CC4CCC(N5CCC5)CC4)c3n2)cc1] | 1-((4-(azetidin-1- yl)cyclohexyl)methyl)-N-(4- (methylsulfonyl)phenyl)-1H- pyrazolo[3,4-d]pyrimidin-6- amine (stereoisomer A) |
| 61 | 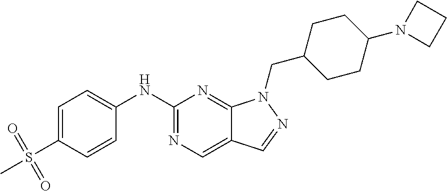 [SMILES: CS(=O)(=O)c1ccc(Nc2ncc3cnn(CC4CCC(N5CCC5)CC4)c3n2)cc1] | 1-((4-(azetidin-1- yl)cyclohexyl)methyl)-N-(4- (methylsulfonyl)phenyl)-1H- pyrazolo[3,4-d]pyrimidin-6- amine (stereoisomer B) |
| 62 | 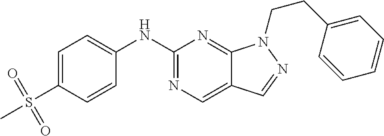 [SMILES: CS(=O)(=O)c1ccc(Nc2ncc3cnn(CCc4ccccc4)c3n2)cc1] | N-(4- (methylsulfonyl)phenyl)-1- phenethyl-1H-pyrazolo[3,4- d]pyrimidin-6-amine |
| 63 | 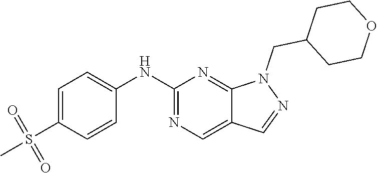 [SMILES: CS(=O)(=O)c1ccc(Nc2ncc3cnn(CC4CCOCC4)c3n2)cc1] | N-(4- (methylsulfonyl)phenyl)-1- ((tetrahydro-2H-pyran-4- yl)methyl)-1H-pyrazolo[3,4- d]pyrimidin-6-amine |
| 64 | 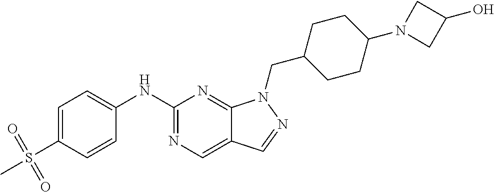 [SMILES: CS(=O)(=O)c1ccc(Nc2ncc3cnn(CC4CCC(N5CC(O)C5)CC4)c3n2)cc1] | 1-(4-((6-((4- (methylsulfonyl)phenyl) amino)-1H-pyrazolo[3,4- d]pyrimidin-1- yl)methyl)cyclohexyl) azetidin-3-ol (stereoisomer A) |
| 65 | 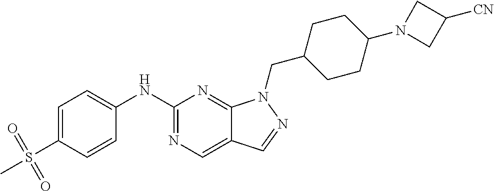 [SMILES: CS(=O)(=O)c1ccc(Nc2ncc3cnn(CC4CCC(N5CC(C#N)C5)CC4)c3n2)cc1] | 1-(4-((6-((4- (methylsulfonyl)phenyl) amino)-1H-pyrazolo[3,4- d]pyrimidin-1- yl)methyl)cyclohexyl) azetidine-3-carbonitrile (stereoisomer A) |
| 66 | 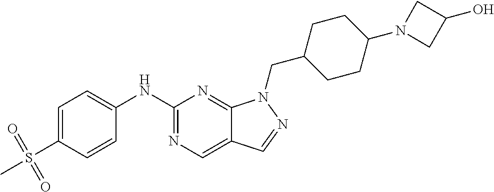 [SMILES: CS(=O)(=O)c1ccc(Nc2ncc3cnn(CC4CCC(N5CC(O)C5)CC4)c3n2)cc1] | 1-(4-((6-((4- (methylsulfonyl)phenyl) amino)-1H-pyrazolo[3,4- d]pyrimidin-1- yl)methyl)cyclohexyl) azetidin-3-ol (stereoisomer B) |
| 67 | 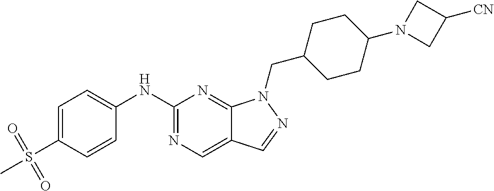 [SMILES: CS(=O)(=O)c1ccc(Nc2ncc3cnn(CC4CCC(N5CC(C#N)C5)CC4)c3n2)cc1] | 1-(4-((6-((4- (methylsulfonyl)phenyl) amino)-1H-pyrazolo[3,4- d]pyrimidin-1- yl)methyl)cyclohexyl) azetidine-3-carbonitrile (stereoisomer B) |
| 68 | 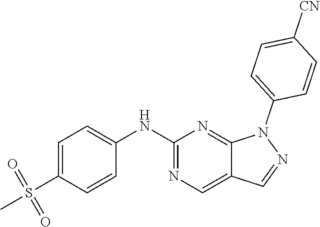 [SMILES: CS(=O)(=O)c1ccc(Nc2ncc3cnn(-c4ccc(C#N)cc4)c3n2)cc1] | 4-(6-((4- (methylsulfonyl)phenyl) amino)-1H-pyrazolo[3,4- d]pyrimidin-1- yl)benzonitrile |
| 69 | 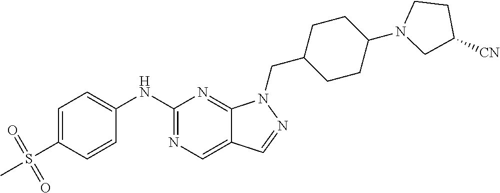 [SMILES: CS(=O)(=O)c1ccc(Nc2ncc3cnn(CC4CCC(N5CC[C@H](C#N)C5)CC4)c3n2)cc1] | (S)-1-(4-((6-((4- (methylsulfonyl)phenyl) amino)-1H-pyrazolo[3,4- d]pyrimidin-1- yl)methyl)cyclohexyl) pyrrolidine-3-carbonitrile (stereoisomer A) |
| 70 | 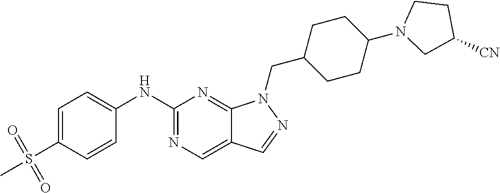 [SMILES: CS(=O)(=O)c1ccc(Nc2ncc3cnn(CC4CCC(N5CC[C@H](C#N)C5)CC4)c3n2)cc1] | (S)-1-(4-((6-((4- (methylsulfonyl)phenyl) amino)-1H-pyrazolo[3,4- d]pyrimidin-1- yl)methyl)cyclohexyl) pyrrolidine-3-carbonitrile (stereoisomer B) |
| 71 | 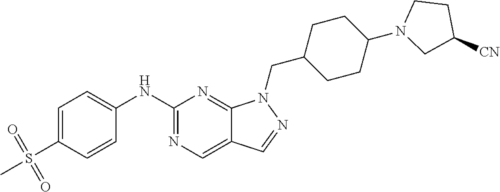 [SMILES: CS(=O)(=O)c1ccc(Nc2ncc3cnn(CC4CCC(N5CC[C@@H](C#N)C5)CC4)c3n2)cc1] | (R)-1-(4-((6-((4- (methylsulfonyl)phenyl)ami no)-1H-pyrazolo[3,4- d]pyrimidin-1- yl)methyl)cyclohexyl)pyrroli dine-3-carbonitrile (stereoisomer A) |
| 72 | 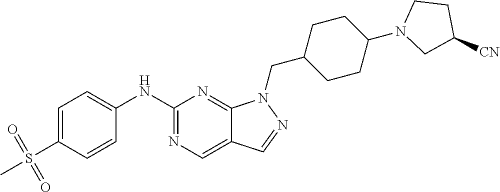 [SMILES: CS(=O)(=O)c1ccc(Nc2ncc3cnn(CC4CCC(N5CC[C@@H](C#N)C5)CC4)c3n2)cc1] | (R)-1-(4-((6-((4- (methylsulfonyl)phenyl) amino)-1H-pyrazolo[3,4- d]pyrimidin-1- yl)methyl)cyclohexyl) pyrrolidine-3-carbonitrile (stereoisomer B) |
| 73 | 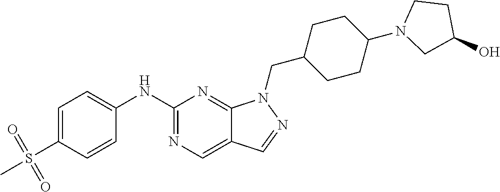 [SMILES: CS(=O)(=O)c1ccc(Nc2ncc3cnn(CC4CCC(N5CC[C@@H](O)C5)CC4)c3n2)cc1] | (R)-1-(4-((6-((4- (methylsulfonyl)phenyl) amino)-1H-pyrazolo[3,4- d]pyrimidin-1- yl)methyl)cyclohexyl) pyrrolidin-3-ol (stereoisomer A) |
| 74 | 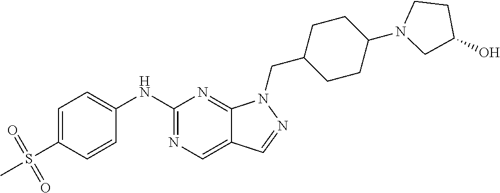 [SMILES: CS(=O)(=O)c1ccc(Nc2ncc3cnn(CC4CCC(N5CC[C@H](O)C5)CC4)c3n2)cc1] | (S)-1-(4-((6-((4- (methylsulfonyl)phenyl) amino)-1H-pyrazolo[3,4- d]pyrimidin-1- yl)methyl)cyclohexyl) pyrrolidin-3-ol (stereoisomer A) |
| 75 | 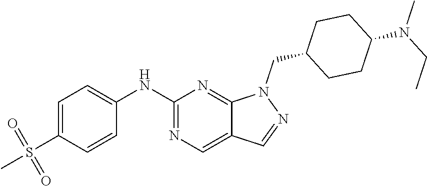 [SMILES: CCN(C)[C@H]1CC[C@@H](Cn2ncc3cnc(Nc4ccc(S(C)(=O)=O)cc4)nc32)CC1] | 1-(((1s,4s)-4- (ethyl(methyl)amino) cyclohexyl)methyl)-N-(4- (methylsulfonyl)phenyl)-1H- pyrazolo[3,4-d]pyrimidin-6- amine |
| 76 | 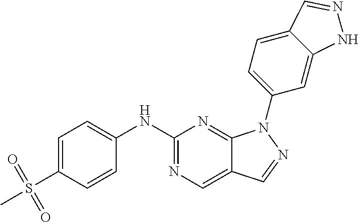 [SMILES: CS(=O)(=O)c1ccc(Nc2ncc3cnn(-c4ccc5cn[nH]c5c4)c3n2)cc1] | 1-(1H-indazol-6-yl)-N-(4- (methylsulfonyl)phenyl)-1H- pyrazolo[3,4-d]pyrimidin-6- amine |
| 77 | 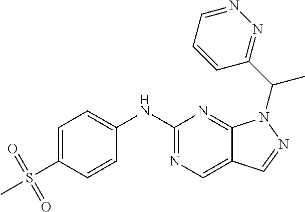 [SMILES: CC(c1cccnn1)n1ncc2cnc(Nc3ccc(S(C)(=O)=O)cc3)nc21] | N-(4- (methylsulfonyl)phenyl)-1- (1-(pyridazin-3-yl)ethyl)- 1H-pyrazolo[3,4- d]pyrimidin-6-amine |
| 78 | 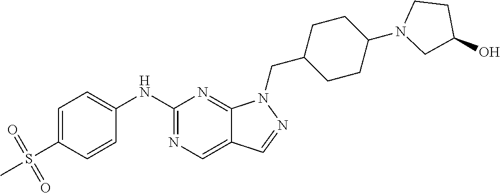 [SMILES: CS(=O)(=O)c1ccc(Nc2ncc3cnn(CC4CCC(N5CC[C@@H](O)C5)CC4)c3n2)cc1] | (R)-1-(4-((6-((4- (methylsulfonyl)phenyl) amino)-1H-pyrazolo[3,4- d]pyrimidin-1- yl)methyl)cyclohexyl) pyrrolidin-3-ol (stereoisomer B) |
| 79 | 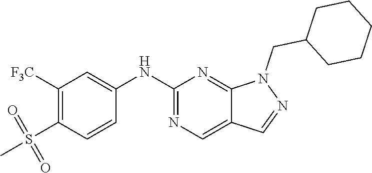 [SMILES: CS(=O)(=O)c1ccc(Nc2ncc3cnn(CC4CCCCC4)c3n2)cc1C(F)(F)F] | 1-(cyclohexylmethyl)-N-(4- (methylsulfonyl)-3- (trifluoromethyl)phenyl)-1H- pyrazolo[3,4-d]pyrimidin-6- amine |
| 80 | 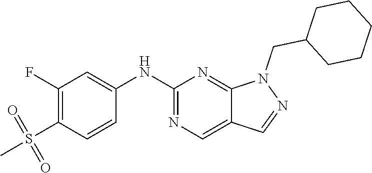 [SMILES: CS(=O)(=O)c1ccc(Nc2ncc3cnn(CC4CCCCC4)c3n2)cc1F] | 1-(cyclohexylmethyl)-N-(3- fluoro-4- (methylsulfonyl)phenyl)-1H- pyrazolo[3,4-d]pyrimidin-6- amine |
| 81 | 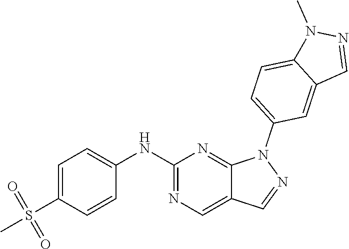 [SMILES: Cn1ncc2cc(-n3ncc4cnc(Nc5ccc(S(C)(=O)=O)cc5)nc43)ccc21] | 1-(1-methyl-1H-indazol-5- yl)-N-(4- (methylsulfonyl)phenyl)-1H- pyrazolo[3,4-d]pyrimidin-6- amine |
| 82 | 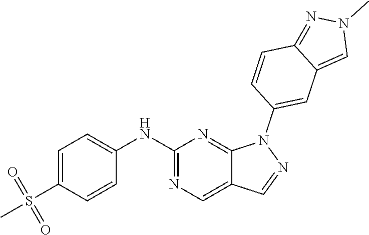 | 1-(2-methyl-2H-indazol-5- yl)-N-(4- (methylsulfonyl)phenyl)-1H- pyrazolo[3,4-d]pyrimidin-6- amine |
| 83 | 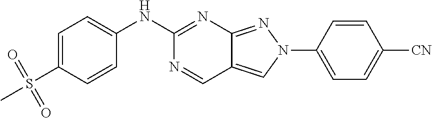 [SMILES: CS(=O)(=O)c1ccc(Nc2ncc3cn(-c4ccc(C#N)cc4)nc3n2)cc1] | 4-(6-((4- (methylsulfonyl)phenyl) amino)-2H-pyrazolo[3,4- d]pyrimidin-2- yl)benzonitrile |
| 84 | 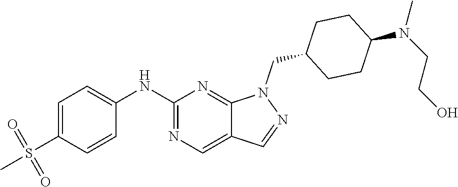 [SMILES: CN(CCO)[C@H]1CC[C@H](Cn2ncc3cnc(Nc4ccc(S(C)(=O)=O)cc4)nc32)CC1] | 2-(methyl((1r,4r)-4-((6-((4- (methylsulfonyl)phenyl) amino)-1H-pyrazolo[3,4- d]pyrimidin-1- yl)methyl)cyclohexyl) amino)ethan-1-ol |

### CDK2 Assay

A CDK homogeneous time-resolved fluorescence (HTRF) assay was used to assess the potency of the compounds described herein.

To an untreated 384-well microtiter plate (ProxiPlate) was added 100 nL 100× test compound in DMSO and 5 μL 2×CDK enzyme in assay buffer (50 mM HEPES, 1 mM EGTA, 10 mM MgCl₂, 0.01% Tween 20, 2 mM DTT). The plate was centrifuged at 800 RPM for 1 min then incubated for 20 min at 23° C. 2.5 μL per well of 4×ATP and 2.5 μL 4×ULight™-eIF4E-binding protein 1 (Thr37/46) Peptide) substrate were added to all wells. After addition, the plates were centrifuged at 800 RPM for 1 min. The plates were incubated at 23° C. for 60 min. 5 μL per well 3×EDTA solution were added to each well to stop the reaction. 2.5 μL 7×LANCE Ultra Europium-anti-phospho-eTF4E-binding protein 1 (Thr37/46 antibody) were added to each well and the plate was centrifuged for 1 min at 800 RPM. The plate was incubated for 60 min at 23° C. Fluorescence was read at 340 nm excitation and 665 nm emission on an EnVision 2105 fluorescence reader. The final concentrations of reagents in the activity assay were 3 nM CDK2/CCNE1, 1 mM ATP, 50 nM ULight™-eIF4E-binding protein 1 (Thr37/46) Peptide substrate, 1% DMSO, 10 mM EDTA, and 2 nM LANCE Ultra Europium-anti-phospho-eTF4E-binding protein 1 (Thr37/46).

Dose-response titrations were conducted by using an 11-point, three-fold serial dilution scheme typically starting at 1 μM. Signals were reported as fluorescence units at 665 nm or the ratio of fluorescence units at 665 nm/fluorescence units at 615 nm. Signals are referred to below as “Lance signals”. Compound potencies were determined by first calculating % inhibition at each compound concentration according to the following equation: % inhibition=(Lance signal DMSO control−Lance signal value of sample)*100/Lance DMSO control−Lance signal value of background), where Lance signal DMSO control represents an average of 2-16 replicates, Lance signal value of sample represents an average of 2 replicates, and Lance signal of background represents an average of 2-16 replicates.

Using this assay, the IC₅₀ values of example compounds were determined.

TABLE 10
illustrates the IC₅₀ for various exemplary compounds of Formula I.

| | CDK2/CCNE IC₅₀ | | | | CDK2/CCNE IC₅₀ | | |
|---|---|---|---|---|---|---|---|
| | <25 | 25-150 | >150 | | <25 | 25-150 | >150 |
| Cmpd | nM | nM | nM | Cmpd | nM | nM | nM |
| 1 | X | | | 43 | | | X |
| 2 | X | | | 44 | | | X |
| 3 | | X | | 45 | X | | |
| 4 | | | X | 46 | X | | |
| 5 | | | X | 47 | | | X |
| 6 | | X | | 48 | | | X |
| 7 | | | X | 49 | X | | |
| 8 | | X | | 50 | | X | |
| 9 | | | X | 51 | X | | |
| 10 | | | X | 52 | | X | |
| 11 | | X | | 53 | | X | |
| 12 | | X | | 54 | | X | |
| 13 | | X | | 55 | | X | |
| 14 | | X | | 56 | | X | |
| 15 | | | X | 57 | | | X |
| 16 | | | X | 58 | | X | |
| 17 | | X | | 59 | | | X |
| 18 | | | X | 60 | | | X |
| 19 | | | X | 61 | | | X |
| 20 | X | | | 62 | | | X |
| 21 | | X | | 63 | | | X |
| 22 | | X | | 64 | | | X |
| 23 | | X | | 65 | | | X |
| 24 | | X | | 66 | | | X |
| 25 | | X | | 67 | | | X |
| 26 | X | | | 68 | | X | |
| 27 | | X | | 69 | | | X |
| 28 | | X | | 70 | | | X |
| 29 | | X | | 71 | | | X |
| 30 | | X | | 72 | | | X |
| 31 | | | X | 73 | | | X |
| 32 | X | | | 74 | | | X |
| 33 | X | | | 75 | | X | |
| 34 | X | | | 76 | | X | |
| 35 | X | | | 77 | | | X |
| 36 | | X | | 78 | | | X |
| 37 | | X | | 79 | | | X |
| 38 | | | X | 80 | | | X |
| 39 | | | X | 81 | | X | |
| 40 | | X | | 82 | | | X |
| 41 | | X | | 83 | | X | |
| 42 | | X | | 84 | | | X |

### CDK1 Assay

A CDK homogeneous time-resolved fluorescence (HTRF) assay is used to assess the potency of the compounds described herein.

To an untreated 384-well microtiter plate (ProxiPlate) is added 100 nL 100×test compound in DMSO and 5 μL 2×CDK enzyme in assay buffer (50 mM HEPES, 1 mM EGTA, 10 mM MgCl₂, 0.01% Tween 20, 2 mM DTT). The plate is centrifuged at 800 RPM for 1 min then incubated for 20 min at 23° C. 2.5 μL per well of 4×ATP and 2.5 μL 4×ULight™-eIF4E-binding protein 1 (Thr37/46) Peptide substrate are added to all wells. After addition, the plates are centrifuged at 800 RPM for 1 min. The plates are incubated at 23° C. for 60 min. 5 μL per well 4×EDTA solution are added to each well to stop the reaction. 5 μL 4×LANCE Ultra Europium-anti-phospho-eIF4E-binding protein 1 (Thr37/46) antibody are added to each well and the plate is centrifuged for 1 min at 800 RPM. The plate is incubated for 60 min at 23° C. Fluorescence is read at 340 nm excitation and 665 nm emission on an EnVision 2105 fluorescence reader. The final concentrations of reagents in the activity assay is 0.5 nM CDK1/CCNB1, 1 mM ATP, 50 nM ULight™-eIF4E-binding protein 1 (Thr37/46) Peptide substrate, 1% DMSO, 10 mM EDTA, and 2 nM LANCE Ultra Europium-anti-phospho-eIF4E-binding protein 1 (Thr37/46).

Dose-response titrations are conducted by using an 11-point, half-logarithmic serial dilution scheme typically starting at 10 μM. Signals are reported as fluorescence units at 665 nm or the ratio of fluorescence units at 665 nm/fluorescence units at 615 nm. Signals are referred to as “Lance signals”. Compound potencies are determined by first calculating % inhibition at each compound concentration according to the following equation: % inhibition=(Lance signal DMSO control−Lance signal value of sample)*100/Lance DMSO control−Lance signal value of background), where Lance signal DMSO control represents an average of 8-16 replicates, Lance signal value of sample represents an average of 2 replicates, and Lance signal of background represents an average of 8-16 replicates.

### CDK4 Assay

A CDK homogeneous time-resolved fluorescence (HTRF) assay is used to assess the potency of the compounds described herein.

To an untreated 384-well microtiter plate (ProxiPlate) is added 100 nL 100×test compound in DMSO and 5 μL 2×CDK enzyme in assay buffer (50 mM HEPES, 1 mM EGTA, 10 mM MgCl₂, 0.01% Tween 20, 2 mM DTT). The plate is centrifuged at 800 RPM for 1 min then incubated for 20 min at 23° C. 2.5 μL per well of 4×ATP and 2.5 μL 4×ULight™-eIF4E-binding protein 1 (Thr37/46) Peptide substrate are added to all wells. After addition, the plates are centrifuged at 800 RPM for 1 min. The plates are incubated at 23° C. for 60 min. 5 μL per well 4×EDTA solution are added to each well to stop the reaction. 5 μL 4×LANCE Ultra Europium-anti-phospho-eIF4E-binding protein 1 (Thr37/46) antibody are added to each well and the plate is centrifuged for 1 min at 800 RPM. The plate is incubated for 60 min at 23° C. Fluorescence is read at 340 nm excitation and 665 nm emission on an EnVision 2105 fluorescence reader. The final concentrations of reagents in the activity assay are 1.5 nM CDK4/CCND3, 1 mM ATP, 50 nM ULight™-eIF4E-binding protein 1 (Thr37/46) Peptide substrate, 1% DMSO, 10 mM EDTA, and 2 nM LANCE Ultra Europium-anti-phospho-eIF4E-binding protein 1 (Thr37/46).

Dose-response titrations are conducted by using an 11-point, half-logarithmic serial dilution scheme typically starting at 10 μM. Signals are reported as fluorescence units at 665 nm or the ratio of fluorescence units at 665 nm/fluorescence units at 615 nm. Signals are referred to as “Lance signals”. Compound potencies are determined by first calculating % inhibition at each compound concentration according to the following equation: % inhibition=(Lance signal DMSO control−Lance signal value of sample)*100/Lance DMSO control−Lance signal value of background), where Lance signal DMSO control represents an average of 8-16 replicates, Lance signal value of sample represents an average of 2 replicates, and Lance signal of background represents an average of 8-16 replicates.

### CDK6 Assay

A CDK homogeneous time-resolved fluorescence (HTRF) assay are used to assess the potency of the compounds described herein.

To an untreated 384-well microtiter plate (ProxiPlate) is added 100 nL 100×test compound in DMSO and 5 μL 2×CDK enzyme in assay buffer (50 mM HEPES, 1 mM EGTA, 10 mM MgCl₂, 0.01% Tween 20, 2 mM DTT). The plate is centrifuged at 800 RPM for 1 min then incubated for 20 min at 23° C. 2.5 μL per well of 4×ATP and 2.5 μL 4×ULight™-eIF4E-binding protein 1 (Thr37/46) Peptide substrate are added to all wells. After addition, the plates are centrifuged at 800 RPM for 1 min. The plates are incubated at 23° C. for 60 min. 5 μL per well 4×EDTA solution are added to each well to stop the reaction. 5 μL 4×LANCE Ultra Europium-anti-phospho-eIF4E-binding protein 1 (Thr37/46) antibody are added to each well and the plate is centrifuged for 1 min at 800 RPM. The plate is incubated for 60 min at 23° C. Fluorescence is read at 340 nm excitation and 665 nm emission on an EnVision 2105 fluorescence reader. The final concentrations of reagents in the activity assay are 1 nM CDK6/CCND1, 1 mM ATP, 50 nM ULight™-eIF4E-binding protein 1 (Thr37/46) Peptide substrate, 1% DMSO, 10 mM EDTA, and 2 nM LANCE Ultra Europium-anti-phospho-eIF4E-binding protein 1 (Thr37/46).

Dose-response titrations are conducted by using an 11-point, half-logarithmic serial dilution scheme typically starting at 10 μM. Signals are reported as fluorescence units at 665 nm or the ratio of fluorescence units at 665 nm/fluorescence units at 615 nm. Signals are referred to below as “Lance signals”. Compound potencies are determined by first calculating % inhibition at each compound concentration according to the following equation: % inhibition=(Lance signal DMSO control−Lance signal value of sample)*100/Lance DMSO control−Lance signal value of background), where Lance signal DMSO control represents an average of 8-16 replicates, Lance signal value of sample represents an average of 2 replicates, and Lance signal of background represents an average of 8-16 replicates.

Using this assay, the IC₅₀ values of example compounds of Formula α are determined.

### 6. EQUIVALENTS AND INCORPORATION BY REFERENCE

Although the foregoing disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

The scope of the present disclosure, therefore, is not intended to be limited to the exemplary embodiments shown and described herein. Rather, the scope and spirit of present disclosure is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

All publications, patents, patent applications, and other documents cited in this application, including U.S. Provisional Appl. No. 63/502,379 filed May 15, 2023, are hereby incorporated by reference in their entireties for all purposes to the same extent as if each individual publication, patent, patent application, or other document were individually indicated to be incorporated by reference for all purposes.

What is claimed is:

1. A compound of formula (I): 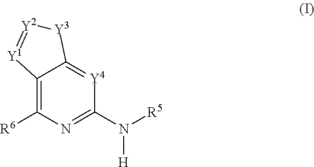
or a pharmaceutically acceptable salt, solvate or prodrug thereof,
wherein:
Y¹ is N or CR¹, and
Y² is N or CR², or
—Y¹═Y²— is an optionally substituted, fused 5-membered or 6-membered ring B: 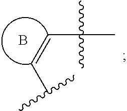
Y³ is NR³ or CR³R^{3′};
Y⁴ is N or CR⁴;
R¹ is H, (C₁₋₅)alkyl, substituted (C₁₋₅)alkyl, (C₁₋₅)alkoxyl, substituted (C₁₋₅)alkoxyl, haloalkyl, halogen, OH, or CN;
R² is H, (C₁₋₅)alkyl, substituted (C₁₋₅)alkyl, (C₁₋₅)alkoxyl, substituted (C₁₋₅)alkoxyl, haloalkyl, halogen, OH, or CN;
R³ is selected from H, alkyl, carbocycle, heterocycle, aryl, heteroaryl, alkylene-carbocycle, alkylene-heterocycle, alkylene-aryl, alkylene-heteroaryl, and substituted versions thereof; and
R^{3′} is H, or (C₁₋₅)alkyl;
R⁴ is H, (C₁₋₅)alkyl, substituted (C₁₋₅)alkyl, haloalkyl, halogen, or CN;
R⁵ is 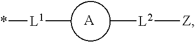
wherein:
A is a cyclic group selected from carbocyclyl, heterocyclyl, aryl, heteroaryl, and substituted versions thereof;
Z is an optional hydrophilic group;
L¹ and L² are optional linkers; and
* represents the point of attachment of R⁵ to the nitrogen atom; and
R⁶ is H, (C₁₋₅)alkyl, substituted (C₁₋₅)alkyl, (C₁₋₅)alkoxyl, substituted (C₁₋₅)alkoxyl, haloalkyl, halogen, OH, or CN.

2. The compound of claim 1, wherein one or more of Y¹ to Y³ is N, or NR³.

3. The compound of claim 1 or 2, wherein two or three of Y¹ to Y³ are N, or NR³.

4. The compound of claim 2 or 3, wherein Y³ is NR³.

5. The compound of any one of claims 1 to 4, wherein Y⁴ is N.

6. The compound of any one of claims 1 to 4, wherein Y⁴ is CR⁴.

7. The compound of any one of claims 1 to 6, wherein the compound is of one of Formula (IA) to Formula (IH):

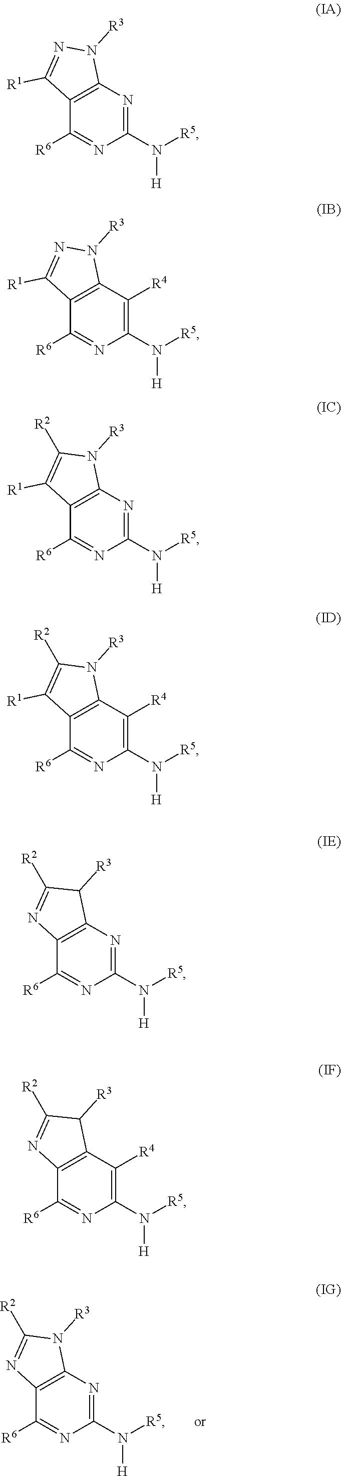

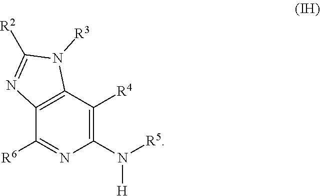

8. The compound of claim 1, wherein —Y¹═Y²— is the optionally substituted fused 5-membered or 6-membered ring B, and the compound is of Formula (IJ′), or Formula (IK′): 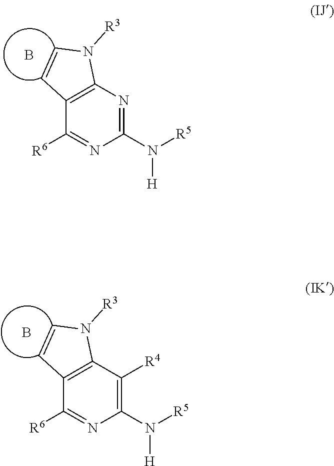
wherein:
the fused 5-membered or 6-membered ring B is selected from cycloalkenyl, aryl, heterocycle, heteroaryl, and substituted versions thereof.

9. The compound of claim 7, wherein the compound is of Formula (IA) or Formula (IB), wherein R⁴ is H.

10. The compound of claim 7, wherein the compound is of Formula (IC) or Formula (ID), wherein R⁴ is H.

11. The compound of claim 7, wherein the compound is of Formula (IG) or Formula (IH), wherein R⁴ is H.

12. The compound of any one of claims 1 to 11, wherein A is selected from cycloalkyl, cycloalkenyl, heterocyclyl, aryl, heteroaryl, and substituted versions thereof.

13. The compound of claim 12, wherein A is optionally substituted heterocyclyl.

14. The compound of claim 13, wherein the compound is of formula (IIA): 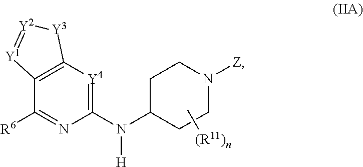
wherein,
each R¹¹ is independently selected from (C₁₋₅)alkyl, substituted (C₁₋₅)alkyl, (C₁₋₅)alkoxy, substituted (C₁₋₅)alkoxy, halogen, OH, and CN; and
n is 0 to 9.

15. The compound of claim 14, wherein the compound is of the Formula (IIIA):

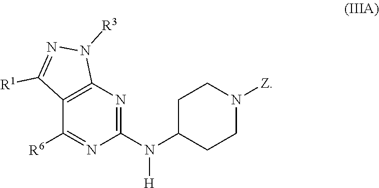

16. The compound of claim 12, wherein A is optionally substituted aryl or optionally substituted heteroaryl.

17. The compound of claim 16, wherein A is optionally substituted phenyl or optionally substituted pyridyl.

18. The compound of claim 16, wherein the compound is of Formula (IIB): 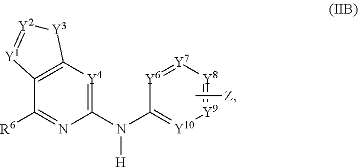
wherein:
Y⁶ to Y¹⁰ are each independently C—Z, CR¹² or N; and
each R¹² is independently selected from H, (C₁₋₅)alkyl, substituted (C₁₋₅)alkyl, (C₁₋₅)alkoxy, substituted (C₁₋₅)alkoxy, halogen, OH, and CN.

19. The compound of claim 18, wherein one and only one of Y⁶ to Y¹⁰ is C—Z.

20. The compound of claim 18, wherein Y⁷ is C—Z.

21. The compound of claim 18, wherein Y⁸ is C—Z.

22. The compound of any one of claims 18 to 21, wherein one or two of Y⁶ to Y¹⁰ is N.

23. The compound of any one of claims 18 to 22, wherein four of Y⁶ to Y¹⁰ are CR¹².

24. The compound of claim 22 or 23, wherein each R¹² is H.

25. The compound of any one of claims 18 to 24, wherein the compound is of one of Formula (IIIB) to Formula (IIIJ): 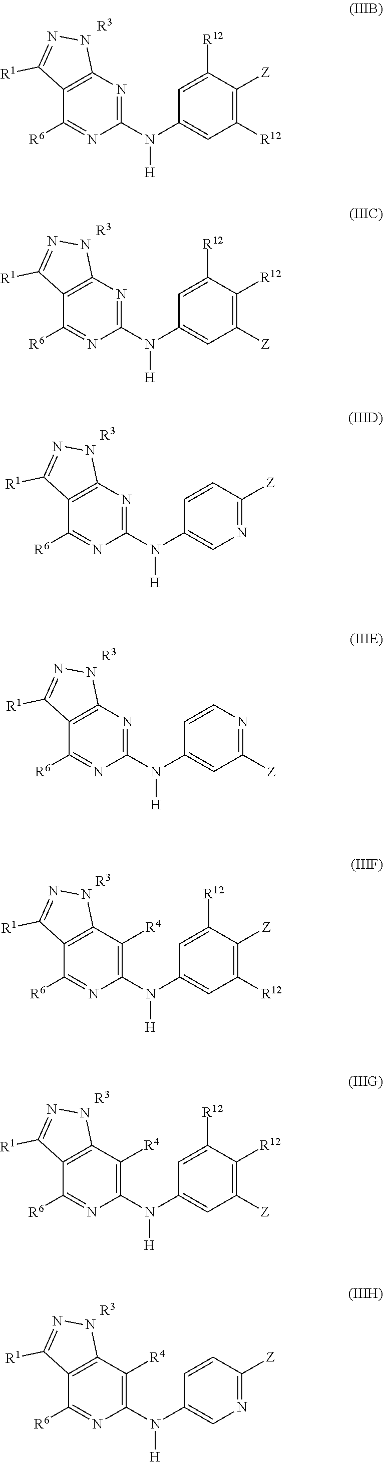 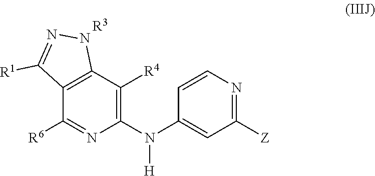
wherein:
R¹² is H, (C₁₋₅)alkyl, substituted (C₁₋₅)alkyl, (C₁₋₅)alkoxy, substituted (C₁₋₅)alkoxy, halogen, OH, or CN.

26. The compound of any one of claims 1 to 25, wherein Z comprises a group selected from sulfone, sulfonate, carboxamide, carboxy, hydroxy, hydroxyalkyl, aldehyde, ester, alkoxy, nitrile, halogen, thiolactone, and any combination thereof.

27. The compound of any one of claims 1 to 26, wherein L¹ and L² are independently absent or (C₁₋₆)alkylene (e.g., CH₂, (CH₂)₂, or (CH₂)₃).

28. The compound of any one of claims 1 to 27, wherein -L²-Z or Z is of one of Z1-Z7: 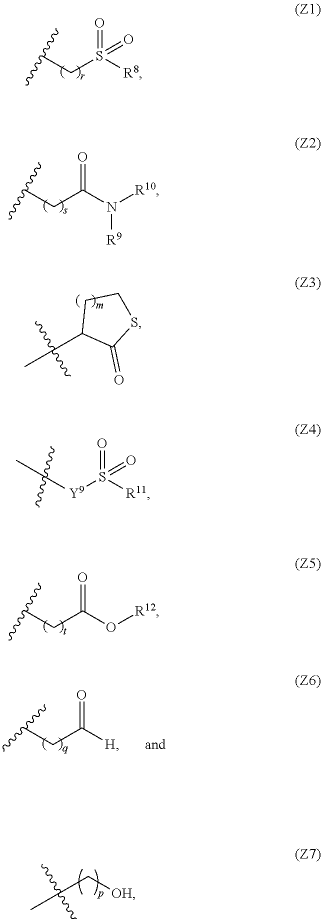
wherein:
R⁸ is (C₁₋₆)alkyl (e.g., (C₃₋₆)cycloalkyl), (C₂₋₆)alkenyl, (C₂₋₆)alkynyl, or a substituted version thereof;
R⁹ is H or optionally substituted (C₁₋₆)alkyl;
R¹⁰ is H, optionally substituted (C₁₋₆)alkyl, a sulfone (e.g., of formula Z1), or a thiolactone (e.g., of formula Z3); and
Y⁹ is absent, —O—, —NR⁹—, —(C₁₋₆)alkylene-O—, —(C₁₋₆)alkylene-NR⁹—;
R¹¹ is halogen, —NR⁹R¹⁰, (C₁₋₆)alkyl, (C₂₋₆)alkenyl, (C₂₋₆)alkynyl, or a substituted version thereof;
R¹² is H, optionally substituted (C₁₋₆)alkyl, optionally substituted aryl, or optionally substituted heterocycle;
m is 0 to 2; and
p, q, r, s and t are independently 0, 1, 2, 3, 4, 5 or 6.

29. The compound of claim 28, wherein the -L²-Z or Z is of formula Z1.

30. The compound of claim 29, wherein R⁸ is (C₁-C₆)alkyl.

31. The compound of claim 30, wherein R⁸ is methyl.

32. The compound of claim 28, wherein R⁸ is C₂-C₆ alkenyl.

33. The compound of claim 33, wherein R⁸ is —CH═CH₂ or —C(CH₃)═CH₂.

34. The compound of claim 28, wherein R⁸ is C₂-C₆ alkynyl.

35. The compound of claim 34, wherein R⁸ is —CCH or —CC—CH₃.

36. The compound of claim 28, wherein -L²-Z or Z is of the formula Z2.

37. The compound of claim 36, wherein -L²-Z or Z is of Formula (Z2A): 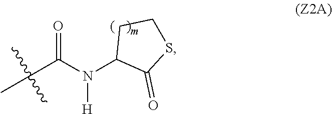
wherein m is 0 to 2.

38. The compound of claim 37, wherein m is 1.

39. The compound of claim 37 or 38, wherein R⁹ is H.

40. The compound of claim 28, wherein -L²-Z or Z is of the formula Z3.

41. The compound of claim 40, wherein m is 1.

42. The compound of claim 28, wherein -L²-Z or Z is of the formula Z6.

43. The compound of claim 42, wherein Z is CHO, CH₂CHO, (CH₂)₂CHO, or (CH₂)₃CHO.

44. The compound of claim 28, wherein -L²-Z or Z is of the formula Z7.

45. The compound of claim 44, wherein -L²-Z or Z is OH, CH₂OH, (CH₂)₂OH, or (CH₂)₃OH.

46. The compound of any one of claims 1 to 28, wherein Z is a moiety that is substantially biologically inert.

47. The compound of any one of claims 1 to 28, wherein Z comprises an electrophilic reactive moiety capable of covalently conjugating a biological target.

48. The compound of claim 28, wherein the compound is of one of formulae (IVA)-(IVC): 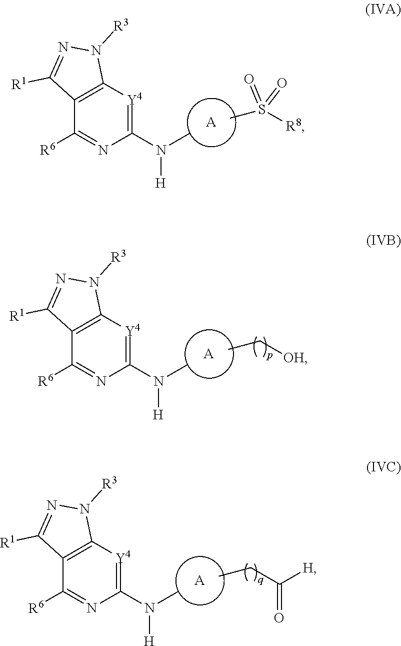
wherein:
R⁸ is optionally substituted (C₁-C₆)alkyl, optionally substituted (C₂-C₆)alkenyl, or optionally substituted (C₂-C₆)alkenyl; and
p is 0, 1, 2 or 3; and
q is 0, 1, 2 or 3.

49. The compound of claim 48, wherein A is phenyl, 3-pyridyl, 4-pyridyl, 4-piperidinyl, 4-indolyl, 5-indolyl, pyrrolidin-2-one, or a substituted version thereof.

50. The compound of claim 28, wherein the compound is of one of formulae (VA)-(VC): 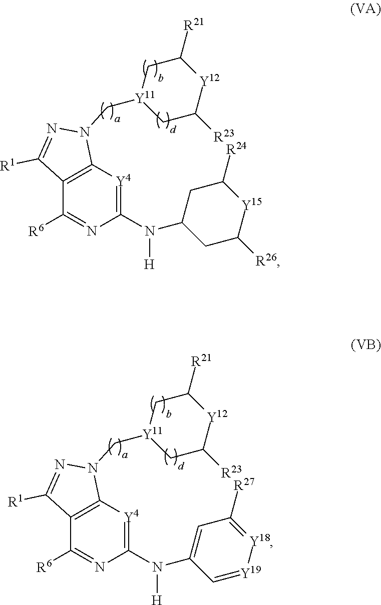
wherein:
Y¹¹ is N or CH;
Y¹² is NR²², C(R^{22′})₂, O or C═O;
Y¹⁵ is NR²⁵ or C(R^{25′})₂;
Y¹⁸ is N or CR²⁸;
Y¹⁹ is N or CR²⁹;
a is 0, 1 or 2;
b is 0 or 1;
d is 0 or 1;
R²¹, R²², each R^{22′}, and R²³ are independently selected from H, (C₁₋₅)alkyl, (C₁₋₅)haloalkyl, OH, (C₁₋₅)alkylene-OH, O(C₁₋₅)alkyl, CHO, CN, CO₂R³², CONR³⁰R³¹, NR³⁰R³¹, halogen, and (C₁₋₅)alkylene-X;
R²⁴, R²⁵, each R^{25′}, and R²⁶ are independently selected from Z, H, OH, (C₁₋₅)alkylene-OH, O(C₁₋₅)alkyl, CHO, CN, CO₂R³², CONR³⁰R³¹, NR³⁰R³¹, halogen, and (C₁₋₅)alkylene-X; and
R²⁷, R²⁸, and R²⁹ are independently selected from Z, H, (C₁₋₅)alkyl, (C₁₋₅)haloalkyl, OH, (C₁₋₅)alkylene-OH, CN, CO₂R³², (C₁₋₅)alkylene-CHO, CHO, CONR³⁰R³¹, NR³⁰R³¹, halogen, and (C₁₋₅)alkylene-X;
R³⁰ and R³¹ are independently H, or (C₁₋₅)alkyl, or R³⁰ and R³¹ are cyclically linked and together with the nitrogen atom to which they are attached provide an optionally substituted heterocyclyl ring;
R³² is H, optionally substituted (C₁₋₅)alkyl, optionally substituted heterocyclyl, or optionally substituted aryl; and
X is halogen.

51. The compound of claim 50, wherein the compound is of one of Formula (VIA)-(VIB):

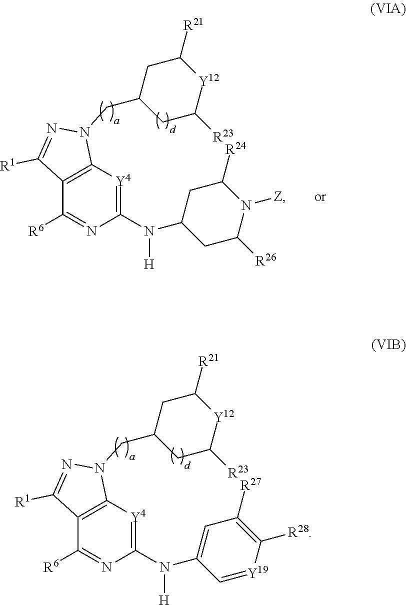

52. The compound of claim 50 or 51, wherein a is 0.

53. The compound of claim 50 or 51, wherein a is 1.

54. The compound of any one of claims 50 to 53, wherein d is 0.

55. The compound of any one of claims 50 to 53, wherein d is 1.

56. The compound of any one of claims 50 to 55, wherein:
R²¹ is H; and
R^{22′}, and R²³ are independently selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₅)alkylene-X.

57. The compound of any one of claims 50 to 56, wherein Y¹⁹ is N.

58. The compound of any one of claims 50 to 56, wherein Y¹⁹ is CH.

59. The compound of any one of claims 50 to 58, wherein the compound is of Formula (VIA), wherein:
R²⁴ is H; and
R²⁶ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X.

60. The compound of any one of claims 50 to 58, wherein the compound is of Formula (VIB), wherein:
R²⁷ is H or Z; and
R²⁸ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X.

61. The compound of any one of claims 50 to 58, wherein the compound is of Formula (VIB), wherein:
R²⁷ is selected from H, (C₁₋₃)alkyl, (C₁₋₃)haloalkyl, OH, (C₁₋₃)alkylene-OH, O(C₁₋₃)alkyl, CHO, CN, CO₂R³², CONR³⁰R³¹, NR³⁰R³¹, halogen, and (C₁₋₃)alkylene-X; and
R²⁸ is H or Z.

62. The compound of any one of claims 50 to 61, wherein Y⁴ is N.

63. The compound of any one of claims 50 to 61, wherein Y⁴ is CR⁴ or CH.

64. The compound of any one of claims 50 to 63, wherein R⁶ is H, CH₃, CF₃, or halogen.

65. The compound of claim 64, wherein R⁶ is H.

66. The compound of any one of claims 1 to 65, wherein R¹ is H, CH₃, CF₃, or halogen.

67. The compound of claim 66, wherein R¹ is H.

68. The compound of any one of claims 50 to 67, wherein the Z is SO₂R⁸, wherein R⁸ is optionally substituted (C₁-C₆)alkyl, optionally substituted (C₂-C₆)alkenyl, or optionally substituted (C₂-C₆)alkynyl.

69. The compound of claim 68, wherein R⁸ is (C₁-C₆)alkyl.

70. The compound of claim 69, wherein R⁸ is methyl.

71. The compound of claim 68, wherein R⁸ is C₂-C₆ alkenyl.

72. The compound of claim 71, wherein R⁸ is —CH═CH₂ or —C(CH₃)═CH₂.

73. The compound of claim 68, wherein R⁸ is C₂-C₆ alkynyl.

74. The compound of claim 73, wherein R⁸ is —CCH or —CC—CH₃.

75. The compound of any one of claims 50 to 67, wherein Z is CHO, CH₂CHO, (CH₂)₂CHO, or (CH₂)₃CHO.

76. The compound of any one of claims 50 to 67, wherein Z is OH, CH₂OH, (CH₂)₂OH, or (CH₂)₃OH.

77. The compound of any one of claims 1-76, wherein the compound is selected from Tables 1-9, or a pharmaceutically acceptable salt thereof.

78. A pharmaceutical composition comprising a compound of any one of claims 1 to 77 and a pharmaceutically acceptable excipient.

79. A method of inhibiting cyclin-dependent kinase 2 (CDK2), the method comprising contacting a biological sample containing CDK2 with an effective amount of a compound according to any one of claims 1 to 77, or a pharmaceutical composition according to claim 53 to inhibit CDK2.

80. A method of inhibiting CDK2 in a subject, comprising administering to the subject a compound of any one of claims 1 to 77, or a pharmaceutical composition according to claim 78 to inhibit CDK2 in the subject.

81. A method of treating a disease or disorder associated with CDK2 in a subject, comprising administering to the subject suffering from a disease or disorder associated with CDK2 a therapeutically effective amount of a compound of any one of claims 1 to 77, or a pharmaceutical composition according to claim 78.
